(19) United States
(12) Patent Application Publication
(10) Pub. No.: US 2024/0252586 A1
(43) Pub. Date: Aug. 1, 2024

(54) ENGINEERED ANTIMICROBIAL PEPTIDES AND USAGE THEREOF

(71) Applicant: Peptilogics, Inc., Pittsburgh, PA (US)

(72) Inventors: Jonathan D. STECKBECK, Pittsburgh, PA (US); Bradd N. PICONE, Pittsburgh, PA (US); Despina X. DOBBINS, Pittsburgh, PA (US)

(21) Appl. No.: 18/606,023

(22) Filed: Mar. 15, 2024

Related U.S. Application Data
(63) Continuation of application No. PCT/US2022/076560, filed on Sep. 16, 2022.
(60) Provisional application No. 63/245,774, filed on Sep. 17, 2021.

(51) Int. Cl. A61K 38/16; A61P 31/04

(52) U.S. Cl. CPC A61K 38/162; A61P 31/04

(57) ABSTRACT

Provided in this disclosure are pharmaceutical formulations comprising antimicrobial peptides with a basic pH. Further provided herein are methods of treating or preventing an infection comprising administering pharmaceutical formulations comprising antimicrobial peptides when administered to a subject.

### CROSS-REFERENCE

This application is a continuation of International Application No. PCT/US2022/76560, filed Sep. 16, 2022, which claims the benefit of U.S. Provisional Application No. 63/245,774, filed on Sep. 17, 2021, which is incorporated by reference herein in its entirety.

### STATEMENT AS TO FEDERALLY SPONSORED RESEARCH

This disclosure was made with governmental support under BARDA Award No. 6 IDSEP160030 awarded by Department of Health and Human services. The government has certain rights in the disclosure.

### SUMMARY

Described herein are pharmaceutical formulations comprising: (a) a peptide or pharmaceutically acceptable salt thereof comprising at least about 70% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and (b) aqueous sodium bicarbonate.

Described herein are pharmaceutical formulations consisting of: a peptide or pharmaceutically acceptable salt thereof comprising at least about 70% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and (b) aqueous sodium bicarbonate. In some embodiments, the pharmaceutical formulation comprises a pH from about 7 to about 11. In some embodiments, the pharmaceutical formulation comprises the pH from about 8 to about 11. In some embodiments, the pharmaceutical formulation comprises the pH from about 7 to about 10. In some embodiments, the pharmaceutical formulation comprises the pH at least about 7.5 or at least about 7.9. In some embodiments, the peptide or pharmaceutically acceptable salt thereof is present in the pharmaceutical formulation at a concentration from about 0.1 mg/mL to about 100 mg/mL. In some embodiments, the peptide or pharmaceutically acceptable salt thereof is present in the pharmaceutical formulation at a concentration from about 1 mg/mL to about 10 mg/mL. In some embodiments, the peptide or pharmaceutically acceptable salt thereof is present in the pharmaceutical formulation at a concentration of about 1 mg/mL, about 3 mg/mL, about 5 m/mL, or about 10 mg/mL. In some embodiments, the aqueous sodium bicarbonate is present in the pharmaceutical formulation at a concentration from about 25 mM to about 300 mM. In some embodiments, the aqueous sodium bicarbonate is present in the pharmaceutical formulation at a concentration of about 150 mM. In some embodiments, the aqueous sodium bicarbonate is present in the pharmaceutical formulation at a concentration from about 5 mg/mL to about 50 mg/mL. In some embodiments, the aqueous sodium bicarbonate is present in the pharmaceutical formulation at a concentration at about 12.6 mg/mL. In some embodiments, the aqueous sodium bicarbonate has a concentration of about 1 mEq/L to about 200 mEq/mL. In some embodiments, the aqueous sodium bicarbonate has a concentration of about 50 mEq/mL. In some embodiments, the pharmaceutical formulation comprises an osmolality of at least about 30 milliosmoles per kilogram (mOsm/kg) to at least about 800 mOsm/kg. In some embodiments, the pharmaceutical formulation comprises an osmolality of about 50 mOsm/kg to about 500 mOsm/kg. In some embodiments, the pharmaceutical formulation comprises an osmolality of about 200 mOsm/kg to about 500 mOsm/kg. In some embodiments, the peptide or pharmaceutically acceptable salt thereof comprises the polypeptide of sequence Arg Arg Trp Val Arg Arg Val Arg Arg Val Trp Arg Arg Val Val Arg Val Val Arg Arg Trp Val Arg Arg (SEQ ID NO: 1). In some embodiments, the peptide or pharmaceutically acceptable salt thereof comprises the polypeptide of sequence Arg-Trp-Trp-Arg-Trp-Trp-Arg-Arg-Trp-Trp-Arg-Arg (SEQ ID NO: 17). In some embodiments, the aqueous sodium bicarbonate provides a synergistic effect with the peptide or pharmaceutically acceptable salt thereof. In some embodiments, the synergistic effect comprises reduces a bacterial burden a greater extend as compared to administering (a) or (b) alone to a subject, reduces the incident of abscesses in a subject, or increases the survivability of a subject.

Described herein are kits comprising: a first container comprising a peptide or pharmaceutically acceptable salt thereof comprising at least about 70% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and (ii) a second container comprising aqueous sodium bicarbonate. In some embodiments, the kit further comprising (iii) a third container comprising water. In some embodiments, the kit further comprising (iv) a mixing container. In some embodiments, the mixing container is an intravenous bag, a lavage bottle, or a joint irrigation system. In some embodiments, the peptide or pharmaceutically acceptable salt thereof is present at a concentration from about 0.1 mg/mL to about 100 mg/mL. In some embodiments, the peptide or pharmaceutically acceptable salt thereof is present at a concentration at about 15 mg/mL, about 30 mg/mL, about 40 mg/mL, about 70 mg/mL, or about 80 mg/mL. In some embodiments, the peptide or pharmaceutically acceptable salt thereof is present at a pH of about 3 to about 7. In some embodiments, the peptide or pharmaceutically acceptable salt thereof is present at a pH of about 5. In some embodiments, the aqueous sodium bicarbonate is present a concentration from about 25 mM to about 300 mM. In some embodiments, the aqueous sodium bicarbonate is present in at a concentration from about 50 mM to about 200 mM. In some embodiments, the aqueous sodium bicarbonate is 8.4% sodium bicarbonate. In some embodiments, the aqueous sodium bicarbonate is at a concentration of about 1 mEq/L to about 200 mEq/L. In some embodiments, the aqueous sodium bicarbonate is at a concentration of about 50 mEq/L. In some embodiments, the peptide or pharmaceutically acceptable salt thereof comprises the polypeptide of sequence Arg Arg Trp Val Arg Arg Val Arg Arg Val Trp Arg Arg Val Val Arg Val Val Arg Arg Trp Val Arg Arg (SEQ ID NO: 1). In some embodiments, the peptide or pharmaceutically acceptable salt thereof comprises the polypeptide of sequence Arg-Trp-Trp-Arg-Trp-Trp-Arg-Arg-Trp-Trp-Arg-Arg (SEQ ID NO: 17).

Described herein are methods of making a pharmaceutical formulation from a kit, wherein the kit comprises: a first container comprising a peptide or pharmaceutically acceptable salt thereof comprising at least about 70% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); or Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25); (ii) a second container comprising aqueous sodium bicarbonate; (iii) a third container comprising water; and (iv) a mixing container; wherein method comprises adding the first container, the second container, and the third container into the mixing container, thereby resulting in the pharmaceutical formulation. In some embodiments, the pharmaceutical formulation comprises a pH from about 5 to about 11. In some embodiments, the pharmaceutical formulation comprises the pH from about 7 to about 11. In some embodiments, the pharmaceutical formulation comprises the pH from about 7 to about 10. In some embodiments, the pharmaceutical formulation comprises the pH at least about 8. In some embodiments, the peptide or pharmaceutically acceptable salt thereof is present in the pharmaceutically formulation at a concentration from about 1 mg/mL to about 10 mg/mL. In some embodiments, the peptide or pharmaceutically acceptable salt thereof is present in the pharmaceutically formulation at a concentration of about 1 mg/mL, about 3 mg/mL, about 5 m/mL, or about 10 mg/mL. In some embodiments, the aqueous sodium bicarbonate is present in the pharmaceutically formulation at a concentration from about 25 mM to about 300 mM. In some embodiments, the aqueous sodium bicarbonate is present in the pharmaceutically formulation at a concentration from about 50 mM to about 200 mM. In some embodiments, the aqueous sodium bicarbonate is present in the pharmaceutically formulation at a concentration from about 5 mg/mL to about 50 mg/mL. In some embodiments, the aqueous sodium bicarbonate is present in the pharmaceutically formulation at a concentration at about 12.6 mg/mL. In some embodiments, the aqueous sodium bicarbonate is at a concentration of about 1 mEq/L to about 200 mEq/L. In some embodiments, the aqueous sodium bicarbonate is at a concentration of about 50 mEq/L. In some embodiments, the pharmaceutical formulation comprises an osmolality of at least about 30 milliosmoles per kilogram (mOsm/kg) to at least about 800 mOsm/kg. In some embodiments, the pharmaceutical formulation comprises an osmolality of about 50 mOsm/kg to about 500 mOsm/kg. In some embodiments, the pharmaceutical formulation comprises an osmolality of about 200 mOsm/kg to about 500 mOsm/kg. In some embodiments, the peptide or pharmaceutically acceptable salt thereof comprises the polypeptide of sequence Arg Arg Trp Val Arg Arg Val Arg Arg Val Trp Arg Arg Val Val Arg Val Val Arg Arg Trp Val Arg Arg (SEQ ID NO: 1). In some embodiments, the peptide or pharmaceutically acceptable salt thereof comprises the polypeptide of sequence Arg-Trp-Trp-Arg-Trp-Trp-Arg-Arg-Trp-Trp-Arg-Arg (SEQ ID NO: 17).

Described herein are methods of preventing or treating an infection in a subject in need thereof, wherein the method comprising locally administering of a pharmaceutical formulation to a site of infection, wherein administration comprises washing, irrigating, debriding, aspirating, or a combination thereof the site of infection, thereby preventing or treating the infection, wherein the pharmaceutical formulation comprises: a peptide or pharmaceutically acceptable salt thereof comprising at least about 70% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and (b) an aqueous carrier, wherein the aqueous carrier provides a synergistic effect with the peptide or pharmaceutically acceptable salt thereof, wherein the synergistic effect comprises reduces a bacterial burden in the subject to a greater extend as compared to administering (a) or (b) alone.

Described herein are methods of preventing or treating an infection in a subject in need thereof, wherein the method comprising locally administering of a pharmaceutical formulation to a site of infection, wherein administration comprises washing, irrigating, debriding, aspirating, or a combination thereof the site of infection, thereby preventing or treating the infection, wherein the pharmaceutical formulation consists of: a peptide or pharmaceutically acceptable salt thereof comprising at least about 70% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or combination thereof; and (b) an aqueous carrier, wherein the aqueous carrier provides a synergistic effect with the peptide or pharmaceutically acceptable salt thereof, wherein the synergistic effect comprises reduces a bacterial burden in the subject to a greater extend as compared to administering (a) or (b) alone. In some embodiments, the aqueous carrier comprises sodium bicarbonate, chlorocyclohexidine, chlorhexidine gluconate, normal saline, phosphate buffered saline, povidone-iodine (PVP-I), ethanol, acetic acid, sodium acetate, benzalkonium chloride, sodium lauryl sulfate, citric acid, sodium citrate; or any combination thereof.

Descried herein are methods of preventing or treating an infection in a subject in need thereof, wherein the method comprising locally administering of a pharmaceutical formulation to a site of infection, wherein locally administration comprises washing, irrigating, debriding, aspirating, or a combination thereof of the site of infection, thereby preventing or treating the infection, wherein the pharmaceutical formulation comprises: a peptide or pharmaceutically acceptable salt thereof comprising at least about 70% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and (b) aqueous sodium bicarbonate.

Described herein are methods of preventing or treating an infection in a subject in need thereof, wherein the method comprising locally administering of a pharmaceutical formulation to a site of infection, wherein locally administration comprises washing, irrigating, debriding, aspirating, or a combination thereof of the site of infection, thereby preventing or treating the infection, wherein the pharmaceutical formulation consists of: a peptide or pharmaceutically acceptable salt thereof comprising at least about 70% sequence identity to a polypeptide sequence of: Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1); Arg-Val-Val-Arg-Val-Val-Arg-Arg-Val-Val-Arg-Arg (SEQ ID NO: 15); Arg-Val-Val-Arg-Val-Val-Arg-Arg-Trp-Val-Arg-Arg (SEQ ID NO: 16); Arg-Trp-Trp-Arg-Trp-Trp-Arg-Arg-Trp-Trp-Arg-Arg (SEQ ID NO: 17); Trp-Arg-Arg-Trp-Trp-Arg-Arg-Trp-Trp-Arg-Trp-Trp-Arg-Arg-Trp-Trp-Arg-Arg (SEQ ID NO: 18); Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19); Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20); Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21); Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22); Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23); Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24); Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and (b) aqueous sodium bicarbonate.

In some embodiments, the infection further comprises a biofilm. In some embodiments, the method prevents formation of a biofilm. In some embodiments, administration of the pharmaceutical formulation results in at least partially penetrating, inhibiting formation of, or destroys the biofilm. In some embodiments, the site of infection does not comprise a prosthesis. In some embodiments, the site of infection comprises a prosthesis implanted in the subject. In some embodiments, the washing, irrigating, debridement, aspirating, or a combination thereof of the site of infection occurs on the implanted prosthesis at the site of infection. In some embodiments, the implanted prosthesis is a knee prosthesis, shoulder prosthesis, hip prosthesis, elbow prosthesis, ankle prosthesis, wrist prosthesis, or spine prosthesis. In some embodiments, the infection is periprosthetic joint infection (PJI). In some embodiments, the periprosthetic joint infection is first stage periprosthetic joint infection or second stage periprosthetic joint infection. In some embodiments, the infection is a shoulder infection, knee infection, acute infection, chronic infection, or any combination thereof. In some embodiments, the method occurs prior, during, or subsequent to a total knee arthroplasty. In some embodiments, the method occurs prior, during, or subsequent to a debridement, antibiotics, and implant retention (DAIR) procedure. In some embodiments, the infection is a bacterial infection, wherein the bacterial species is selected from the group consisting of: Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, and any combination thereof. In some embodiments, the pharmaceutical formulation comprises a pH from about 7 to about 11. In some embodiments, the pharmaceutical formulation comprises the pH from about 8 to about 11. In some embodiments, the pharmaceutical formulation comprises the pH from about 7 to about 10. In some embodiments, the pharmaceutical formulation comprises the pH at least about 7.5 or at least about 7.9. In some embodiments, the peptide or pharmaceutically acceptable salt thereof is present in the pharmaceutically formulation at a concentration from about 0.1 mg/mL to about 100 mg/mL. In some embodiments, the peptide or pharmaceutically acceptable salt thereof is present in the pharmaceutically formulation at a concentration from about 1 mg/mL to about 10 mg/mL. In some embodiments, the peptide or pharmaceutically acceptable salt thereof is present in the pharmaceutically formulation at a concentration of about 1 mg/mL, about 3 mg/mL, about 5 m/mL, or about 10 mg/mL. In some embodiments, the aqueous sodium bicarbonate is present in the pharmaceutically formulation at a concentration from about 25 mM to about 300 mM. In some embodiments, the aqueous sodium bicarbonate is present in the pharmaceutically formulation at a concentration from about 50 mM to about 200 mM. In some embodiments, the aqueous sodium bicarbonate is present in the pharmaceutically formulation at a concentration from about 5 mg/mL to about 50 mg/mL. In some embodiments, the aqueous sodium bicarbonate is present in the pharmaceutically formulation at a concentration at about 12.6 mg/mL. In some embodiments, the aqueous sodium bicarbonate is at a concentration of about 1 mEq/L to about 200 mEq/L. In some embodiments, the aqueous sodium bicarbonate is at a concentration of about 50 mEq/L. In some embodiments, the pharmaceutical formulation comprises an osmolality of at least about 30 milliosmoles per kilogram (mOsm/kg) to at least about 800 mOsm/kg. In some embodiments, the pharmaceutical formulation comprises an osmolality of about 50 mOsm/kg to about 500 mOsm/kg. In some embodiments, the pharmaceutical formulation comprises an osmolality of about 200 mOsm/kg to about 500 mOsm/kg. In some embodiments, the peptide or pharmaceutically acceptable salt thereof comprises the polypeptide of sequence Arg Arg Trp Val Arg Arg Val Arg Arg Val Trp Arg Arg Val Val Arg Val Val Arg Arg Trp Val Arg Arg (SEQ ID NO: 1). In some embodiments, the peptide or pharmaceutically acceptable salt thereof comprises the polypeptide of sequence Arg-Trp-Trp-Arg-Trp-Trp-Arg-Arg-Trp-Trp-Arg-Arg (SEQ ID NO: 17). In some embodiments, pharmaceutical formulation is administered once. In some embodiments, pharmaceutical formulation is administered more than one time. In some embodiments, pharmaceutical formulation is administered two or more times.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims.

A better understanding of the features and advantages of the present disclosure will be obtained by reference to the following detailed description that sets forth illustrative embodiments, in which the principles of the disclosure are utilized, and the accompanying drawings of which:

FIG. 1A depicts Log-kill (log₁₀ CFU/mL) of S. aureus NRS 382 by SEQ ID NO: 1 over time. MIC 4 μg/mL.

FIG. 1B depicts Log-kill (log₁₀ CFU/mL) of S. aureus NRS 382 by SEQ ID NO: 1 over time in PBS. MIC 4 μg/mL.

FIG. 1C depicts Log-kill (log₁₀ CFU/mL) of S. aureus NRS 382 by SEQ ID NO: 1 over time in H₂O. MIC 4 μg/mL

FIG. 1D depicts Log-kill (log₁₀ CFU/mL) of S. aureus NRS 382 by SEQ ID NO: 1 over time in CAMHB. MIC 4 μg/mL

FIG. 2A depicts Log-kill (log₁₀ CFU/mL) of S. aureus NRS 382 by SEQ ID NO: 1 over time. MIC 8 μg/mL.

FIG. 2B depicts Log-kill (log₁₀ CFU/mL) of S. aureus NRS 382 by SEQ ID NO: 1 over time in PBS. MIC 8 μg/mL.

FIG. 2C depicts Log-kill (log₁₀ CFU/mL) of S. aureus NRS 382 by SEQ ID NO: 1 over time in H₂O. MIC 8 μg/mL

FIG. 2D depicts Log-kill (log₁₀ CFU/mL) of S. aureus NRS 382 by SEQ ID NO: 1 over time in CAMHB. MIC 8 μg/mL

FIG. 3A depicts Log-kill (log₁₀ CFU/mL) of S. epidermidis MMX 8655 by SEQ ID NO: 1 over time. MIC 1 μg/mL.

FIG. 3B depicts Log-kill (log₁₀ CFU/mL) of S. epidermidis MMX 8655 by SEQ ID NO: 1 over time in PBS. MIC 1 μg/mL.

FIG. 3C depicts Log-kill (log₁₀ CFU/mL) of S. epidermidis MMX 8655 by SEQ ID NO: 1 over time in H₂O. MIC 1 μg/mL

FIG. 3D depicts Log-kill (log₁₀ CFU/mL) of S. epidermidis MMX 8655 by SEQ ID NO: 1 over time in CAMHB. MIC 1 μg/mL

FIG. 4A depicts Log-kill (log₁₀ CFU/mL) of E. Coli CDC 0451 by SEQ ID NO: 1 over time. MIC 1 μg/mL.

FIG. 4B depicts Log-kill (log₁₀ CFU/mL) of E. Coli CDC 0451 by SEQ ID NO: 1 over time in PBS. MIC 1 μg/mL.

FIG. 4C depicts Log-kill (log₁₀ CFU/mL) of E. Coli CDC 0451 by SEQ ID NO: 1 over time in H₂O. MIC 1 μg/mL

FIG. 4D depicts Log-kill (log₁₀ CFU/mL) of E. Coli CDC 0451 by SEQ ID NO: 1 over time in CAMHB. MIC 1 μg/mL

FIG. 5A depicts Log-kill (log₁₀ CFU/mL) of P. Aeruginosa CDC 0451 by SEQ ID NO: 1 over time. MIC 16 μg/mL.

FIG. 5B depicts Log-kill (log₁₀ CFU/mL) of P. Aeruginosa CDC 0451 by SEQ ID NO: 1 over time in PBS. MIC 16 μg/mL.

FIG. 5C depicts Log-kill (log₁₀ CFU/mL) of P. Aeruginosa CDC 0451 by SEQ ID NO: 1 over time in H₂O. MIC 16 μg/mL

FIG. 5D depicts Log-kill (log₁₀ CFU/mL) of P. Aeruginosa CDC 0451 by SEQ ID NO: 1 over time in CAMHB. MIC 16 μg/mL

FIG. 6A depicts time-kill activity of SEQ ID NO: 1 against Staphylococcus aureus USA 100 in wire biofilm study using PBS as the vehicle.

FIG. 6B depicts time-kill activity of SEQ ID NO: 1 against Escherichia coli CDC 0451 in the wire biofilm study using PBS as the vehicle.

FIG. 7A depicts time-kill activity of SEQ ID NO: 1 against Staphylococcus aureus USA 100 in the wire biofilm study using 50 mM bicarbonate as the vehicle.

FIG. 7B depicts time-kill activity of SEQ ID NO: 1 Escherichia coli CDC 0451 in the wire biofilm study using 50 mM bicarbonate as the vehicle.

FIG. 8A depicts time-kill activity of SEQ ID NO: 1 Staphylococcus aureus USA 100 in the wire biofilm study using 100 mM bicarbonate as the vehicle.

FIG. 8B depicts time-kill activity of SEQ ID NO: 1 against Escherichia coli CDC 0451 in the wire biofilm study using 100 mM bicarbonate as the vehicle.

FIG. 9A depicts Time-kill activity of SEQ ID NO: 1 against Staphylococcus aureus USA 100 in wire biofilm study using 50 mM sodium bicarbonate as the vehicle.

FIG. 9B depicts Time-kill activity of SEQ ID NO: 1 against Escherichia coli CDC 0451 in the wire biofilm study using 50 mM sodium bicarbonate as the vehicle.

FIG. 9C depicts Time-kill activity of SEQ ID NO: 1 against Pseudomonas aeruginosa CDC 0515 in the wire biofilm study using 50 mM sodium bicarbonate as the vehicle.

FIG. 10A depicts Time-kill activity of SEQ ID NO: 1 against Staphylococcus aureus USA 100 in the wire biofilm study using 100 mM sodium bicarbonate as the vehicle.

FIG. 10B depicts Time-kill activity of SEQ ID NO: 1 against Escherichia coli CDC 0451 in the wire biofilm study using 100 mM sodium bicarbonate as the vehicle.

FIG. 10C depicts Time-kill activity of SEQ ID NO: 1 against Pseudomonas aeruginosa CDC 0515 in the wire biofilm study using 100 mM sodium bicarbonate as the vehicle.

FIG. 11A depicts Time-kill activity of SEQ ID NO: 1 against Staphylococcus aureus USA 100 in the wire biofilm study using 150 mM sodium bicarbonate as the vehicle.

FIG. 11B depicts Time-kill activity of SEQ ID NO: 1 against Escherichia coli CDC 0451 in the wire biofilm study using 150 mM sodium bicarbonate as the vehicle.

FIG. 11C depicts Time-kill activity of SEQ ID NO: 1 against Pseudomonas aeruginosa CDC 0515 in the wire biofilm study using 150 mM sodium bicarbonate as the vehicle.

FIG. 12A depicts Time-kill activity of SEQ ID NO: 1 against Staphylococcus aureus USA 100 in the wire biofilm study using 200 mM sodium bicarbonate as the vehicle.

FIG. 12B depicts Time-kill activity of SEQ ID NO: 1 against Escherichia coli CDC 0451 in the wire biofilm study using 200 mM sodium bicarbonate as the vehicle.

FIG. 12C depicts Time-kill activity of SEQ ID NO: 1 against Pseudomonas aeruginosa CDC 0515 in the wire biofilm study using 200 mM sodium bicarbonate as the vehicle.

FIG. 13A depicts Time-kill activity by treatment of SEQ ID NO: 1 against Staphylococcus aureus NRS382 in the wire biofilm study in various concentrations of sodium bicarbonate

FIG. 13B depicts Time-kill activity by treatment of SEQ ID NO: 1 against Escherichia coli CDC 0451 in the wire biofilm study in various concentrations of sodium bicarbonate.

FIG. 13C depicts Time-kill activity by treatment of SEQ ID NO: 1 against Pseudomonas aeruginosa CDC 0515 in the wire biofilm study in various concentrations of sodium bicarbonate

FIG. 14 depicts exemplary kit for formulating a pharmaceutical composition described herein containing a peptide described herein, aqueous sodium bicarbonate, sterile water for injection, and a mixing container (intravenous bag).

FIG. 15 shows the mean body weight of the 3 groups tested in a PJI Rabbit Model.

FIG. 16 shows the mean body weight changes of the 3 groups tested in a PJI Rabbit Model.

FIG. 17 shows the mean temperature of the 3 groups tested in a PJI Rabbit Model.

FIG. 18 shows the mean temperature changes of the 3 groups tested in a PJI Rabbit Model.

FIG. 19 shows the implant colony forming unit bacterial burden of the 3 groups tested in a PJI Rabbit Model.

FIG. 20 shows the bone colony forming unit bacterial burden of the 3 groups tested in a PJI Rabbit Model.

FIG. 21 shows total bacterial burden of the 3 groups tested in a PJI Rabbit Model.

FIG. 22 shows survival percentage of the 3 groups tested in a PJI Rabbit Model.

FIG. 23 shows the erythrocyte sedimentation rate of the 3 groups tested in a PJI Rabbit Model.

FIG. 24 shows the C-reactive protein levels of the 3 groups tested in a PJI Rabbit Model.

### DETAILED DESCRIPTION

### Pharmaceutical Formulation

Described herein are pharmaceutical formulations comprising a peptide or pharmaceutically acceptable salt thereof as described herein and an aqueous carrier. Described herein are pharmaceutical formulations consisting of a peptide or pharmaceutically acceptable salt thereof as described herein and an aqueous carrier. In some embodiments, a pharmaceutical formulation consists only of a peptide or pharmaceutically acceptable salt thereof as described herein and an aqueous carrier. In some embodiments, a pharmaceutical composition comprising a peptide or pharmaceutically acceptable salt thereof as described herein and an aqueous carrier may also further comprise additional excipients and the like. Described herein are methods of treating a disease or condition by administering to a subject a pharmaceutical formulation comprising a peptide or pharmaceutically acceptable salt thereof as disclosed therein and an aqueous carrier. Described herein are methods of treating a disease or condition by administering to a subject a pharmaceutical formulation consisting of a peptide as disclosed therein and an aqueous carrier. In some embodiments, the disease or condition is an infection from a pathogen and the administration of a pharmaceutical formulation described herein reduces the infection. For example, a pharmaceutical formulation comprising a peptide or pharmaceutically acceptable salt as described herein and an aqueous carrier can be administered as an antimicrobial agent in order to at least partially inhibit the growth of a pathogen, such as bacteria, through disruption of the structural integrity of the bacterial cell membrane. A peptide described herein can be screened for broad spectrum activity against a variety of pathogens for broad utility when administered to a subject.

### Peptides

The development of antimicrobial agents is paramount due to the emergence of pathogens resistant to traditional antimicrobial compounds. Disclosed herein are peptides that comprise antimicrobial, antiviral, antifungal or antitumor activity when administered to a subject. A peptide described herein can be used to disrupt the integrity of a membrane by (a) binding to a negatively charged surface on a membrane; and/or (b) integrating into a membrane. The ability of a peptide disclosed herein to bind to a negatively charged surface on a membrane and/or integrate into a membrane can allow a peptide to act as a toxic agent to cells with a negatively charged surface by disrupting membrane integrity. In other embodiments, a peptide disclosed herein can have anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, and/or protozoicidal properties.

The antimicrobial peptides may be derived from, and are analogs of, the LLP-1 peptide parent sequence corresponding to amino acids 828-856 of the HIV-1 viral isolate HXB2R Env, (see Table 1 below). The antimicrobial activity of other LLP-1 peptide analogues has been previously described (see, Tencza et al., 1999, Journal of Antimicrobial Chemotherapy 44:33-41, U.S. Pat. No. 5,714,577 of Montelaro et al. and U.S. Pat. No. 5,945,507 of Montelaro et al., the disclosures of which are incorporated herein by reference). The antimicrobial peptides may be LLP-1 analogs having modifications based on the following principles: (i) optimizing amphipathicity, (ii) substituting arginine (Arg) on the charged face and/or valine (Val) or tryptophan (Trp) on the hydrophobic face with another amino acid, and (iii) increasing peptide length; see Table 1). Amino acid sequences are provided, left-to-right, from their N-terminus to their C-terminus in 1 letter designations and 3 letter designations.

TABLE 1
Antimicrobial Peptides

| SEQ ID | |
|---|---|
| NO: | Amino Acid Sequence |
| 1 | RRWVRRVRRVWRRVVRVVRRWVRR |
| | Arg-Arg-Trp-Val-Arg-Arg-Val-Arg-Arg-Val-Trp-Arg-Arg-Val-Val-Arg-Val- |
| | Val-Arg-Arg-Trp-Val-Arg-Arg |
| 2 | IRRRRRRIRRRRRR |
| | Ile-Arg-Arg-Arg-Arg-Arg-Arg-Ile-Arg-Arg-Arg-Arg-Arg-Arg |
| 3 | IRRRIRRIRRRIRRIRRRIRR |
| | Ile-Arg-Arg-Arg-Ile-Arg-Arg-Ile-Arg-Arg-Arg-Ile-Arg-Arg-Ile-Arg-Arg-Arg- |
| | Ile-Arg-Arg |
| 4 | IRRIIRRIRRIIRRIRRIIRR |
| | Ile-Arg-Arg-Ile-Ile-Arg-Arg-Ile-Arg-Arg-Ile-Ile-Arg-Arg-Ile-Arg-Arg-Ile-Ile- |
| | Arg-Arg |
| 5 | VWRWVRRVWRWVRRVWRWVRR |
| | Val-Trp-Arg-Trp-Val-Arg-Arg-Val-Trp-Arg-Trp-Val-Arg-Arg-Val-Trp-Arg-Trp- |
| | Val-Arg-Arg |
| 6 | VWRWVRRVWRWVRR |
| | Val-Trp-Arg-Trp-Val-Arg-Arg-Val-Trp-Arg-Trp-Val-Arg-Arg |
| 7 | VVRVVRRVVRVVRR |
| | Val-Val-Arg-Val-Val-Arg-Arg-Val-Val-Arg-Val-Val-Arg-Arg |
| 8 | VVRVVRVVVRVVRVVVRVVRV |
| | Val-Val-Arg-Val-Val-Arg-Val-Val-Val-Arg-Val-Val-Arg-Val-Val-Val-Arg-Val- |
| | Val-Arg-Val |
| 9 | RSRVVRSWSRV |
| | Arg-Ser-Arg-Val-Val-Arg-Ser-Trp-Ser-Arg-Val |
| 10 | RFVRRVRRFVRRVRRFVRRVRRFVRRVRRFVRRVRRFVRRVRRFVRRVR |
| | RFVRRVRRFVRRVRRFVRRVRRFVRRVRRFVRRVR |
| | Arg-Phe-Val-Arg-Arg-Val-Arg-Arg-Phe-Val-Arg-Arg-Val-Arg-Arg-Phe-Val- |
| | Arg-Arg-Val-Arg-Arg-Phe-Val-Arg-Arg-Val-Arg-Arg-Phe-Val-Arg-Arg-Val- |
| | Arg-Arg-Phe-Val-Arg-Arg-Val-Arg-Arg-Phe-Val-Arg-Arg-Val-Arg-Arg-Phe- |
| | Val-Arg-Arg-Val-Arg-Arg-Phe-Val-Arg-Arg-Val-Arg-Arg-Phe-Val-Arg-Arg- |
| | Val-Arg-Arg-Phe-Val-Arg-Arg-Val-Arg-Arg-Phe-Val-Arg-Arg-Val-Arg |
| 11 | RRTYSRSRRTYSRSRRTYSR |
| | Arg-Arg-Thr-Tyr-Ser-Arg-Ser-Arg-Arg-Thr-Tyr-Ser-Arg-Ser-Arg-Arg-Thr-Tyr- |
| | Ser-Arg |
| 12 | KVVSSIIEIISSVVKVVSSIIEIISSVV |
| | Lys-Val-Val-Ser-Ser-Ile-Ile-Glu-Ile-Ile-Ser-Ser-Val-Val-Lys-Val-Val-Ser-Ser- |
| | Ile-Ile-Glu-Ile-Ile-Ser-Ser-Val-Val |
| 13 | KKTHTKTKKTHTKTKKTHTK |
| | Lys-Lys-Thr-His-Thr-Lys-Thr-Lys-Lys-Thr-His-Thr-Lys-Thr-Lys-Lys-Thr-His- |
| | Thr-Lys |
| 14 | VVRVVRRVVRVVRRVVRVVRR |
| | Val-Val-Arg-Val-Val-Arg-Arg-Val-Val-Arg-Val-Val-Arg-Arg-Val-Val-Arg-Val- |
| | Val-Arg-Arg |
| 15 | RVVRVVRRVVRR |
| | Arg-Val-Val-Arg-Val-Val-Arg-Arg-Val-Val-Arg-Arg |
| 16 | RVVRVVRRWVRR |
| | Arg-Val-Val-Arg-Val-Val-Arg-Arg-Trp-Val-Arg-Arg |
| 17 | RWWRWWRRWWRR |
| | Arg-Trp-Trp-Arg-Trp-Trp-Arg-Arg-Trp-Trp-Arg-Arg |
| 18 | WRRWWRRWWRWWRRWWRR |
| | Trp-Arg-Arg-Trp-Trp-Arg-Arg-Trp-Trp-Arg-Trp-Trp-Arg-Arg-Trp-Trp-Arg-Arg |
| 19 | RRVVRRVRRVVRRVVRVVRRVVRR |
| | Arg-Arg-Val-Val-Arg-Arg-Val-Arg-Arg-Val-Val-Arg-Arg-Val-Val-Arg-Val- |
| | Val-Arg-Arg-Val-Val-Arg-Arg |
| 20 | RRWWRRWRRWWRRWWRWWRRWWRR |
| | Arg-Arg-Trp-Trp-Arg-Arg-Trp-Arg-Arg-Trp-Trp-Arg-Arg-Trp-Trp-Arg-Trp- |
| | Trp-Arg-Arg-Trp-Trp-Arg-Arg |
| 21 | VRRVVRRVVRVVRRVVRRVRRVVRRVVRVVRRVVRR |
| | Val-Arg-Arg-Val-Val-Arg-Arg-Val-Val-Arg-Val-Val-Arg-Arg-Val-Val-Arg- |
| | Arg-Val-Arg-Arg-Val-Val-Arg-Arg-Val-Val-Arg-Val-Val-Arg-Arg-Val-Val- |
| | Arg-Arg |
| 22 | VRRVWRRVVRVVRRWVRRVRRVWRRVVRVVRRWVRR |
| | Val-Arg-Arg-Val-Trp-Arg-Arg-Val-Val-Arg-Val-Val-Arg-Arg-Trp-Val-Arg- |
| | Arg-Val-Arg-Arg-Val-Trp-Arg-Arg-Val-Val-Arg-Val-Val-Arg-Arg-Trp-Val- |
| | Arg-Arg |
| 23 | RRVVRRVRRVVRRVVRVVRRVVRRVRRVVRRVVRVVRRVVRR |
| | Arg-Arg-Val-Val-Arg-Arg-Val-Arg-Arg-Val-Val-Arg-Arg-Val-Val-Arg-Val- |
| | Val-Arg-Arg-Val-Val-Arg-Arg-Val-Arg-Arg-Val-Val-Arg-Arg-Val-Val-Arg- |
| | Val-Val-Arg-Arg-Val-Val-Arg-Arg |
| 24 | RVVRVVRRVVRRVRRVVRRVVRVVRRVVRRVRRVVRRVVRVVRRVVR |
| | R |
| | Arg-Val-Val-Arg-Val-Val-Arg-Arg-Val-Val-Arg-Arg-Val-Arg-Arg-Val-Val- |
| | Arg-Arg-Val-Val-Arg-Val-Val-Arg-Arg-Val-Val-Arg-Arg-Val-Arg-Arg-Val- |
| | Val-Arg-Arg-Val-Val-Arg-Val-Val-Arg-Arg-Val-Val-Arg-Arg |
| 25 | RVVRVVRRWVRRVRRVWRRVVRVVRRWVRRVRRVWRRVVRVVRRW |
| | RVV |
| | Arg-Val-Val-Arg-Val-Val-Arg-Arg-Trp-Val-Arg-Arg-Val-Arg-Arg-Val-Trp- |
| | Arg-Arg-Val-Val-Arg-Val-Val-Arg-Arg-Trp-Val-Arg-Arg-Val-Arg-Arg-Val- |
| | Trp-Arg-Arg-Val-Val-Arg-Val-Val-Arg-Arg-Trp-Arg-Val-Val |

In some embodiments, the peptide or pharmaceutically acceptable salt thereof as described herein comprises SEQ ID NO: 1. In some embodiments, the peptide or pharmaceutically acceptable salt thereof as described herein comprises SEQ ID NO: 2. In some embodiments, the peptide or pharmaceutically acceptable salt thereof as described herein comprises SEQ ID NO: 3. In some embodiments, the peptide or pharmaceutically acceptable salt thereof as described herein comprises SEQ ID NO: 4. In some embodiments, the peptide or pharmaceutically acceptable salt thereof as described herein comprises SEQ ID NO: 5. In some embodiments, the peptide or pharmaceutically acceptable salt thereof as described herein comprises SEQ ID NO: 6. In some embodiments, the peptide or pharmaceutically acceptable salt thereof as described herein comprises SEQ ID NO: 7. In some embodiments, the peptide or pharmaceutically acceptable salt thereof as described herein comprises SEQ ID NO: 8. In some embodiments, the peptide or pharmaceutically acceptable salt thereof as described herein comprises SEQ ID NO: 9. In some embodiments, the peptide or pharmaceutically acceptable salt thereof as described herein comprises SEQ ID NO: 10. In some embodiments, the peptide or pharmaceutically acceptable salt thereof as described herein comprises SEQ ID NO: 11. In some embodiments, the peptide or pharmaceutically acceptable salt thereof as described herein comprises SEQ ID NO: 12. In some embodiments, the peptide or pharmaceutically acceptable salt thereof as described herein comprises SEQ ID NO: 13. In some embodiments, the peptide or pharmaceutically acceptable salt thereof as described herein comprises SEQ ID NO: 14. In some embodiments, the peptide or pharmaceutically acceptable salt thereof as described herein comprises SEQ ID NO: 15. In some embodiments, the peptide or pharmaceutically acceptable salt thereof as described herein comprises SEQ ID NO: 16. In some embodiments, the peptide or pharmaceutically acceptable salt thereof as described herein comprises SEQ ID NO: 17. In some embodiments, the peptide or pharmaceutically acceptable salt thereof as described herein comprises SEQ ID NO: 18. In some embodiments, the peptide or pharmaceutically acceptable salt thereof as described herein comprises SEQ ID NO: 19. In some embodiments, the peptide or pharmaceutically acceptable salt thereof as described herein comprises SEQ ID NO: 20. In some embodiments, the peptide or pharmaceutically acceptable salt thereof as described herein comprises SEQ ID NO: 21. In some embodiments, the peptide or pharmaceutically acceptable salt thereof as described herein comprises SEQ ID NO: 22. In some embodiments, the peptide or pharmaceutically acceptable salt thereof as described herein comprises SEQ ID NO: 23. In some embodiments, the peptide or pharmaceutically acceptable salt thereof as described herein comprises SEQ ID NO: 24. In some embodiments, the peptide or pharmaceutically acceptable salt thereof as described herein comprises SEQ ID NO: 25.

In some embodiments, the peptide or pharmaceutically acceptable salt thereof has at least 70% sequence identify to a polypeptide sequence of SEQ ID NO: 1, at least 70% sequence identify to a polypeptide sequence of SEQ ID NO: 2, at least 70% sequence identify to a polypeptide sequence of SEQ ID NO: 3, at least 70% sequence identify to a polypeptide sequence of SEQ ID NO: 4, at least 70% sequence identify to a polypeptide sequence of SEQ ID NO: 5, at least 70% sequence identify to a polypeptide sequence of SEQ ID NO: 6, at least 70% sequence identify to a polypeptide sequence of SEQ ID NO: 7, at least 70% sequence identify to a polypeptide sequence of SEQ ID NO: 8, at least 70% sequence identify to a polypeptide sequence of SEQ ID NO: 9, at least 70% sequence identify to a polypeptide sequence of SEQ ID NO: 10, at least 70% sequence identify to a polypeptide sequence of SEQ ID NO: 11, at least 70% sequence identify to a polypeptide sequence of SEQ ID NO: 12, at least 70% sequence identify to a polypeptide sequence of SEQ ID NO: 13, at least 70% sequence identify to a polypeptide sequence of SEQ ID NO: 14, at least 70% sequence identify to a polypeptide sequence of SEQ ID NO: 15, at least 70% sequence identify to a polypeptide sequence of SEQ ID NO: 16, at least 70% sequence identify to a polypeptide sequence of SEQ ID NO: 17, at least 70% sequence identify to a polypeptide sequence of SEQ ID NO: 18, at least 70% sequence identify to a polypeptide sequence of SEQ ID NO: 19, at least 70% sequence identify to a polypeptide sequence of SEQ ID NO: 20, at least 70% sequence identify to a polypeptide sequence of SEQ ID NO: 21, at least 70% sequence identify to a polypeptide sequence of SEQ ID NO: 22, at least 70% sequence identify to a polypeptide sequence of SEQ ID NO: 23, at least 70% sequence identify to a polypeptide sequence of SEQ ID NO: 24, or at least 70% sequence identify to a polypeptide sequence of SEQ ID NO: 25. In some embodiments, the peptide or pharmaceutically acceptable salt thereof has at least 70%, 75%, 80%, 85%, 90%, 95%, or 99% sequence identify to a polypeptide sequence listed in Table 1 and any increments of percentage therebetween.

In some embodiments, the pharmaceutical formulation comprises at least one peptide described herein as listed in Table 1. In some embodiments, the pharmaceutical formulation comprises one or more peptides described herein as listed in Table 1. In some embodiments, the pharmaceutical formulation comprises two or more peptides described herein as listed in Table 1. In some embodiments, the pharmaceutical formulation comprises three or more peptides described herein as listed in Table 1. In some embodiments, the pharmaceutical formulation comprises four or more peptides described herein as listed in Table 1. In some embodiments, the pharmaceutical formulation comprises five or more peptides described herein as listed in Table 1. In some embodiments, the pharmaceutical formulation comprises six or more peptides described herein as listed in Table 1. In some embodiments, the pharmaceutical formulation comprises seven or more peptides described herein as listed in Table 1. In some embodiments, the pharmaceutical formulation comprises eight or more peptides described herein as listed in Table 1. In some embodiments, the pharmaceutical formulation comprises nine or more peptides described herein as listed in Table 1. In some embodiments, the pharmaceutical formulation comprises ten or more peptides described herein as listed in Table 1. In some embodiments, the pharmaceutical formulation comprises eleven or more peptides described herein as listed in Table 1. In some embodiments, the pharmaceutical formulation comprises twelve or more peptides described herein as listed in Table 1. In some embodiments, the pharmaceutical formulation comprises thirteen or more peptides described herein as listed in Table 1. In some embodiments, the pharmaceutical formulation comprises fourteen or more peptides described herein as listed in Table 1. In some embodiments, the pharmaceutical formulation comprises fifteen or more peptides described herein as listed in Table 1.

A peptide disclosed herein can be a salt thereof. In some embodiments, recitation of the phrases “peptide” or “polypeptide” should be construed to include a salt thereof even if not explicitly recited. In some embodiments, a salt can include a carboxylate salt (e.g. formate, acetate, trifluoroacetate, trichloroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, pamoate, nicotinate, isonicotinate, cinnamate, oxalate, malonate, succinate, suberate, sebacate, fumarate, malate, maleate, hydroxymaleate, hippurate, phthalate or a terephthalate salts); a halide salt (e.g. chloride, bromide or iodide salts); a sulfonate salt (e.g. benzene sulfonate, methyl-, bromo- or chloro-benzenesulfonate, xylenesulfonate, methanesulfonate, trifluoromethanesulfonate, ethanesulfonate, propanesulfonate, hydroxyethanesulfonate, 1- or 2-naphthalene-sulfonate or 1,5-naphthalenedisulfonate salts); a sulfate salt; a pyrosulfate salt; a bisulfate salt; a sulfite salt; a bisulfite salt; a phosphate salt; a monohydrogenphosphate salt; a dihydrogenphosphate salt; a metaphosphate salt; a pyrophosphate salt; a nitrate salt; a chromium salt (e.g., octanoic acid); and the like.

In some embodiments, amino acids of the peptides described herein can be L-amino acids. In some embodiments, amino acids of the peptides described herein can be D-amino acids. In some embodiments, the peptides can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 D-amino acids and the rest are L-amino acids within the peptide sequence. In some embodiments, the peptides can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 L-amino acids and the rest are D-amino acids within the peptide sequence.

In some embodiments, a peptide can be formulated with one or more pharmaceutically acceptable salts. In some embodiments, a pharmaceutically acceptable salt can be a salt described in Berge et al, J. Pharm. Sci, 1977. In some embodiments, a pharmaceutically acceptable salts can include those salts prepared by reaction of a peptide with a mineral, organic acid or inorganic base, such salts including, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate and xylenesulfonate. In some embodiment, the pharmaceutically acceptable salt is an acetate salt.

In some embodiments, a peptide can be formulated as a cleavable prodrug. The term “prodrug” as used herein, can refer to a drug precursor that, following administration to a subject and subsequent absorption, can be converted to an active, or a more active species via some process, such as conversion by a metabolic pathway. Thus, the term can encompass a derivative, which, upon administration to a recipient, can be capable of providing, either directly or indirectly, a peptide, pharmaceutically acceptable salt or a metabolite or residue thereof. Some prodrugs can have a chemical group present on a prodrug that renders it less active and/or confers solubility or some other property to the drug. Once the chemical group has been cleaved and/or modified from the prodrug the active drug can be generated. a prodrugs can be a prodrug that can increase the bioavailability of a peptide when administered to a subject (e.g., by allowing an administered peptide to be more readily absorbed) or which enhance delivery of the peptide to a biological compartment (e.g., the brain or lymphatic system).

### Aqueous Carriers

In some embodiments, the aqueous carrier is lactated Ringer's solution, normal saline (0.9% w/v), or aqueous sodium carbonate. In some embodiments, the aqueous carrier is sodium bicarbonate, chlorocyclohexidine, chlorhexidine gluconate, normal saline, phosphate buffered saline, povidone-iodine (PVP-I), ethanol, acetic acid, sodium acetate, benzalkonium chloride, sodium lauryl sulfate, citric acid, or sodium citrate. In some embodiments, the aqueous carrier is lactated Ringer's solution. In some embodiments, the aqueous carrier is normal saline (0.9% w/v). In some embodiments, the aqueous carrier is aqueous sodium bicarbonate. In some embodiments, the aqueous carrier is physiologically isotonic, physiologically hypotonic, or physiologically hypertonic. In some embodiments, the aqueous carrier is physiologically isotonic. In some embodiments, the aqueous carrier is physiologically hypotonic. In some embodiments, the aqueous carrier is physiologically hypotonic (sub-physiologic osmolarity or osmolality), for example, modified versions of lactated Ringer's solution, normal saline (0.9% w/v), or aqueous sodium bicarbonate diluted with water. In some embodiments, the aqueous carrier is physiologically hypertonic.

In some embodiments, the aqueous carrier is present in the pharmaceutical formulation at a concentration of about 25 mM to about 300 mM. In some embodiments, the aqueous carrier is present in the pharmaceutical formulation at a concentration of about 25 mM to about 300 mM, about 50 mM to about 300 mM, about 100 mM to about 300 mM, about 150 mM to about 300 mM, about 200 mM to about 300 mM, about 25 mM to about 250 mM, about 25 mM to about 200 mM, about 25 mM to about 150 mM, about 25 mM to about 100 mM, about 50 mM to about 250 mM, about 100 mM to about 200 mM, or about 100 mM to about 250 mM. In some embodiments, aqueous carrier is present in the pharmaceutical formulation at a concentration at about 25 mM, about 50 mM, about 100 mM, about 150 mM, about 200 mM, about 250 mM, or about 300 mM. In some embodiments, the aqueous carrier is present in the pharmaceutical formulation at a concentration of about 50 mM. In some embodiments, the aqueous carrier is present in the pharmaceutical formulation at a concentration of about 100 mM. In some embodiments, the aqueous carrier is present in the pharmaceutical formulation at a concentration of about 150 mM. In some embodiments, the aqueous carrier is present in the pharmaceutical formulation at a concentration of about 200 mM. In some embodiments, the aqueous carrier is present in the pharmaceutical formulation at a concentration of about 250 mM. In some embodiments, the aqueous carrier is present in the pharmaceutical formulation at a concentration of about 300 mM.

In some embodiments, the aqueous carrier has a concentration of about 25 mM to about 300 mM. In some embodiments, the aqueous carrier has a concentration of about of about 25 mM to about 300 mM, about 50 mM to about 300 mM, about 100 mM to about 300 mM, about 150 mM to about 300 mM, about 200 mM to about 300 mM, about 25 mM to about 250 mM, about 25 mM to about 200 mM, about 25 mM to about 150 mM, about 25 mM to about 100 mM, about 50 mM to about 250 mM, about 100 mM to about 200 mM, or about 100 mM to about 250 mM. In some embodiments, the aqueous carrier has a concentration of about at about 25 mM, about 50 mM, about 100 mM, about 150 mM, about 200 mM, about 250 mM, or about 300 mM. In some embodiment, the aqueous carrier has a concentration of about 25 mM. In some embodiment, the aqueous carrier has a concentration of about 50 mM. In some embodiment, the aqueous carrier has a concentration of about 100 mM. In some embodiment, the aqueous carrier has a concentration of about 150 mM. In some embodiment, the aqueous carrier has a concentration of about 200 mM.

In some embodiment, the aqueous carrier has a concentration of about 250 mM. In some embodiment, the aqueous carrier has a concentration of about 300 mM. In some embodiments, the aqueous carrier is present in the pharmaceutical formulation at a concentration of about 5 mg/mL to about 50 mg/mL. In some embodiments, the aqueous carrier is present in the pharmaceutical formulation at a concentration of about 5 mg/mL to about 50 mg/mL, about 5 mg/mL to about 40 mg/mL, about 5 mg/mL to about 30 mg/mL, about 5 mg/mL to about 20 mg/mL, about 5 mg/mL to about 10 mg/mL, about 5 mg/mL to about 15 mg/mL, about 10 mg/mL to about 40 mg/mL, about 10 mg/mL to about 30 mg/mL, about 10 mg/mL to about 20 mg/mL, about 15 mg/mL to about 35 mg/mL, about 15 mg/mL to about 25 mg/mL. In some embodiments, the aqueous carrier is present in the pharmaceutical formulation at a concentration of about 5 mg/mL, about 10 mg/mL, about 12.5 mg/mL, about 12.6 mg/mL, about 15 mg/mL, about 20 mg/mL, about 25 mg/mL, about 30 mg/mL, about 35 mg/mL, about 40 mg/mL, about 45 mg/mL, or about 50 mg/mL.

In some embodiments, the aqueous carrier has a concentration of about 5 mg/mL to about 50 mg/mL. In some embodiments, the aqueous carrier has a concentration of about 5 mg/mL to about 50 mg/mL, about 5 mg/mL to about 40 mg/mL, about 5 mg/mL to about 30 mg/mL, about 5 mg/mL to about 20 mg/mL, about 5 mg/mL to about 10 mg/mL, about 5 mg/mL to about 15 mg/mL, about 10 mg/mL to about 40 mg/mL, about 10 mg/mL to about 30 mg/mL, about 10 mg/mL to about 20 mg/mL, about 15 mg/mL to about 35 mg/mL, about 15 mg/mL to about 25 mg/mL. In some embodiments, the aqueous carrier has a concentration of about 5 mg/mL, about 10 mg/mL, about 12.5 mg/mL, about 12.6 mg/mL, about 15 mg/mL, about 20 mg/mL, about 25 mg/mL, about 30 mg/mL, about 35 mg/mL, about 40 mg/mL, about 45 mg/mL, or about 50 mg/mL.

In some embodiments, the aqueous carrier is only aqueous sodium bicarbonate. In some embodiments, the pharmaceutical formulation comprises of a peptide or pharmaceutically acceptable salt thereof as described herein and aqueous sodium bicarbonate, and water. In some embodiments, the pharmaceutical formulation consists of a peptide described herein and aqueous sodium bicarbonate, water, and no additional excipients.

In some embodiment, the aqueous carrier has a total osmolality ranging from about 1 milliosmole per kilogram (mOsm/kg) to about 5000 mOsm/kg. In some embodiment, the aqueous carrier has a total osmolality ranging from about 30 mOsm/kg to about 800 mOsm/kg, about 50 to about 500 mOsm/kg, 200 mOsm/kg to about 500 mOsm/kg. In some embodiments, the aqueous carrier has a total osmolarity of about 1 mOsm/kg, about 50 mOsm/kg, about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, about 500 mOsm/kg, about 800 mOsm/kg, about 1000 mOsm/kg, about 1500 mOsm/kg, about 2000 mOsm/kg, about 2500 mOsm/kg, about 3000 mOsm/kg, about 3500 mOsm/kg, about 4000 mOsm/kg, about 4500 mOsm/kg, or about 5000 mOsm/kg.

In some embodiments, the aqueous carrier has a total osmolarity ranging from about 1 milliosmoles per one liter (mOsm/L) to about 5,000 mOsm/L. In some embodiments, the aqueous carrier has a total osmolarity of about 1 mOsm/L, about 50 mOsm/L, about 100 mOsm/L, about 150 mOsm/L, about 200 mOsm/L, about 250 mOsm/L, about 300 mOsm/L, about 350 mOsm/L, about 400 mOsm/L, about 450 mOsm/L, about 500 mOsm/L, about 1000 mOsm/L, about 1500 mOsm/L, about 2000 mOsm/L, about 2500 mOsm/L, about 3000 mOsm/L, about 3500 mOsm/L, about 4000 mOsm/L, about 4500 mOsm/L, or about 5000 mOsm/L.

In some embodiments, the aqueous carrier may have a total ionic strength ranging from about 0.001 molar (M) and 1.0 M. In some embodiments, aqueous carrier may have a total ionic strength of about 0.001 M, about 0.01 M, about 0.015 M, about 0.02 M, about 0.025 M, about 0.03 M, about 0.035 M, about 0.04 M, about 0.05 M, about 0.055 M, about 0.06 M, about 0.065 M, about 0.07 M, about 0.075 M, about 0.08 M, about 0.085 M, about 0.09 M, about 0.1 M, about 0.12 M, about 0.14 M, about 0.15 M, about 0.16 M, about 0.18 M, about 0.2 M, about 0.22 M, about 0.24 M, about 0.25 M, about 0.26 M, about 0.28 M, about 0.03 M, about 0.35 M, about 0.4 M, about 0.45 M, about 0.5 M, about 0.55 M, about 0.6 M, about 0.65 M, about 0.7 M, about 0.75 M about 0.8 M, about 0.85 M, about 0.9 M, about 0.95 M, or about 1.0 M.

In some embodiments, the aqueous carrier may have a concentration in milliequivalents per liter (mEq/L) ranging about 0.1 mEq/L to about 200 mEq/L. In some embodiments, the aqueous carrier has a concentration of about 0.1 mEq/L to about 200 mEq/L, 1 mEq/L to about 175 mEq/L, about 1 mEq/L to about 150 mEq/L, about 1 mEq/L to about 125 mEq/L, about 1 mEq/L to about 100 mEq/L, about 1 mEq/L to about 75 mEq/L, about 25 mEq/L to about 200 mEq/L, about 50 mEq/L to about 200 mEq/L, about 75 mEq/L to about 200 mEq/L, about 100 mEq/L to about 200 mEq/L, about 125 mEq/L to about 200 mEq/L, about 25 mEq/L to about 175 mEq/L, about 50 mEq/L to about 150 mEq/L, about 25 mEq/L to about 100 mEq/L, or about 25 mEq/L to about 150 mEq/L. In some embodiments, the aqueous carrier has a concentration of about 0.1 mEq/L, about 1 mEq/L, about 25 mEq/L, about 50 mEq/L, about 75 mEq/L, about 100 mEq/L, about 125 mEq/L, about 150 mEq/L, about 175 mEq/L, or about mEq/L. In some embodiments, the aqueous carrier has a concentration of about 50 mEq/L.

In some embodiments, the pharmaceutical formulation is physiologically isotonic, physiologically hypotonic, or physiologically hypertonic. In some embodiments, the pharmaceutical formulation is physiologically isotonic. In some embodiments, the pharmaceutical formulation is physiologically hypotonic. In some embodiments, the pharmaceutical formulation is physiologically hypertonic.

In some embodiments, the pharmaceutical formulation may have an osmolarity of from at least 1 mOsm/kg to at least 10 osmoles per kilogram (Osm/kg), at least 1 mOsm/kg to at least 5 Osm/kg, at least 1 mOsm/kg to at least 4 Osm/kg, at least 1 mOsm/kg to at least 3 Osm/kg, at least 1 mOsm/kg to at least 2 Osm/kg, at least 1 mOsm/kg to at least 1 Osm/kg, at least 1 mOsm/kg to at least 900 mOsm/kg, at least 1 mOsm/kg to at least 800 mOsm/kg, at least 1 mOsm/kg to at least 700 mOsm/kg, at least 1 mOsm/kg to at least 600 mOsm/kg, at least 1 mOsm/kg to at least 500 mOsm/kg, at least 1 mOsm/kg to at least 400 mOsm/kg, at least 1 mOsm/kg to at least 300 mOsm/kg, at least 1 mOsm/kg to at least 200 mOsm/kg, at least 1 mOsm/kg to at least 100 mOsm/kg, at least 1 mOsm/kg to at least 500 mOsm/kg, at least 1 mOsm/kg to at least 30 mOsm/kg, at least 30 mOsm/kg to at least 400 mOsm/kg, or at least 275 mOsm/kg to at least 295 mOsm/kg. In some embodiments, the pharmaceutical formulation may have an osmolarity of from at least 30 mOsm/kg to at least 400 mOsm/kg.

In some embodiments, the pharmaceutical formulation has a total osmolality ranging from about 1 milliosmole per kilogram (mOsm/kg) from 5000 mOsm/kg. In some embodiments, the pharmaceutical formulation has a total osmolarity of about 1 mOsm/kg, about 50 mOsm/kg, about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, about 500 mOsm/kg, about 1000 mOsm/kg, about 1500 mOsm/kg, about 2000 mOsm/kg, about 2500 mOsm/kg, about 3000 mOsm/kg, about 3500 mOsm/kg, about 4000 mOsm/kg, about 4500 mOsm/kg, about 5000 mOsm/kg, or any ranges therebetween. In some embodiment, the pharmaceutical formulation has a total osmolality ranging from about 1 mOsm/kg to about 5000 mOsm/kg. In some embodiment, the aqueous carrier has a total osmolality ranging from about 30 mOsm/kg to about 800 mOsm/kg, about 50 to about 500 mOsm/kg, 200 mOsm/kg to about 500 mOsm/kg. In some embodiments, the aqueous carrier has a total osmolarity of about 1 mOsm/kg, about 50 mOsm/kg, about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, about 500 mOsm/kg, about 800 mOsm/kg, about 1000 mOsm/kg, about 1500 mOsm/kg, about 2000 mOsm/kg, about 2500 mOsm/kg, about 3000 mOsm/kg, about 3500 mOsm/kg, about 4000 mOsm/kg, about 4500 mOsm/kg, or about 5000 mOsm/kg.

In some embodiments, the pharmaceutical formulation has a total osmolarity ranging from about 1 mOsm/L to about 5,000 mOsm/L. In some embodiments, the pharmaceutical formulation has a total osmolarity of about 1 mOsm/L, about 50 mOsm/L, about 100 mOsm/L, about 150 mOsm/L, about 200 mOsm/L, about 250 mOsm/L, about 300 mOsm/L, about 350 mOsm/L, about 400 mOsm/L, about 450 mOsm/L, about 500 mOsm/L, about 1000 mOsm/L, about 1500 mOsm/L, about 2000 mOsm/L, about 2500 mOsm/L, about 3000 mOsm/L, about 3500 mOsm/L, about 4000 mOsm/L, about 4500 mOsm/L, about 5000 mOsm/L, or any ranges therebetween. In some embodiments, the pharmaceutical formulation has a total osmolarity ranging from about 1 mOsm/L to about 5,000 mOsm/L. In some embodiments, the aqueous carrier has a total osmolarity of about 1 mOsm/L, about 50 mOsm/L, about 100 mOsm/L, about 150 mOsm/L, about 200 mOsm/L, about 250 mOsm/L, about 300 mOsm/L, about 350 mOsm/L, about 400 mOsm/L, about 450 mOsm/L, about 500 mOsm/L, about 1000 mOsm/L, about 1500 mOsm/L, about 2000 mOsm/L, about 2500 mOsm/L, about 3000 mOsm/L, about 3500 mOsm/L, about 4000 mOsm/L, about 4500 mOsm/L, or about 5000 mOsm/L.

In some embodiments, the pharmaceutical formulation has a total ionic strength ranging from about 0.001 molar (M) and 1.0 M. The aqueous carrier may have a total ionic strength of about 0.001 M, about 0.01 M, about 0.015 M, about 0.02 M, about 0.025 M, about 0.03 M, about 0.035 M, about 0.04 M, about 0.05 M, about 0.055 M, about 0.06 M, about 0.065 M, about 0.07 M, about 0.075 M, about 0.08 M, about 0.085 M, about 0.09 M, about 0.1 M, about 0.12 M, about 0.14 M, about 0.15 M, about 0.16 M, about 0.18 M, about 0.2 M, about 0.22 M, about 0.24 M, about 0.25 M, about 0.26 M, about 0.28 M, about 0.03 M, about 0.35 M, about 0.4 M, about 0.45 M, about 0.5 M, about 0.55 M, about 0.6 M, about 0.65 M, about 0.7 M, about 0.75 M about 0.8 M, about 0.85 M, about 0.9 M, about 0.95 M, about 1.0 M, or any ranges therebetween.

### Synergy

In some embodiments, a pharmaceutical formulation comprises a synergistic effect between the peptide of salt thereof and the aqueous carrier. In some embodiments, administration of a pharmaceutical composition comprising a peptide described herein and an aqueous carrier together provides a synergistic effect. In some embodiments, the synergistic effect comprises decreasing a bacterial burden to a greater extent compared to administering a peptide described herein alone or an aqueous carrier alone. In some embodiments, a pharmaceutical formulation comprising a peptide described herein and an aqueous carrier may have a synergistic effect in reducing the bacterial burden when administered to a subject. In some embodiments, a pharmaceutical formulation comprising a peptide described herein and sodium bicarbonate may have a synergistic effect in reducing the bacterial burden when administered to a subject.

In some embodiments, the synergistic effect may comprise reducing incidence of abscesses. In some embodiments, the synergistic effect may comprise reducing the incidence of abscesses at a site of infection. In some embodiments, the synergistic effect is improved survivability of a patient.

pH

In some embodiments, the pharmaceutical formulation described herein may further comprise a pH value of about 7 to about 10, about 8.0 to about 13, from about 5.5 to about 7.0, from about 7.0 to about 8.0, about 7 to about 11, about 8 to about 11, or from about 5.5 to about 13. In some embodiments, the pharmaceutical composition comprises a pH from about 7 to about 10. In some embodiments, the pharmaceutical formulation may comprise a pH value from 8.0 to 13, including increments therebetween, such as 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0.

In some embodiments, the pharmaceutical formulation may comprise a pH value from 5.5 to 7.0, including increments therebetween, such as 5.5, 5.6, 5.7, 5.8, 5.9, 6.0, 6.1, 6.2, 6.3, 6.4, 6.5, 6.6, 6.7, 6.8, 6.9, 7.0.

In some embodiments, the pharmaceutical formulation may comprise a pH value from about 7.0 to about 8.0, including increments therebetween, such as 7.0, 7.1, 7.2, 7.3, 7.4, 7.5, 7.6, 7.7, 7.8, 7.9, 8.0. In some embodiments, the pharmaceutical formulation has a pH of about 7.0. In some embodiments, the pharmaceutical formulation has a pH of about 7.1. In some embodiments, the pharmaceutical formulation has a pH of about 7.2. In some embodiments, the pharmaceutical formulation has a pH of about 7.3. In some embodiments, the pharmaceutical formulation has a pH of about 7.4. In some embodiments, the pharmaceutical formulation has a pH of about 7.5. In some embodiments, the pharmaceutical formulation has a pH of about 7.6. In some embodiments, the pharmaceutical formulation has a pH of about 7.7. In some embodiments, the pharmaceutical formulation has a pH of about 7.8. In some embodiments, the pharmaceutical formulation has a pH of about 7.9. In some embodiments, the pharmaceutical formulation has a pH of about 8.0.

In some embodiments, the pharmaceutical formulation has a pH of about 8.1. In some embodiments, the pharmaceutical formulation has a pH of about 8.2. In some embodiments, the pharmaceutical formulation has a pH of about 8.3. In some embodiments, the pharmaceutical formulation has a pH of about 8.4. In some embodiments, the pharmaceutical formulation has a pH of about 8.5. In some embodiments, the pharmaceutical formulation has a pH of about 8.6. In some embodiments, the pharmaceutical formulation has a pH of about 8.7 In some embodiments, the pharmaceutical formulation has a pH of about 8.8. In some embodiments, the pharmaceutical formulation has a pH of about 8.9. In some embodiments, the pharmaceutical formulation has a pH of about 9.0. In some embodiments, the pharmaceutical formulation has a pH of about 9.1. In some embodiments, the pharmaceutical formulation has a pH of about 9.2. In some embodiments, the pharmaceutical formulation has a pH of about 9.3. In some embodiments, the pharmaceutical formulation has a pH of about 9.4. In some embodiments, the pharmaceutical formulation has a pH of about 9.5. In some embodiments, the pharmaceutical formulation has a pH of about 9.6. In some embodiments, the pharmaceutical formulation has a pH of about 9.7. In some embodiments, the pharmaceutical formulation has a pH of about 9.8. In some embodiments, the pharmaceutical formulation has a pH of about 9.9. In some embodiments, the pharmaceutical formulation has a pH of about 10.0. In some embodiments, the pharmaceutical formulation has a pH of about 10.1. In some embodiments, the pharmaceutical formulation has a pH of about 10.2. In some embodiments, the pharmaceutical formulation has a pH of about 10.3. In some embodiments, the pharmaceutical formulation has a pH of about 10.4. In some embodiments, the pharmaceutical formulation has a pH of about 10.5. In some embodiments, the pharmaceutical formulation has a pH of about 10.6. In some embodiments, the pharmaceutical formulation has a pH of about 10.7. In some embodiments, the pharmaceutical formulation has a pH of about 10.8. In some embodiments, the pharmaceutical formulation has a pH of about 10.9. In some embodiments, the pharmaceutical formulation has a pH of about 11.0. In some embodiments, the pharmaceutical formulation has a pH of about 11.1. In some embodiments, the pharmaceutical formulation has a pH of about 11.2. In some embodiments, the pharmaceutical formulation has a pH of about 11.3. In some embodiments, the pharmaceutical formulation has a pH of about 11.4. In some embodiments, the pharmaceutical formulation has a pH of about 11.5. In some embodiments, the pharmaceutical formulation has a pH of about 11.6. In some embodiments, the pharmaceutical formulation has a pH of about 11.7. In some embodiments, the pharmaceutical formulation has a pH of about 11.8. In some embodiments, the pharmaceutical formulation has a pH of about 11.9. In some embodiments, the pharmaceutical formulation has a pH of about 12.0. In some embodiments, the pharmaceutical formulation has a pH of about 12.1. In some embodiments, the pharmaceutical formulation has a pH of about 12.2. In some embodiments, the pharmaceutical formulation has a pH of about 12.3. In some embodiments, the pharmaceutical formulation has a pH of about 12.4. In some embodiments, the pharmaceutical formulation has a pH of about 12.5. In some embodiments, the pharmaceutical formulation has a pH of about 12.6. In some embodiments, the pharmaceutical formulation has a pH of about 12.7. In some embodiments, the pharmaceutical formulation has a pH of about 12.8. In some embodiments, the pharmaceutical formulation has a pH of about 12.9. In some embodiments, the pharmaceutical formulation has a pH of about 13.0.

In some embodiments, the pharmaceutical formulation has a pH of about 5.5. In some embodiments, the pharmaceutical formulation has a pH of about 5.6. In some embodiments, the pharmaceutical formulation has a pH of about 5.7. In some embodiments, the pharmaceutical formulation has a pH of about 5.8. In some embodiments, the pharmaceutical formulation has a pH of about 5.9. In some embodiments, the pharmaceutical formulation has a pH of about 6.0. In some embodiments, the pharmaceutical formulation has a pH of about 6.1. In some embodiments, the pharmaceutical formulation has a pH of about 6.2. In some embodiments, the pharmaceutical formulation has a pH of about 6.3. In some embodiments, the pharmaceutical formulation has a pH of about 6.4. In some embodiments, the pharmaceutical formulation has a pH of about 6.5. In some embodiments, the pharmaceutical formulation has a pH of about 6.6. In some embodiments, the pharmaceutical formulation has a pH of about 6.7. In some embodiments, the pharmaceutical formulation has a pH of about 6.8. In some embodiments, the pharmaceutical formulation has a pH of about 6.9.

In some embodiments, the pharmaceutical formulation comprising a peptide or pharmaceutically acceptable salt and an aqueous carrier is at a pH value of at least 8.0 to at least 13, at least 8.0 to at least 13, at least 8.1 to at least 13, at least 8.2 to at least 13, at least 8.3 to at least 13, at least 8.4 to at least 13, at least 8.5 to at least 13, at least 8.6 to at least 13 at least 8.7 to at least 13 at least 8.8 to at least 13, at least 8.9 to at least 13, at least 9.0 to at least 13, at least 9.1 to at least 13, at least 9.2 to at least 13, at least 9.3 to at least 13, at least 9.4 to at least 13, at least 9.5 to at least 13, at least 9.6 to at least 13, at least 9.7 to at least 13, at least 9.8 to at least 13, at least 9.9 to at least 13, at least 10.0 to at least 13, at least 10.1 to at least 13, at least 10.2 to at least 13, at least 10.3 to at least 13, at least 10.4 to at least 13, at least 10.5 to at least 13, at least 10.6 to at least 13, at least 10.7 to at least 13, at least 10.8 to at least 13, at least 10.7 to at least 13, at least 10.7 to at least 13, at least 10.8 to at least 13, at least 10.9 to at least 13, at least 11 to at least 13, at least 11.1 to at least 13, at least 11.2 to at least 13, at least 11.2 to at least 13, at least 11.3 to at least 13, at least 11.4 to at least 13, at least 11.5 to at least 13, at least 11.6 to at least 13, at least 11.7 to at least 13, at least 11.8 to at least 13, at least 11.9 to at least 13, at least 12.0 to at least 13, at least 12.1 to at least 13, at least 12.2 to at least 13, at least 12.3 to at least 13, at least 12.4 to at least 13, at least 12.5 to at least 13, at least 12.6 to at least 13, at least 12.7 to at least 13, at least 12.8 to at least 13, or at least 12.9 to at least 13. In some embodiments, the pharmaceutical formulation comprising a peptide or pharmaceutically acceptable salt and an aqueous carrier is at a pH value of at least 5.5 to at least 7.0, at least 5.6 to at least 7.0, at least 5.7 to at least 7.0, at least 5.8 to at least 7.0, at least 5.9 to at least 7.0, at least 6.0 to at least 7.0, at least 6.1 to at least 7.0, at least 6.2 to at least 7.0, at least 6.3 to at least 7.0, at least 6.4 to at least 7.0, at least 6.5 to at least 7.0, at least 6.6 to at least 7.0, at least 6.7 to at least 7.0, at least 6.8 to at least 7.0, or at least 6.9 to at least 7.0. In some embodiments, the pharmaceutical formulation comprising a peptide or pharmaceutically acceptable salt and an aqueous carrier is at a pH value of at least 7.0 to at least 8.0, at least 7.1 to at least 8.0, at least 7.2 to at least 8.0, at least 7.3 to at least 8.0, at least 7.4 to at least 8.0, at least 7.5 to at least 8.0, at least 7.6 to at least 8.0, at least 7.7 to at least 8.0, at least 7.8 to at least 8.0, or at least 7.9 to at least 8.0. In some embodiments, the pharmaceutical formulation comprising a peptide or pharmaceutically acceptable salt and an aqueous carrier is at a pH value of at least 8.0 to at least 13.0, at least 8.0 to at least 12.9, at least 8.0 to at least 12.8, at least 8.0 to at least 12.7, at least 8.0 to at least 12.6, at least 8.0 to at least 12.5, at least 8.0 to at least 12.4, at least 8.0 to at least 12.3, at least 8.0 to at least 12.2, at least 8.0 to at least 12.1, at least 8.0 to at least 12.0, at least 8.0 to at least 11.9, at least 8.0 to at least 11.8, at least 8.0 to at least 11.7, at least 8.0 to at least 11.6, at least 8.0 to at least 11.5, at least 8.0 to at least 11.4, at least 8.0 to at least 11.3, at least 8.0 to at least 11.2, at least 8.0 to at least 11.1, at least 8.0 to at least 11.0, at least 8.0 to at least 10.9, at least 8.0 to at least 10.8, at least 8.0 to at least 10.8, at least 8.0 to at least 10.7, at least 8.0 to at least 10.6, at least 8.0 to at least 10.5, at least 8.0 to at least 10.4, at least 8.0 to at least 10.3, at least 8.0 to at least 10.2, at least 8.0 to at least 10.1, at least 8.0 to at least 10.0, at least 8.0 to at least 9.9, at least 8.0 to at least 9.8, at least 8.0 to at least 9.7, at least 8.0 to at least 9.6, at least 8.0 to at least 9.5, at least 8.0 to at least 9.4, at least 8.0 to at least 9.3, at least 8.0 to at least 9.2, at least 8.0 to at least 9.1, at least 8.0 to at least 9.0, at least 8.0 to at least 8.9, at least 8.0 to at least 8.8, at least 8.0 to at least 8.7, at least 8.0 to at least 8.6, at least 8.0 to at least 8.5, at least 8.0 to at least 8.4, at least 8.0 to at least 8.3, at least 8.0 to at least 8.2, at least 8.0 to at least 8.1.

In some embodiments, the pharmaceutical formulation comprising a peptide or pharmaceutically acceptable salt and an aqueous carrier is at a pH value of at least 5.5 to at least 7.0, at least 5.5 to at least 6.9, at least 5.5 to at least 6.8, at least 5.5 to at least 6.7, at least 5.5 to at least 6.6, at least 5.5 to at least 6.5, at least 5.5 to at least 6.4, at least 5.5 to at least 6.3, at least 5.5 to at least 6.2, at least 5.5 to at least 6.1, at least 5.5 to at least 6.0, at least 5.5 to at least 5.9, at least 5.5 to at least 5.8, at least 5.5 to at least 5.7, at least 5.5 to 5.6.

In some embodiments, the pharmaceutical formulation comprising a peptide or pharmaceutically acceptable salt and an aqueous carrier is at a pH value of at least 7.0 to at least 8.0, at least 7.0 to at least 7.9, at least 7.0 to at least 7.8, at least 7.0 to at least 7.7, at least 7.0 to at least 7.6, at least 7.0 to at least 7.5, at least 7.0 to at least 7.4, at least 7.0 to at least 7.3, at least 7.0 to at least 7.2, at least 7.0 to at least 7.1. In some embodiments, the pharmaceutical formulation comprising a peptide or pharmaceutically acceptable salt and an aqueous carrier is at a pH value of at least 8.0 to at least 13.0, at least 8.1 to at least 12.9, at least 8.2 to at least 12.8, at least 8.3 to at least 12.7, at least 8.4 to at least 12.6, at least 8.5 to at least 12.5, at least 8.6 to at least 12.4, at least 8.7 to at least 12.3, at least 8.8 to at least 12.2, at least 8.9 to at least 12.1, at least 9.0 to at least 12.0, at least 9.1 to at least 11.9, at least 9.2 to at least 11.8, at least 9.3 to at least 11.7, at least 9.4 to at least 11.6, at least 9.5 to at least 11.5, at least 9.6 to at least 11.4, at least 9.7 to at least 11.3, at least 9.8 to at least 11.2, at least 9.9 to at least 11.1, at least 10.0 to at least 11.0, at least 10.1 to at least 10.9, at least 10.2 to 10.8, at least 10.3 to 10.7, at least 10.4 to 10.6, at least 10.5.

In some embodiments, the pharmaceutical formulation comprising a peptide or pharmaceutically acceptable salt and an aqueous carrier is at a pH value of at least 5.5 to at least 7.0, at least 5.6 to at least 6.9, at least 5.7 to at least 6.8, at least 5.8 to at least 6.7, at least 5.9 to at least 6.6, at least 6.0 to at least 6.5, at least 6.1 to at least 6.4, at least 6.2 to at least 6.3. In some embodiments, the pharmaceutical formulation comprising a peptide or pharmaceutically acceptable salt is at a pH value of at least 7.0 to at least 8.0, at least 7.1 to at least 7.9, at least 7.2 to at least 7.8, at least 7.3 to at least 7.7, at least 7.4 to at least 7.6, at least 7.5.

In some embodiments, the pharmaceutical formulation comprising a peptide or pharmaceutically acceptable salt and an aqueous carrier is at a pH value of at most 8.0 to at most 13, at most 8.0 to at most 13, at most 8.1 to at most 13, at most 8.2 to at most 13, at most 8.3 to at most 13, at most 8.4 to at most 13, at most 8.5 to at most 13, at most 8.6 to at most 13 at most 8.7 to at most 13 at most 8.8 to at most 13, at most 8.9 to at most 13, at most 9.0 to at most 13, at most 9.1 to at most 13, at most 9.2 to at most 13, at most 9.3 to at most 13, at most 9.4 to at most 13, at most 9.5 to at most 13, at most 9.6 to at most 13, at most 9.7 to at most 13, at most 9.8 to at most 13, at most 9.9 to at most 13, at most 10.0 to at most 13, at most 10.1 to at most 13, at most 10.2 to at most 13, at most 10.3 to at most 13, at most 10.4 to at most 13, at most 10.5 to at most 13, at most 10.6 to at most 13, at most 10.7 to at most 13, at most 10.8 to at most 13, at most 10.9 to at most 13, at most 11 to at most 13, at most 11.1 to at most 13, at most 11.2 to at most 13, at most 11.2 to at most 13, at most 11.3 to at most 13, at most 11.4 to at most 13, at most 11.5 to at most 13, at most 11.6 to at most 13, at most 11.7 to at most 13, at most 11.8 to at most 13, at most 11.9 to at most 13, at most 12.0 to at most 13, at most 12.1 to at most 13, at most 12.2 to at most 13, at most 12.3 to at most 13, at most 12.4 to at most 13, at most 12.5 to at most 13, at most 12.6 to at most 13, at most 12.7 to at most 13, at most 12.8 to at most 13, or at most 12.9 to at most 13.

In some embodiments, the pharmaceutical formulation comprising a peptide or pharmaceutically acceptable salt and an aqueous carrier is at a pH value of at most 5.5 to at most 7.0, at most 5.6 to at most 7.0, at most 5.7 to at most 7.0, at most 5.8 to at most 7.0, at most 5.9 to at most 7.0, at most 6.0 to at most 7.0, at most 6.1 to at most 7.0, at most 6.2 to at most 7.0, at most 6.3 to at most 7.0, at most 6.4 to at most 7.0, at most 6.5 to at most 7.0, at most 6.6 to at most 7.0, at most 6.7 to at most 7.0, at most 6.8 to at most 7.0, or at most 6.9 to at most 7.0.

In some embodiments, the pharmaceutical formulation comprising a peptide or pharmaceutically acceptable salt and an aqueous carrier is at a pH value of at most 7.0 to at most 8.0, at most 7.1 to at most 8.0, at most 7.2 to at most 8.0, at most 7.3 to at most 8.0, at most 7.4 to at most 8.0, at most 7.5 to at most 8.0, at most 7.6 to at most 8.0, at most 7.7 to at most 8.0, at most 7.8 to at most 8.0, or at most 7.9 to at most 8.0. In some embodiments, the pharmaceutical formulation comprising a peptide or pharmaceutically acceptable salt is at a pH value of at most 8.0 to at most 13, at most 8.0 to at most 12.9, at most 8.0 to at most 12.8, at most 8.0 to at most 12.7, at most 8.0 to at most 12.6, at most 8.0 to at most 12.5, at most 8.0 to at most 12.4, at most 8.0 to at most 12.3, at most 8.0 to at most 12.2, at most 8.0 to at most 12.1, at most 8.0 to at most 12.0, at most 8.0 to at most 11.9, at most 8.0 to at most 11.8, at most 8.0 to at most 11.7, at most 8.0 to at most 11.6, at most 8.0 to at most 11.5, at most 8.0 to at most 11.4, at most 8.0 to at most 11.3, at most 8.0 to at most 11.2, at most 8.0 to at most 11.1, at most 8.0 to at most 11.0, at most 8.0 to at most 10.9, at most 8.0 to at most 10.8, at most 8.0 to at most 10.8, at most 8.0 to at most 10.7, at most 8.0 to at most 10.6, at most 8.0 to at most 10.5, at most 8.0 to at most 10.4, at most 8.0 to at most 10.3, at most 8.0 to at most 10.2, at most 8.0 to at most 10.1, at most 8.0 to at most 10.0, at most 8.0 to at most 9.9, at most 8.0 to at most 9.8, at most 8.0 to at most 9.7, at most 8.0 to at most 9.6, at most 8.0 to at most 9.5, at most 8.0 to at most 9.4, at most 8.0 to at most 9.3, at most 8.0 to at most 9.2, at most 8.0 to at most 9.1, at most 8.0 to at most 9.0, at most 8.0 to at most 8.9, at most 8.0 to at most 8.8, at most 8.0 to at most 8.7, at most 8.0 to at most 8.6, at most 8.0 to at most 8.5, at most 8.0 to at most 8.4, at most 8.0 to at most 8.3, at most 8.0 to at most 8.2, at most 8.0 to at most 8.1.

In some embodiments, the pharmaceutical formulation comprising a peptide or pharmaceutically acceptable salt and an aqueous carrier is at a pH value of at most 5.5 to at most 7.0, at most 5.5 to at most 6.9, at most 5.5 to at most 6.8, at most 5.5 to at most 6.7, at most 5.5 to at most 6.6, at most 5.5 to at most 6.5, at most 5.5 to at most 6.4, at most 5.5 to at most 6.3, at most 5.5 to at most 6.2, at most 5.5 to at most 6.1, at most 5.5 to at most 6.0, at most 5.5 to at most 5.9, at most 5.5 to at most 5.8, at most 5.5 to at most 5.7, at most 5.5 to at most 5.6.

In some embodiments, the pharmaceutical formulation comprising a peptide or pharmaceutically acceptable salt is at a pH value of at most 7.0 to at most 8.0, at most 7.0 to at most 7.9, at most 7.0 to at most 7.8, at most 7.0 to at most 7.7, at most 7.0 to at most 7.6, at most 7.0 to at most 7.5, at most 7.0 to at most 7.4, at most 7.0 to at most 7.3, at most 7.0 to at most 7.2, at most 7.0 to at most 7.1.

In some embodiments, the pharmaceutical formulation comprising a peptide or pharmaceutically acceptable salt and an aqueous carrier is at a pH value of at most 8.0 to at most 13.0, at most 8.1 to at most 12.9, at most 8.2 to at most 12.8, at most 8.3 to at most 12.7, at most 8.4 to at most 12.6, at most 8.5 to at most 12.5, at most 8.6 to at most 12.4, at most 8.7 to at most 12.3, at most 8.8 to at most 12.2, at most 8.9 to at most 12.1, at most 9.0 to at most 12.0, at most 9.1 to at most 11.9, at most 9.2 to at most 11.8, at most 9.3 to at most 11.7, at most 9.4 to at most 11.6, at most 9.5 to at most 11.5, at most 9.6 to at most 11.4, at most 9.7 to at most 11.3, at most 9.8 to at most 11.2, at most 9.9 to at most 11.1, at most 10 to at most 11, at most 10.1 to at most 10.9, at most 10.2 to 10.8, at most 10.3 to 10.7, at most 10.4 to 10.6, at most 10.5.

In some embodiments, the pharmaceutical formulation comprising a peptide or pharmaceutically acceptable salt and an aqueous carrier is at a pH value of at most 5.5 to at most 7.0, at most 5.6 to at most 6.9, at most 5.7 to at most 6.8, at most 5.8 to at most 6.7, at most 5.9 to at most 6.6, at most 6.0 to at most 6.5, at most 6.1 to at most 6.4, at most 6.2 to at most 6.3.

In some embodiments, the pharmaceutical formulation comprising a peptide or pharmaceutically acceptable salt and an aqueous carrier is at a pH value of at most 7.0 to at most 8.0, at most 7.1 to at most 7.9, at most 7.2 to at most 7.8, at most 7.3 to at most 7.7, at most 7.4 to at most 7.6, at most 7.5.

In some embodiments, the pharmaceutical formulation may further comprise a pH adjusting agent, such as hydrochloric acid, sodium hydroxide, ammonium hydroxide, other pH adjusting agents known to those skilled in the art, or combinations thereof to the aqueous carrier. In some embodiments, the pH adjusting agent is hydrochloric acid. In some embodiments, the pH adjusting agent is sodium hydroxide. In some embodiments, the pH adjusting agent is ammonium hydroxide. In some embodiments, the pH adjusting agent is hydrochloric acid, sodium hydroxide, or any combination thereof.

In some embodiments, the pharmaceutical formulation further comprises a pH buffer or pH buffering agent. Non-limiting examples of suitable pH buffers or pH buffering agents includes sodium citrate, citric acid, sodium acetate, acetic acid, phosphoric acid, trisodium phosphate, lactic acid, sodium lactate, tartaric acid, monosodium tartrate, sodium tartrate dibasic, sodium hypochlorite, boric acid, 4-(2-hydroxyethyl)-1-piperazineethanesulfonic acid (HEPES), piperazine-N,N′-bis(2-ethanesulfonic acid) (PIPES), 2-(N-morpholino)ethanesulfonic acid (MES), 1,3-(bis(tris(hydroxymethyl)methylamino)propane (BTP), 3-(N-morphino) propanesulfonic acid (MOPS), 3-[N,N-bis(2-hydroxyethylamino)-2-hydroxy-1-proanesulfonic acid (DIPSO), 4-(N-morphino)butanesulfonic acid (MOBS), 3-[N-Tris(hydroxymethyl)methylamino]-2-hydroxypropanesulfonic acid (TAPSO), HEPPSO, POPSO, TEA, EPPS, Tricine, Glycylglycine, Bicine, HEPBS, TAPS, AMPD, TABs, AMPSO, CHES, CAPSO, AMP, CAPS, CABS, Dakin's solution, diluted bleach, other pH buffers known to those skilled in the art, or combinations thereof. In some embodiments, the pH buffer or pH buffering agent comprises sodium citrate. In some embodiments, the pH buffer or pH buffering agent comprises citric acid. In some embodiments, the pH buffer or pH buffering agent comprises sodium acetate. In some embodiments, the pH buffer or pH buffering agent comprises acetic acid. In some embodiments, the pH buffer or pH buffering agent comprises phosphoric acid. In some embodiments, the pH buffer or pH buffering agent comprises trisodium phosphate. In some embodiments, the pH buffer or pH buffering agent comprises lactic acid. In some embodiments, the pH buffer or pH buffering agent comprises sodium lactate. In some embodiments, the pH buffer or pH buffering agent comprises tartaric acid. In some embodiments, the pH buffer or pH buffering agent comprises monosodium tartrate. In some embodiments, the pH buffer or pH buffering agent comprises sodium tartrate dibasic. In some embodiments, the pH buffer or pH buffering agent comprises 4-(2-hydroxyethyl)-1-piperazineethanesulfonic acid (HEPES). In some embodiments, the pH buffer or pH buffering agent comprises piperazine-N,N′-bis(2-ethanesulfonic acid) (PIPES). In some embodiments, the pH buffer or pH buffering agent comprises 2-(N-morpholino)ethanesulfonic acid (MES). In some embodiments, the pH buffer or pH buffering agent comprises sodium hydrogen phosphate, sodium dihydrogen phosphate, potassium dihydrogen phosphate, potassium hydrogen phosphate, glycine, tris(hydroxymethyl)aminomethane, and any combination thereof. In some embodiments, the pH buffer or pH buffering agent comprises sodium hydrogen phosphate. In some embodiments, the pH buffer or pH buffering agent comprises sodium dihydrogen phosphate. In some embodiments, the pH buffer or pH buffering agent comprises potassium dihydrogen phosphate. In some embodiments, the pH buffer or pH buffering agent comprises potassium hydrogen phosphate. In some embodiments, the pH buffer or pH buffering agent comprises glycine. In some embodiments, the pH buffer or pH buffering agent comprises tris(hydroxymethyl)aminomethane. In some embodiments, the pH buffer comprises a phosphate buffer. In some embodiments, the phosphate buffer comprises Dulbecco's phosphate buffered saline (dPBS).

In some embodiments, the pharmaceutical formulation can be free of a pH buffering agent or pH buffer.

### Pharmaceutically Acceptable Excipients

In some embodiments, a pharmaceutical formulation can comprise a peptide described herein, an aqueous carrier, and further comprise at least one excipient. By “pharmaceutically acceptable”, it is meant that the carrier, diluent, or excipient must be compatible with the other ingredients of the formulation and not deleterious to the recipient thereof. The term “compatible”, as used herein, means that the components of the formulation are capable of being commingled with the subject compound, and with each other, in a manner such that there is no interaction that would substantially reduce the pharmaceutical efficacy of the formulation under ordinary use situations.

In some embodiments, a pharmaceutical formulation can comprise an excipient. An excipient can be an excipient described in the Handbook of Pharmaceutical Excipients, American Pharmaceutical Association (1986).

Non-limiting examples of suitable excipients can include a preservative, a stabilizer, a lubricant, a chelator, a dispersion enhancer, a coloring agent, isotonicity agent, and/or surfactant. In some embodiments, the pharmaceutical formulation further comprises one or more additional pharmaceutically acceptable excipients. See, e.g., Remington: The Science and Practice of Pharmacy (Gennaro, 21st Ed. Mack Pub. Co., Easton, PA (2005) for a list of pharmaceutically acceptable excipients. In some embodiments, the pharmaceutically acceptable excipient is of sufficiently high purity and sufficiently low toxicity to render them suitable for administration preferably to an animal, preferably a mammal, being treated.

In some embodiments, an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments, an excipient can comprise a lubricant. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), and talc or a combination thereof.

In some embodiments, an excipient can comprise a coloring agent. Non-limiting examples of suitable color agents can include food, drug and cosmetic colors (FD&C), drug and cosmetic colors (D&C), and external drug and cosmetic colors (Ext. D&C). A coloring agent can be used as dyes.

In some embodiments, an excipient can comprise an isotonicity agent. Examples can include, but are not limited to: sodium chloride, calcium chloride, potassium chloride, sodium lactate, copper chloride, copper sulfate, monopotassium phosphate, sucrose, dextrose, or glucose. In some embodiments, the isotonicity agent is sodium chloride.

In some embodiments, an excipient can comprise a chelator. In some embodiments, a chelator can be a fungicidal chelator. Examples can include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11] pentatriacontane hexahydrobromide; or triethylenetetramine-N,N,N′,N″,N′″,N′″-hexaacetic acid.

In other embodiments, an excipient can comprise a surfactant. Surfactants can be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

A weight fraction of an excipient or combination of excipients in a pharmaceutical formulation can be less than about 80%, 70%, 60%, 50%, 45%, 40%, 35%, 30%, 25%, 20%, 15%, 10%, 5%, 4%, 3%, 2%, or 1% as compared to a total weight of a pharmaceutical formulation. See, e.g., Remington: The Science and Practice of Pharmacy (Gennaro, 21st Ed. Mack Pub. Co., Easton, PA (2005).

### Methods of Treatment

### Administration

A pharmaceutical formulation disclosed herein can be formulated into a variety of forms and administered by a number of different means.

A pharmaceutical formulation containing a peptide or salt thereof and an aqueous carrier can be administered to a subject in order to at least partially ameliorate a disease or condition. A subject can be in need of a treatment of a disease or condition. In some embodiments, a subject may have been previously diagnosed with a disease or condition described herein, and/or may be at risk of developing a disease or condition as described herein.

Described herein are methods of preventing or treating an infection, wherein the method comprising locally administering of a pharmaceutical formulation described herein to a site of infection, wherein administration comprises washing, irrigating, debriding, aspirating, or a combination thereof the site of infection.

In some embodiments, administration of a pharmaceutical formulation comprising a peptide or pharmaceutically acceptable salt thereof as described and an aqueous carrier prevents or treat an infection to a greater extent compared to administration of the peptide or aqueous carrier alone. In some embodiments, administration of a pharmaceutical formulation results in decrease in bacterial burden, improves survivability of the patient, reduces incidence of abscesses from the infection, or any combination thereof. In some embodiments, a bacterial burden can be an implant bacterial burden, bone bacterial burden, or both (e.g., total bacterial burden). In some embodiments, a bacterial burden can be an implant bacterial burden. In some embodiments, a bacterial burden can be a bone bacterial burden. In some embodiments, a bacterial burden is measured as colony-forming unit per milliliter (CFU/mL) by colony-forming unit analysis.

In some embodiments, the administration of a pharmaceutical formulation described herein can be after a surgical procedure or before, during, or after a care regiment of a surgical procedure (e.g., debridement, antibiotics, and imponent retention (DAIR)). In some embodiments, the administration of a pharmaceutical formulation described herein occurs prior, during or subsequent to a total knee arthroplasty.

In some embodiments, a pharmaceutical formulation comprising a peptide or pharmaceutically acceptable salt thereof as described and an aqueous carrier may be used in a lavage system. The lavage system may comprise an irrigation actuator configured for washing or irrigating a wound in a patient with an irrigation liquid (e.g., a pharmaceutical formulation) and a reservoir comprising an irrigation liquid (e.g., a pharmaceutical formulation). The irrigation actuator and the reservoir are fluidly connected with each other.

Additionally, pharmaceutical formulations can be administered orally, rectally, or parenterally, in formulations containing conventionally acceptable carriers, adjuvants, and vehicles as desired. The term “parenteral” as used herein can include subcutaneous, intravenous, intramuscular, intra-articular, or intrasternal injection and infusion techniques. Administration can include injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, intra-articular, epidural and subcutaneous), transdermal, transmucosal, sublingual, buccal and topical (including epicutaneous, dermal, enema, eye drops, ear drops, intranasal, vaginal) administration. In some exemplary embodiments, a route of administration can be via an injection such as an intramuscular, intravenous, subcutaneous, intra-articular or intraperitoneal injection.

In some embodiments, the pharmaceutical formulation comprising a peptide or pharmaceutically acceptable salt thereof as described and an aqueous carrier can be administered in a formulation for topical administration. For topical administration, an active agent may be formulated as is known in the art for direct application to a target area.

In some embodiments, the method of administration may last over a course of at least about 1 hour, 5 hours, 12 hours, 24 hours, 48 hours, 72 hours, 4 days, 5 days, 1 week, 2 weeks, 3 week, 4 weeks, 2 months, 3 months, 4 months, 5 months, 6 months, 7 months, 8 months, 9 months, 10 months, 11 months, 12 months, 2 years, 3 years, 4 years, 5 years, 6 years, 7 years, 8 years, 9 years, 10 years, 20 years, 25 years, 30 years, 35 years, 40 years, 45 years, 50 years, 55 years, 60 years, 65 years, 70 years, 75 years, or 80 years.

Administration of a pharmaceutical formulation comprising a peptide or pharmaceutically acceptable salt thereof and an aqueous carrier to a subject can be used to at least partially ameliorate a bacterial infection in a subject. Administration of a pharmaceutical formulation comprising a peptide or pharmaceutically acceptable salt thereof and an aqueous carrier can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days.

In some embodiments, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration of a pharmaceutical formulation comprising a peptide or pharmaceutically acceptable salt thereof and an aqueous carrier can be performed at least 1, 2, 3, 4, 5, 6, 7, 8, 9, 10, 11, 12, 13, 14, 15, 16, 17, 18, 19, 20, 21, 22, 23, or 24 times a day. In some embodiments, administration of a pharmaceutical formulation comprising a peptide or pharmaceutically acceptable salt thereof and an aqueous carrier can be performed at least 1, 2, 3, 4, 5, 6, 7, 8, 9, 10, 11, 12, 13, 14, 15, 16, 17, 18, 19, 20, or 21 times a week. In some embodiments, administration of a pharmaceutical formulation comprising a peptide or pharmaceutically acceptable salt thereof and an aqueous carrier can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some embodiments, administration of a pharmaceutical formulation comprising a peptide or pharmaceutically acceptable salt and an aqueous carrier may occurs over a time period of from at least about 0.5 min to at least about 1 min, from at least about 1 min to at least about 2 min, from at least about 2 min to at least about 3 min, from at least about 3 min to at least about 4 min, from at least about 4 min to at least about 5 min, from at least about 5 min to at least about 6 min, from at least about 6 min to at least about 7 min, from at least about 7 min to at least about 8 min, from at least about 8 min to at least about 9 min, from at least about 9 min to at least about 10 min, from at least about 10 min to at least about 11 min, from at least about 11 min to at least about 12 min, from at least about 12 min to at least about 13 min, from at least about 13 min to at least about 14 min, from at least about 14 min to at least about 15 min, from at least about 15 min to at least about 16 min, from at least about 16 min to at least about 17 min, from at least about 17 min to at least about 18 min, from at least about 18 min to at least about 19 min, from at least about 19 min to at least about 20 min, from at least about 21 min to at least about 22 min, from at least about 22 min to at least about 23 min, from at least about 23 min to at least about 24 min, from at least about 24 min to at least about 25 min, from at least about 25 min to at least about 26 min, from at least about 26 min to at least about 27 min, from at least about 27 min to at least about 28 min, from at least about 28 min to at least about 29 min, or from at least about 29 min to at least about 30 min.

### Dosage

In some embodiments, the pharmaceutical formulations described herein is in the form of a unit dose. In some embodiments, a pharmaceutical formulation can be formulated to optimize pharmacokinetics/pharmacodynamics of a peptide or salt thereof contained therein.

In some embodiments, a peptide or pharmaceutically acceptable salt is present at a concentration from at least about 0.01 μg/mL to at least about 100 mg/mL in a pharmaceutical formulation described herein. In some embodiments, a peptide or pharmaceutically acceptable salt is present at a concentration from at least about at least about 0.1 mg/mL to at least about 5 mg/mL in a pharmaceutical formulation described herein. In some embodiments, a peptide or pharmaceutically acceptable salt is present at a concentration from at least about at least about 0.5 mg/mL to at least about 1 mg/mL in a pharmaceutical formulation described herein. In some embodiments, a peptide or pharmaceutically acceptable salt is present at a concentration about 1 mg/mL in a pharmaceutical formulation described herein. In some embodiments, a peptide or pharmaceutically acceptable salt is present at a concentration about 3 mg/mL in a pharmaceutical formulation described herein. In some embodiments, the peptide or pharmaceutically acceptable salt is present at a concentration about 5 mg/mL. In some embodiments, a peptide or pharmaceutically acceptable salt is present at a concentration about 10 mg/mL in a pharmaceutical formulation described herein.

In some embodiments, a pharmaceutical formulation comprising a peptide or salt thereof described herein can be administered at a dose of from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg, wherein the dose is refers to the amount of peptide or pharmaceutically acceptable salt thereof. In some embodiments, a peptide, pharmaceutically acceptable salt thereof, or pharmaceutical formulation comprising a peptide or salt thereof described herein can be administered at a dose of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg, wherein the dose is refers to the amount of peptide or pharmaceutically acceptable salt thereof.

In some embodiments, effective amounts of a peptide or pharmaceutically acceptable salt for treating or preventing an infection in the pharmaceutical formulation as described herein can be a concentration from at least about 0.01 μg/mL to at least about 100 mg/mL. In some embodiments, effective amounts of a peptide or pharmaceutically acceptable salt is at a concentration from at least about at least about 0.1 mg/mL to at least about 5 mg/mL. In some embodiments, effective amounts of a peptide or pharmaceutically acceptable salt is at a concentration from at least about at least about 0.5 mg/mL to at least about 1 mg/mL. In some embodiments, effective amounts of a peptide or pharmaceutically acceptable salt is at a concentration about 1 mg/mL. In some embodiments, effective amounts of a peptide or pharmaceutically acceptable salt is at a concentration about 3 mg/mL. In some embodiments, effective amounts of a peptide or pharmaceutically acceptable salt is at a concentration about 5 mg/mL. In some embodiments, effective amounts of a peptide or pharmaceutically acceptable salt is at a concentration about 10 mg/mL.

In some embodiments, a pharmaceutical formulation comprising a peptide or pharmaceutically acceptable salt and an aqueous carrier can exhibit antimicrobial activity against an infection at a concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some embodiments, a pharmaceutical formulation comprising a peptide or pharmaceutically acceptable salt and an aqueous carrier can exhibit inhibit the growth of a microbe (e.g., bacterium, virus, fungus, parasite) at a inhibitory concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some embodiments, a pharmaceutical formulation comprising a peptide or pharmaceutically acceptable salt and an aqueous carrier can inhibit at least about 10%, about 20%, about 30%, about 40%, about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, or more of the growth of a microbe. In some embodiments, a pharmaceutical formulation comprising a peptide or pharmaceutically acceptable salt can inhibit not more than about 99%, about 98%, about 97%, about 96%, about 95%, about 94%, about 93%, about 92%, about 91%, about 90%, about 89%, about 88%, about 87%, about 86%, about 85%, about 80%, about 70%, about 60%, about 50%, about 40%, about 30%, about 20%, about 10%, or less of the growth of microbe. Inhibition of the growth of a microbe can be measured by, e.g., bacterial analysis comprising, e.g., colony formation unit (CFU) enumeration, agar dilution, broth dilution, or other methods.

In some embodiments, effective amounts of a peptide or pharmaceutically acceptable salt in a pharmaceutical formulation described herein for treating or preventing an infection may be a concentration from at least about 0.01 μg/mL to at least about 0.02 μg/mL, from at least about 0.02 μg/mL to at least about 0.03 μg/mL, from at least about 0.03 μg/mL to at least about 0.04 μg/mL, from at least about 0.04 μg/mL to at least about 0.05 μg/mL, from at least about 0.05 μg/mL to at least about 0.06 μg/mL, from at least about 0.06 μg/mL to at least about 0.07 μg/mL, from at least about 0.07 μg/mL to at least about 0.08 μg/mL, from at least about 0.08 μg/mL to at least about 0.09 μg/mL, from at least about 0.09 μg/mL to at least about 0.1 μg/mL, from at least about 0.1 μg/mL to at least about 0.2 μg/mL, from at least about 0.2 μg/mL to at least about 0.3 μg/mL, from at least about 0.3 μg/mL to at least about 0.4 μg/mL, from at least about 0.4 μg/mL to at least about 0.5 μg/mL, from at least about 0.5 μg/mL to at least about 0.6 μg/mL, from at least about 0.6 μg/mL to at least about 0.7 μg/mL, from at least about 0.7 μg/mL to at least about 0.8 μg/mL, from at least about 0.8 μg/mL to at least about 0.9 μg/mL, from at least about 0.9 μg/mL to at least about 1 μg/mL, from at least about 1 μg/mL to at least about 2 μg/mL, from at least about 2 μg/mL to at least about 3 μg/mL, from at least about 3 μg/mL to at least about 4 μg/mL, from at least about 4 μg/mL to at least about 5 μg/mL, from at least about 5 μg/mL to at least about 6 μg/mL, from at least about 6 μg/mL to at least about 7 μg/mL, from at least about 7 μg/mL to at least about 8 μg/mL, from at least about 8 μg/mL to at least about 9 μg/mL, from at least about 9 μg/mL to at least about 10 μg/mL, from at least about 10 μg/mL to at least about 20 μg/mL, from at least about 20 μg/mL to at least about 30 μg/mL, from at least about 30 μg/mL to at least about 40 μg/mL, from at least about 40 μg/mL to at least about 50 μg/mL, from at least about 50 μg/mL to at least about 60 μg/mL, from at least about 60 μg/mL to at least about 70 μg/mL, from at least about 70 μg/mL to at least about 80 μg/mL, from at least about 80 μg/mL to at least about 90 μg/mL, from at least about 90 μg/mL to at least about 0.1 mg/mL, from at least about 0.1 mg/mL to at least about 0.2 mg/mL, from at least about 0.2 mg/mL to at least about 0.3 mg/mL, from at least about 0.3 mg/mL to at least about 0.4 mg/mL, from at least about 0.4 mg/mL to at least about 0.5 mg/mL, from at least about 0.5 mg/mL to at least about 0.6 mg/mL, from at least about 0.6 mg/mL to at least about 0.7 mg/mL, from at least about 0.7 mg/mL to at least about 0.8 mg/mL, from at least about 0.8 mg/mL to at least about 0.9 mg/mL, from at least about 0.9 mg/mL to at least about 1 mg/mL, from at least about 1 mg/mL to at least about 2 mg/mL, from at least about 2 mg/mL to at least about 3 mg/mL, from at least about 3 mg/mL to at least about 4 mg/mL, from at least about 4 mg/mL to at least about 5 mg/mL, from at least about 5 mg/mL to at least about 6 mg/mL, from at least about 6 mg/mL to at least about 7 mg/mL, from at least about 7 mg/mL to at least about 8 mg/mL, from at least about 8 mg/mL to at least about 9 mg/mL, from at least about 9 mg/mL to at least about 10 mg/mL, from at least about 10 mg/mL to at least about 20 mg/mL, from at least about 20 mg/mL to at least about 30 mg/mL, from at least about 30 mg/mL to at least about 40 mg/mL, from at least about 40 mg/mL to at least about 50 mg/mL, from at least about 50 mg/mL to at least about 60 mg/mL, from at least about 60 mg/mL to at least about 70 mg/mL, from at least about 70 mg/mL to at least about 80 mg/mL, from at least about 80 mg/mL to at least about 90 mg/mL, or from at least about 90 mg/mL to at least about 100 mg/mL.

In some embodiments, effective amounts of a peptide or pharmaceutically acceptable salt in a pharmaceutical formulation described herein for treating or preventing an infection may be from at least about 1 μL to at least about 2 μL, from at least about 2 μL to at least about 3 μL, from at least about 3 μL to at least about 4 μL, from at least about 4 μL to at least about 5 μL, from at least about 5 μL to at least about 6 μL, from at least about 6 μL to at least about 7 μL, from at least about 7 μL to at least about 8 μL, from at least about 8 μL to at least about 9 μL, from at least about 9 μL to at least about 10 μL, from at least about 10 μL to at least about 20 μL, from at least about 20 μL to at least about 30 μL, from at least about 30 μL to at least about 40 μL, from at least about 40 μL to at least about 50 μL, from at least about 50 μL to at least about 60 μL, from at least about 60 μL to at least about 70 μL, from at least about 70 μL to at least about 80 μL, from at least about 80 μL to at least about 90 μL, from at least about 90 μL to at least about 100 μL, from at least about 100 μL to at least about 200 μL, from at least about 200 μL to at least about 300 μL, from at least about 300 μL to at least about 400 μL, from at least about 400 μL to at least about 500 μL, from at least about 500 μL to at least about 600 μL, from at least about 600 μL to at least about 700 μL, from at least about 700 μL to at least about 800 μL, from at least about 800 μL to at least about 900 μL, from at least about 900 μL to at least about 1 mL, from at least about 1 mL to at least about 2 mL, from at least about 2 mL to at least about 3 mL, from at least about 3 mL to at least about 4 mL, from at least about 4 mL to at least about 5 mL, from at least about 5 mL to at least about 6 mL, from at least about 6 mL to at least about 7 mL, from at least about 7 mL to at least about 8 mL, from at least about 8 mL to at least about 9 mL, from at least about 9 mL to at least about 10 mL, from at least about 10 mL to at least about 20 mL, from at least about 20 mL to at least about 30 mL, from at least about 30 mL to at least about 40 mL, from at least about 40 mL to at least about 50 mL, from at least about 50 mL to at least about 60 mL, from at least about 60 mL to at least about 70 mL, from at least about 70 mL to at least about 80 mL, from at least about 80 mL to at least about 90 mL, from at least about 90 mL to at least about 100 mL, from at least about 100 mL to at least about 200 mL, from at least about 200 mL to at least about 300 mL, from at least about 300 mL to at least about 400 mL, from at least about 400 mL to at least about 500 mL, from at least about 500 mL to at least about 600 mL, from at least about 600 mL to at least about 700 mL, from at least about 700 mL to at least about 800 mL, from at least about 800 mL to at least about 900 mL, from at least about 900 mL to at least about 1 L, from at least about 1 L to at least about 2 L, from at least about 2 L to at least about 3 L, from at least about 3 L to at least about 4 L, from at least about 4 L to at least about 5 L, from at least about 5 L to at least about 6 L, from at least about 6 L to at least about 7 L, from at least about 7 L to at least about 8 L, from at least about 8 L to at least about 9 L, from at least about 9 L to at least about 10 L, from at least about 10 L to at least about 20 L, from at least about 20 L to at least about 30 L, from at least about 30 L to at least about 40 L, from at least about 40 L to at least about 50 L, from at least about 50 L to at least about 60 L, from at least about 60 L to at least about 70 L, from at least about 70 L to at least about 80 L, from at least about 80 L to at least about 90 L, from at least about 90 L to at least about 100 L, from at least about 100 L to at least about 200 L, from at least about 200 L to at least about 300 L, from at least about 300 L to at least about 400 L, from at least about 400 L to at least about 500 L, from at least about 500 L to at least about 600 L, from at least about 600 L to at least about 700 L, from at least about 700 L to at least about 800 L, from at least about 800 L to at least about 900 L, from at least about 900 L to at least about 1 kL, from at least about 1 kL to at least about 2 kL, from at least about 2 kL to at least about 3 kL, from at least about 3 kL to at least about 4 kL, from at least about 4 kL to at least about 5 kL, from at least about 5 kL to at least about 6 kL, from at least about 6 kL to at least about 7 kL, from at least about 7 kL to at least about 8 kL, from at least about 8 kL to at least about 9 kL, or from at least about 9 kL to at least about 10 kL.

### Combination Administration

Also contemplated are combination products that include one or more peptides disclosed herein in a pharmaceutical formulation described herein and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein.

In addition, it is contemplated that a peptide can be combined with topical antifungal agents such as ciclopirox olamine, haloprogin, tolnaftate, undecylenate, topical nysatin, amorolfine, butenafine, naftifine, terbinafine, and other topical agents. In some embodiments, a pharmaceutical formulation can comprise an additional agent. In some embodiments, an additional agent can be present in a therapeutically effective amount in a pharmaceutical formulation. In some embodiments, an additional pharmaceutical agent can be an antibiotic agent. An antibiotic agent can of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides and tetracycline. Alternatively, or additionally an antibiotic agent may be effective against mycobacteria. In some embodiments, an antibiotic agent may be an aminoglycoside such as Amikacin, Gentamicin, Kanamycin, Neomycin, Netilmicin, Tobramycin or Paromomycin. According to one embodiment, an antibiotic agent may be an Ansamycin such as Geldanamycin and Herbimycin. In some embodiments, an antibiotic agent may be a carbacephem such as Loracarbef. In some embodiments, an antibiotic agent can be a carbapenem such as Ertapenem, Doripenem, Imipenem/Cilastatin or Meropenem.

In some embodiments, an antibiotic agent may be a beta lactam antibiotic or pharmaceutically acceptable salt thereof may include but are not limited to Cephalexin, Cephradine, Cefadroxil, Cefazolin, B-lactam antibiotic C, Cephalothin, Cefapirin, Cefuroxime, Cefprozil, Loracarbef, Cefuroxime, Cefoxitin, Cefotetan, Cefaclor, Cefamandole, Ceftriaxone, Cefdinir, Cefixime, Cefpodoxime, Cefditoren, Ceftibuten, Ceftazidime, Cefotaxime, Cefoperazone, Ceftizoxime, Cefepime, Cefiderocol, Cefpirome, Ceftaroline, Benzathine, Benzylpenicillin, Phenoxymethylpenicillin, Procaine penicillin, Pheneticillin, Cloxacillin, Dicloxacillin, Flucloxacillin, Methicillin, Nafcillin, Oxacillin, Temocillin, Amoxillin, Ampicillin, Mecillinam, Piperacillin, Carbenicillin, Ticarillin, Carbenicillin, Ticarcillin, Azlocillin, Mezlocillin, Piperacillin, Biapenem, Doripenem, Ertapenem, Faropenem, Imipenem, Meropenem, Panipenem, Razupenem, Tebipenem, Thienamycin, Aztreonam, Tigermonam, Nocardicin A, Tabtoxinine beta-lactam, Clavulanic acid, Tazobactam, Sulbactam, or Avibactam. In some embodiments, an antibiotic agent may be a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. Alternatively, an antibiotic agent may be a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime and Ceftobiprole. In some embodiments, an antibiotic agent may be a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin. In some embodiments, an antibiotic agent may be a monobactams such as Aztreonam, or a nitrofuran such as Furazolidone or Nitrofurantoin. In some embodiments, an antibiotic agent may be a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin. In some embodiments, an antibiotic agent may be a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, and Trimethoprim-Sulfamethoxazole (Co-trimoxazole) (TMP-SMX). In some embodiments, an antibiotic agent may be a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin. In some embodiments, an antibiotic agent may be a polypeptide such as Bacitracin, Colistin and Polymyxin B. In some embodiments, an antibiotic agent may be a tetracycline such as Demeclocycline, Doxycycline, Minocycline and Oxytetracycline. In some embodiments, an antibiotic agent may be effective against mycobacteria. An antibiotic agent may be Clofazimine, Lamprene, Dapsone, Capreomycin, Cycloserine, Ethambutol, Ethionamide, Isoniazid, Pyrazinamide, Rifampicin, Rifabutin, Rifapentine or Streptomycin. In some exemplary embodiments, an antibiotic agent can include Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Methicillin, Linezolid, a Streptogramin, Tigecycline, Daptomycin, a salt of any of these, or any combination thereof. In some embodiments, a pharmaceutical formulations further comprises an antibiotic. In some embodiments, the antibiotic is cefazolin.

In some embodiments, an additional pharmaceutical agent can be an antimicrobial agent disclosed herein. In some embodiments, an antimicrobial agent can be cysteamine or a salt thereof. While cysteamine can be typically used to treat conditions such as cystinosis that are not derived from an infection, the use of cysteamine as an antimicrobial compound has shown promise. For example, WO2010112848 describes the use of formulations containing cysteamine for as antimicrobial agents capable of inhibiting the formation of a bacterial biofilm for a broad range of bacterial strains, including Pseudomonas spp., Staphylococcus spp., Haemophilus spp., Burkholderia spp., Streptococcus spp., Propionibacterium spp.

In some embodiments, an additional pharmaceutical agent can be an antiviral agent. In some embodiments, an antiviral agent can be Acyclovir, Brivudine, Cidofovir, Docosanol, Famciclovir, Foscarnet, Fomivirsen, Ganciclovir, Idoxuridine, Penciclovir, Peramivir, Trifluridine, Valacyclovir, Vidarabine, Lamivudine, Ribavirin Amantadine, Rimantadine, a neuraminidase inhibitor, Oseltamivir, Zanamivir, a salt of any of these, or any combination thereof.

In some embodiments, an additional pharmaceutical agent can be an antineoplastic. In some embodiments, an antineoplastic can be selected from the group consisting of cyclophosphamide, methotrexate, 5-fluorouracil, doxorubicin, procarbazine, prednisolone, bleomycin, vinblastine, dacarbazine, cisplatin, epirubicin, a salt of any of these, and any combination thereof.

In some embodiments, a pharmaceutical formulation containing a peptide or pharmaceutically acceptable salt thereof and an aqueous carrier can be an antiviral agent.

In some embodiments, a pharmaceutical formulation containing a peptide or pharmaceutically acceptable salt thereof and an aqueous carrier can be administered in combination with an antibiotic or an additional antiviral agent disclosed herein. In some embodiments, a pharmaceutical formulation described herein is administered at different times and/or different routes of administration with an antibiotic. In some embodiments, a pharmaceutical formulation described herein is administered at the same time and/or same route of administration with an antibiotic.

### Infection

Provided herein are pharmaceutical formulations comprising a peptide and an aqueous carrier and method of treating or preventing a disease or condition comprising administering the pharmaceutical formulation. In some embodiments, the condition or disease is an infection. In other embodiments, the infection is a microbial infection. In some embodiments, the infection is a bacterial infection, viral infection, fungal infection, or a combination thereof. In some embodiments, a bacterial species is isolated from a subject that is suffering an infection. In some embodiments, the term bacterial burden may refer to the amount of bacteria or the microbial load from a bacterial infection. The pharmaceutical composition comprising a peptide described herein and an aqueous carrier may reduce the bacterial burden in a bacterial infection. In some embodiments, the infection is periprosthetic joint infection (PJI).

In some embodiments, bacterial infection may be derived from a bacterial species selected from the group, but not exclusive to the group, consisting of: Staphylococcus spp., e.g. Staphylococcus aureus (e.g. Staphylococcus aureus NCTC 10442 and Staphylococcus aureus ATCC25923), Staphylococcus epidermidis; Chlamydia spp., e.g. Chlamydia trachomatis, Chlamydia pneumoniae, Chlamydia psittaci; Enterococcus spp., e.g. Enterococcus faecalis; Streptococcus pyogenes; Listeria spp.; Pseudomonas spp.; Mycobacterium spp., e.g. Mycobacterium tuberculosis complex; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori, Helicobacter felis; Neisseria spp., e.g. Neisseria gonorrhoea, Neisseria meningitidis; Borrelia burgdorferi; Shigella spp., e.g. Shigella flexneri; Escherichia coli (E. coli 0157:H7 NCTC 12900); Haemophilus spp., e.g. Haemophilus influenzae; Francisella tularensis; Bacillus spp., e.g. Bacillus anthraces; Clostridia spp., e.g. Clostridium botulinum, Clostridium difficile; Yersinia spp., e.g. Yersinia pestis; Treponema spp.; Burkholderia spp., e.g. Burkholderia cepacia complex, B. mallei, B pseudomallei; Propionibacterium spp., e.g. P. acnes, Acinetobacter species, an Actinomyces species, a Campylobacter species, a Candida species, Corynebacterium minutissium, Corynebacterium pseudodiphtheriae, Corynebacterium stratium, Corynebacterium group G1, Corynebacterium group G2, Enterobacteriaceae, an Enterococcus species, Klebsiella pneumoniae, a Moraxella species, a non-tuberculous mycobacteria species, a Porphyromonas species, Prevotella melaninogenicus, Salmonella typhimurium, Serratia marcescens Streptococcus agalactiae, Staphylococcus salivarius, Streptococcus mitis, Streptococcus sanguis, Streptococcus pneumoniae, Vibrio cholerae, a Coccidioides species, or a Cryptococcus species. In some embodiments, a peptide or pharmaceutically acceptable salt thereof described herein can reduce infection of bacteria against at least one of Staphylococcus aureus, methicillin resistant Staphylococcus aureus, Streptococcus pneumonia, carbapenem-resistant Enteroacteriaceae, Staphylococcus epidermidis, Staphylococcus salivarius, Corynebacterium minutissium, Corynebacterium pseudodiphtheriae, Corynebacterium stratium, Corynebacterium group G1, Corynebacterium group G2, Streptococcus pneumonia, Streptococcus mitis, Streptococcus sanguis, Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaninogenicus, Helicobacter pylori, Helicobacter felis, or Campylobacter jejuni. In some embodiments, bacterial infection may be derived from a bacterial species selected from the group Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, and any combination thereof. In some embodiments, the bacterial may be antibiotic-tolerant or antibiotics-resistant. A bacterial strain can also be an antibiotic-resistant variant or a bacterial strain described herein. In some embodiments, a bacterial strain can be resistant to an antibiotic described herein. In some embodiments, a bacterial strain can be resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or any combination thereof.

In some embodiments, a bacterial infection can arise from a wound, surgical procedure, an implanted medical device or a prosthesis, a biological transplant, or any other cause of infection known to those skilled in the art. In some embodiments, the bacterial infection from an implanted device or prosthesis occurs at the location of the implanted device or prosthesis. In some embodiments, an implanted device or prosthesis can lead to periprosthetic joint infection (PJI). In some embodiments the prosthetic joint infection is first stage periprosthetic joint infection or second stage periprosthetic joint infection. In some embodiments, the bacterial infection is a periprosthetic joint infection. In some embodiments, the prosthesis is knee prosthesis, shoulder prosthesis, hip prosthesis, elbow prosthesis, ankle prosthesis, wrist prosthesis, or spine prosthesis. In some embodiments, the infection is a shoulder infection, knee infection, acute infection, chronic infection, or any combination thereof. In some embodiments, a bacterial infection can lead to a biofilm. In some embodiments, the bacterial infection is caused by antibiotic-tolerant bacteria.

In some embodiments, the site of infection does not comprise a prosthesis.

A microbial biofilm, also referred to as a biological biofilm, can be a community of microbial cells embedded in an extracellular matrix of polymeric substances and adherent to a biological or a non-biotic surface. A range of microorganisms (bacteria, fungi, and/or protozoa, with associated bacteriophages and other viruses) can be found in these biofilms. Biofilms are ubiquitous in nature, are commonly found in a wide range of environments. Biofilms are being increasingly recognized by the scientific and medical community as being implicated in many infections, and especially their contribution to the recalcitrance of infection treatment. Biofilms can be etiologic agents for a number of disease states in mammals and are involved in 80% of infections in humans. Examples can include skin and wound infections, middle-ear infections, gastrointestinal tract infections, peritoneal membrane infections, urogenital tract infections, oral soft tissue infections, formation of dental plaque, eye infections (including contact lens contamination), endocarditis, infections in cystic fibrosis, and infections of indwelling medical devices such as joint prostheses, dental implants, catheters and cardiac implants. Microbes in biofilms can be significantly more resistant to antimicrobial treatment than their planktonic counterparts. Biofilm formation is not limited solely to the ability of microbes to attach to a surface. Microbes growing in a biofilm can interact more between each other than with the actual physical substratum on which the biofilm initially developed. The suggested mechanisms by which biofilm-associated microorganisms elicit diseases in their host can include the following: (i) delayed penetration of the antimicrobial agent through the biofilm matrix, (ii) detachment of cells or cell aggregates from indwelling medical device biofilms, (iii) production of endotoxins, (iv) resistance to the host immune system, (v) provision of a niche for the generation of resistant organisms through horizontal gene transfer of antimicrobial resistance &/or virulence determinant genes, and (vi) altered growth rate (i.e. metabolic dormancy).

In some embodiments, bacteria, fungi, and/or protozoa, with associated bacteriophages and other viruses described herein can secrete a biofilm. In some embodiments, bacteria, fungi, and/or protozoa, with associated bacteriophages and other viruses described herein can form a biofilm. A pharmaceutical formulation comprising a peptide or salt thereof described herein and an aqueous carrier can be administered to at least partially penetrate, inhibit formation of, or destroy a biological biofilm. In some embodiments, additional agents can be added to at least partially inhibit formation of, or destroy, a biological biofilm.

In some embodiments, the infection is a viral infection. In some embodiments, a virus can be a DNA virus, a RNA virus, or a reverse transcriptase (retro) virus. A virus can be a dsDNA (double stranded DNA) virus, a ssDNA (single stranded DNA) virus, a dsRNA (double stranded RNA) virus, a +ssRNA (+strand or sense single stranded RNA) virus, a -ssRNA (-strand or antisense RNA) virus, a ssRNA-RT (single stranded RNA reverse transcriptase) virus, or a dsDNA-RT (double stranded DNA reverse transcriptase) virus. As described herein, a peptide described herein can be engineered to disrupt the integrity of a viral envelope of an enveloped virus. Such a disruption can at least partially reduce a viability of a virus, which can ameliorate an infection brought about by a virus. A virus may be derived from the group, but not exclusive to the group, of a herpesvirus, a poxvirus, a hepadnavirus, a flavivirus, a togavirus, a coronavirus, hepatitis C, hepatitis D, an orthomyxovirus, a papillomavirus, a polyomaviridae, a parvovirus, a cytomegalovirus, an Epstein-Barr virus, a small pox virus, a cow pox virus, a sheep pox virus, an orf virus, a monkey pox virus, a vaccinia virus, a paramyxovirus, a retrovirus, an adenovirus, a rhabdovirus, a bunyavirus, a filovirus, an alphavirus, an arenavirus, a lentivirus, and any combination thereof. In some embodiments, the virus can be an enveloped virus. Examples of an enveloped viruses can include: a poxvirus, a hepadnavirus, a flavivirus, a togavirus, a coronavirus, hepatitis C, hepatitis D, an orthomyxovirus, a cytomegalovirus, an Epstein-Barr virus, a small pox virus, a cow pox virus, a sheep pox virus, an orf virus, a monkey pox virus, a vaccinia virus, a rhabdovirus, a bunyavirus, a filovirus, an alphavirus, an arenavirus, a lentivirus, and the like.

Also envisaged are treatments of fungal, protozoal, or other parasitic infections by administration of a peptide described herein, salt thereof, or formulation containing a peptide or salt thereof. In some embodiments, a pathogen can be a drug-resistant fungal, protozoal, or other parasitic organism.

A parasitic pathogen may be derived from a parasite selected from, but not limited to, the group consisting of Trypanosoma spp. (Trypanosoma cruzi, Trypansosoma brucei), Leishmania spp., Giardia spp., Trichomonas spp., Entamoeba spp., Naegleria spp., Acanthanioeba spp., Schistosoma spp., Plasmodium spp., Crytosporidium spp., Isospora spp., Balantidium spp., Loa Loa, Ascaris lumbricoides, Dirofilaria immitis, and Toxoplasma ssp., e.g., Toxoplasma gondii.

A fungal pathogen may be derived from a fungus (including yeast) selected from, but not limited to, the genera Candida spp., (e.g. C. albicans), Epidermophyton spp., Exophiala spp., Microsporum spp., Trichophyton spp., (e.g. T. rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremoniwn spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphiwn spp., Leptosphaeria spp., Malassezia spp. (e.g Malassezia Furfur), Mucor spp., Neotestudina spp., Nocardia spp., Nocardiopsis spp., Paecilomyces spp., Phoma spp., Piedraia spp., Pneunwcystis spp., Pseudallescheria spp., Pyrenochaeta spp., Rhizoinucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., and Wangiella spp.

A fungal, bacterial, or viral infection may be a systemic, topical, subcutaneous, cutaneous or mucosal infection. Topical fungal infections of nails and skin are generally caused by detinatophytes although some non-dermatophytes such as yeast can also cause skin infections. A dermatophyte infection may include a Tinea infection for example Tinea barbae (beard), Tinea capitis (head), Tinea corporis (body), Tinea cruris (groin), Tinea faciei (face), Tinea manuum (hand), Tinea pedis (foot) Tinea unguium (nail), Tinea (Pityriasis) versicolor, Tinea incognito or Tinea nigra. An infection may be derived from fungi of the genera Epidermophyton, Microsporum or Trichophyton spp. (e.g., T. rubrum and T interdigitale). Exemplary dermatophytes can include Epidermophyton floccosum, Microsporum canis, Microsporum audouinii, Microsporum gypseum, Microsporum nanum, Microsporum ferrugineum, Microsporum distortum, Microsporum fulvum, Trichophyton rubrum, Trichophyton tnentagrophytes var. interdigitale, Trichophyton mentagrophytes var. nodulare, Trichophyton tonsurans, Trichophyton soudanese, Trichophyton violaceum, Trichophyton megnini, Trichophyton schoenlenii, Trichophyton gallinae, Trichophyton krajdenii, Trichophyton yaoundei, Trichophyton equinum, Trichophyton erinacei and Trichophyton verrucosum. In some embodiments, a dermatophytic infection can be onychomycosis. The term “onychomycosis” can include, but is not limited to, distal lateral subungual, superficial white, proximal white subungual, secondary dystrophic, primary dystrophic, endonyx, candidal (e.g., onycholysis & chronic mucocutaneous disease) types of onychomycosis and Tinea ungium. Non-dermatophytic fungi associated with onychomycosis can include Aspergillus spp., Cephalosporum spp., Fusarium oxysporum, Scopularis brevicaulis, and Scytalidium spp.

In some embodiments, the pharmaceutical composition disclosed herein for treating a microbial film may lead to at least about 10% reduction in bacterial burden, at least about 20% reduction in bacterial burden, at least about 30% reduction in bacterial burden, at least about 40% reduction in bacterial burden, at least about 50% reduction in bacterial burden, at least about 60% reduction in bacterial burden, at least about 70% reduction in bacterial burden, at least about 80% reduction in bacterial burden, or at least about 90% reduction in bacterial burden and any increments therebetween. In some embodiments, the antibiotic disclosed herein for treating a microbial film may lead to at least about 10% reduction in bacterial burden, at least about 20% reduction in bacterial burden, at least about 30% reduction in bacterial burden, at least about 40% reduction in bacterial burden, at least about 50% reduction in bacterial burden, at least about 60% reduction in bacterial burden, at least about 70% reduction in bacterial burden, at least about 80% reduction in bacterial burden, or at least about 90% reduction in bacterial burden and any increments therebetween. In some embodiments, the pharmaceutical composition comprising a peptide disclosed herein administered with the antibiotic for the method of treating a microbial film may lead to at least about 10% reduction in bacterial burden, at least about 20% reduction in bacterial burden, at least about 30% reduction in bacterial burden, at least about 40% reduction in bacterial burden, at least about 50% reduction in bacterial burden, at least about 60% reduction in bacterial burden, at least about 70% reduction in bacterial burden, at least about 80% reduction in bacterial burden, or at least about 90% reduction in bacterial burden and any increments therebetween. In some embodiments, the antibiotic disclosed herein for treating a microbial film may lead to at least about 1 log reduction in bacterial burden, about 2 log reduction in bacterial burden, about 3 log reduction in bacterial burden, about 4 log reduction in bacterial burden, about 5 log reduction in bacterial burden, about 6 log reduction in bacterial burden, 7 log reduction in bacterial burden, about 8 log reduction in bacterial burden, 9 log reduction in bacterial burden, or about 10 log reduction in bacterial burden. In some embodiments, the bacterial burden may be measured by colony forming units (CFU) analysis, which may involve taking a sample from a microbial film, diluting the sample, growing cell cultures from the sample on a Petri dish for a predetermined amount of time, and counting the number of colonies formed. In some embodiment, the pharmaceutical composition comprising a peptide disclosed herein for treating a microbial film may lead partially disrupts or destroys a microbial film. In some embodiment, the antibiotic disclosed herein for treating a microbial film may lead partially disrupts or destroys a microbial film. In some embodiment, the pharmaceutical composition disclosed herein for treating a microbial film may lead partially disrupts or destroys a microbial biofilm.

In some embodiments, the peptide disclosed herein or pharmaceutically acceptable salt thereof may reduce the mass of the microbial biofilm. In some embodiments, the peptide disclosed herein or pharmaceutically acceptable salt thereof may lead to at least about 0.5%, 1%, 2%, 3%, 4%, 5%, 6%, 7%, 8%, 9%, 10%, 12.5%, 15%, 17.5%, 20%, 22.5%, 25%, 27.5%, 30%, 32.5%, 35%, 37.5%, 40%, 42.5%, 45%, 47.5%, 50%, 60%, 70%, 80%, 90%, 91%, 92%, 93%, 94%, 95%, 96%, 97%, 98%, or 99% reduction in mass and any increments of percentage therebetween.

### Kits

Disclosed herein are kits. A kit can comprise a peptide or pharmaceutically acceptable salt thereof as described herein in a container and an aqueous carrier in a second container. In some embodiments, the aqueous carrier in a kit is sodium bicarbonate. In some embodiments, the kit further comprises a third container containing water. In some embodiments, a kit further comprises a mixing container. In some embodiments, the mixing container is an intravenous (IV) bag, a lavage bottle, or a joint irrigation system. In some embodiments, the peptide or pharmaceutically acceptable salt thereof in the container is at a concentration of about 0.1 mg/mL to about 100 mg/mL. In some embodiments, the peptide or pharmaceutically acceptable salt thereof in the container is at a concentration of about 15 mg/mL, about 30 mg/mL, about 40 mg/mL, about 70 mg/mL, or about 80 mg/mL. In some embodiments, the peptide or pharmaceutically acceptable salt thereof in the container has a pH of about 3 to about 7. In some embodiments, the peptide or pharmaceutically acceptable salt thereof in the container has a pH of about 5. In some aspects, a kit can further comprise instructions that direct administration of a unit dose of a pharmaceutical formulation to a subject. In some aspects, a kit can comprise instructions for the use thereof.

Methods of making a kit can include placing a peptide or pharmaceutically acceptable salt thereof in a first container, placing an aqueous carrier (e.g., sodium bicarbonate) in a second container, placing water in a third container, and including a mixing container. In some embodiments, the method further comprises adding the first container, the second container, and the third container into the mixing container, thereby resulting in the pharmaceutical formulation.

A method can further comprise an inclusion of instructions for use. In some embodiments, instructions for use can direct administration of a unit dose of a pharmaceutical formulation to a subject.

### Terminology

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, the terms “biofilm”, “microbial film”, “microbial biofilm”, “bacterial film”, refers to any film comprising microorganisms and their excretions.

As used herein, the terms “comprising,” “comprise” or “comprised,” and variations thereof, in reference to elements of an item, formulation, apparatus, method, process, system, claim etc. are intended to be open-ended, meaning that the item, formulation, apparatus, method, process, system, claim etc. includes those elements and other elements can be included and still fall within the scope/definition of the described item, formulation, apparatus, method, process, system, claim etc. As used herein, “a” or “an” means one or more. As used herein “another” may mean at least a second or more.

As used herein, the term “object” refers to any object with a surface. Some embodiments in the present disclosure may be applied to the surface of an object to prevent or to treat microbial biofilm. In some embodiments, the object can a solid object, a liquid object, a hard object, a soft object, a metallic object, a polymeric object, a ceramic object, a composite object, a biological object, members of the animal kingdom, a human being, a biological transplant object, a replaced joint, or any other object with a surface on which some of the disclosed methods and the formulations can be applied.

As used herein, the terms “patient” or “subject” generally refer to any individual that has, may have, or may be suspected of having a disease condition (e.g., a bacterial infection). In some embodiments, the bacterial infection may be caused by surgeries, physical wounds, etc. The subject may be an animal. The animal can be a mammal, such as a human, non-human primate, a rodent such as a mouse or rat, a dog, a cat, pig, sheep, or rabbit. Animals can be fish, reptiles, or others. Animals can be neonatal, infant, adolescent, or adult animals. The subject may be a living organism. The subject may be a human. Humans can be greater than or equal to 1, 2, 5, 10, 20, 30, 40, 50, 60, 65, 70, 75, 80 or more years of age. A human may be from about 18 to about 90 years of age. A human may be from about 18 to about 30 years of age. A human may be from about 30 to about 50 years of age. A human may be from about 50 to about 90 years of age. The subject may have one or more risk factors of a condition and be asymptomatic. The subject may be asymptomatic of a condition. The subject may have one or more risk factors for a condition. The subject may be symptomatic for a condition. The subject may be symptomatic for a condition and have one or more risk factors of the condition. The subject may have or be suspected of having a disease, such as an infection. The subject may be a patient being treated for a disease, such as an infection. The subject may be predisposed to a risk of developing a disease such as a bacterial infection. The subject may be in remission from a disease, such as a bacterial infection. The subject may not have a bacterial infection. The subject may be healthy.

As used herein, a “pharmaceutically acceptable excipient”, “aqueous carrier” or “pharmaceutically acceptable aqueous carrier” refer to solvents or dispersion media, and the like, that are physiologically compatible and known to those skilled in the art. Examples of pharmaceutically acceptable carriers include one or more of water, saline, phosphate buffered saline, dextrose, glycerol, ethanol, and the like, as well as combinations thereof. Pharmaceutically acceptable carriers may further comprise minor amounts of auxiliary substances such as wetting or emulsifying agents, preservatives or buffers, which enhance the shelf life or effectiveness of the active agent.

As used herein, a “effective amount” of an active agent can refer to an amount that is effective to achieve a desired result. An effective amount of a given active agent can vary with respect to factors such as the type and severity of the disorder or disease being treated and the age, gender, and weight of the patient.

The term “homology” can refer to a % identity of a polypeptide to a reference polypeptide. As a practical matter, whether any particular polypeptide can be at least 50%, 60%, 70%, 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters can be set such that the percentage of identity is calculated over the full length of the reference amino acid sequence and that gaps in homology of up to 5% of the total number of amino acid residues in the reference sequence are allowed.

For example, in a specific embodiment the identity between a reference sequence (query sequence, i.e., a sequence of the present invention) and a subject sequence, also referred to as a global sequence alignment, may be determined using the FASTDB computer program based on the algorithm of Brutlag et al. (Comp. App. Biosci. 6:237-245 (1990)). In some embodiments, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some embodiments, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 amino acid residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

The terms “co-administration”, “administered in combination with” and their grammatical equivalents or the like, as used herein, can encompass administration of selected therapeutic agents to a subject, and can include treatment regimens in which agents are administered by the same or different route of administration or at the same or different times. In some embodiments, a peptide disclosed herein can be co-administered with other agents. These terms can encompass administration of two or more agents to a subject so that both agents and/or their metabolites are present in the subject at the same time. They can include simultaneous administration, administration at different times, and/or administration in a formulation in which both agents are present. Thus, in some embodiments, a peptide and an additional agent(s) can be administered in a single formulation. In some embodiments, a peptide and an additional agent(s) can be admixed in the formulation. In some embodiments, a same peptide or agent can be administered via a combination of different routes of administration. In some embodiments, each agent administered can be in a therapeutically effective amount.

As used herein, the term “room temperature” or “monitored room temperature” may be defined as about 20° C. to about 22° C.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Activity of Peptide Formulations Against Isolates Commonly Found in Periprosthetic Joint Infections (PJI)

A formulation of peptide SEQ ID NO:1 was evaluated by broth microdilution against 104 isolates of Staphylococcus epidermidis, 53 other coagulase-negative staphylococci (CoNS), 3 S. aureus and 66 Gram-negative isolates consisting of Enterobacterales, Pseudomonas aeruginosa, and Acinetobacter baumannii.

Imipenem, levofloxacin, tigecycline, linezolid, vancomycin, oxacillin, ceftazidime, colistin, and amikacin were tested as comparators. Testing was conducted in accordance with guidelines from the Clinical and Laboratory Standards Institute (CLSI; M7 and M100). Test organisms comprised reference strains from the American Type Culture Collection, the Centers for Disease Control Antibiotic Reference Bank and clinical isolates from the Micromyx repository. The media employed for testing in the broth microdilution MIC assay for all organisms were cation-adjusted Mueller Hinton Broth and for the formulation of SEQ ID NO:1 only included RPMI-1640 medium supplemented with 0.002% P-80.

TABLE 2
SEQ ID NO: 1 in RPMI against CoNS and
resistant Gram-negative* pathogens.

| | MIC₅₀ | MIC₉₀ |
|---|---|---|
| | (μg/mL) | (μg/mL) |
| All S. epidermidis (N = 104) | 0.12 | 0.12 |
| MSSE (N = 46) | 0.12 | 0.12 |
| MRSE (N = 58) | 0.12 | 0.12 |
| CoNS non-epidermidis (N = 53) | 0.06 | 0.06 |
| S. aureus (N = 3) | 0.12 | 0.12 |
| Enterobacterales (N = 22) | 0.5 | 0.5 |
| P. aeruginosa (N = 20) | 1 | 1 |
| A. baumannii (N = 24) | 0.25 | 0.25 |

Abbreviations: MSSE, methicillin-sensitive Staphylococcus epidermidis; MRSE, methicillin-resistant Staphylococcus epidermidis.
*Approximately 90% of the Gram-negative isolates tested were carbapenem-resistant.

The formulation of SEQ ID NO: 1 was found to have potent antimicrobial activity when evaluated in RPMI against S. epidermidis, CoNS non-epidermidis, S. aureus, Enterobacterales, P. aeruginosa, and A. baumannii, including isolates with multi-drug resistance.

### Example 2: Osmolality Analysis of Peptide Formulations

Formulations of the peptide corresponding to SEQ ID NO: 1 comprising sodium bicarbonate solutions were tested for osmolality. Formulations tested vary based on strength (concentration of peptide in formulation) as well as concentration of sodium bicarbonate. The formulations and corresponding measured osmolalities in mOsmol/kg are listed in Table 3.

TABLE 3
Osmolality of SEQ ID NO: 1 formulations in sodium bicarbonate.

| Description | Strength | mOsmol/kg |
|---|---|---|
| 10 mg SEQ ID NO: 1 w/200 mM NaHCO₃ | 10 mg/mL | 373 |
| | in 3 mL | |
| 10 mg SEQ ID NO: 1 w/150 mM NaHCO₃ | 10 mg/mL | 299 |
| | in 3 mL | |
| 10 mg SEQ ID NO: 1 w/100 mM NaHCO₃ | 10 mg/mL | 207 |
| | in 3 mL | |
| 10 mg SEQ ID NO: 1 w/50 mM NaHCO₃ | 10 mg/mL | 123 |
| | in 3 mL | |
| 3 mg SEQ ID NO: 1 w/200 mM NaHCO₃ | 3 mg/mL | 350 |
| | in 3 mL | |
| 3 mg SEQ ID NO: 1 w/150 mM NaHCO₃ | 3 mg/mL | 277 |
| | in 3 mL | |
| 3 mg SEQ ID NO: 1 w/100 mM NaHCO₃ | 3 mg/mL | 188 |
| | in 3 mL | |
| 3 mg SEQ ID NO: 1 w/50 mM NaHCO₃ | 3 mg/mL | 115 |
| | in 3 mL | |
| 10 mg SEQ ID NO: 1 w/200 mM NaHCO₃ | 1 mg/mL | 345 |
| | in 3 mL | |
| 10 mg SEQ ID NO: 1 w/150 mM NaHCO₃ | 1 mg/mL | 269 |
| | in 3 mL | |
| 10 mg SEQ ID NO: 1 w/100 mM NaHCO₃ | 1 mg/mL | 181 |
| | in 3 mL | |
| 10 mg SEQ ID NO: 1 w/50 mM NaHCO₃ | 1 mg/mL | 92 |
| | in 3 mL | |
| 10 mg SEQ ID NO: 1 w/200 mM NaHCO₃ | 0 mg/mL | 338 |
| | in 3 mL | |
| 10 mg SEQ ID NO: 1 w/150 mM NaHCO₃ | 0 mg/mL | 267 |
| | in 3 mL | |
| 10 mg SEQ ID NO: 1 w/100 mM NaHCO₃ | 0 mg/mL | 177 |
| | in 3 mL | |
| 10 mg SEQ ID NO: 1 w/50 mM NaHCO₃ | 0 mg/mL | 104 |
| | in 3 mL | |

### Example 3. Synergic Effects Between SEQ ID NO: 1 and Aqueous Carriers

Study 1. In this study, SEQ ID NO: 1 and sodium bicarbonate alone was first evaluated by microdilution MIC testing in both cation-adjusted Mueller-Hinton broth (CAMHB) medium and Roswell Park Memorial Institute (RPMI-1640) medium. Additionally, the activity of SEQ ID NO: 1 and sodiumbicarbonate were evaluated in alone and in combination by determining the Fractional Inhibitory Concentrations (FIC) in checkerboard assay. Based on the FIC index (FICI) values, it was determined whether there was a synergistic, antagonistic, or indifferent interaction between SEQ ID NO: 1 and sodium bicarbonate.

### Materials and Methods

TABLE 4
Agents tested, diluent, and tested concentrations

| | | Concentration | Conentration |
|---|---|---|---|
| | | Ranges Tested | Ranges Tested |
| Agent | Solvent/Diluent | (MIC) | (FIC) |
| SEQ ID NO: 1 | Water (pH 5; adjusted | 0.03-32 μg/mL | 0.015-16 μg/ |
| | with 1% glacial | | mL (CAMHB) |
| | AcOH)/Saline 0.0025 | | 0.004-4 μg/ |
| | P-80 | | mL (RPMI) |
| NaCHO₃ | Test Medium | 2-200 mM | 1.5-100 mM |
| (Sodium | | | (All media |
| Bicarbonate) | | | types) |

SEQ ID NO: 1 stocks were made at 5.12 mg/mL in water adjusted to pH of 5.0 with 1% glacial acetic acid. The SEQ ID NO: 1 stock was diluted to 20× the top testing concentration for MIC and FIC testing. Sodium bicarbonate was dissolved directly into the test media at the top concentration indicated and was diluted in test medium as needed for both MIC and FIC.

### Organisms.

The test organisms (Table 7) consisted of clinical isolates from the Micromyx (MMX) repository, a reference isolate from the American Type Culture Collection (ATCC, Manassas, VA) and the Centers for Disease Control (CDC, Atlanta GA). Upon initial receipt, the isolates were streaked under suitable conditions onto agar medium appropriate to each organism. The organisms were incubated for 18-24 hr at 35° C. in ambient atmosphere. Colonies harvested from these growth plates were resuspended in the appropriate medium containing a cryoprotectant. Aliquots of each suspension were then stored frozen at −80° C. Prior to testing, all organisms were cultured onto trypticase soy agar containing 5% sheep blood. The agar plates were incubated as described above.

### Test Media.

The Test media used for MIC and FIC were testing were cation-adjusted Mueller-Hinton broth (CAMHB) and Roswell Park Memorial Institute (RPMI-1640), buffered with 3-(N-morpholino)propanesulfonic acid (MOPS). Each test media was supplemented with 0.002% Tween-80 (P-80) prior to testing.

Sodium bicarbonate was dissolved in CAMHB and RPMI at the highest concentration tested; pH was adjusted to 7.0 with NaOH. The media containing sodium bicarbonate was further diluted in sterile media to achieve other testing concentration.

### MIC Assay Methodology.

MIC assay plates were prepared for broth microdilution in accordance with guidelines from the Clinical and Laboratory Standards Institute (CLSI); automated liquid handlers (Biomek 3000 and Biomek FX) and multi-channel pipettes were used to conduct serial dilutions and liquid transfers. MIC assay plates were prepared using a 96-well, deep-well microdilution plate that mimicked the layout of the daughter plates. Using the Biomek FX, 85 μL of the test media (CAMHB and RPMI, with and without sodium bicarbonate) was distributed to each well.

For the mother plate, wells across the standard 96-well microdilution plate were filled with 150 μL of sterile saline 0.002% P-80 in columns 2 through 12. A 300 μL aliquot at 20× the highest final concentrations of the MIC range to be tested was added to the corresponding well in column 1 of the plate. The Biomek 300 was used to make eleven 2-fold serial dilutions horizontally across each row of the plate from columns 2 through 11. The Biomek FX was then used to transfer 5 μL of the SEQ ID NO: 1 and saline 0.002% P-80 to the daughter plates. Saline 0.002% P-80 was used in place of drug for the rows containing sodium bicarbonate, as the intended active agent was already dissolved into the media.

A standardized inoculum of each organism was prepared per CLSI methods. Suspensions were prepared to equal a 0.5 McFarland standard followed by a 1:10 dilution into test medium. The inocula were dispensed into sterile reservoirs divided by length and the Biomek 3000 was used to inoculate the plates. Daughter plates were placed on the Biomek 3000 work surface in reverse orientation so that inoculation took place from low to high drug concentration. The Biomek 3000 delivered 10 μL of the diluted suspension into each well. These inoculations yielded a final cell concentration in the daughter plates of approximately 5×10⁵ CFU/mL in each well. Plates were incubated at 35° C. for 16-20 hr. The microplates were viewed from the bottom using a plate viewer and the MIC was recorded as the lowest concentration of drug that inhibited visible growth of the organism. Uninoculated solubility control plates were also observed for evidence of drug precipitation.

### FIC Checkerboard Assay Procedure.

FIC test ranges were set based on broth microdilution MIC test data. FIC assay plates were prepared in accordance with CLSI guidelines for broth microdilution susceptibility testing; automated liquid handlers, and multi-channel pipettes were used to conduct serial dilutions and liquid transfers.

SEQ ID NO: 1 was tested at two concentration ranges depending on the test media. All organisms tested in CAMHB 0.002% P-80 were tested from 0.015-16 μg/mL; when tested in RPMI 0.002% P-80, the range tested was 0.004-4 μg/mL. For each SEQ ID NO: 1 mother, wells across the standard 96-well microdilution plate were filled with 150 μL of sterile saline 0.002% P-80 in columns 2 through 12. A 300 μL aliquot at 20× the highest final concentration of each dilution range to be tested, was added to each well in column 1 of the plate. The Biomek 3000 was used to make eleven 2-fold serial dilutions horizontally across each row of the plate columns 2 through 11. This resulted in two SEQ ID NO: 1 mothers, CAMHB and RPMI.

For sodium bicarbonate, a serial 2-fold dilution series was prepared in large volume of media so that the final FIC volume (100 μL) yielded 100 mM to 1.5 mM down the columns of the plate. The wells of a 96-well, deep-well plates were filled 1.8 mL of the serial dilution set of sodium bicarbonate in rows A through G, with normal media in row H. The daughter well plates containing media with sodium bicarbonate were prepared using an 85 μL transfer from the deep-well to 96-well microtiter plates on the Biomek FX. The daughter plates were then completed using the Biomek FX to transfer 5 μL of drug solution from each well of the SEQ ID NO: 1 mother plates to the corresponding well in each daughter plate in a single step. In the checkerboard panels, row H and column 12 each contained serial dilutions of SEQ ID NO: 1 and sodium bicarbonate alone, respectively, and for determined of the MIC. The preparation of the daughter plates was performed for each media type; two times in total as all organisms were tested in both media types.

A standard inoculum for each organism was prepared per CLSI method. Colonies were picked up from the primary plate and a suspension was prepared equal to 0.5 McFarland turbidity standard. The suspensions were additionally diluted 1:10 in the appropriate broth. A 10 μL standardized inoculum was delivered into each well using the Biomek 3000 from low to high concentration. These inoculations yielded a final cell concentration in the daughter plates of approximately 5×10⁵ CFU/mL in each well. The test format resulted in the creation of an 8×12 checkerboard where each agent was tested alone (column 12 and row H) and in combination at variation rations of the drug concentration. Exemplary checkboard panels are seen in Table 5 and Table 6. Plates were incubated at 35° C. for 16-20 hr.

TABLE 5
Checkerboard Panel of CAMHB for SEQ ID NO: 1 and Sodium Bicarbonate

| | | SEQ ID NO: 1 | | | | | | | | | | | |
|---|---|---|---|---|---|---|---|---|---|---|---|---|---|
| CAMHB | | 1 | 2 | 3 | 4 | 5 | 6 | 7 | 8 | 9 | 10 | 11 | 12 |
| NaCHO₃ | A | 16/100 | 8/100 | 4/100 | 2/100 | 1/100 | 0.5/100 | 0.25/100 | 0.12/100 | 0.06/100 | 0.03/100 | 0.015/100 | 0/100 |
| (mM) | B | 16/50 | 8/50 | 4/50 | 2/50 | 1/50 | 0.5/50 | 0.25/50 | 0.12/50 | 0.06/50 | 0.03/50 | 0.015/50 | 0/50 |
| | C | 16/25 | 8/25 | 4/25 | 2/25 | 1/25 | 0.5/25 | 0.25/25 | 0.12/25 | 0.06/25 | 0.03/25 | 0.015/25 | 0/25 |
| | D | 16/12.5 | 8/12.5 | 4/12.5 | 2/12.5 | 1/12.5 | 0.5/12.5 | 0.25/12.5 | 0.12/12.5 | 0.06/12.5 | 0.03/12.5 | 0.015/12.5 | 0/12.5 |
| | E | 16/6.25 | 8/6.25 | 4/6.25 | 2/6.25 | 1/6.25 | 0.5/6.25 | 0.25/6.25 | 0.12/6.25 | 0.06/6.25 | 0.03/6.25 | 0.015/6.25 | 0/6.25 |
| | F | 16/3.12 | 8/3.12 | 4/3.12 | 2/3.12 | 1/3.12 | 0.5/3.12 | 0.25/3.12 | 0.12/3.12 | 0.06/3.12 | 0.03/3.12 | 0.015/3.12 | 0/3.12 |
| | G | 16/1.56 | 8/1.56 | 4/1.56 | 2/1.56 | 1/1.56 | 0.5/1.56 | 0.25/1.56 | 0.12/1.56 | 0.06/1.56 | 0.03/1.56 | 0.015/1.56 | 0/1.56 |
| | H | 16/0 | 8/0 | 4/0 | 2/0 | 1/0 | 0.5/0 | 0.25/0 | 0.12/0 | 0.06/0 | 0.03/0 | 0.015/0 | 0/0 |

TABLE 6
Checkerboard Panel of RPMI for SEQ ID NO: 1 and Sodium Bicarbonate

| | | SEQ ID NO: 1 | | | | | | | | | | | |
|---|---|---|---|---|---|---|---|---|---|---|---|---|---|
| RPMI | | 1 | 2 | 3 | 4 | 5 | 6 | 7 | 8 | 9 | 10 | 11 | 12 |
| NaCHO₃ | A | 4/100 | 2/100 | 1/100 | 0.5/100 | 0.25/100 | 0.12/100 | 0.06/100 | 0.03/100 | 0.015/100 | 0.008/100 | 0.004/100 | 0/100 |
| (mM) | B | 4/50 | 2/50 | 1/50 | 0.5/50 | 0.25/50 | 0.12/50 | 0.06/50 | 0.03/50 | 0.015/50 | 0.008/50 | 0.004/50 | 0/50 |
| | C | 4/25 | 2/25 | 1/25 | 0.5/25 | 0.25/25 | 0.12/25 | 0.06/25 | 0.03/25 | 0.015/25 | 0.008/25 | 0.004/25 | 0/25 |
| | D | 4/12.5 | 2/12.5 | 1/12.5 | 0.5/12.5 | 0.25/12.5 | 0.12/12.5 | 0.06/12.5 | 0.03/12.5 | 0.015/12.5 | 0.008/12.5 | 0.004/12.5 | 0/12.5 |
| | E | 4/6.25 | 2/6.25 | 1/6.25 | 0.5/6.25 | 0.25/6.25 | 0.12/6.25 | 0.06/6.25 | 0.03/6.25 | 0.015/6.25 | 0.008/6.25 | 0.004/6.25 | 0/6.25 |
| | F | 4/3.12 | 2/3.12 | 1/3.12 | 0.5/3.12 | 0.25/3.12 | 0.32/3.12 | 0.06/3.12 | 0.03/3.12 | 0.015/3.12 | 0.008/3.12 | 0.004/3.12 | 0/3.12 |
| | G | 4/1.56 | 2/1.56 | 1/1.56 | 0.5/1.56 | 0.25/1.56 | 0.12/1.56 | 0.06/1.56 | 0.03/1.56 | 0.015/1.56 | 0.008/1.56 | 0.004/1.56 | 0/1.56 |
| | H | 4/0 | 2/0 | 1/0 | 0.5/0 | 0.25/0 | 0.12/0 | 0.03/0 | 0.03/0 | 0.015/0 | 0.008/0 | 0.004/0 | 0/0 |

The MIC of the horizontal agent (row H) and the MIC of the vertical agent (column 12) were recorded and the wells of the growth-no growth interface for wells containing agents in combination at varying ratios were recorded by row. The FIC was read and recorded as the lowest concentrations of drug exhibited no growth of the organism by row where agents were tested in combination (row A through G). Pinpoint trailing was not interpreted as growth.

### FIC/FIC Calculations.

FIC values were calculated essentially as described in Eliopoulos G and Moellering R. 1991. Antimicrobial combinations. In Antibiotic in Laboratory Medicine, Third edition, edited by V Lorian. Williams and Wilkins, Baltimore, MD, pp. 432-492.

For each relevant row of the panel, the FICI was calculated as:

FIC_{drug} A/MIC_{drug A}+FIC_{drug B}/MIC_{drug}=FICI

Mean FICI values were determined for each combination tested by averaging the FICI values as observed on each row of the checkerboard assay (See Table 9, e.g., if there were FICI values for B-G, those FICI values were added together and divided by the number of rows the FICI values which in this case is 6 to get the mean FICI value for that combination).

In the instance where the MIC for one of the test agents was off-scale (greater than the highest concentration evaluated, (e.g., >32 μg/mL, the FIC was calculated based on a MIC of 64 μg/mL). In instances where the MIC was less than or equal to the lowest concentration tested (e.g., ≤0.015 μg/mL) the MIC was set to the lowest tested concentration (e.g., if the MIC was ≤0.015 μg/mL, the FIC was calculated based on a MIC of 0.015 μg/mL).

Using the criteria described by: Odds FC. 2003. Synergy, antagonism, and what the chequerboard puts between them. J Antimicrob Chemother 52(1):1, the mean FICI for the combination was interpreted as follows: ≤0.5=synergy, >0.5-4=additive/indifferent, and >4=antagonism. An interpretation of synergy is consistent with the inhibition of organism growth by combinations at concentrations significantly below (>4-fold) the MIC of either agent alone, resulting in a low FICI value (≤0.5). An interpretation of indifference is consistent with growth inhibition at concentrations at or slightly below/above the MICs of the individual agents alone, resulting in a FICI value of >0.5 but ≤0.4. An interpretation of antagonism resulting when the concentrations of the compounds in combination that are required to inhibit organism growth are substantially greater (>4-fold) than those for agents individually, resulting in a FICI value of >4.

### Results and Discussion

An overall summary of the activity of SEQ ID NO: 1 and sodium bicarbonate in CAMHB and RPMI is shown in Table 7.

TABLE 7
Activity of SEQ ID NO: 1 alone and Sodium Bicarbonate alone as
observed during initial MIC testing in CAMHB and RPMI media

| | | | MIC | | | |
|---|---|---|---|---|---|---|
| | | | CAMHB | | RPMI | |
| | | | SEQ ID NO: 1 | NaCHO₃ | SEQ ID NO: 1 | NaCHO₃ |
| Organism | Type | Isolate | (μg/mL) | (mM) | (μg/mL) | (mM) |
| S. aureus | QC; MSSA | ATCC | 4 | >200 | 0.5 | 200 |
| | | 29213 | | | | |
| | USA 300 | NRS 384 | 4 | 200 | 0.5 | 200 |
| | CA-MRSA | | | | | |
| | USA 100 | NRS 382 | 4 | 200 | 0.5 | 200 |
| | HA-MRSA | | | | | |
| S. epidermidis | MSSE | MMX 8655 | 0.5 | 200 | 0.12 | 200 |
| | MRSE | MMX 5129 | 1 | 200 | 0.12 | 200 |
| | MRSE | MMX 3628 | 0.25 | 100 | 0.12 | 200 |
| E. Coli | QC; non- | ATCC | 4 | 200 | 0.5 | 200 |
| | ESBL | 25922 | | | | |
| | KPC | CDC 0451 | 2 | 100 | 0.5 | 200 |
| | NDM-1 | ATCC | 4 | 200 | 0.5 | 200 |
| | | BAA-2469 | | | | |
| P. aeruginosa | QC | ATCC | 8 | 200 | 2 | 200 |
| | | 27853 | | | | |
| | PDC-8; | CDC 0509 | 8 | 200 | 4 | 200 |
| | VIM-2 | | | | | |
| | FOX-14; | CDC 0515 | 8 | 200 | 1 | 200 |
| | PDC-1 | | | | | |

QC: quality control,
CA: community-acquired,
HA: hospital acquired,
MSSA/MSSE: methicillin-susceptible S. aureus/S. epidermidis,
MRSA/MRSE: methicillin-resistant S. aureus/S. epidermidis,
ESBL: extended-spectrum beta-lactamase,
KPC: K. pneumoniae carbapenemase,
NDM-1: New Delhi Metallo-beta-lactamase

TABLE 8
Summary of FICI data from checkerboard assays
of SEQ ID NO: 1 and Sodium Bicarbonate against
target pathogens in CAMHB and RPMI

| | | | MIC | |
|---|---|---|---|---|
| Organism | Type | Number | CAMHB | RPMI |
| S. aureus | QC; MSSA | ATCC | 0.52* | 0.89 |
| | | 29213 | | |
| | USA 300 CA- | NRS 384 | 0.60* | 0.93 |
| | MRSA | | | |
| | USA 100 HA- | NRS 382 | 0.46** | 0.96 |
| | MRSA | | | |
| S. epidermidis | MSSE | MMX 8655 | 0.76* | 1.14 |
| | MRSE | MMX 5129 | 0.59* | 1.14 |
| | MRSE | MMX 3628 | 0.78* | 1.57 |
| E. Coli | QC; non-ESBL | ATCC | 0.34** | 1.07 |
| | | 25922 | | |
| | KPC | CDC 0451 | 0.55** | 1.28 |
| | NDM-1 | ATCC | 1.07 | 1.14 |
| | | BAA-2469 | | |
| P. aeruginosa | QC | ATCC | 1.07 | 1.28 |
| | | 27853 | | |
| | PDC-8; VIM-2 | CDC 0509 | 10.7 | 1.07 |
| | FOX-14; PDC-1 | CDC 0515 | 0.64 | 1.43 |

QC: quality control,
CA: community-acquired,
HA: hospital acquired,
MSSA/MSSE: methicillin-susceptible S. aureus/S. epidermidis,
MRSA/MRSE: methicillin-resistant S. aureus/S. epidermidis,
ESBL: extended-spectrum beta-lactamase,
KPC: K. pneumoniae carbapenemase,
NDM-1: New Delhi Metallo-beta-lactamase
*Cells have at least one row on the checkerboard panel with a FICI value indicative of synergy
**Cells have mean FICI values indicative of synergy

The MIC of SEQ ID NO: 1 in CAMHB and RPMI was consistent with SEQ ID NO: 1 being several-fold more active in RPMI relative to CAMHB. In CAMHB, there was a distinct precipitation of SEQ ID NO: 1 at 16 and 32 μg/mL and trace precipitation at 4 and 8 μg/mL; no precipitation was noted with SEQ ID NO: 1 RPMI. Distinct precipitated was noted with sodium bicarbonate in both RPMI and CAMHB at 200 mM and trace precipitation was noted at 50 and 100 mM sodium bicarbonate in CAMHB.

Mean FICI values as observed in CAMHB and RPMI are summarized in Table 8. The raw data from the checkerboards are shown in Table 9A-9X. Table 9A-9X. MIC, FIC, FICI data from the Checkerboard Panels

TABLE 9A
CAMHB
Organism: S. aureus ATCC 29213 FICI (N): 7
Drug A: Sodium BicarbonateDrug A MIC: >100 SUM FICI: 3.65
Drug B: SEQ ID NO: 1 Drug B MIC: 4 MEAN FICI: 0.52

| Row | MIC_{A} | FIC_{A} | MIC_{B} | FIC_{B} | FICI |
|---|---|---|---|---|---|
| A | 100 | 0.5 | 0.12 | 0.03 | 0.53 |
| B | 50^{a} | 0.25^{a} | 0.5^{a} | 0.125^{a} | 0.375^{a} |
| C | 25^{a} | 0.125^{a} | 1^{a} | 0.25^{a} | 0.375^{a} |
| D | 12.5^{a} | 0.063^{a} | 1^{a} | 0.25^{a} | 0.313^{a} |
| E | 6.25 | 0.031 | 2 | 0.5 | 0.531 |
| F | 3.12 | 0.016 | 2 | 0.5 | 0.516 |
| G | 1.56 | 0.008 | 4 | 1 | 1.008 |
| H | | | | | |

^{a}Cells have a FICI values indicative of synergy

TABLE 9B
CAMHB
Organism: S. aureus NRS 384 FICI (N): 7
Drug A: Sodium BicarbonateDrug A MIC: >100 SUM FICI: 4.21
Drug B: SEQ ID NO: 1 Drug B MIC: 4 MEAN FICI: 0.60

| Row | MIC_{A} | FIC_{A} | MIC_{B} | FIC_{B} | FICI |
|---|---|---|---|---|---|
| A | 100 | 0.5 | 0.12 | 0.03 | 0.53 |
| B | 50^{a} | 0.25^{a} | 0.25^{a} | 0.063^{a} | 0.313^{a} |
| C | 25^{a} | 0.125^{a} | 0.5^{a} | 0.125^{a} | 0.25^{a} |
| D | 12.5 | 0.063 | 2 | 0.5 | 0.563 |
| E | 6.25 | 0.031 | 4 | 1 | 1.031 |
| F | 3.12 | 0.016 | 2 | 0.5 | 0.516 |
| G | 1.56 | 0.008 | 4 | 1 | 1.008 |
| H | | | | | |

^{a}Cells have a FICI values indicative of synergy

TABLE 9C
CAMHB
Organism: S. aureus NRS 382 FICI (N): 7
Drug A: Sodium BicarbonateDrug A MIC: >100 SUM FICI: 3.21
Drug B: SEQ ID NO: 1 Drug B MIC: 4 MEAN FICI: 0.46^{a}

| Row | MIC_{A} | FIC_{A} | MIC_{B} | FIC_{B} | FICI |
|---|---|---|---|---|---|
| A | 100 | 0.5 | 0.12 | 0.03 | 0.53 |
| B | 50^{a} | 0.25^{a} | 0.25^{a} | 0.063^{a} | 0.313^{a} |
| C | 25^{a} | 0.125^{a} | 0.5^{a} | 0.125^{a} | 0.25^{a} |
| D | 12.5^{a} | 0.063^{a} | 1^{a} | 0.25^{a} | 0.313^{a} |
| E | 6.25^{a} | 0.031^{a} | 1^{a} | 0.25^{a} | 0.281^{a} |
| F | 3.12 | 0.016 | 2 | 0.5 | 0.516 |
| G | 1.56 | 0.008 | 4 | 1 | 1.008 |
| H | | | | | |

^{a}Cells have a FICI values indicative of synergy

TABLE 9D
CAMHB
Organism: S. epidermidis MMX 8655 FICI (N): 7
Drug A: Sodium BicarbonateDrug A MIC: >100 SUM FICI: 5.35
Drug B: SEQ ID NO: 1 Drug B MIC: 0.5 MEAN FICI: 0.76

| Row | MIC_{A} | FIC_{A} | MIC_{B} | FIC_{B} | FICI |
|---|---|---|---|---|---|
| A | 100 | 0.5 | 0.06 | 0.12 | 0.62 |
| B | 50^{a} | 0.25^{a} | 0.12^{a} | 0.24^{a} | 0.49^{a} |
| C | 25 | 0.125 | 0.25 | 0.5 | 0.625 |
| D | 12.5 | 0.063 | 0.25 | 0.5 | 0.563 |
| E | 6.25 | 0.031 | 0.5 | 1 | 1.031 |
| F | 3.12 | 0.016 | 0.5 | 1 | 1.016 |
| G | 1.56 | 0.008 | 0.5 | 1 | 1.008 |
| H | | | | | |

^{a}Cells have a FICI values indicative of synergy

TABLE 9E
CAMHB
Organism: S. epidermidis MMX 5129 FICI (N): 7
Drug A: Sodium BicarbonateDrug A MIC: >100 SUM FICI: 4.11
Drug B: SEQ ID NO: 1 Drug B MIC: 1 MEAN FICI: 0.59

| Row | MIC_{A} | FIC_{A} | MIC_{B} | FIC_{B} | FICI |
|---|---|---|---|---|---|
| A | 100 | 0.5 | 0.12 | 0.12 | 0.62 |
| B | 50^{a} | 0.25^{a} | 0.25^{a} | 0.25^{a} | 0.5^{a} |
| C | 25^{a} | 0.125^{a} | 0.25^{a} | 0.25^{a} | 0.375^{a} |
| D | 12.5 | 0.063 | 0.5 | 0.5 | 0.563 |
| E | 6.25 | 0.031 | 0.5 | 0.5 | 0.531 |
| F | 3.12 | 0.016 | 0.5 | 0.5 | 0.516 |
| G | 1.56 | 0.008 | 1 | 1 | 1.008 |
| H | | | | | |

^{a}Cells have a FICI values indicative of synergy

TABLE 9F
CAMHB
Organism: S. epidermidis MMX 3628 FICI (N): 7
Drug A: Sodium BicarbonateDrug A MIC: >100 SUM FICI: 5.47
Drug B: SEQ ID NO: 1 Drug B MIC: 0.5 MEAN FICI: 0.78

| Row | MIC_{A} | FIC_{A} | MIC_{B} | FIC_{B} | FICI |
|---|---|---|---|---|---|
| A | 100 | 0.5 | 0.12 | 0.24 | 0.74 |
| B | 50^{a} | 0.25^{a} | 0.12^{a} | 0.24^{a} | 0.49^{a} |
| C | 25 | 0.125 | 0.25 | 0.5 | 0.625 |
| D | 12.5 | 0.063 | 0.5 | 1 | 1.063 |
| E | 6.25 | 0.031 | 0.25 | 0.5 | 0.531 |
| F | 3.12 | 0.016 | 0.5 | 1 | 1.016 |
| G | 1.56 | 0.008 | 0.5 | 1 | 1.008 |
| H | | | | | |

^{a}Cells have a FICI values indicative of synergy

TABLE 9G
CAMHB
Organism: E. coli ATCC 25922 FICI (N): 7
Drug A: Sodium BicarbonateDrug A MIC: >100 SUM FICI: 2.38
Drug B: SEQ ID NO: 1 Drug B MIC: 8 MEAN FICI: 0.34^{a}

| Row | MIC_{A} | FIC_{A} | MIC_{B} | FIC_{B} | FICI |
|---|---|---|---|---|---|
| A | 100 | 0.5 | 0.12 | 0.015 | 0.515 |
| B | 50^{a} | 0.25^{a} | 0.5^{a} | 0.0625^{a} | 0.3125^{a} |
| C | 25^{a} | 0.125^{a} | 0.5^{a} | 0.0625^{a} | 0.1875^{a} |
| D | 12.5^{a} | 0.063^{a} | 1^{a} | 0.125^{a} | 0.188^{a} |
| E | 6.25^{a} | 0.031^{a} | 1^{a} | 0.125^{a} | 0.156^{a} |
| F | 3.12 | 0.016 | 4 | 0.5 | 0.516 |
| G | 1.56 | 0.008 | 4 | 0.5 | 0.508 |
| H | | | | | |

^{a}Cells have a FICI values indicative of synergy

TABLE 9H
CAMHB
Organism: E. coli CDC 0451FICI (N): 7
Drug A: Sodium BicarbonateDrug A MIC: >100 SUM FICI: 3.52
Drug B: SEQ ID NO: 1 Drug B MIC: 4 MEAN FICI: 0.50^{a}

| Row | MIC_{A} | FIC_{A} | MIC_{B} | FIC_{B} | FICI |
|---|---|---|---|---|---|
| A | 100 | 0.5 | 0.12 | 0.03 | 0.53 |
| B | 50^{a} | 0.25^{a} | 1^{a} | 0.25^{a} | 0.5^{a} |
| C | 25 | 0.125 | 2 | 0.5 | 0.625 |
| D | 12.5 | 0.063 | 2 | 0.5 | 0.563 |
| E | 6.25^{a} | 0.031^{a} | 1^{a} | 0.25^{a} | 0.281^{a} |
| F | 3.12 | 0.016 | 2 | 0.5 | 0.516 |
| G | 1.56 | 0.008 | 2 | 0.5 | 0.508 |
| H | | | | | |

^{a}Cells have a FICI values indicative of synergy

TABLE 9I
CAMHB
Organism: E. coli ATCC BAA-2496 FICI (N): 7
Drug A: Sodium BicarbonateDrug A MIC: >100 SUM FICI: 7.52
Drug B: SEQ ID NO: 1 Drug B MIC: 4 MEAN FICI: 1.07

| Row | MIC_{A} | FIC_{A} | MIC_{B} | FIC_{B} | FICI |
|---|---|---|---|---|---|
| A | 100 | 0.5 | 0.12 | 0.03 | 0.53 |
| B | 50 | 0.25 | 2 | 0.5 | 0.75 |
| C | 25 | 0.125 | 4 | 1 | 1.125 |
| D | 12.5 | 0.063 | 4 | 1 | 1.063 |
| E | 6.25 | 0.031 | 8 | 2 | 1.063 |
| F | 3.12 | 0.016 | 4 | 1 | 2.031 |
| G | 1.56 | 0.008 | 4 | 1 | 1.008 |
| H | | | | | |

TABLE 9J
CAMHB
Organism: P. aeruginosa ATCC 27853 FICI (N): 7
Drug A: Sodium BicarbonateDrug A MIC: >100 SUM FICI: 7.49
Drug B: SEQ ID NO: 1 Drug B MIC: 8 MEAN FICI: 1.07

| Row | MIC_{A} | FIC_{A} | MIC_{B} | FIC_{B} | FICI |
|---|---|---|---|---|---|
| A | 100 | 0.5 | 4 | 0.5 | 1 |
| B | 50 | 0.25 | 8 | 1 | 1.25 |
| C | 25 | 0.125 | 8 | 1 | 1.125 |
| D | 12.5 | 0.063 | 8 | 1 | 1.063 |
| E | 6.25 | 0.031 | 8 | 1 | 1.031 |
| F | 3.12 | 0.016 | 8 | 1 | 1.016 |
| G | 1.56 | 0.008 | 8 | 1 | 1.008 |
| H | | | | | |

TABLE 9K
CAMHB
Organism: P. aeruginosa CDC 0509 FICI (N): 7
Drug A: Sodium BicarbonateDrug A MIC: >100 SUM FICI: 7.49
Drug B: SEQ ID NO: 1 Drug B MIC: 8 MEAN FICI: 1.07

| Row | MIC_{A} | FIC_{A} | MIC_{B} | FIC_{B} | FICI |
|---|---|---|---|---|---|
| A | 100 | 0.5 | 4 | 0.5 | 1 |
| B | 50 | 0.25 | 8 | 1 | 1.25 |
| C | 25 | 0.125 | 8 | 1 | 1.125 |
| D | 12.5 | 0.063 | 8 | 1 | 1.063 |
| E | 6.25 | 0.031 | 8 | 1 | 1.031 |
| F | 3.12 | 0.016 | 8 | 1 | 1.016 |
| G | 1.56 | 0.008 | 8 | 1 | 1.008 |
| H | | | | | |

TABLE 9L
CAMHB
Organism: P. aeruginosa CDC 0515 FICI (N): 7
Drug A: Sodium BicarbonateDrug A MIC: >100 SUM FICI: 4.49
Drug B: SEQ ID NO: 1 Drug B MIC: 8 MEAN FICI: 0.64

| Row | MIC_{A} | FIC_{A} | MIC_{B} | FIC_{B} | FICI |
|---|---|---|---|---|---|
| A | 100 | 0.5 | 4 | 0.5 | 1 |
| B | 50 | 0.25 | 4 | 0.5 | 0.75 |
| C | 25 | 0.125 | 4 | 0.5 | 0.625 |
| D | 12.5 | 0.063 | 4 | 0.5 | 0.563 |
| E | 6.25 | 0.031 | 4 | 0.5 | 0.531 |
| F | 3.12 | 0.016 | 4 | 0.5 | 0.516 |
| G | 1.56 | 0.008 | 4 | 0.5 | 0.508 |
| H | | | | | |

TABLE 9M
RPMI
Organism: S. aureus ATCC 29213 FICI (N): 7
Drug A: Sodium BicarbonateDrug A MIC: >100 SUM FICI: 6.23
Drug B: SEQ ID NO: 1 Drug B MIC: 0.5 MEAN FICI: 0.89

| Row | MIC_{A} | FIC_{A} | MIC_{B} | FIC_{B} | FICI |
|---|---|---|---|---|---|
| A | 100 | 0.5 | 0.12 | 0.24 | 0.74 |
| B | 50 | 0.25 | 0.25 | 0.5 | 0.75 |
| C | 25 | 0.125 | 0.25 | 0.5 | 0.625 |
| D | 12.5 | 0.063 | 0.5 | 1 | 1.063 |
| E | 6.25 | 0.031 | 0.5 | 1 | 1.031 |
| F | 3.12 | 0.016 | 0.5 | 1 | 1.016 |
| G | 1.56 | 0.008 | 0.5 | 1 | 1.008 |
| H | | | | | |

TABLE 9N
RPMI
Organism: S. aureus NRS 384 FICI (N): 7
Drug A: Sodium BicarbonateDrug A MIC: >100 SUM FICI: 6.49
Drug B: SEQ ID NO: 1 Drug B MIC: 0.5 MEAN FICI: 0.93

| Row | MIC_{A} | FIC_{A} | MIC_{B} | FIC_{B} | FICI |
|---|---|---|---|---|---|
| A | 100 | 0.5 | 0.25 | 0.5 | 1 |
| B | 50 | 0.25 | 0.25 | 0.5 | 0.75 |
| C | 25 | 0.125 | 0.25 | 0.5 | 0.625 |
| D | 12.5 | 0.063 | 0.5 | 1 | 1.063 |
| E | 6.25 | 0.031 | 0.5 | 1 | 1.031 |
| F | 3.12 | 0.016 | 0.5 | 1 | 1.016 |
| G | 1.56 | 0.008 | 0.5 | 1 | 1.008 |
| H | | | | | |

TABLE 9O
RPMI
Organism: S. aureus NRS 382 FICI (N): 7
Drug A: Sodium BicarbonateDrug A MIC: >100 SUM FICI: 6.73
Drug B: SEQ ID NO: 1 Drug B MIC: 0.5 MEAN FICI: 0.96

| Row | MIC_{A} | FIC_{A} | MIC_{B} | FIC_{B} | FICI |
|---|---|---|---|---|---|
| A | 100 | 0.5 | 0.12 | 0.24 | 0.74 |
| B | 50 | 0.25 | 0.25 | 0.5 | 0.75 |
| C | 25 | 0.125 | 0.5 | 1 | 0.625 |
| D | 12.5 | 0.063 | 0.5 | 1 | 1.063 |
| E | 6.25 | 0.031 | 0.5 | 1 | 1.031 |
| F | 3.12 | 0.016 | 0.5 | 1 | 1.016 |
| G | 1.56 | 0.008 | 0.5 | 1 | 1.008 |
| H | | | | | |

TABLE 9P
RPMI
Organism: S. epidermidis MMX 8655 FICI (N): 7
Drug A: Sodium BicarbonateDrug A MIC: >100 SUM FICI: 7.99
Drug B: SEQ ID NO: 1 Drug B MIC: 0.12 MEAN FICI: 1.14

| Row | MIC_{A} | FIC_{A} | MIC_{B} | FIC_{B} | FICI |
|---|---|---|---|---|---|
| A | 100 | 0.5 | 0.12 | 1 | 1.5 |
| B | 50 | 0.25 | 0.12 | 1 | 1.25 |
| C | 25 | 0.125 | 0.12 | 1 | 1.125 |
| D | 12.5 | 0.063 | 0.12 | 1 | 1.063 |
| E | 6.25 | 0.031 | 0.12 | 1 | 1.031 |
| F | 3.12 | 0.016 | 0.12 | 1 | 1.016 |
| G | 1.56 | 0.008 | 0.12 | 1 | 1.008 |
| H | | | | | |

TABLE 9Q
RPMI
Organism: S. epidermidis MMX 5129 FICI (N): 7
Drug A: Sodium BicarbonateDrug A MIC: >100 SUM FICI: 7.99
Drug B: SEQ ID NO: 1 Drug B MIC: 0.12 MEAN FICI: 1.14

| Row | MIC_{A} | FIC_{A} | MIC_{B} | FIC_{B} | FICI |
|---|---|---|---|---|---|
| A | 100 | 0.5 | 0.12 | 1 | 1.5 |
| B | 50 | 0.25 | 0.12 | 1 | 1.25 |
| C | 25 | 0.125 | 0.12 | 1 | 1.125 |
| D | 12.5 | 0.063 | 0.12 | 1 | 1.063 |
| E | 6.25 | 0.031 | 0.12 | 1 | 1.031 |
| F | 3.12 | 0.016 | 0.12 | 1 | 1.016 |
| G | 1.56 | 0.008 | 0.12 | 1 | 1.008 |
| H | | | | | |

TABLE 9R
RPMI
Organism: S. epidermidis MMX 3628 FICI (N): 7
Drug A: Sodium BicarbonateDrug A MIC: >100 SUM FICI: 10.99
Drug B: SEQ ID NO: 1 Drug B MIC: 0.06 MEAN FICI: 1.57

| Row | MIC_{A} | FIC_{A} | MIC_{B} | FIC_{B} | FICI |
|---|---|---|---|---|---|
| A | 100 | 0.5 | 0.12 | 2 | 2.5 |
| B | 50 | 0.25 | 0.06 | 1 | 1.25 |
| C | 25 | 0.125 | 0.06 | 1 | 1.125 |
| D | 12.5 | 0.063 | 0.06 | 1 | 1.063 |
| E | 6.25 | 0.031 | 0.12 | 2 | 2.031 |
| F | 3.12 | 0.016 | 0.06 | 1 | 1.016 |
| G | 1.56 | 0.008 | 0.12 | 2 | 2.008 |
| H | | | | | |

TABLE 9S
RPMI
Organism: E. coli ATCC 25922 FICI (N): 7
Drug A: Sodium BicarbonateDrug A MIC: >100 SUM FICI: 7.49
Drug B: SEQ ID NO: 1 Drug B MIC: 0.5 MEAN FICI: 1.07

| Row | MIC_{A} | FIC_{A} | MIC_{B} | FIC_{B} | FICI |
|---|---|---|---|---|---|
| A | 100 | 0.5 | 0.5 | 0.5 | 1 |
| B | 50 | 0.25 | 0.25 | 1 | 1.25 |
| C | 25 | 0.125 | 0.125 | 1 | 1.125 |
| D | 12.5 | 0.063 | 0.063 | 1 | 1.063 |
| E | 6.25 | 0.031 | 0.031 | 1 | 1.031 |
| F | 3.12 | 0.016 | 0.016 | 1 | 1.016 |
| G | 1.56 | 0.008 | 0.008 | 1 | 1.008 |
| H | | | | | |

TABLE 9T
RPMI
Organism: E. coli CDC 0451 FICI (N): 7
Drug A: Sodium BicarbonateDrug A MIC: >100 SUM FICI: 8.99
Drug B: SEQ ID NO: 1 Drug B MIC: 0.5 MEAN FICI: 1.28

| Row | MIC_{A} | FIC_{A} | MIC_{B} | FIC_{B} | FICI |
|---|---|---|---|---|---|
| A | 100 | 0.5 | 1 | 0.5 | 2 |
| B | 50 | 0.25 | 0.5 | 1 | 1.25 |
| C | 25 | 0.125 | 0.5 | 1 | 1.125 |
| D | 12.5 | 0.063 | 0.5 | 1 | 1.063 |
| E | 6.25 | 0.031 | 0.5 | 1 | 1.031 |
| F | 3.12 | 0.016 | 0.5 | 1 | 1.016 |
| G | 1.56 | 0.008 | 0.5 | 1 | 1.008 |
| H | | | | | |

TABLE 9U
RPMI
Organism: E. coli ATCC BAA-2496 FICI (N): 7
Drug A: Sodium BicarbonateDrug A MIC: >100 SUM FICI: 7.99
Drug B: SEQ ID NO: 1 Drug B MIC: 0.5 MEAN FICI: 1.14

| Row | MIC_{A} | FIC_{A} | MIC_{B} | FIC_{B} | FICI |
|---|---|---|---|---|---|
| A | 100 | 0.5 | 0.5 | 1 | 1.5 |
| B | 50 | 0.25 | 0.5 | 1 | 1.25 |
| C | 25 | 0.125 | 0.5 | 1 | 1.125 |
| D | 12.5 | 0.063 | 0.5 | 1 | 1.063 |
| E | 6.25 | 0.031 | 0.5 | 1 | 1.031 |
| F | 3.12 | 0.016 | 0.5 | 1 | 1.016 |
| G | 1.56 | 0.008 | 0.5 | 1 | 1.008 |
| H | | | | | |

TABLE 9V
RPMI
Organism: P. aeruginosa ATCC 27853 FICI (N): 7
Drug A: Sodium BicarbonateDrug A MIC: >100 SUM FICI: 8.99
Drug B: SEQ ID NO: 1 Drug B MIC: 1 MEAN FICI: 1.28

| Row | MIC_{A} | FIC_{A} | MIC_{B} | FIC_{B} | FICI |
|---|---|---|---|---|---|
| A | 100 | 0.5 | 2 | 2 | 1.5 |
| B | 50 | 0.25 | 1 | 1 | 1.25 |
| C | 25 | 0.125 | 1 | 1 | 1.125 |
| D | 12.5 | 0.063 | 1 | 1 | 1.063 |
| E | 6.25 | 0.031 | 1 | 1 | 1.031 |
| F | 3.12 | 0.016 | 1 | 1 | 1.016 |
| G | 1.56 | 0.008 | 1 | 1 | 1.008 |
| H | | | | | |

TABLE 9W
RPMI
Organism: P. aeruginosa CDC 0509 FICI (N): 7
Drug A: Sodium BicarbonateDrug A MIC: >100 SUM FICI: 7.47
Drug B: SEQ ID NO: 1 Drug B MIC: 4 MEAN FICI: 1.07

| Row | MIC_{A} | FIC_{A} | MIC_{B} | FIC_{B} | FICI |
|---|---|---|---|---|---|
| A | 100 | 0.5 | 4 | 1 | 1.5 |
| B | 50 | 0.25 | 2 | 0.5 | 0.75 |
| C | 25 | 0.125 | 4 | 1 | 1.125 |
| D | 12.5 | 0.063 | 4 | 1 | 1.063 |
| E | 6.25 | 0.031 | 4 | 1 | 1.031 |
| F | 3.12 | 0.016 | 4 | 1 | 1.016 |
| G | 1.56 | 0.008 | 4 | 1 | 1.008 |
| H | | | | | |

TABLE 9X
RPMI
Organism: P. aeruginosa CDC 0515 FICI (N): 7
Drug A: Sodium BicarbonateDrug A MIC: >100 SUM FICI: 9.99
Drug B: SEQ ID NO: 1 Drug B MIC: 0.5 MEAN FICI: 1.43

| Row | MIC_{A} | FIC_{A} | MIC_{B} | FIC_{B} | FICI |
|---|---|---|---|---|---|
| A | 100 | 0.5 | 1 | 2 | 2.5 |
| B | 50 | 0.25 | 1 | 2 | 2.25 |
| C | 25 | 0.125 | 0.5 | 1 | 1.125 |
| D | 12.5 | 0.063 | 0.5 | 1 | 1.063 |
| E | 6.25 | 0.031 | 0.5 | 1 | 1.031 |
| F | 3.12 | 0.016 | 0.5 | 1 | 1.016 |
| G | 1.56 | 0.008 | 0.5 | 1 | 1.008 |
| H | | | | | |

When testing in RPMI, there was no apparent interaction between SEQ ID NO: 1 and sodium bicarbonate with mean FICI values indicative of indifference and no individual FICI values on checkerboards showing either synergy or antagonism (Table 8). In contrast, there were several instances where synergy between SEQ ID NO: 1 and sodium bicarbonate was observed when testing in CAMHB, with the exception of one isolate of E. coli (ATCC BAA-2469) and all three P. aeruginosa where the interaction was indifferent (Table 8). A mean FICI indicative of synergy was observed for the two remaining E. coli and one of the evaluated S. aureus (NRS 382). For the remaining S. aureus and S. epidermidis there was at least one row on the checkerboard panel with an FICI indicative synergy. In all cases with the exception of P. aeruginosa and the single E. coli ATCC BAA-2469 isolate, testing in CAMHB with 50 mM sodium bicarbonate reduced the SEQ ID NO: 1 MIC at least 4-fold.

In summary, there was evidence of synergy between SEQ ID NO: 1 and sodium bicarbonate when testing CAMHB against S. aureus, S. epidermidis, and E. coli but not against P. aeruginosa. This synergy was not apparent when testing in RPMI. Of note, the synergy between SEQ ID NO: 1 and sodium bicarbonate observed in CAMHB reduced SEQ ID NO: 1 MIC values to levels seen when testing SEQ ID NO: 1 in RPMI alone.

Study 2. In this study, the killing of 5 test isolates (Staphylococcus aureus NRS 382 & NRS 384, Staphylococcus epidermidis MMX 8655, Escherichia coli CDC 0451, and Pseudomonas aeruginosa CDC 0509) by SEQ ID NO: 1 (drug) in PBS, water, and CAMHB with and without 50 mM sodium bicarbonate was evaluated. SEQ ID NO: 1 was tested at 3 test concentrations (0.25×, 0.5×, and 1× the MIC) and viable cells were enumerated at 0, 2.5, 5, 10, 15, and 30 minutes. The ability of 2×D/E broth to neutralize SEQ ID NO: 1 in PBS was confirmed prior to testing.

SEQ ID NO: 1 was stored at −20° C. prior to testing. The solvent was water and the solution was adjusted to pH of 5 with 1.0% glacial acetic acid or the diluent was PBS at a pH of 7.4.

Test organisms were either reference strains from the American Type Culture Collection, the Centers for Disease Control Antibiotic Resistance Bank, the Network on Antimicrobial Resistance in Staphylococcus aureus, or clinical isolates from the Micromyx collection. Upon receipt at Micromyx, the isolates were streaked under suitable conditions onto agar medium appropriate to each organism and were incubated for 18-24 hr at 35° C.

Colonies harvested from these growth plates were resuspended in the appropriate medium containing a cryoprotectant. Aliquots of each suspension were then frozen at −80° C.

Prior to testing, the isolates were sub-cultured onto trypticase soy agar with 5% sheep blood and incubated under optimal conditions for growth.

### Test Media.

Base test media for the TK assay were phosphate-buffered saline (PBS), water, and cation-adjusted Mueller-Hinton broth (CAMHB), all adjusted to pH 7.4 with sodium hydroxide (NaOH) or hydrochloric acid (HCl) and then filter-sterilized. Each base medium was evaluated with and without supplementation with sodium bicarbonate (NaCHO3) by dissolving NaCHO3 directly into each medium at 1.12× the desired final concentration of 50 mM and then adjusted to pH 7.4 with NaOH or HCl.

Double strength (2×) Dey/Engley neutralizing broth (D/E) was used as a neutralizer for dilution and plating during determination of CFU/mL at each TK assay time point. Track plating was conducted on TSAB for Staphylococcus aureus NRS382 and on TSA without sheep's blood for all other organisms. TSA was used for spread plate enumeration of CFU/mL during a pilot study testing toxicity of D/E to bacteria and neutralization of SEQ ID NO: 1 in D/E.

### Broth Microdilution MIC Assay

The activity of SEQ ID NO: 1 stocks against relevant ATCC reference isolates was determined by broth microdilution for QC purposes in accordance with guidelines described by the Clinical and Laboratory Standards Institute.

The wells in columns 2-12 in a standard 96-well microdilution plate were filled with 150 μL of 0.002% P-80 PBS. The drug (300 μL at 101× the desired top concentration in the test plates) was added to column 1. Serial two-fold dilutions were conducted through column 11. The wells of column 12 contained no drug and served as the organism growth control well. This would become the “mother” plate from which “daughter” or test plates would be prepared. The daughter plates were loaded with 190 μL of CAMHB+0.002% polysorbate-80 (P-80), or RPMI with 0.002% P-80 by hand-pipetting. The daughter plates received 2 μL of 101× drug solution from each well of the mother plate via hand-transfer using a multichannel pipette.

A standardized inoculum of each evaluated organism was prepared per CLSI methods. Growth from agar plates was used to prepare a 0.5 McFarland suspension in saline for each organism. The 0.5 McFarland suspensions were diluted 1:20 in saline, and the diluted suspensions were used as the test inocula. The inocula were then transferred to daughter plates by delivering 10 μL of standardized inoculum into each well of the daughter plate, resulting in a final concentration ranging between 2.5×10⁵ to 5×105 CFU/mL per well.

Plates were stacked and covered with a lid on the top plate, placed into plastic bags, and incubated at 35° C. for 16-20 hr in ambient air. Growth in the microplates was viewed from the bottom using a plate viewer. An un-inoculated solubility control was observed for evidence of drug (SEQ ID NO: 1) precipitation and to assess media sterility. The MIC was read and recorded as the lowest concentration of drug (SEQ ID NO: 1) that inhibited visible growth of the organism.

### Neutralization of SEQ ID NO: 1 by D/E and Toxicity of D/E

The ability for D/E to neutralize of SEQ ID NO: 1 and the toxicity of D/E to bacteria exposed for a maximum of 15 min was determined with Staphylococcus aureus NRS382 prior to conducting the TK assay. All PBS utilized below was pH 7.4.

The first three rows (rows A-C) of a 96-well microtiter plate were prepared with test media, where each row represented technical replicates for each condition tested. Survival of bacteria in D/E and neutralization of the rapid bactericidal activity of SEQ ID NO: 1 was assessed at 0 (columns 1-4), 5 (columns 5-8), and 15 min (columns 9-12). Columns 1, 5, and 9 were filled with 0.250 mL PBS. Columns 2, 6, and 10 were filled with 0.125 mL PBS and 0.125 mL 2×D/E. Columns 3 & 4, 7 & 8, and 11 & 12 were filled with only 0.125 ml 2×D/E prior to starting the assay.

A cell suspension was prepared from growth of a freshly streaked plate equivalent to the turbidity of a 0.5 McFarland standard (approx. 1.5×108 CFU/ml) in PBS and diluted 1:20,000 in the same. Immediately before inoculation of each test replicate for each test condition, 0.125 mL of drug was added to the appropriate well (columns 3, 7, and 11 for 8 μg/ml in PBS; columns 4, 8, and 12 for 500 μg/ml in PBS). The diluted cell suspension served as the inoculum, where 10 μL was added to the appropriate test wells immediately after addition of the drug to the appropriate wells. The target cell load for inoculation was ≤100 CFU/well. Start times for each replicate were staggered to allow time for subsequent spread plating.

To enumerate viable cells after the indicated incubation times, 100 μL was transferred from the assay microtiter plate to duplicate agar plates filled with approx. ten 2 mm sterile glass beads at the appropriate time points for each test condition and technical replicate. The sample was distributed evenly across the agar surface with vigorous rotational shaking of the plate, and glass beads were removed once the liquid was absorbed by the agar. D/E and PBS alone were also plated to assess media sterility. Plates were inverted, incubated overnight at 35° C., and CFU on each duplicate plate were counted. CFU from conditions containing either D/E or drug were compared directly to the viable cell counts of the appropriate controls to assess the ability for D/E to neutralize the drug and the toxicity of D/E to cells. A cutoff of ≥80% survival for D/E exposed cells and drug-exposed cells relative to the PBS control was used to demonstrate neutralization efficacy and lack of toxicity for D/E.

A second round of testing was also conducted to evaluate the ability for D/E to neutralize intermediate SEQ ID NO: 1 concentrations. The above procedure was followed to evaluate the neutralization of 25 and 50 μg/ml in PBS by D/E. D/E only controls for D/E toxicity assessment were omitted.

### Time-Kill (TK) Assay.

Drug concentrations tested were at 1×, 0.5×, and 0.25× the MIC values determined for SEQ ID NO: 1 when tested by broth microdilution in CAMHB in a Study 1. Drug stocks of 5120 μg/mL in water at pH 5 were diluted in PBS pH-7.4 to 100× the final concentrations desired for each TK assay and frozen at −20° C. until use.

The TK test panel was prepared in a 96-well deep well plate, where 890 μL of each test media and 10 μL of 100× drug stocks were added to the appropriate wells. A growth control containing no drug was included, where 10 μl of PBS (pH 7.4) was added in place of drug.

A cell suspension was prepared from growth of a freshly streaked plate equivalent to the turbidity of a 0.5 McFarland standard in PBS at (pH 7.4). Deep-well test panels prepared as described above were inoculated with 100 μL of the 0.5 McFarland cell suspension, targeting a final inoculum of approximately 1 to 2×10⁷ CFU/mL. During the TK assay, the 96 deep-well plate was left on the bench top at room temperature.

Aliquots of 100 μL were removed from the test panel immediately (0 minutes), and at 2.5, 5, 10, 15, and 30 minutes, and mixed with 100 μL 2×D/E to neutralize the rapid bactericidal activity of SEQ ID NO: 1. Cells were then serially diluted ten-fold in 2× Dey/Engley broth four times yielding a dilution series ranging from 2 to 2×10-4. Ten microliters from each step of the dilution series were track-plated in duplicate on TSA to enumerate viable counts (CFU/mL). Plates were then incubated at 35° C. overnight.

The resulting colonies were counted, and viable counts (CFU/mL) were determined from the average count of the duplicate plates. The limit of detection (LOD) for the assay was 100 CFU/mL. The LOD reflects the average CFU/ml if just one colony grew from the least diluted sample (DF=2) between the two track plate replicates, where 10 μL had been plated. Log-transformed viable counts (log10 CFU/mL) were then plotted vs. time.

### Results

Time-kill (TK) analysis was performed on 5 antibiotic-resistant isolates challenged with varying concentrations of SEQ ID NO: 1 in three different types of media (PBS, H₂O, CAMHB) in both the presence and absence of sodium bicarbonate. The evaluated strains were S. aureus NRS382 (hospital-acquired methicillin-resistant S. aureus; USA100), S. aureus NRS384 (community-acquired methicillin-resistant S. aureus; USA300), S. epidermidis MMX 8655 (methicillin-resistant S. epidermidis), E. coli CDC 0451 (KPC; MDR, multi-drug resistant), and P. aeruginosa CDC 0509 (VIM, MDR). During QC assessment of SEQ ID NO: 1 suspended in CAMHB, precipitation was observed at >8 μg/mL, however this precipitation did not interfere with the ability to determine the MIC. Precipitate was also apparent for 500 μg/mL SEQ ID NO: 1 suspended in D/E during neutralization pilot testing. No other solubility issues were encountered in the study. SEQ ID NO: 1 MIC values observed in CAMHB and RPMI during QC assessment (Table 12) were comparable to Study 1 for the QC isolates tested.

### Dey-Engley Toxicity and Neutralization of SEQ ID NO: 1 Pilot

Cell survival of S. aureus NRS382 in D/E compared to PBS was ≥80% for up to 15 minutes, showing that D/E was not toxic to this strain in these conditions. SEQ ID NO: 1 concentrations ≤50 μg/mL were neutralized for up to 15 minutes by D/E, thus validating the neutralization of up to 50 μg/mL SEQ ID NO: 1 for the TK evaluation. Dey-Engley toxicity and SEQ ID NO: 1 neutralization data can be found in Table 13A and 13B.

### TK Analysis

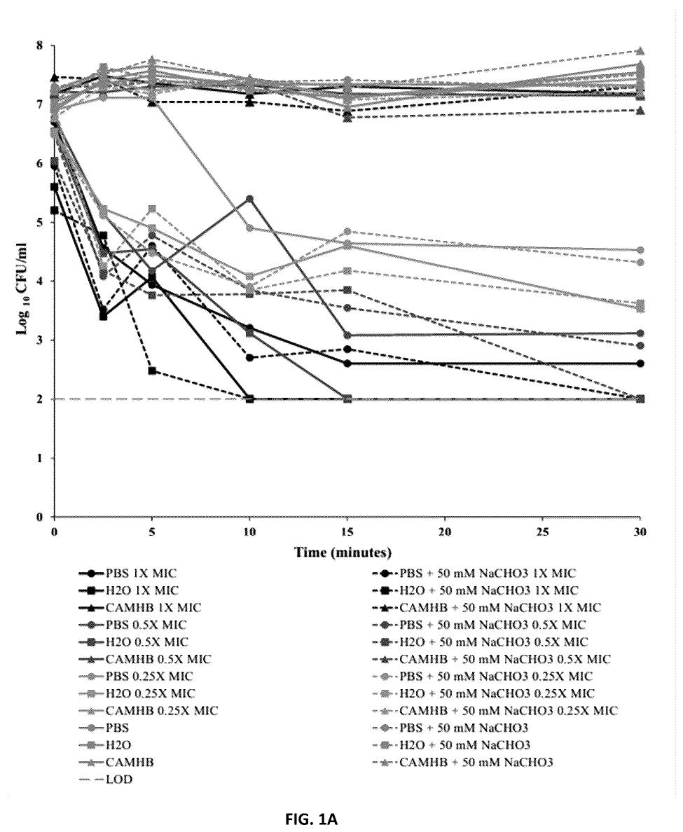

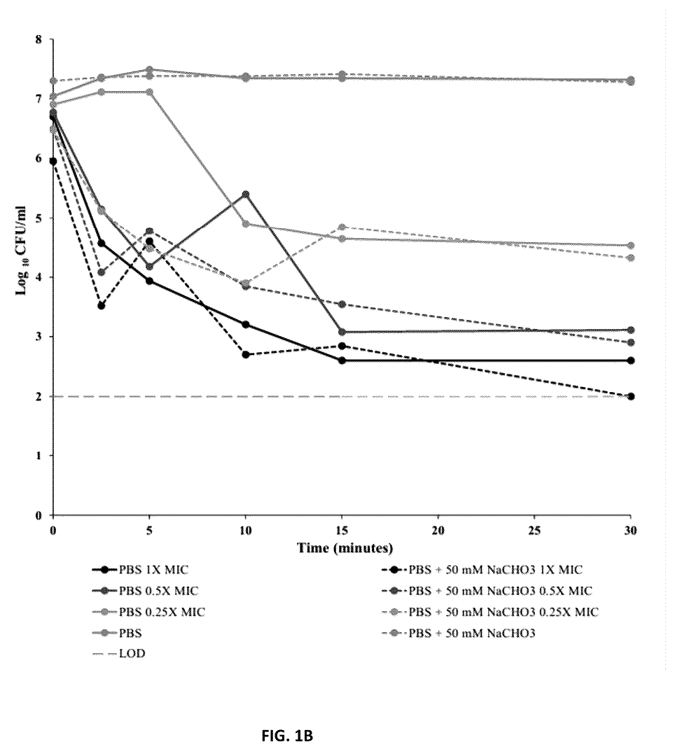

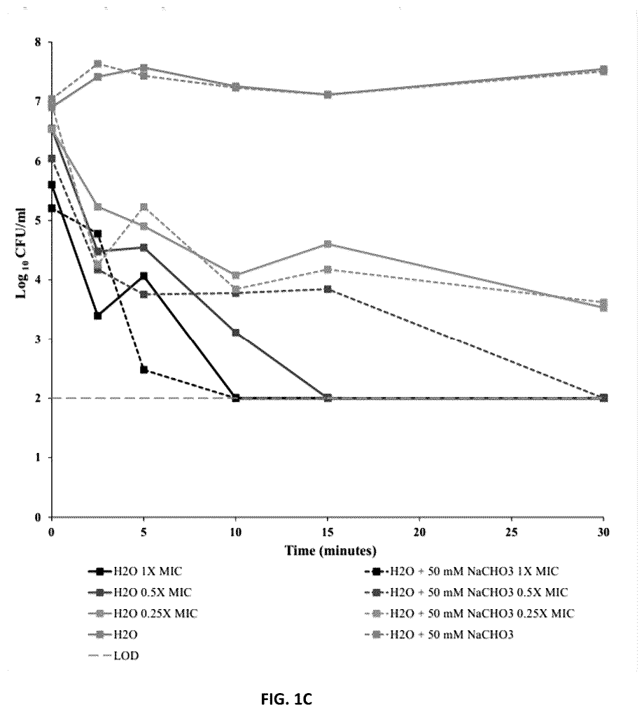

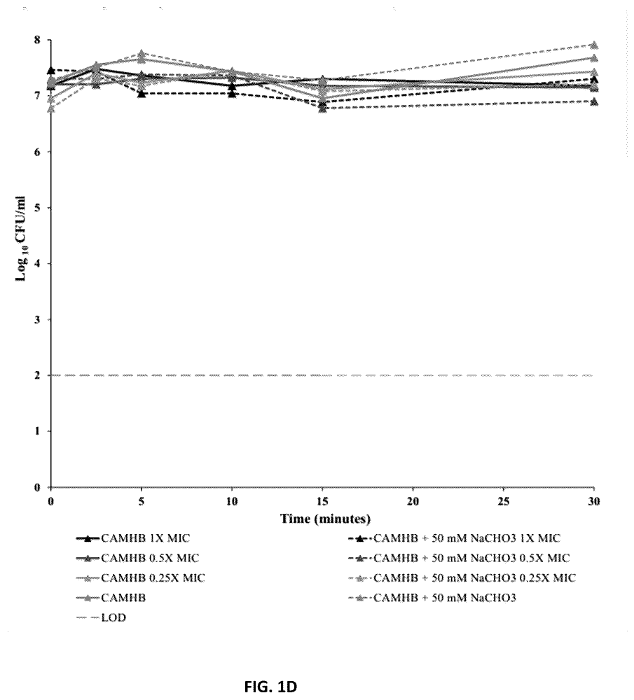

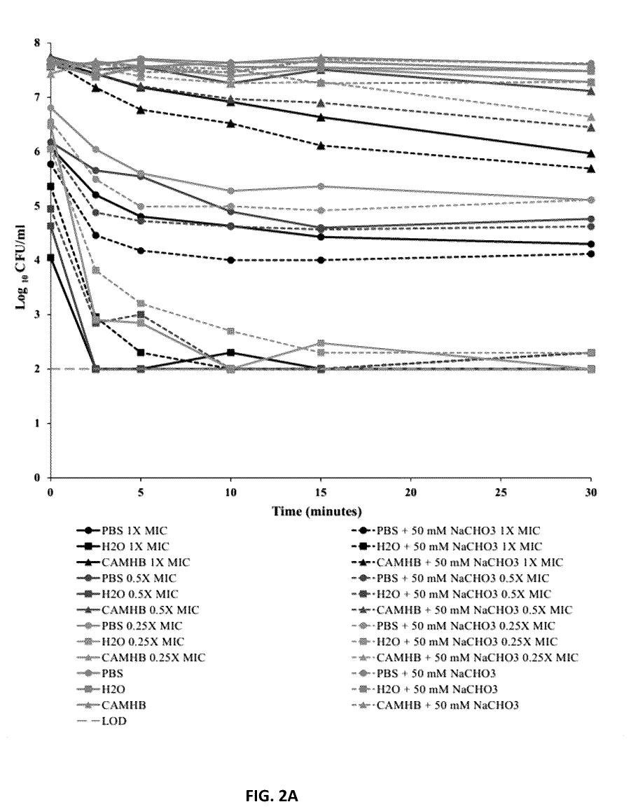

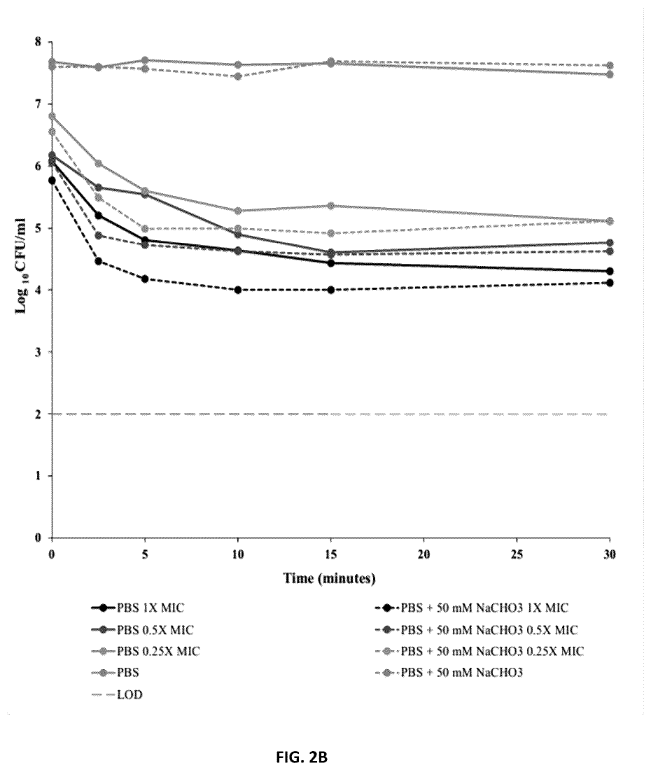

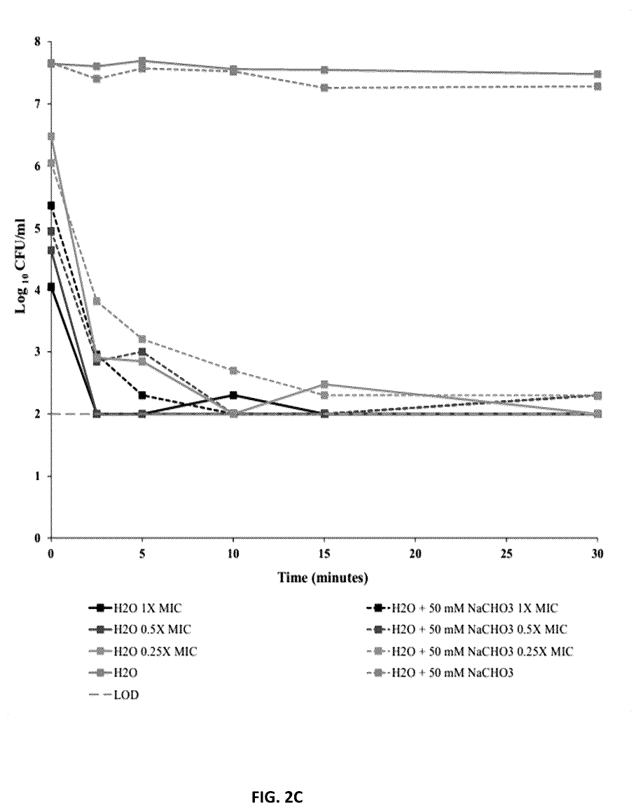

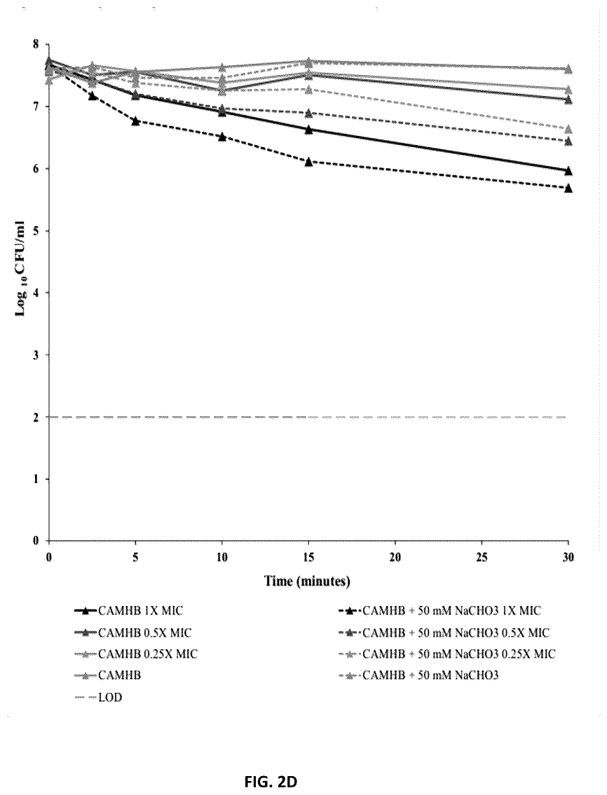

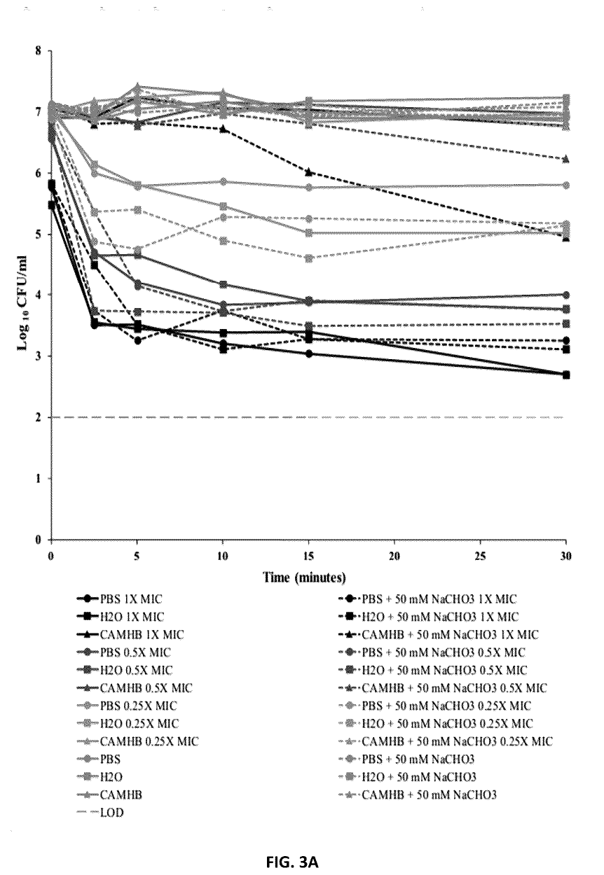

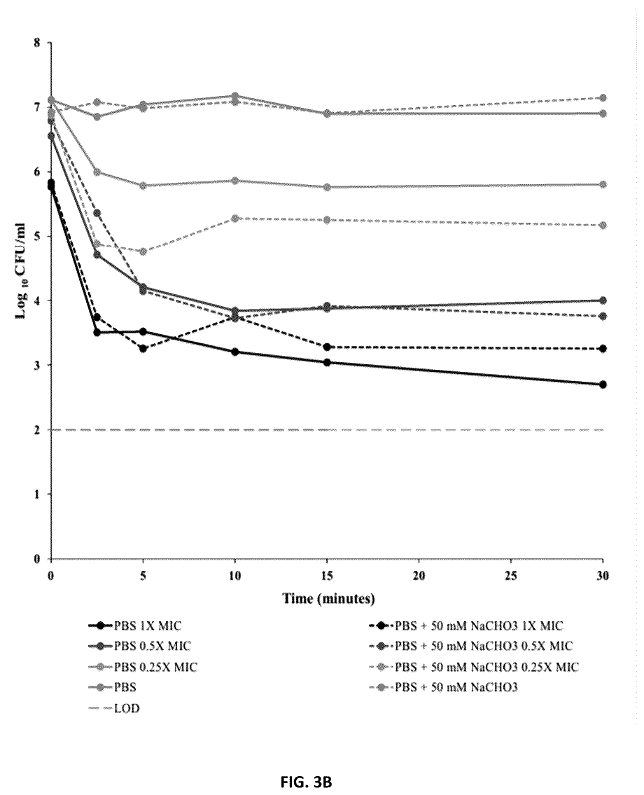

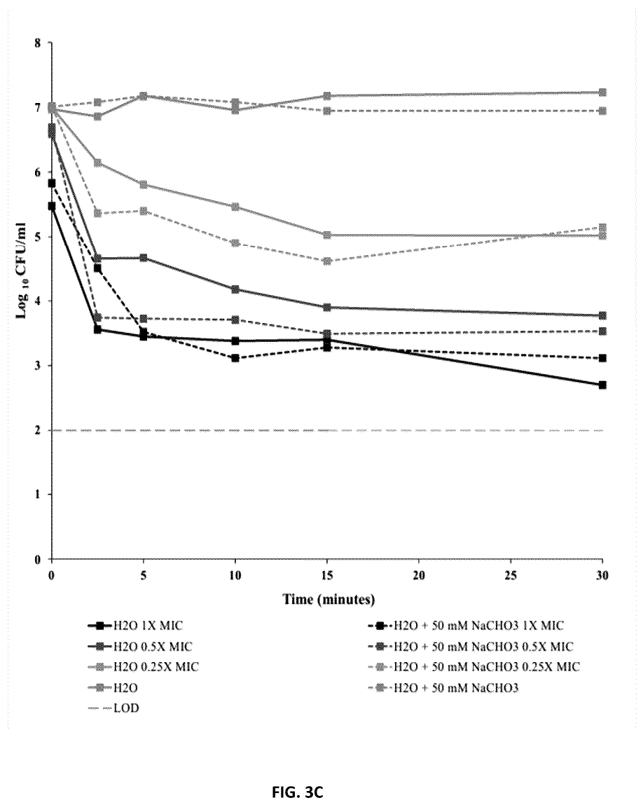

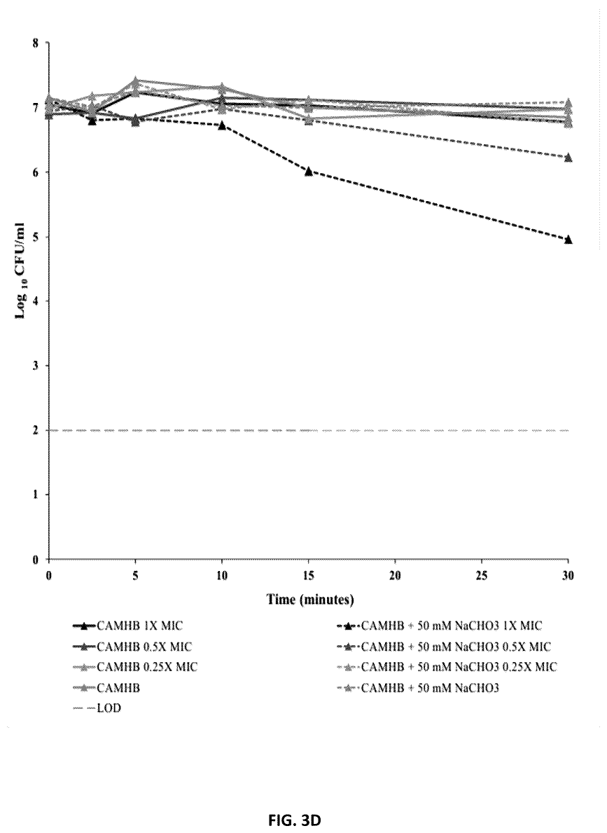

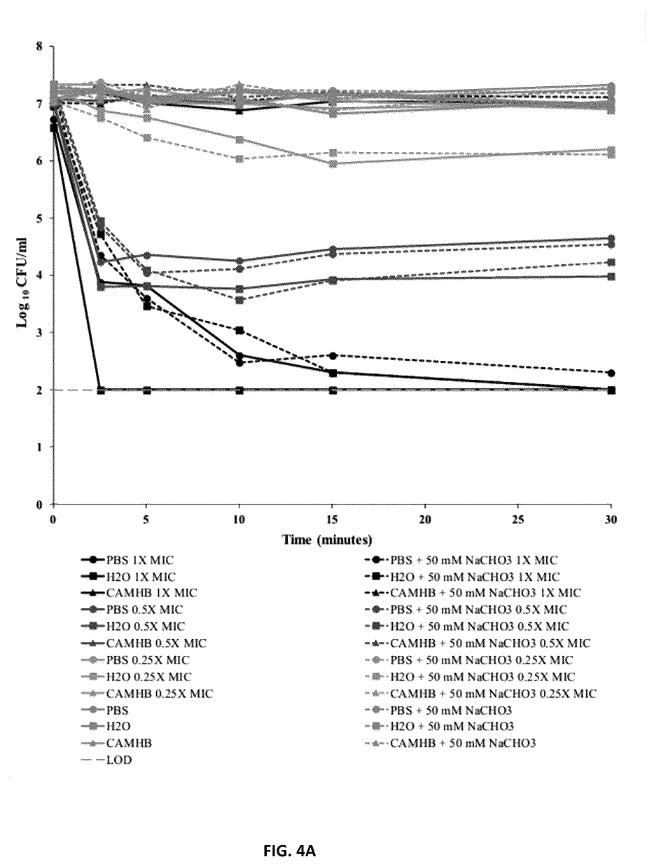

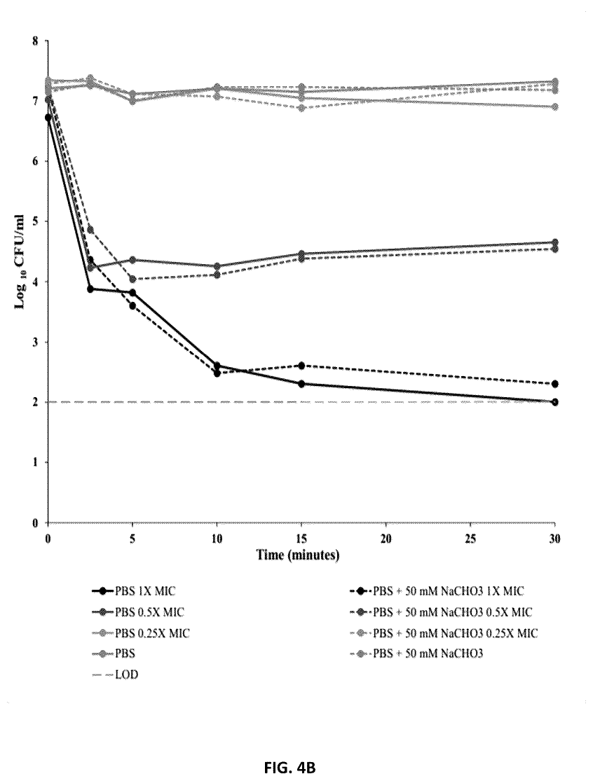

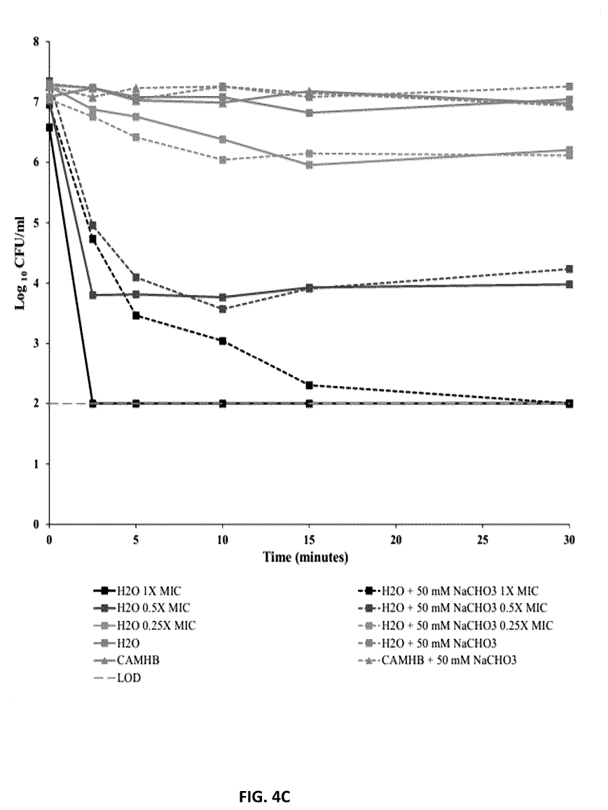

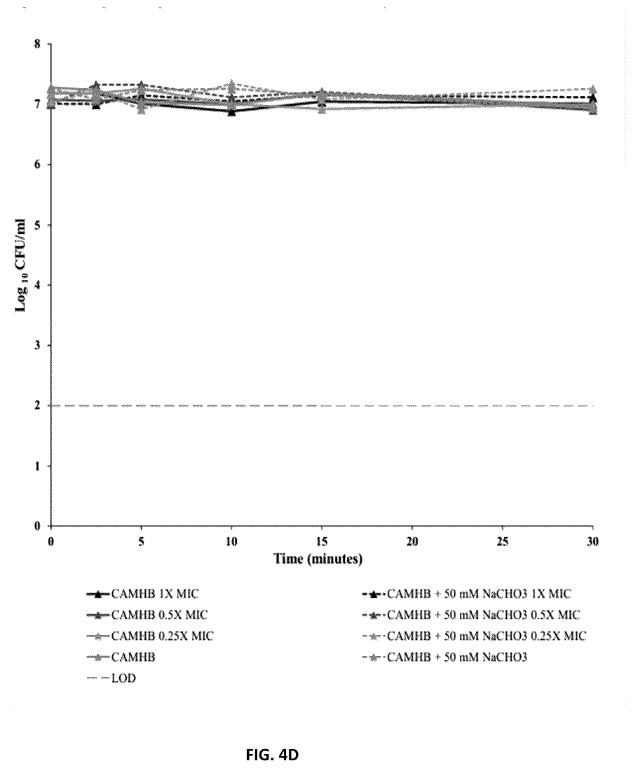

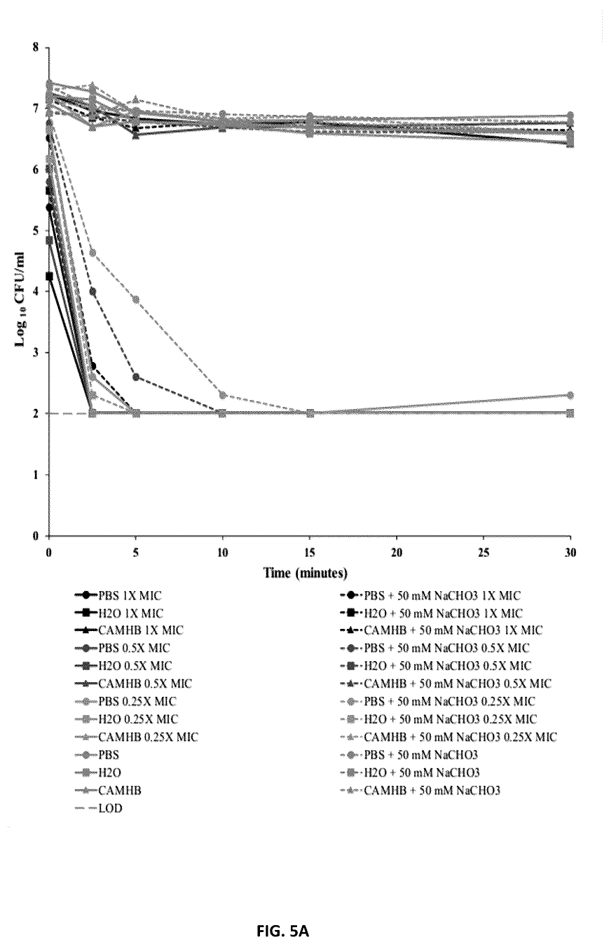

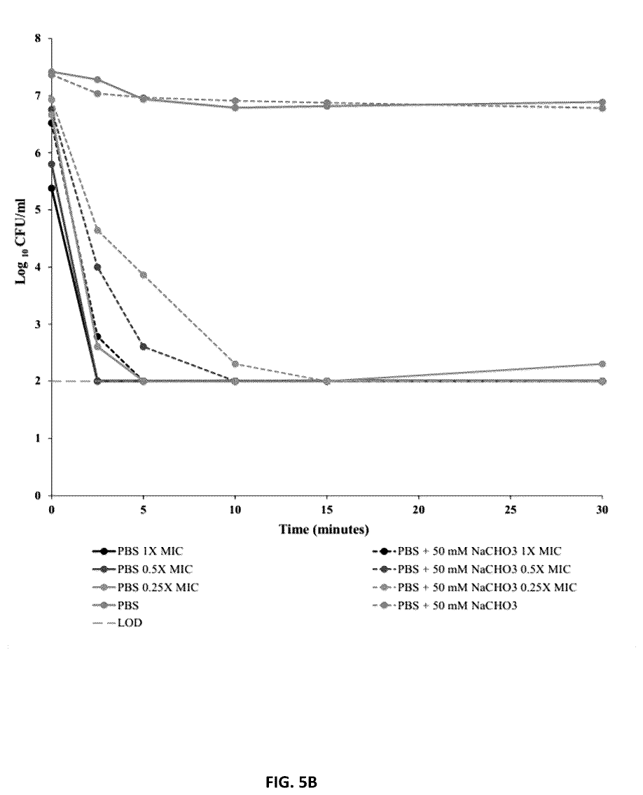

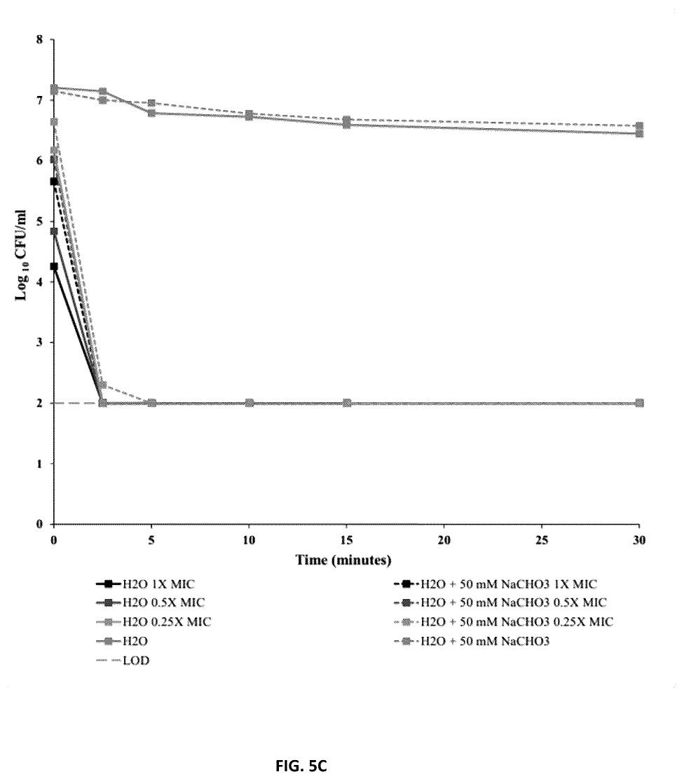

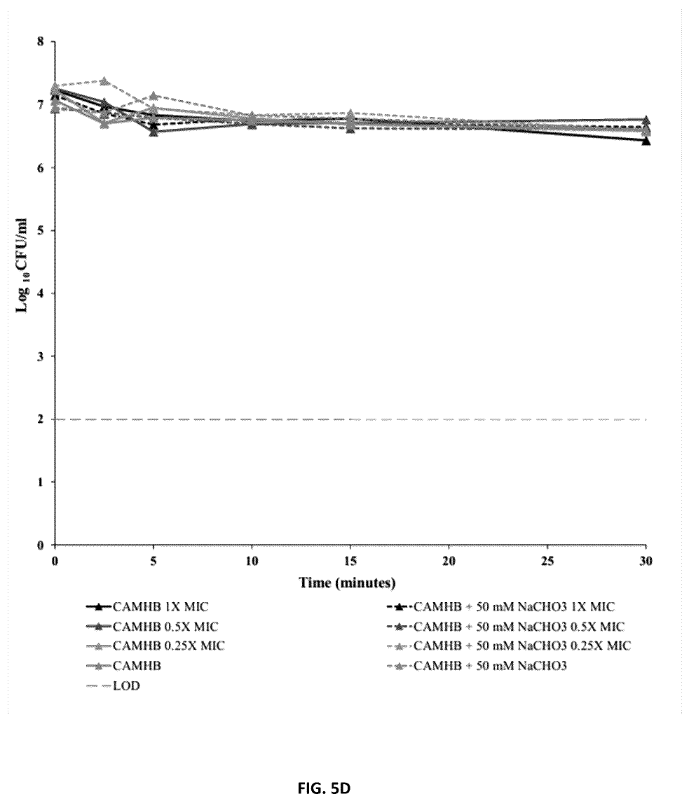

A summary of the SEQ ID NO: 1 killing of the 5 test isolates relative to the initial inoculum (viable counts at 0 min in the corresponding medium) is shown in Table 10A-E. Log-differences in CFU in base medium containing 50 mM sodium bicarbonate relative to the same base medium without sodium bicarbonate at each time point is summarized in Table 11A-D. Viable counts were plotted over time by test isolate in FIG. 1-FIG. 5. Corresponding TK analysis data is shown in Table 14A-E.

Greater than 1-log killing was evident at 2.5 min with all evaluated organisms in PBS and H₂O at 1× and 0.5× the SEQ ID NO: 1 MIC (Table 10A-E, FIG. 1-FIG. 5). In general, sodium bicarbonate addition had no observable impact on the log-kill of SEQ ID NO: 1 (Table 11A-E). Compared to the other conditions tested, SEQ ID NO: 1 demonstrated very little killing of the evaluated organisms when tested in CAMHB. Therefore, although sodium bicarbonate and SEQ ID NO: 1 were synergistic by MIC in CAMHB in a Study 1, synergistic killing was not observed over the short 30 minute course of the assay. MIC values are read after a 16-hour exposure to SEQ ID NO: 1, and perhaps a longer exposure in the TK assay would provide a better comparison.

The kinetics of killing varied for each organism and test medium. The most rapid killing was observed when testing SEQ ID NO: 1 against P. aeruginosa CDC 0509, where log-kill was at or near the 3-log threshold by 2.5 min for all SEQ ID NO: 1 concentrations in H₂O and PBS with and without sodium bicarbonate. Interestingly, most of the PBS and H₂O TK samples were already at the limit of detection (LOD) at this 2.5 min timepoint. Rapid killing may make it difficult to evaluate the contribution of sodium bicarbonate.

SEQ ID NO: 1 had greater killing in water when tested against the 2 S. aureus isolates. For S. aureus NRS 384, when SEQ ID NO: 1 was tested at 0.5 and 1× the MIC in H₂O, a ≥3-log-kill was achieved at 0 min in the absence of sodium bicarbonate and by 2.5 min in the presence of sodium bicarbonate. In contrast, killing of S. aureus NRS 384 by SEQ ID NO: 1 at 1× the MIC in PBS+/sodium bicarbonate was slightly slower, where log-kill was at or near the 3-log threshold after 2.5 min, and viable cells remained constant through remainder of the assay and never reached the LOD. Similar differences were observed between PBS and H₂O for S. aureus NRS 382 where log-kill by SEQ ID NO: 1 tested at 1× the MIC reached the LOD by 10 min in H₂O, while killing ≥LOD was achieved at 30 minutes only with PBS supplemented with sodium bicarbonate when SEQ ID NO: 1 was at 1× the MIC.

S. epidermidis MMX 8655 and E. coli CDC 0451 both showed similar kill kinetics in PBS and H₂O. For S. epidermidis MMX 8655 when SEQ ID NO: 1 was tested at 1× the MIC, log-kill was at or near the 3-log threshold by 2.5 min after which viable cells remained relatively constant over the course of the assay and never reached the LOD. For E. coli CDC 0451 without sodium bicarbonate when SEQ ID NO: 1 was at 1× the MIC, killing was ≥LOD at 2.5 min when testing in H₂O and was >3 at 2.5 min in PBS.

### Assessment of the Effect of Sodium Bicarbonate on SEQ ID NO: 1 Killing

Despite some observed kinetic differences (Table 11A-E), sodium bicarbonate did not have a strong impact on the bactericidal activity of SEQ ID NO: 1 enduring the course of the 30 min assay relative to that observed without bicarbonate. Log-CFU differences between media with sodium bicarbonate relative to media without sodium bicarbonate were typically less than 1-log across the evaluated organisms with some exceptions.

For S. aureus NRS382 and S. aureus NRS384 in H₂O at 0.5× and 1× the SEQ ID NO: 1 MIC, there were differences between log-CFU with and without sodium bicarbonate for individual data points, but no notable trends were observed. For S. aureus NRS382 in PBS where SEQ ID NO: 1 was at 0.25× the MIC, differences in log-CFU with sodium bicarbonate relative to log-CFU without sodium bicarbonate were 2 at 2.5 and 5 min, but the log-CFU difference dwindled to 1 by 10 min and did not persist over the course of the assay (Table 11A). Log-CFU differences between conditions with and without sodium bicarbonate were ≤−2 at 2.5 min for E. coli CDC 0451 in H₂O with SEQ ID NO: 1 at 1× the MIC (Table 11D; difference=−2.73), and for P. aeruginosa CDC 0509 in H₂O with SEQ ID NO: 1 at 0.25× and 0.5× (Table 11E; −2.00 and −2.04, respectively), but these differences did not persist over the course of the assay. The sole trend of sodium bicarbonate enhancing SEQ ID NO: 1 activity over the course of the assay was observed for S. epidermidis MMX 8655 in CAMHB+sodium bicarbonate, and the difference between the log-CFU with sodium bicarbonate relative to its absence only increased moderately over time (Change in log-CFU of 1.81 at 30 minutes).

In summary, SEQ ID NO: 1 demonstrated rapid bactericidal activity against all isolates in PBS and H₂O, while displaying little to no killing in CAMHB. While sodium bicarbonate addition to SEQ ID NO: 1 lowered the MIC values after overnight exposure for all tested isolates except P. aeruginosa CDC 0509 in a checkerboard panel in Study 1 relative to those observed in the absence of sodium bicarbonate, and in this study, the synergistic effect on the rate of killing was observed during TK evaluation over a 30-minute exposure was not present.

TABLE 10A
Summary of SEQ ID NO: 1 killing of S. aureus NRS 382 in various conditions over time

| Concentration | Multiple | | Log-kill (log10 CFU/mL) relative to base media at 0 min | | | | | |
|---|---|---|---|---|---|---|---|---|
| (μg/mL) | of MIC¹ | Media | 0 min | 2.5 min | 5 min | 10 min | 15 min | 30 min |
| 4 | 1X | PBS | 0.34 | 2.47 | 3.11^{A} | 3.84^{A} | 4.44^{A} | 4.44^{A} |
| 2 | 0.5X | | 0.27 | 1.90 | 2.87 | 1.64 | 3.96^{A} | 3.93^{A} |
| 1 | 0.25X | | 0.14 | −0.07 | −0.07 | 2.14 | 2.40 | 2.51 |
| 4 | 1X | PBS + | 1.35 | 3.78^{A} | 2.70 | 4.60^{A} | 4.46^{A} | 25.30^{B} |
| 2 | 0.5X | 50 mM NaCHO₃ | 0.81 | 3.22^{A} | 2.52 | 3.46^{A} | 3.76^{A} | 4.40^{A} |
| 1 | 0.25X | | 0.82 | 2.19 | 2.82 | 3.40^{A} | 2.46 | 2.98 |
| 4 | 1X | H2O | 1.30 | 3.51^{A} | 2.84 | 24.90^{B} | 24.90^{B} | 24.90^{B} |
| 2 | 0.5X | | 0.36 | 2.43 | 2.36 | 3.79^{A} | 24.90^{B} | 24.90^{B} |
| 1 | 0.25X | | 0.37 | 1.67 | 2.00 | 2.82 | 2.30 | 3.37^{A} |
| 4 | 1X | H2O + | 1.84 | 2.26 | 4.56^{A} | 25.04^{B} | 25.04^{B} | 25.04^{B} |
| 2 | 0.5X | 50 mM NaCHO₃ | 1.00 | 2.87 | 3.29^{A} | 3.26^{A} | 3.20^{A} | 25.04^{B} |
| 1 | 0.25X | | 0.09 | 2.79 | 1.81 | 3.20^{A} | 2.87 | 3.42^{A} |
| 4 | 1X | CAMHB | 0.05 | −0.25 | −0.13 | 0.05 | −0.07 | 0.05 |
| 2 | 0.5X | | 0.03 | 0.03 | −0.07 | −0.09 | 0.05 | 0.08 |
| 1 | 0.25X | | 0.28 | −0.17 | 0.00 | −0.22 | 0.12 | −0.20 |
| 4 | 1X | CAMHB + | −0.18 | −0.15 | 0.24 | 0.24 | 0.39 | −0.02 |
| 2 | 0.5X | 50 mM NaCHO₃ | −0.02 | −0.02 | −0.10 | −0.08 | 0.50 | 0.38 |
| 1 | 0.25X | | 0.50 | −0.02 | 0.10 | −0.17 | 0.20 | 0.07 |

¹SEQ ID NO: 1 MIC by broth microdilution in CAMHB | SEQ ID NO: 1, MIC = 4 μg/mL
^{A}Instances where 99.9% (3-log) kill was achieved
^{B}Instances where killing was at or beyond the limit of detection of the assay (LOD)

TABLE 10B
Summary of SEQ ID NO: 1 killing of S. aureus NRS 384 in various conditions over time

| Concentration | Multiple | | Log-kill (log10 CFU/mL) relative to base media at 0 min | | | | | |
|---|---|---|---|---|---|---|---|---|
| (μg/mL) | of MIC¹ | Media | 0 min | 2.5 min | 5 min | 10 min | 15 min | 30 min |
| 8 | 1X | PBS | 1.60 | 2.48 | 2.88 | 3.05^{A} | 3.25^{A} | 3.38^{A} |
| 4 | 0.5X | | 1.51 | 2.03 | 2.14 | 2.78 | 3.08^{A} | 2.92 |
| 2 | 0.25X | | 0.88 | 1.64 | 2.08 | 2.40 | 2.32 | 2.57 |
| 8 | 1X | PBS + | 1.83 | 3.14^{A} | 3.43^{A} | 3.60^{A} | 3.60^{A} | 3.49^{A} |
| 4 | 0.5X | 50 mM NaCHO₃ | 1.54 | 2.72 | 2.88 | 2.98 | 3.03^{A} | 2.98 |
| 2 | 0.25X | H2O | 1.05 | 2.11 | 2.61 | 2.61 | 2.68 | 2.49 |
| 8 | 1X | | 3.60^{A} | 25.64^{B} | 25.64^{B} | 5.34^{A} | 25.64^{B} | 25.64^{B} |
| 4 | 0.5X | | 3.01^{A} | 25.64^{B} | 25.64^{B} | 25.64^{B} | 25.64^{B} | 25.64^{B} |
| 2 | 0.25X | | 1.17 | 4.74^{A} | 4.80^{A} | 25.64^{B} | 5.17^{A} | 25.64^{B} |
| 8 | 1X | H2O + | 2.29 | 4.70^{A} | 5.35^{A} | 25.65^{B} | 25.65^{B} | 5.35^{A} |
| 4 | 0.5X | 50 mM NaCHO₃ | 2.70 | 4.81^{A} | 4.65^{A} | 25.65^{B} | 25.65^{B} | 5.35^{A} |
| 2 | 0.25X | | 1.60 | 3.84^{A} | 4.45^{A} | 4.95^{A} | 5.35^{A} | 5.35^{A} |
| 8 | 1X | CAMHB | −0.05 | 0.19 | 0.44 | 0.71 | 0.99 | 1.65 |
| 4 | 0.5X | | −0.12 | 0.12 | 0.07 | 0.37 | 0.12 | 0.51 |
| 2 | 0.25X | | 0.19 | −0.04 | 0.06 | 0.24 | 0.08 | 0.34 |
| 8 | 1X | CAMHB + | −0.05 | 0.44 | 0.84 | 1.09 | 1.50 | 1.92 |
| 4 | 0.5X | 50 mM NaCHO₃ | 0.04 | 0.18 | 0.41 | 0.64 | 0.72 | 1.17 |
| 2 | 0.25X | | 0.02 | 0.07 | 0.23 | 0.36 | 0.33 | 0.97 |

¹SEQ ID NO: 1 MIC by broth microdilution in CAMHB | SEQ ID NO: 1, MIC = 8 μg/mL
^{A}Instances where 99.9% (3-log) kill was achieved
^{B}Instances where killing was at or beyond the limit of detection of the assay (LOD)

TABLE 10C
Summary of SEQ ID NO: 1 killing of S. epidermidis MMX 8655 in various conditions over time

| Concentration | Multiple | | Log-kill (log10 CFU/mL) relative to base media at 0 min | | | | | |
|---|---|---|---|---|---|---|---|---|
| (μg/mL) | of MIC¹ | Media | 0 min | 2.5 min | 5 min | 10 min | 15 min | 30 min |
| 1 | 1X | PBS | 1.34 | 3.61^{A} | 3.60^{A} | 3.91^{A} | 4.07^{A} | 4.41^{A} |
| 0.5 | 0.5X | | 0.56 | 2.41 | 2.91 | 3.28^{A} | 3.24^{A} | 3.11^{A} |
| 0.25 | 0.25X | | 0.00 | 1.12 | 1.33 | 1.25 | 1.35 | 1.31 |
| 1 | 1X | PBS + | 1.09 | 3.18^{A} | 3.66^{A} | 3.18^{A} | 3.64^{A} | 3.66^{A} |
| 0.5 | 0.5X | 50 mM NaCHO₃ | 0.13 | 1.56 | 2.77 | 3.19^{A} | 3.01^{A} | 3.16^{A} |
| 0.25 | 0.25X | | 0.04 | 2.04 | 2.16 | 1.64 | 1.66 | 1.74 |
| 1 | 1X | H2O | 1.49 | 3.41^{A} | 3.52^{A} | 3.59^{A} | 3.57^{A} | 4.27^{A} |
| 0.5 | 0.5X | | 0.38 | 2.32 | 2.31 | 2.79 | 3.07^{A} | 3.20^{A} |
| 0.25 | 0.25X | | −0.03 | 0.83 | 1.16 | 1.51 | 1.94 | 1.95 |
| 1 | 1X | H2O + | 1.18 | 2.50 | 3.49^{A} | 3.89^{A} | 3.73^{A} | 3.89^{A} |
| 0.5 | 0.5X | 50 mM NaCHO₃ | 0.32 | 3.27^{A} | 3.28^{A} | 3.30^{A} | 3.52^{A} | 3.48^{A} |
| 0.25 | 0.25X | | −0.01 | 1.65 | 1.61 | 2.11 | 2.40 | 1.86 |
| 1 | 1X | CAMHB | 0.11 | 0.24 | −0.08 | 0.09 | 0.12 | 0.38 |
| 0.5 | 0.5X | | 0.25 | 0.23 | 0.32 | 0.00 | 0.03 | 0.17 |
| 0.25 | 0.25X | | 0.17 | −0.03 | −0.08 | −0.18 | 0.32 | 0.18 |
| 1 | 1X | CAMHB + | 0.03 | 0.35 | 0.32 | 0.42 | 1.13 | 2.19 |
| 0.5 | 0.5X | 50 mM NaCHO₃ | 0.21 | 0.12 | 0.37 | 0.17 | 0.35 | 0.92 |
| 0.25 | 0.25X | | 0.10 | 0.15 | −0.22 | 0.18 | 0.03 | 0.39 |

¹SEQ ID NO: 1 MIC by broth microdilution in CAMHB | SEQ ID NO: 1, MIC = 1 μg/mL
^{A}Instances where 99.9% (3-log) kill was achieved

TABLE 10D
Summary of SEQ ID NO: 1 killing of E. coli CDC 0451 in various conditions over time

| Concentration | Multiple | | Log-kill (log10 CFU/mL) relative to base media at 0 min | | | | | |
|---|---|---|---|---|---|---|---|---|
| (μg/mL) | of MIC¹ | Media | 0 min | 2.5 min | 5 min | 10 min | 15 min | 30 min |
| 1 | 1X | PBS | 0.48 | 3.32^{A} | 3.38^{A} | 4.60^{A} | 4.90^{A} | ≥5.20^{B} |
| 0.5 | 0.5X | | 0.18 | 2.97 | 2.84 | 2.95 | 2.74 | 2.55 |
| 0.25 | 0.25X | | −0.14 | −0.12 | 0.21 | 0.00 | 0.16 | 0.30 |
| 1 | 1X | PBS + | −0.08 | 2.78 | 3.54^{A} | 4.67^{A} | 4.54^{A} | 4.85^{A} |
| 0.5 | 0.5X | 50 mM NaCHO₃ | −0.11 | 2.28 | 3.10^{A} | 3.03^{A} | 2.77 | 2.60 |
| 0.25 | 0.25X | | −0.13 | −0.23 | 0.03 | 0.07 | 0.27 | −0.13 |
| 1 | 1X | H2O | 0.50 | ≥5.08^{B} | ≥5.08^{B} | ≥5.08^{B} | ≥5.08^{B} | ≥5.08^{B} |
| 0.5 | 0.5X | | 0.07 | 3.28^{A} | 3.27^{A} | 3.32^{A} | 3.16^{A} | 3.11^{A} |
| 0.25 | 0.25X | | −0.17 | 0.21 | 0.33 | 0.70 | 1.13 | 0.88 |
| 1 | 1X | H2O + | 0.34 | 2.57 | 3.84^{A} | 4.26^{A} | 5.00^{A} | ≥5.30^{B} |
| 0.5 | 0.5X | 50 mM NaCHO₃ | −0.04 | 2.35 | 3.21^{A} | 3.73^{A} | 3.39^{A} | 3.07^{A} |
| 0.25 | 0.25X | | 0.26 | 0.55 | 0.89 | 1.26 | 1.15 | 1.19 |
| 1 | 1X | CAMHB | 0.10 | 0.10 | 0.27 | 0.40 | 0.24 | 0.27 |
| 0.5 | 0.5X | | 0.20 | 0.23 | 0.20 | 0.28 | 0.10 | 0.38 |
| 0.25 | 0.25X | | 0.10 | 0.10 | 0.02 | 0.28 | 0.36 | 0.28 |
| 1 | 1X | CAMHB + | 0.25 | 0.26 | 0.11 | 0.21 | 0.11 | 0.14 |
| 0.5 | 0.5X | 50 mM NaCHO₃ | 0.26 | −0.07 | −0.07 | 0.14 | 0.05 | 0.30 |
| 0.25 | 0.25X | | 0.21 | 0.05 | 0.34 | −0.09 | 0.18 | 0.00 |

¹SEQ ID NO: 1 MIC by broth microdilution in CAMHB | SEQ ID NO: 1, MIC = 1 μg/mL
^{A}Instances where 99.9% (3-log) kill was achieved
^{B}Instances where killing was at or beyond the limit of detection of the assay (LOD)

TABLE 10E
Summary of SEQ ID NO: 1 killing of P. aeruginosa CDC 0509 in various conditions over time

| Concentration | Multiple | | Log-kill (log10 CFU/mL) relative to base media at 0 min | | | | | |
|---|---|---|---|---|---|---|---|---|
| (μg/mL) | of MIC¹ | Media | 0 min | 2.5 min | 5 min | 10 min | 15 min | 30 min |
| 16 | 1X | PBS | 2.03 | 25.41^{B} | 25.41^{B} | 25.41^{B} | 25.41^{B} | 25.41^{B} |
| 8 | 0.5X | | 1.62 | 25.41^{B} | 25.41^{B} | 25.41^{B} | 25.41^{B} | 25.41^{B} |
| 4 | 0.25X | | 0.75 | 4.81^{A} | 25.41^{B} | 25.41^{B} | 25.41^{B} | 5.11^{A} |
| 16 | 1X | PBS + | 0.84 | 4.58^{A} | 25.36^{B} | 25.36^{B} | 25.36^{B} | 25.36^{B} |
| 8 | 0.5X | 50 mM NaCHO₃ | 0.61 | 3.36^{A} | 4.76^{A} | 25.36^{B} | 25.36^{B} | 25.36^{B} |
| 4 | 0.25X | | 0.43 | 2.72 | 3.50^{A} | 5.06^{A} | 25.36^{B} | 25.36^{B} |
| 16 | 1X | H2O | 2.95 | 25.20^{B} | 25.20^{B} | 25.20^{B} | 25.20^{B} | 25.20^{B} |
| 8 | 0.5X | | 2.37 | 25.20^{B} | 25.20^{B} | 25.20^{B} | 25.20^{B} | 25.20^{B} |
| 4 | 0.25X | | 1.03 | 25.20^{B} | 25.20^{B} | 25.20^{B} | 25.20^{B} | 25.20^{B} |
| 16 | 1X | H2O + | 1.48 | 25.15^{B} | 25.15^{B} | 25.15^{B} | 25.15^{B} | 25.15^{B} |
| 8 | 0.5X | 50 mM NaCHO₃ | 1.12 | 25.15^{B} | 25.15^{B} | 25.15^{B} | 25.15^{B} | 25.15^{B} |
| 4 | 0.25X | | 0.50 | 4.85^{A} | 25.15^{B} | 25.15^{B} | 25.15^{B} | 25.15^{B} |
| 16 | 1X | CAMHB | −0.16 | 0.10 | 0.24 | 0.33 | 0.29 | 0.64 |
| 8 | 0.5X | | −0.19 | 0.03 | 0.50 | 0.38 | 0.36 | 0.30 |
| 4 | 0.25X | | −0.16 | 0.36 | 0.12 | 0.29 | 0.38 | 0.49 |
| 16 | 1X | CAMHB + | −0.19 | 0.10 | 0.27 | 0.18 | 0.26 | 0.31 |
| 8 | 0.5X | 50 mM NaCHO₃ | 0.02 | 0.08 | 0.16 | 0.26 | 0.33 | 0.35 |
| 4 | 0.25X | | −0.35 | −0.43 | 0.02 | 0.12 | 0.09 | 0.37 |

¹SEQ ID NO: 1 MIC by broth microdilution in CAMHB | SEQ ID NO: 1, MIC = 16 μg/mL
^{A}Instances where 99.9% (3-log) kill was achieved
^{B}Instances where killing was at or beyond the limit of detection of the assay (LOD)

TABLE 11A
Summary of S. aureus NRS 382 log-CFU in bicarbonate relative
to corresponding media without sodium bicarbonate

| Concentration | Multiple | | Change in CFU (log10 CFU/ml) relative to media w/o NaCHO₃ | | | | | |
|---|---|---|---|---|---|---|---|---|
| (ug/mL) | of MIC¹ | Media | 0 min | 2.5 min | 5 min | 10 min | 15 min | 30 min |
| 4 | 1X | PBS + | 0.74 | 1.05^{A} | −0.67 | 0.51 | −0.24 | 0.60 |
| 2 | 0.5X | 50 mM NaCHO₃ | 0.28 | 1.07^{A} | −0.60 | 1.55^{A} | −0.46 | 0.21 |
| 1 | 0.25X | | 0.43 | 2.00^{B} | 2.64^{B} | 1.00^{A} | −0.20 | 0.21 |
| 0 | NA | | −0.26 | −0.02 | 0.11 | −0.04 | −0.07 | 0.04 |
| 4 | 1X | H2O + | 0.40 | −1.38^{C} | 1.59^{A} | 0.00 | 0.00 | 0.00 |
| 2 | 0.5X | 50 mM NaCHO₃ | 0.50 | 0.30 | 0.79 | −0.66 | −1.85^{C} | 0.00 |
| 1 | 0.25X | | −0.42 | 0.98 | −0.33 | 0.23 | 0.43 | −0.09 |
| 0 | NA | | −0.14 | −0.22 | 0.14 | 0.02 | 0.00 | 0.04 |
| 4 | 1X | CAMHB + | −0.29 | 0.05 | 0.32 | 0.13 | 0.41 | −0.12 |
| 2 | 0.5X | 50 mM NaCHO₃ | −0.10 | −0.10 | −0.08 | −0.04 | 0.40 | 0.24 |
| 1 | 0.25X | | 0.18 | 0.10 | 0.05 | 0.00 | 0.03 | 0.23 |
| 0 | NA | | −0.05 | 0.05 | −0.11 | 0.00 | −0.32 | −0.23 |

¹SEQ ID NO: 1 MIC by broth microdilution in CAMHB
^{A}Instances where the difference in log-CFU with sodium bicarbonate (NaCHO₃) relative to log-CFU without NaCHO₃ ranged from 1 to 2
^{B}Instances where the difference in log-CFU with sodium bicarbonate (NaCHO₃) relative to log-CFU without NaCHO₃ was >2
^{C}Instances where the difference in log-CFU with sodium bicarbonate (NaCHO3) relative to log-CFU without NaCHO3 ranged from −1 to −2

TABLE 11B
Summary of S. aureus NRS 384 log-CFU in bicarbonate relative
to corresponding media without sodium bicarbonate

| Concentration | Multiple | | Change in CFU (log10 CFU/ml) relative to media w/o NaCHO₃ | | | | | |
|---|---|---|---|---|---|---|---|---|
| (μg/mL) | of MIC¹ | Media | 0 min | 2.5 min | 5 min | 10 min | 15 min | 30 min |
| 8 | 1X | PBS + | 0.31 | 0.74 | 0.63 | 0.63 | 0.43 | 0.19 |
| 4 | 0.5X | 50 mM NaCHO₃ | 0.11 | 0.77 | 0.82 | 0.27 | 0.03 | 0.14 |
| 2 | 0.25X | | 0.25 | 0.55 | 0.61 | 0.28 | 0.44 | 0.00 |
| 0 | NA | | 0.08 | −0.01 | 0.14 | 0.19 | −0.04 | −0.15 |
| 8 | 1X | H2O + | −1.32^{C} | −0.95 | −0.30 | 0.30 | 0.00 | −0.30 |
| 4 | 0.5X | 50 mM NaCHO₃ | −0.32 | −0.85 | −1.00^{C} | 0.00 | 0.00 | −0.30 |
| 2 | 0.25X | | 0.43 | −0.91 | −0.36 | −0.70 | 0.18 | −0.30 |
| 0 | NA | | −0.01 | 0.20 | 0.12 | 0.04 | 0.29 | 0.20 |
| 8 | 1X | CAMHB+ | 0.01 | 0.26 | 0.41 | 0.40 | 0.52 | 0.28 |
| 4 | 0.5X | | 0.18 | 0.07 | 0.35 | 0.29 | 0.61 | 0.67 |
| 2 | 0.25X | | −0.16 | 0.12 | 0.19 | 0.12 | 0.27 | 0.64 |
| 0 | NA | | 0.01 | −0.25 | 0.09 | 0.17 | 0.03 | −0.01 |

¹SEQ ID NO: 1 MIC by broth microdilution in CAMHB
^{C}Instances where the difference in log-CFU with sodium bicarbonate (NaCHO3) relative to log-CFU without NaCHO3 ranged from −1 to −2

TABLE 11C
Summary of S. epidermidis MMX 8655 log-CFU in bicarbonate
relative to corresponding media without sodium bicarbonate

| Concentration | Multiple | | Change in CFU (log10 CFU/ml) relative to media w/o NaCHO₃ | | | | | |
|---|---|---|---|---|---|---|---|---|
| (μg/mL) | of MIC¹ | Media | 0 min | 2.5 min | 5 min | 10 min | 15 min | 30 min |
| 1 | 1X | PBS + | −0.06 | −0.24 | 0.26 | −0.54 | −0.24 | −0.56 |
| 0.5 | 0.5X | 50 mM NaCHO₃ | −0.24 | −0.65 | 0.06 | 0.11 | −0.04 | 0.24 |
| 0.25 | 0.25X | | 0.23 | 1.11^{A} | 1.03^{A} | 0.58 | 0.51 | 0.63 |
| 0 | NA | | 0.19 | −0.23 | 0.06 | 0.09 | −0.01 | −0.24 |
| 1 | 1X | H2O + | −0.35 | −0.95 | −0.07 | 0.27 | 0.12 | −0.41 |
| 0.5 | 0.5X | 50 mM NaCHO₃ | −0.10 | 0.91 | 0.94 | 0.47 | 0.41 | 0.24 |
| 0.25 | 0.25X | | −0.02 | 0.78 | 0.41 | 0.56 | 0.41 | −0.13 |
| 0 | NA | | −0.04 | −0.22 | 0.00 | −0.12 | 0.23 | 0.29 |
| 1 | 1X | CAMHB + | −0.08 | 0.11 | 0.40 | 0.33 | 1.01^{A} | 1.81^{A} |
| 0.5 | 0.5X | 50 mM NaCHO₃ | −0.05 | −0.11 | 0.05 | 0.17 | 0.31 | 0.74 |
| 0.25 | 0.25X | | −0.06 | 0.18 | −0.13 | 0.35 | −0.29 | 0.21 |
| 0 | NA | | 0.00 | −0.08 | 0.16 | 0.25 | 0.00 | −0.23 |

¹SEQ ID NO: 1 MIC by broth microdilution in CAMHB
^{A}Instances where the difference in log-CFU with sodium bicarbonate (NaCHO₃) relative to log-CFU without NaCHO₃ ranged from 1 to 2

TABLE 11D
Summary of E. coli CDC 0451 log-CFU in bicarbonate relative
to corresponding media without sodium bicarbonate

| Concentration | Multiple | | Change in CFU (log10 CFU/ml) relative to media w/o NaCHO3 | | | | | |
|---|---|---|---|---|---|---|---|---|
| (μg/mL) | of MIC¹ | Media | 0 min | 2.5 min | 5 min | 10 min | 15 min | 30 min |
| 1 | 1X | PBS + | −0.51 | −0.48 | 0.22 | 0.12 | −0.30 | −0.30 |
| 0.5 | 0.5X | 50 mM NaCHO3 | −0.23 | −0.63 | 0.32 | 0.14 | 0.08 | 0.11 |
| 0.25 | 0.25X | | 0.06 | −0.06 | −0.12 | 0.13 | 0.16 | −0.38 |
| 0 | NA | | 0.06 | −0.02 | 0.11 | −0.03 | −0.08 | 0.15 |
| 1 | 1X | H2O + | −0.38 | −2.73^{D} | −1.46^{C} | −1.04^{C} | −0.30 | 0.00 |
| 0.5 | 0.5X | 50 mM NaCHO3 | −0.33 | −1.15^{C} | −0.28 | 0.20 | 0.02 | −0.25 |
| 0.25 | 0.25X | | 0.21 | 0.12 | 0.34 | 0.34 | −0.19 | 0.09 |
| 0 | NA | | −0.22 | 0.00 | 0.04 | −0.18 | −0.26 | −0.21 |
| 1 | 1X | CAMHB + | 0.17 | 0.18 | −0.14 | −0.16 | −0.10 | −0.11 |
| 0.5 | 0.5X | 50 mM NaCHO3 | 0.08 | −0.27 | −0.24 | −0.11 | −0.03 | −0.05 |
| 0.25 | 0.25X | | 0.13 | −0.03 | 0.34 | −0.34 | −0.16 | −0.26 |
| 0 | NA | | 0.02 | 0.15 | −0.21 | −0.27 | 0.03 | 0.03 |

¹SEQ ID NO: 1 MIC by broth microdilution in CAMHB
^{C}Instances where the difference in log-CFU with sodium bicarbonate (NaCHO₃) relative to log-CFU without NaCHO₃ ranged from −1 to −2
^{D}Instances where the difference in log-CFU with sodium bicarbonate (NaCHO₃) relative to log-CFU without NaCHO₃ was <−2

TABLE 11E
Summary of P. aeruginosa CDC 0509 log-CFU in bicarbonate
relative to corresponding media without sodium bicarbonate

| Concentration | Multiple | | Change in CFU (log10 CFU/ml) relative to media w/o NaCHO₃ | | | | | |
|---|---|---|---|---|---|---|---|---|
| (μg/mL) | of MIC¹ | Media | 0 min | 2.5 min | 5 min | 10 min | 15 min | 30 min |
| 16 | 1X | PBS + | −1.14^{C} | −0.78 | 0.00 | 0.00 | 0.00 | 0.00 |
| 8 | 0.5X | 50 mM NaCHO₃ | −0.96 | −2.00^{D} | −0.60 | 0.00 | 0.00 | 0.00 |
| 4 | 0.25X | | −0.27 | −2.04^{D} | −1.86^{C} | −0.30 | 0.00 | 0.30 |
| 0 | NA | | 0.05 | 0.25 | −0.03 | −0.12 | −0.06 | 0.11 |
| 16 | 1X | H2O + | −1.41^{C} | 0.00 | 0.00 | 0.00 | 0.00 | 0.00 |
| 8 | 0.5X | 50 mM NaCHO₃ | −1.18^{C} | 0.00 | 0.00 | 0.00 | 0.00 | 0.00 |
| 4 | 0.25X | | −0.47 | −0.30 | 0.00 | 0.00 | 0.00 | 0.00 |
| 0 | NA | | 0.06 | 0.15 | −0.17 | −0.05 | −0.09 | −0.13 |
| 16 | 1X | CAMHB + | 0.08 | 0.11 | 0.15 | −0.03 | 0.08 | −0.21 |
| 8 | 0.5X | 50 mM NaCHO₃ | 0.32 | 0.17 | −0.22 | 0.00 | 0.08 | 0.16 |
| 4 | 0.25X | | −0.07 | −0.67 | 0.01 | −0.05 | −0.18 | 0.00 |
| 0 | NA | | 0.11 | −0.18 | −0.35 | −0.08 | −0.09 | 0.00 |

¹SEQ ID NO: 1 MIC by broth microdilution in CAMHB
^{C}Instances where the difference in log-CFU with sodium bicarbonate (NaCHO₃) relative to log-CFU without NaCHO₃ ranged from −1 to −2
^{D}Instances where the difference in log-CFU with sodium bicarbonate (NaCHO₃) relative to log-CFU without NaCHO₃ was <−2

TABLE 12
Quality Control of SEQ ID NO: 1 stock used

| | | MIC |
|---|---|---|
| Organism | Media type | (μg/ml) |
| Staphylococcus aureus ATCC 29213 | CAMHB + | 4 |
| Escherichia coli ATCC 25922 | 0.002% P-80 | 2 |
| Pseudomonas aeruginosa ATCC 27853 | | 4 |
| Staphylococcus aureus ATCC 29213 | RPMI + | 0.12 |
| Escherichia coli ATCC 25922 | 0.002% P-80 | 0.5 |
| Pseudomonas aeruginosa ATCC 27853 | | 0.5 |

TABLE 13A
Neutralization of SEQ ID NO: 1 and Dey-Engley Broth
D/E Toxicity and Neutralization of SEQ ID NO: 1 by D/E

| | | SEQ ID | Average CFU | | | Mean | % CFU relative |
|---|---|---|---|---|---|---|---|
| Minutes | Media | NO: 1 | Rep No. 1 | Rep No. 2 | Rep No. 3 | CFU | to control |
| 0 | PBS | 0 | 37 | 33 | 47 | 39 | NA |
| | D/E | 0 | 45 | 31 | 29 | 35 | 90^{A} |
| | | 8 | 39 | 28 | 30 | 32 | 93^{A} |
| | | 500 | 15 | 17 | 17 | 16 | 47^{B} |
| 5 | PBS | 0 | 34 | 44 | 33 | 37 | NA |
| | D/E | 0 | 32 | 28 | 33 | 31 | 84^{A} |
| | | 8 | 37 | 31 | 33 | 34 | 109^{A} |
| | | 500 | 3 | 9 | 5 | 5 | 17^{B} |
| 15 | PBS | 0 | 37 | 38 | 45 | 40 | NA |
| | D/E | 0 | 39 | 40 | 30 | 36 | 90^{A} |
| | | 8 | 48 | 35 | 36 | 40 | 109^{A} |
| | | 500 | 1 | 4 | 1 | 2 | 5^{B} |

^{A}Refers to where % CFU relative to control ≥80
^{B}Refers to where % CFU relative to control ≤80

TABLE 13B
Neutralization of SEQ ID NO: 1 and Dey-Engley Broth
Neutralization of SEQ ID NO: 1 by D/E

| | | SEQ ID | | | | | |
|---|---|---|---|---|---|---|---|
| | | NO: 1 | Average CFU | | | Mean | % CFU relative |
| Minutes | Media | (μg/mL) | Rep No. 1 | Rep No. 2 | Rep No. 3 | CFU | to control |
| 0 | PBS | 0 | 14 | 14 | 11 | 13 | NA |
| | D/E | 25 | 12 | 21 | 17 | 17 | 127^{A} |
| | | 50 | 21 | 25 | 21 | 22 | 172^{A} |
| 5 | PBS | 0 | 12 | 24 | 18 | 18 | NA |
| | D/E | 25 | 17 | 16 | 20 | 18 | 99^{A} |
| | | 50 | 23 | 23 | 23 | 23 | 130^{A} |
| 15 | PBS | 0 | 17 | 21 | 17 | 18 | NA |
| | D/E | 25 | 17 | 16 | 18 | 17 | 91^{A} |
| | | 50 | 18 | 14 | 18 | 16 | 90^{A} |

^{A}Refers to where % CFU relative to control ≥80

TABLE 14A
Data for time-kill study with SEQ ID NO: 1. Isolate: Staphylococcus aureus NRS 382 (HA-MRSA)

| | | | | | | | | Log-kill | Log-CFU |
|---|---|---|---|---|---|---|---|---|---|
| | | | | | | | | (log10 | (log10 |
| | | | | | | | | CFU/ml) | CFU/ml) |
| | | Count | Count | | | | | relative to | relative to |
| | Time | 1 | 2 | | Average | Average | log10 | base media | media without |
| Condition | (min) | CFU | CFU | DF | CFU | CFU/ml | CFU/ml | with no drug | NaCHO3 at |
| PBS | 0 | 4 | 7 | 2000000 | 5.5 | 1.1E+07 | 7.04 | — | — |
| | 2.5 | 12 | 10 | 2000000 | 11 | 2.2E+07 | 7.34 | −0.30 | — |
| | 5 | 15 | 16 | 2000000 | 15.5 | 3.1E+07 | 7.49 | −0.45 | — |
| | 10 | 12 | 10 | 2000000 | 11 | 2.2E+07 | 7.34 | −0.30 | — |
| | 15 | 9 | 13 | 2000000 | 11 | 2.2E+07 | 7.34 | −0.30 | — |
| | 30 | 10 | 11 | 2000000 | 10.5 | 2.1E+07 | 7.32 | −0.28 | — |
| PBS + | 0 | 11 | 9 | 2000000 | 10 | 2.0E+07 | 7.30 | — | −0.26 |
| 50 mM | 2.5 | 17 | 6 | 2000000 | 11.5 | 2.3E+07 | 7.36 | −0.06 | −0.02 |
| NaCHO3 | 5 | 13 | 11 | 2000000 | 12 | 2.4E+07 | 7.38 | −0.08 | 0.11 |
| | 10 | 14 | 10 | 2000000 | 12 | 2.4E+07 | 7.38 | −0.08 | −0.04 |
| | 15 | 13 | 13 | 2000000 | 13 | 2.6E+07 | 7.41 | −0.11 | −0.07 |
| | 30 | 11 | 8 | 2000000 | 9.5 | 1.9E+07 | 7.28 | 0.02 | 0.04 |
| H2O | 0 | 5 | 3 | 2000000 | 4 | 8.0E+06 | 6.90 | — | — |
| | 2.5 | 13 | 13 | 2000000 | 13 | 2.6E+07 | 7.41 | −0.51 | — |
| | 5 | 21 | 16 | 2000000 | 18.5 | 3.7E+07 | 7.57 | −0.67 | — |
| | 10 | 7 | 11 | 2000000 | 9 | 1.8E+07 | 7.26 | −0.35 | — |
| | 15 | 7 | 6 | 2000000 | 6.5 | 1.3E+07 | 7.11 | −0.21 | — |
| | 30 | 18 | 17 | 2000000 | 17.5 | 3.5E+07 | 7.54 | −0.64 | — |
| H2O + | 0 | 5 | 6 | 2000000 | 5.5 | 1.1E+07 | 7.04 | — | −0.14 |
| 50 mM | 2.5 | 15 | 28 | 2000000 | 21.5 | 4.3E+07 | 7.63 | −0.59 | 0.22 |
| NaCHO3 | 5 | 11 | 16 | 2000000 | 13.5 | 2.7E+07 | 7.43 | −0.39 | 0.14 |
| | 10 | 6 | 11 | 2000000 | 8.5 | 1.7E+07 | 7.23 | −0.19 | 0.02 |
| | 15 | 6 | 7 | 2000000 | 6.5 | 1.3E+07 | 7.11 | −0.07 | 0.00 |
| | 30 | 14 | 18 | 2000000 | 16 | 3.2E+07 | 7.51 | −0.46 | 0.04 |
| CAMHB | 0 | 9 | 8 | 2000000 | 8.5 | 1.7E+07 | 7.23 | — | — |
| | 2.5 | 17 | 19 | 2000000 | 18 | 3.6E+07 | 7.56 | −0.33 | — |
| | 5 | 21 | 24 | 2000000 | 22.5 | 4.5E+07 | 7.65 | −0.42 | — |
| | 10 | 18 | 9 | 2000000 | 13.5 | 2.7E+07 | 7.43 | −0.20 | — |
| | 15 | 2 | 7 | 2000000 | 4.5 | 9.0E+06 | 6.95 | 0.28 | — |
| | 30 | 28 | 20 | 2000000 | 24 | 4.8E+07 | 7.68 | −0.45 | — |
| CAMHB + | 0 | 10 | 9 | 2000000 | 9.5 | 1.9E+07 | 7.28 | — | −0.05 |
| 50 mM | 2.5 | 15 | 17 | 2000000 | 16 | 3.2E+07 | 7.51 | −0.23 | 0.05 |
| NaCHO3 | 5 | 42 | 16 | 2000000 | 29 | 5.8E+07 | 7.76 | −0.48 | −0.11 |
| | 10 | 7 | 20 | 2000000 | 13.5 | 2.7E+07 | 7.43 | −0.15 | 0.00 |
| | 15 | 12 | 7 | 2000000 | 9.5 | 1.9E+07 | 7.28 | 0.00 | −0.32 |
| | 30 | 37 | 45 | 2000000 | 41 | 8.2E+07 | 7.91 | −0.64 | −0.23 |
| PBS | 0 | 1 | 7 | 2000000 | 4 | 8.0E+06 | 6.90 | 0.14 | — |
| 0.25X | 2.5 | 6 | 7 | 2000000 | 6.5 | 1.3E+07 | 7.11 | −0.07 | — |
| MIC | 5 | 7 | 6 | 2000000 | 6.5 | 1.3E+07 | 7.11 | −0.07 | — |
| | 10 | 4 | 4 | 20000 | 4 | 8.0E+04 | 4.90 | 2.14 | — |
| | 15 | 23 | 21 | 2000 | 22 | 4.4E+04 | 4.64 | 2.40 | — |
| | 30 | 23 | 11 | 2000 | 17 | 3.4E+04 | 4.53 | 2.51 | — |
| PBS + | 0 | 2 | 1 | 2000000 | 1.5 | 3.0E+06 | 6.48 | 0.82 | 0.43 |
| 50 mM | 2.5 | 7 | 6 | 20000 | 6.5 | 1.3E+05 | 5.11 | 2.19 | 2.00 |
| NaCHO3 | 5 | 13 | 17 | 2000 | 15 | 3.0E+04 | 4.48 | 2.82 | 2.64 |
| 0.25X | 10 | 4 | 4 | 2000 | 4 | 8.0E+03 | 3.90 | 3.40 | 1.00 |
| MIC | 15 | 4 | 3 | 20000 | 3.5 | 7.0E+04 | 4.85 | 2.46 | −0.20 |
| | 30 | 9 | 12 | 2000 | 10.5 | 2.1E+04 | 4.32 | 2.98 | 0.21 |
| H2O | 0 | 19 | 15 | 200000 | 17 | 3.4E+06 | 6.53 | 0.37 | — |
| 0.25X | 2.5 | 7 | 10 | 20000 | 8.5 | 1.7E+05 | 5.23 | 1.67 | — |
| MIC | 5 | 3 | 5 | 20000 | 4 | 8.0E+04 | 4.90 | 2.00 | — |
| | 10 | 9 | 3 | 2000 | 6 | 1.2E+04 | 4.08 | 2.82 | — |
| | 15 | 3 | 1 | 20000 | 2 | 4.0E+04 | 4.60 | 2.30 | — |
| | 30 | 14 | 20 | 200 | 17 | 3.4E+03 | 3.53 | 3.37 | — |
| H2O + | 0 | 4 | 5 | 2000000 | 4.5 | 9.0E+06 | 6.95 | 0.09 | −0.42 |
| 50 mM | 2.5 | 3 | 15 | 2000 | 9 | 1.8E+04 | 4.26 | 2.79 | 0.98 |
| NaCHO3 | 5 | 7 | 10 | 20000 | 8.5 | 1.7E+05 | 5.23 | 1.81 | −0.33 |
| 0.25X | 10 | 4 | 3 | 2000 | 3.5 | 7.0E+03 | 3.85 | 3.20 | 0.23 |
| MIC | 15 | 10 | 5 | 2000 | 7.5 | 1.5E+04 | 4.18 | 2.87 | 0.43 |
| | 30 | 22 | 20 | 200 | 21 | 4.2E+03 | 3.62 | 3.42 | −0.0 |
| CAMHB | 0 | 4 | 5 | 2000000 | 4.5 | 9.0E+06 | 6.95 | 0.28 | — |
| 0.25X | 2.5 | 12 | 13 | 2000000 | 12.5 | 2.5E+07 | 7.40 | −0.17 | — |
| MIC | 5 | 11 | 6 | 2000000 | 8.5 | 1.7E+07 | 7.23 | 0.00 | — |
| | 10 | 9 | 19 | 2000000 | 14 | 2.8E+07 | 7.45 | −0.22 | — |
| | 15 | 4 | 9 | 2000000 | 6.5 | 1.3E+07 | 7.11 | 0.12 | — |
| | 30 | 13 | 14 | 2000000 | 13.5 | 2.7E+07 | 7.43 | −0.20 | — |
| CAMHB + | 0 | 4 | 2 | 2000000 | 3 | 6.0E+06 | 6.78 | 0.50 | 0.18 |
| 50 mM | 2.5 | 9 | 11 | 2000000 | 10 | 2.0E+07 | 7.30 | −0.02 | 0.10 |
| NaCHO3 | 5 | 7 | 8 | 2000000 | 7.5 | 1.5E+07 | 7.18 | 0.10 | 0.05 |
| 0.25X | 10 | 18 | 10 | 2000000 | 14 | 2.8E+07 | 7.45 | −0.17 | 0.00 |
| MIC | 15 | 7 | 5 | 2000000 | 6 | 1.2E+07 | 7.08 | 0.20 | 0.03 |
| | 30 | 5 | 11 | 2000000 | 8 | 1.6E+07 | 7.20 | 0.07 | 0.23 |
| PBS | 0 | 25 | 34 | 200000 | 29.5 | 5.9E+06 | 6.77 | 0.27 | — |
| 0.5X MIC | 2.5 | 6 | 8 | 20000 | 7 | 1.4E+05 | 5.15 | 1.90 | — |
| | 5 | 8 | 7 | 2000 | 7.5 | 1.5E+04 | 4.18 | 2.87 | — |
| | 10 | 15 | 10 | 20000 | 12.5 | 2.5E+05 | 5.40 | 1.64 | — |
| | 15 | 4 | 8 | 200 | 6 | 1.2E+03 | 3.08 | 3.96 | — |
| | 30 | 8 | 5 | 200 | 6.5 | 1.3E+03 | 3.11 | 3.93 | — |
| PBS + | 0 | 19 | 12 | 200000 | 15.5 | 3.1E+06 | 6.49 | 0.81 | 0.28 |
| 50 mM | 2.5 | 6 | 6 | 2000 | 6 | 1.2E+04 | 4.08 | 3.22 | 1.07 |
| NaCHO3 | 5 | 3 | 3 | 20000 | 3 | 6.0E+04 | 4.78 | 2.52 | −0.60 |
| 0.5X MIC | 10 | 3 | 4 | 2000 | 3.5 | 7.0E+03 | 3.85 | 3.46 | 1.55 |
| | 15 | 27 | 8 | 200 | 17.5 | 3.5E+03 | 3.54 | 3.76 | −0.46 |
| | 30 | 4 | 4 | 200 | 4 | 8.0E+02 | 2.90 | 4.40 | 0.21 |
| H2O | 0 | 17 | 18 | 200000 | 17.5 | 3.5E+06 | 6.54 | 0.36 | — |
| 0.5X MIC | 2.5 | 2 | 1 | 20000 | 1.5 | 3.0E+04 | 4.48 | 2.43 | — |
| | 5 | 5 | 30 | 2000 | 17.5 | 3.5E+04 | 4.54 | 2.36 | — |
| | 10 | 3 | 10 | 200 | 6.5 | 1.3E+03 | 3.11 | 3.79 | — |
| | 15 | 0 | 1 | 200 | 0.5 | 1.0E+02 | 2.00 | >4.90 | — |
| | 30 | 0 | 0 | NA | 0 | <1.0+02 | <2.0 | >4.90 | — |
| H2O + | 0 | 5 | 6 | 200000 | 5.5 | 1.1E+06 | 6.04 | 1.00 | 0.50 |
| 50 mM | 2.5 | 8 | 7 | 2000 | 7.5 | 1.5E+04 | 4.18 | 2.87 | 0.30 |
| NaCHO3 | 5 | 38 | 19 | 200 | 28.5 | 5.7E+03 | 3.76 | 3.29 | 0.79 |
| 0.5X MIC | 10 | 4 | 2 | 2000 | 3 | 6.0E+03 | 3.78 | 3.26 | −0.66 |
| | 15 | 3 | 4 | 2000 | 3.5 | 7.0E+03 | 3.85 | 3.20 | −1.85 |
| | 30 | 0 | 1 | 200 | 0.5 | 1.0E+02 | 2.00 | >5.04 | 0.00 |
| CAMHB | 0 | 5 | 11 | 2000000 | 8 | 1.6E+07 | 7.20 | 0.03 | — |
| 0.5X MIC | 2.5 | 10 | 6 | 2000000 | 8 | 1.6E+07 | 7.20 | 0.03 | — |
| | 5 | 10 | 10 | 2000000 | 10 | 2.0E+07 | 7.30 | −0.07 | — |
| | 10 | 10 | 11 | 2000000 | 10.5 | 2.1E+07 | 7.32 | −0.09 | — |
| | 15 | 12 | 3 | 2000000 | 7.5 | 1.5E+07 | 7.18 | 0.05 | — |
| | 30 | 9 | 5 | 2000000 | 7 | 1.4E+07 | 7.15 | 0.08 | — |
| CAMHB + | 0 | 7 | 13 | 2000000 | 10 | 2.0E+07 | 7.30 | −0.02 | −0.10 |
| 50 mM | 2.5 | 11 | 9 | 2000000 | 10 | 2.0E+07 | 7.30 | −0.02 | −0.10 |
| NaCHO3 | 5 | 12 | 12 | 2000000 | 12 | 2.4E+07 | 7.38 | −0.10 | −0.08 |
| 0.5X MIC | 10 | 15 | 8 | 2000000 | 11.5 | 2.3E+07 | 7.36 | −0.08 | −0.04 |
| | 15 | 1 | 5 | 2000000 | 3 | 6.0E+06 | 6.78 | 0.50 | 0.40 |
| | 30 | 3 | 5 | 2000000 | 4 | 8.0E+06 | 6.90 | 0.38 | 0.24 |
| PBS | 0 | 3 | 2 | 2000000 | 2.5 | 5.0E+06 | 6.70 | 0.34 | — |
| 1X MIC | 2.5 | 15 | 22 | 2000 | 18.5 | 3.7E+04 | 4.57 | 2.47 | — |
| | 5 | 37 | 49 | 200 | 43 | 8.6E+03 | 3.93 | 3.11 | — |
| | 10 | 8 | 8 | 200 | 8 | 1.6E+03 | 3.20 | 3.84 | — |
| | 15 | 1 | 3 | 200 | 2 | 4.0E+02 | 2.60 | 4.44 | — |
| | 30 | 2 | 2 | 200 | 2 | 4.0E+02 | 2.60 | 4.44 | — |
| PBS + | 0 | 4 | 5 | 200000 | 4.5 | 9.0E+05 | 5.95 | 1.35 | 0.74 |
| 50 mM | 2.5 | 16 | 17 | 200 | 16.5 | 3.3E+03 | 3.52 | 3.78 | 1.05 |
| NaCHO3 | 5 | 1 | 3 | 20000 | 2 | 4.0E+04 | 4.60 | 2.70 | −0.67 |
| 1X MIC | 10 | 3 | 2 | 200 | 2.5 | 5.0E+02 | 2.70 | 4.60 | 0.51 |
| | 15 | 6 | 1 | 200 | 3.5 | 7.0E+02 | 2.85 | 4.46 | −0.24 |
| | 30 | 0 | 0 | NA | 0 | <1.0+02 | <2.0 | >5.30 | 0.60 |
| H2O | 0 | 2 | 2 | 200000 | 2 | 4.0E+05 | 5.60 | 1.30 | — |
| 1X MIC | 2.5 | 13 | 12 | 200 | 12.5 | 2.5E+03 | 3.40 | 3.51 | — |
| | 5 | 79 | 37 | 200 | 58 | 1.2E+04 | 4.06 | 2.84 | — |
| | 10 | 1 | 0 | 200 | 0.5 | 1.0E+02 | 2.00 | >4.90 | — |
| | 15 | 0 | 0 | NA | 0 | <1.0+02 | <2.0 | >4.90 | — |
| | 30 | 0 | 0 | NA | 0 | <1.0+02 | <2.0 | >4.90 | — |
| H2O + | 0 | 11 | 5 | 20000 | 8 | 1.6E+05 | 5.20 | 1.84 | 0.40 |
| 50 mM | 2.5 | 2 | 4 | 20000 | 3 | 6.0E+04 | 4.78 | 2.26 | −1.38 |
| NaCHO3 | 5 | 1 | 2 | 200 | 1.5 | 3.0E+02 | 2.48 | 4.56 | 1.59 |
| 1X MIC | 10 | 1 | 0 | 200 | 0.5 | 1.0E+02 | 2.00 | >5.04 | 0.00 |
| | 15 | 0 | 1 | 200 | 0.5 | 1.0E+02 | 2.00 | >5.04 | 0.00 |
| | 30 | 0 | 0 | NA | 0 | <1.0+02 | <2.0 | >5.04 | 0.00 |
| CAMHB | 0 | 12 | 3 | 2000000 | 7.5 | 1.5E+07 | 7.18 | 0.05 | — |
| 1X MIC | 2.5 | 18 | 12 | 2000000 | 15 | 3.0E+07 | 7.48 | −0.25 | — |
| | 5 | 9 | 14 | 2000000 | 11.5 | 2.3E+07 | 7.36 | −0.13 | — |
| | 10 | 7 | 8 | 2000000 | 7.5 | 1.5E+07 | 7.18 | 0.05 | — |
| | 15 | 15 | 5 | 2000000 | 10 | 2.0E+07 | 7.30 | −0.07 | — |
| | 30 | 8 | 7 | 2000000 | 7.5 | 1.5E+07 | 7.18 | 0.05 | — |
| CAMHB + | 0 | 17 | 12 | 2000000 | 14.5 | 2.9E+07 | 7.46 | −0.18 | −0.29 |
| 50 mM | 2.5 | 11 | 16 | 2000000 | 13.5 | 2.7E+07 | 7.43 | −0.15 | 0.05 |
| NaCHO3 | 5 | 5 | 6 | 2000000 | 5.5 | 1.1E+07 | 7.04 | 0.24 | 0.32 |
| 1X MIC | 10 | 5 | 6 | 2000000 | 5.5 | 1.1E+07 | 7.04 | 0.24 | 0.13 |
| | 15 | 35 | 42 | 200000 | 38.5 | 7.7E+06 | 6.89 | 0.39 | 0.41 |
| | 30 | 3 | 17 | 2000000 | 10 | 2.0E+07 | 7.30 | −0.02 | −0.12 |

TABLE 14B
Data for time-kill study with SEQ ID NO: 1. Isolate:
Staphylococcus aureus NRS 382 (CA-MRSA)

| | | | | | | | | Log- | Log- |
|---|---|---|---|---|---|---|---|---|---|
| | | | | | | | | kill | CFU |
| | | | | | | | | (log10 | (log10 |
| | | | | | | | | CFU/ml) | CFU/ml) |
| | | | | | | | | relative | relative |
| | | | | | | | | to base | to media |
| | | Count | Count | | | Average | log10 | media | without |
| | Time | 1 | 2 | | Average | CFU/ | CFU/ | with no | NaCHO3 |
| Condition | (min) | CFU | CFU | DF | CFU | ml | ml | drug | at |
| PBS | 0 | 21 | 27 | 2000000 | 24 | 4.8E+07 | 7.68 | — | — |
| | 2.5 | 17 | 22 | 2000000 | 19.5 | 3.9E+07 | 7.59 | 0.09 | — |
| | 5 | 23 | 28 | 2000000 | 25.5 | 5.1E+07 | 7.71 | −0.03 | — |
| | 10 | 22 | 21 | 2000000 | 21.5 | 4.3E+07 | 7.63 | 0.05 | — |
| | 15 | 21 | 24 | 2000000 | 22.5 | 4.5E+07 | 7.65 | 0.03 | — |
| | 30 | 17 | 13 | 2000000 | 15 | 3.0E+07 | 7.48 | 0.20 | — |
| PBS | 0 | 20 | 20 | 2000000 | 20 | 4.0E+07 | 7.60 | — | 0.08 |
| + | 2.5 | 23 | 17 | 2000000 | 20 | 4.0E+07 | 7.60 | 0.00 | −0.01 |
| 50 mM | 5 | 18 | 19 | 2000000 | 18.5 | 3.7E+07 | 7.57 | 0.03 | 0.14 |
| NaCHO₃ | 10 | 16 | 12 | 2000000 | 14 | 2.8E+07 | 7.45 | 0.15 | 0.19 |
| | 15 | 21 | 28 | 2000000 | 24.5 | 4.9E+07 | 7.69 | −0.09 | −0.04 |
| | 30 | 19 | 23 | 2000000 | 21 | 4.2E+07 | 7.62 | −0.02 | −0.15 |
| H₂O | 0 | 18 | 26 | 2000000 | 22 | 4.4E+07 | 7.64 | — | — |
| | 2.5 | 18 | 22 | 2000000 | 20 | 4.0E+07 | 7.60 | 0.04 | — |
| | 5 | 24 | 25 | 2000000 | 24.5 | 4.9E+07 | 7.69 | −0.05 | — |
| | 10 | 14 | 22 | 2000000 | 18 | 3.6E+07 | 7.56 | 0.09 | — |
| | 15 | 24 | 11 | 2000000 | 17.5 | 3.5E+07 | 7.54 | 0.10 | — |
| | 30 | 20 | 10 | 2000000 | 15 | 3.0E+07 | 7.48 | 0.17 | — |
| H₂O | 0 | 20 | 25 | 2000000 | 22.5 | 4.5E+07 | 7.65 | — | −0.01 |
| + | 2.5 | 15 | 10 | 2000000 | 12.5 | 2.5E+07 | 7.40 | 0.26 | 0.20 |
| 50 mM | 5 | 20 | 17 | 2000000 | 18.5 | 3.7E+07 | 7.57 | 0.09 | 0.12 |
| NaCHO₃ | 10 | 15 | 18 | 2000000 | 16.5 | 3.3E+07 | 7.52 | 0.13 | 0.04 |
| | 15 | 7 | 11 | 2000000 | 9 | 1.8E+07 | 7.26 | 0.40 | 0.29 |
| | 30 | 10 | 9 | 2000000 | 9.5 | 1.9E+07 | 7.28 | 0.37 | 0.20 |
| CAMHB | 0 | 15 | 27 | 2000000 | 21 | 4.2E+07 | 7.62 | — | — |
| | 2.5 | 12 | 12 | 2000000 | 12 | 2.4E+07 | 7.38 | 0.24 | — |
| | 5 | 17 | 19 | 2000000 | 18 | 3.6E+07 | 7.56 | 0.07 | — |
| | 10 | 27 | 16 | 2000000 | 21.5 | 4.3E+07 | 7.63 | −0.01 | — |
| | 15 | 31 | 23 | 2000000 | 27 | 5.4E+07 | 7.73 | −0.11 | — |
| | 30 | 25 | 15 | 2000000 | 20 | 4.0E+07 | 7.60 | 0.02 | — |
| CAMHB | 0 | 27 | 14 | 2000000 | 20.5 | 4.1E+07 | 7.61 | — | 0.01 |
| + | 2.5 | 22 | 21 | 2000000 | 21.5 | 4.3E+07 | 7.63 | −0.02 | −0.25 |
| 50 mM | 5 | 12 | 17 | 2000000 | 14.5 | 2.9E+07 | 7.46 | 0.15 | 0.09 |
| NaCHO₃ | 10 | 12 | 17 | 2000000 | 14.5 | 2.9E+07 | 7.46 | 0.15 | 0.17 |
| | 15 | 18 | 32 | 2000000 | 25 | 5.0E+07 | 7.70 | −0.09 | 0.03 |
| | 30 | 20 | 21 | 2000000 | 20.5 | 4.1E+07 | 7.61 | 0.00 | −0.01 |
| PBS | 0 | 34 | 30 | 200000 | 32 | 6.4E+06 | 6.81 | 0.88 | — |
| 0.25X | 2.5 | 7 | 4 | 200000 | 5.5 | 1.1E+06 | 6.04 | 1.64 | — |
| MIC | 5 | 17 | 23 | 20000 | 20 | 4.0E+05 | 5.60 | 2.08 | — |
| | 10 | 11 | 8 | 20000 | 9.5 | 1.9E+05 | 5.28 | 2.40 | — |
| | 15 | 11 | 12 | 20000 | 11.5 | 2.3E+05 | 5.36 | 2.32 | — |
| | 30 | 5 | 8 | 20000 | 6.5 | 1.3E+05 | 5.11 | 2.57 | — |
| PBS | 0 | 15 | 21 | 200000 | 18 | 3.6E+06 | 6.56 | 1.05 | 0.25 |
| + | 2.5 | 16 | 15 | 20000 | 15.5 | 3.1E+05 | 5.49 | 2.11 | 0.55 |
| 50 mM | 5 | 50 | 48 | 2000 | 49 | 9.8E+04 | 4.99 | 2.61 | 0.61 |
| NaCHO₃ | 10 | 51 | 48 | 2000 | 49.5 | 9.9E+04 | 5.00 | 2.61 | 0.28 |
| 0.25X | 15 | 38 | 45 | 2000 | 41.5 | 8.3E+04 | 4.92 | 2.68 | 0.44 |
| MIC | 30 | 5 | 8 | 20000 | 6.5 | 1.3E+05 | 5.11 | 2.49 | 0.00 |
| H₂O | 0 | 15 | 15 | 200000 | 15 | 3.0E+06 | 6.48 | 1.17 | — |
| 0.25X | 2.5 | 4 | 4 | 200 | 4 | 8.0E+02 | 2.90 | 4.74 | — |
| MIC | 5 | 7 | 0 | 200 | 3.5 | 7.0E+02 | 2.85 | 4.80 | — |
| | 10 | 0 | 0 | NA | 0 | <1.0 + 02 | <2.0 | >5.64 | — |
| | 15 | 2 | 1 | 200 | 1.5 | 3.0E+02 | 2.48 | 5.17 | — |
| | 30 | 0 | 0 | NA | 0 | <1.0 + 02 | <2.0 | >5.64 | — |
| H₂O | 0 | 52 | 60 | 20000 | 56 | 1.1E+06 | 6.05 | 1.60 | 0.43 |
| + | 2.5 | 29 | 36 | 200 | 32.5 | 6.5E+03 | 3.81 | 3.84 | −0.91 |
| 50 mM | 5 | 6 | 10 | 200 | 8 | 1.6E+03 | 3.20 | 4.45 | −0.36 |
| NaCHO₃ | 10 | 3 | 2 | 200 | 2.5 | 5.0E+02 | 2.70 | 4.95 | −0.70 |
| 0.25X | 15 | 1 | 1 | 200 | 1 | 2.0E+02 | 2.30 | 5.35 | 0.18 |
| MIC | 30 | 0 | 2 | 200 | 1 | 2.0E+02 | 2.30 | 5.35 | −0.30 |
| CAMHB | 0 | 17 | 10 | 2000000 | 13.5 | 2.7E+07 | 7.43 | 0.19 | — |
| 0.25X | 2.5 | 25 | 21 | 2000000 | 23 | 4.6E+07 | 7.66 | −0.04 | — |
| MIC | 5 | 15 | 22 | 2000000 | 18.5 | 3.7E+07 | 7.57 | 0.06 | — |
| | 10 | 5 | 19 | 2000000 | 12 | 2.4E+07 | 7.38 | 0.24 | — |
| | 15 | 16 | 19 | 2000000 | 17.5 | 3.5E+07 | 7.54 | 0.08 | — |
| | 30 | 11 | 8 | 2000000 | 9.5 | 1.9E+07 | 7.28 | 0.34 | — |
| CAMHB | 0 | 21 | 18 | 2000000 | 19.5 | 3.9E+07 | 7.59 | 0.02 | −0.16 |
| + | 2.5 | 20 | 15 | 2000000 | 17.5 | 3.5E+07 | 7.54 | 0.07 | 0.12 |
| 50 mM | 5 | 10 | 14 | 2000000 | 12 | 2.4E+07 | 7.38 | 0.23 | 0.19 |
| NaCHO₃ | 10 | 4 | 14 | 2000000 | 9 | 1.8E+07 | 7.26 | 0.36 | 0.12 |
| 0.25X | 15 | 9 | 10 | 2000000 | 9.5 | 1.9E+07 | 7.28 | 0.33 | 0.27 |
| MIC | 30 | 21 | 23 | 200000 | 22 | 4.4E+06 | 6.64 | 0.97 | 0.64 |
| PBS | 0 | 9 | 6 | 200000 | 7.5 | 1.5E+06 | 6.18 | 1.51 | — |
| 0.5X MIC | 2.5 | 26 | 19 | 20000 | 22.5 | 4.5E+05 | 5.65 | 2.03 | — |
| | 5 | 21 | 14 | 20000 | 17.5 | 3.5E+05 | 5.54 | 2.14 | — |
| | 10 | 42 | 37 | 2000 | 39.5 | 7.9E+04 | 4.90 | 2.78 | — |
| | 15 | 13 | 27 | 2000 | 20 | 4.0E+04 | 4.60 | 3.08 | — |
| | 30 | 24 | 34 | 2000 | 29 | 5.8E+04 | 4.76 | 2.92 | — |
| PBS | 0 | 51 | 65 | 20000 | 58 | 1.2E+06 | 6.06 | 1.54 | 0.11 |
| + | 2.5 | 40 | 36 | 2000 | 38 | 7.6E+04 | 4.88 | 2.72 | 0.77 |
| 50 mM | 5 | 27 | 26 | 2000 | 26.5 | 5.3E+04 | 4.72 | 2.88 | 0.82 |
| NaCHO₃ | 10 | 22 | 20 | 2000 | 21 | 4.2E+04 | 4.62 | 2.98 | 0.27 |
| 0.5X MIC | 15 | 23 | 14 | 2000 | 18.5 | 3.7E+04 | 4.57 | 3.03 | 0.03 |
| | 30 | 17 | 25 | 2000 | 21 | 4.2E+04 | 4.62 | 2.98 | 0.14 |
| H₂O | 0 | 26 | 17 | 2000 | 21.5 | 4.3E+04 | 4.63 | 3.01 | — |
| 0.5X MIC | 2.5 | 1 | 0 | 200 | 0.5 | 1.0E+02 | 2.00 | >5.64 | — |
| | 5 | 0 | 0 | NA | 0) | <1.0 + 02 | <2.0 | >5.64 | — |
| | 10 | 0 | 0 | NA | 0) | <1.0 + 02 | <2.0 | >5.64 | — |
| | 15 | 0 | 0 | NA | 0 | <1.0 + 02 | <2.0 | >5.64 | — |
| | 30 | 0 | 0 | NA | 0 | <1.0 + 02 | <2.0 | >5.64 | — |
| H₂O | 0 | 41 | 48 | 2000 | 44.5 | 8.9E+04 | 4.95 | 2.70 | −0.32 |
| + | 2.5 | 6 | 1 | 200 | 3.5 | 7.0E+02 | 2.85 | 4.81 | −0.85 |
| 50 mM | 5 | 6 | 4 | 200 | 5 | 1.0E+03 | 3.00 | 4.65 | −1.00 |
| NaCHO₃ | 10 | 0 | 0 | NA | 0 | <1.0 + 02 | <2.0 | >5.65 | 0.00 |
| 0.5X MIC | 15 | 0 | 1 | 200 | 0.5 | 1.0E+02 | 2.00 | >5.65 | 0.00 |
| | 30 | 0 | 2 | 200 | 1 | 2.0E+02 | 2.30 | 5.35 | −0.30 |
| CAMHB | 0 | 25 | 31 | 2000000 | 28 | 5.6E+07 | 7.75 | −0.12 | — |
| 0.5X MIC | 2.5 | 16 | 16 | 2000000 | 16 | 3.2E+07 | 7.51 | 0.12 | — |
| | 5 | 20 | 16 | 2000000 | 18 | 3.6E+07 | 7.56 | 0.07 | — |
| | 10 | 9 | 9 | 2000000 | 9 | 1.8E+07 | 7.26 | 0.37 | — |
| | 15 | 18 | 14 | 2000000 | 16 | 3.2E+07 | 7.51 | 0.12 | — |
| | 30 | 5 | 8 | 2000000 | 6.5 | 1.3E+07 | 7.11 | 0.51 | — |
| | 0 | 19 | 18 | 2000000 | 18.5 | 3.7E+07 | 7.57 | 0.04 | 0.18 |
| CAMHB | 2.5 | 13 | 14 | 2000000 | 13.5 | 2.7E+07 | 7.43 | 0.18 | 0.07 |
| + | 5 | 6 | 10 | 2000000 | 8 | 1.6E+07 | 7.20 | 0.41 | 0.35 |
| 50 mM | 10 | 58 | 35 | 200000 | 46.5 | 9.3E+06 | 6.97 | 0.64 | 0.29 |
| NaCHO₃ | 15 | 34 | 45 | 200000 | 39.5 | 7.9E+06 | 6.90 | 0.72 | 0.61 |
| 0.5X MIC | 30 | 18 | 10 | 200000 | 14 | 2.8E+06 | 6.45 | 1.17 | 0.67 |
| PBS | 0 | 60 | 60 | 20000 | 60 | 1.2E+06 | 6.08 | 1.60 | — |
| 1X MIC | 2.5 | 8 | 8 | 20000 | 8 | 1.6E+05 | 5.20 | 2.48 | — |
| | 5 | 29 | 35 | 2000 | 32 | 6.4E+04 | 4.81 | 2.88 | — |
| | 10 | 25 | 18 | 2000 | 21.5 | 4.3E+04 | 4.63 | 3.05 | — |
| | 15 | 12 | 15 | 2000 | 13.5 | 2.7E+04 | 4.43 | 3.25 | — |
| | 30 | 8 | 12 | 2000 | 10 | 2.0E+04 | 4.30 | 3.38 | — |
| PBS | 0 | 32 | 27 | 20000 | 29.5 | 5.9E+05 | 5.77 | 1.83 | 0.31 |
| + | 2.5 | 9 | 20 | 2000 | 14.5 | 2.9E+04 | 4.46 | 3.14 | 0.74 |
| 50 mM | 5 | 7 | 8 | 2000 | 7.5 | 1.5E+04 | 4.18 | 3.43 | 0.63 |
| NaCHO₃ | 10 | 6 | 4 | 2000 | 5 | 1.0E+04 | 4.00 | 3.60 | 0.63 |
| 1X MIC | 15 | 3 | 7 | 2000 | 5 | 1.0E+04 | 4.00 | 3.60 | 0.43 |
| | 30 | 8 | 5 | 2000 | 6.5 | 1.3E+04 | 4.11 | 3.49 | 0.19 |
| H₂O | 0 | 52 | 59 | 200 | 55.5 | 1.1E+04 | 4.05 | 3.60 | — |
| 1X MIC | 2.5 | 1 | 0 | 200 | 0.5 | 1.0E+02 | 2.00 | >5.64 | — |
| | 5 | 1 | 0 | 200 | 0.5 | 1.0E+02 | 2.00 | >5.64 | — |
| | 10 | 1 | 1 | 200 | 1 | 2.0E+02 | 2.30 | 5.34 | — |
| | 15 | 0 | 0 | NA | 0 | <1.0 + 02 | <2.0 | >5.64 | — |
| | 30 | 0 | 0 | NA | 0 | <1.0 + 02 | <2.0 | >5.64 | — |
| H₂O | 0 | 15 | 8 | 20000 | 11.5 | 2.3E+05 | 5.36 | 2.29 | −1.32 |
| + | 2.5 | 5 | 4 | 200 | 4.5 | 9.0E+02 | 2.95 | 4.70 | −0.95 |
| 50 mM | 5 | 1 | 1 | 200 | 1 | 2.0E+02 | 2.30 | 5.35 | −0.30 |
| NaCHO₃ | 10 | 0 | 1 | 200 | 0.5 | 1.0E+02 | 2.00 | >5.65 | 0.30 |
| 1X MIC | 15 | 0 | 0 | NA | 0 | <1.0 + 02 | <2.0 | >5.65 | 0.00 |
| | 30 | 2 | 0 | 200 | 1 | 2.0E+02 | 2.30 | 5.35 | −0.30 |
| CAMHB | 0 | 24 | 23 | 2000000 | 23.5 | 4.7E+07 | 7.67 | −0.05 | — |
| 1X MIC | 2.5 | 15 | 12 | 2000000 | 13.5 | 2.7E+07 | 7.43 | 0.19 | — |
| | 5 | 79 | 73 | 200000 | 76 | 1.5E+07 | 7.18 | 0.44 | — |
| | 10 | 41 | 41 | 200000 | 41 | 8.2E+06 | 6.91 | 0.71 | — |
| | 15 | 19 | 24 | 200000 | 21.5 | 4.3E+06 | 6.63 | 0.99 | — |
| | 30 | 45 | 48 | 20000 | 46.5 | 9.3E+05 | 5.97 | 1.65 | — |
| CAMHB | 0 | 20 | 26 | 2000000 | 23 | 4.6E+07 | 7.66 | −0.05 | 0.01 |
| + | 2.5 | 8 | 7 | 2000000 | 7.5 | 1.5E+07 | 7.18 | 0.44 | 0.26 |
| 50 mM | 5 | 26 | 33 | 200000 | 29.5 | 5.9E+06 | 6.77 | 0.84 | 0.41 |
| NaCHO₃ | 10 | 18 | 15 | 200000 | 16.5 | 3.3E+06 | 6.52 | 1.09 | 0.40 |
| 1X MIC | 15 | 5 | 8 | 200000 | 6.5 | 1.3E+06 | 6.11 | 1.50 | 0.52 |
| | 30 | 25 | 24 | 20000 | 24.5 | 4.9E+05 | 5.69 | 1.92 | 0.28 |

TABLE 14C
Data for time-kill study with SEQ ID NO: 1. Isolate:
Staphylococcus epidermidis MMX 8655 (MRSE)

| | | | | | | | | Log-kill | Log-CFU |
|---|---|---|---|---|---|---|---|---|---|
| | | | | | | | | (log₁₀ | (log₁₀ |
| | | | | | | | | CFU/ml) | CFU/ml) |
| | | | | | | | | relative | relative to |
| | | | | | | | | to base | media |
| | | | | | | | | media | without |
| | | Count | Count | | | | log10 | with | NaCHO₃ |
| | Time | 1 | 2 | | Average | Average | CFU/ | no drug | at each |
| Condition | (min) | CFU | CFU | DF | CFU | CFU/ml | ml | at 0 min | time point |
| PBS | 0 | 8 | 5 | 2000000 | 6.5 | 1.3E+07 | 7.11 | — | — |
| | 2.5 | 31 | 40 | 200000 | 35.5 | 7.1E+06 | 6.85 | 0.26 | — |
| | 5 | 4 | 7 | 2000000 | 5.5 | 1.1E+07 | 7.04 | 0.07 | — |
| | 10 | 5 | 10 | 2000000 | 7.5 | 1.5E+07 | 7.18 | −0.06 | — |
| | 15 | 35 | 44 | 200000 | 39.5 | 7.9E+06 | 6.90 | 0.22 | — |
| | 30 | 45 | 35 | 200000 | 40 | 8.0E+06 | 6.90 | 0.21 | — |
| PBS | 0 | 48 | 35 | 200000 | 41.5 | 8.3E+06 | 6.92 | — | 0.19 |
| + | 2.5 | 6 | 6 | 2000000 | 6 | 1.2E+07 | 7.08 | −0.16 | −0.23 |
| 50 mM | 5 | 42 | 54 | 200000 | 48 | 9.6E+06 | 6.98 | −0.06 | 0.06 |
| NaCHO₃ | 10 | 49 | 72 | 200000 | 60.5 | 1.2E+07 | 7.08 | −0.16 | 0.09 |
| | 15 | 40 | 41 | 200000 | 40.5 | 8.1E+06 | 6.91 | 0.01 | −0.01 |
| | 30 | 7 | 7 | 2000000 | 7 | 1.4E+07 | 7.15 | −0.23 | −0.24 |
| H₂O | 0 | 45 | 48 | 200000 | 46.5 | 9.3E+06 | 6.97 | — | — |
| | 2.5 | 38 | 34 | 200000 | 36 | 7.2E+06 | 6.86 | 0.11 | — |
| | 5 | 10 | 5 | 2000000 | 7.5 | 1.5E+07 | 7.18 | −0.21 | — |
| | 10 | 4 | 5 | 2000000 | 4.5 | 9.0E+06 | 6.95 | 0.01 | — |
| | 15 | 9 | 6 | 2000000 | 7.5 | 1.5E+07 | 7.18 | −0.21 | — |
| | 30 | 9 | 8 | 2000000 | 8.5 | 1.7E+07 | 7.23 | −0.26 | — |
| H₂O | 0 | 49 | 53 | 200000 | 51 | 1.0E+07 | 7.01 | — | −0.04 |
| + | 2.5 | 8 | 4 | 2000000 | 6 | 1.2E+07 | 7.08 | −0.07 | −0.22 |
| 50 mM | 5 | 8 | 7 | 2000000 | 7.5 | 1.5E+07 | 7.18 | −0.17 | 0.00 |
| NaCHO₃ | 10 | 6 | 6 | 2000000 | 6 | 1.2E+07 | 7.08 | −0.07 | −0.12 |
| | 15 | 47 | 41 | 200000 | 44 | 8.8E+06 | 6.94 | 0.06 | 0.23 |
| | 30 | 43 | 45 | 200000 | 44 | 8.8E+06 | 6.94 | 0.06 | 0.29 |
| CAMHB | 0 | 10 | 4 | 2000000 | 7 | 1.4E+07 | 7.15 | — | — |
| | 2.5 | 38 | 46 | 200000 | 42 | 8.4E+06 | 6.92 | 0.22 | — |
| | 5 | 17 | 9 | 2000000 | 13 | 2.6E+07 | 7.41 | −0.27 | — |
| | 10 | 9 | 10 | 2000000 | 9.5 | 1.9E+07 | 7.28 | −0.13 | — |
| | 15 | 48 | 51 | 200000 | 49.5 | 9.9E+06 | 7.00 | 0.15 | — |
| | 30 | 2 | 5 | 2000000 | 3.5 | 7.0E+06 | 6.85 | 0.30 | — |
| | 0 | 5 | 9 | 2000000 | 7 | 1.4E+07 | 7.15 | — | 0.00 |
| | 2.5 | 43 | 59 | 200000 | 51 | 1.0E+07 | 7.01 | 0.14 | −0.08 |
| CAMHB | 5 | 12 | 6 | 2000000 | 9 | 1.8E+07 | 7.26 | −0.11 | 0.16 |
| + | 10 | 53 | 53 | 200000 | 53 | 1.1E+07 | 7.03 | 0.12 | 0.25 |
| 50 mM | 15 | 44 | 54 | 200000 | 49 | 9.8E+06 | 6.99 | 0.15 | 0.00 |
| | 30 | 7 | 5 | 2000000 | 6 | 1.2E+07 | 7.08 | 0.07 | −0.23 |
| PBS | 0 | 8 | 5 | 2000000 | 6.5 | 1.3E+07 | 7.11 | 0.00 | — |
| 0.25X | 2.5 | 51 | 48 | 20000 | 49.5 | 9.9E+05 | 6.00 | 1.12 | — |
| MIC | 5 | 27 | 34 | 20000 | 30.5 | 6.1E+05 | 5.79 | 1.33 | — |
| | 10 | 37 | 36 | 20000 | 36.5 | 7.3E+05 | 5.86 | 1.25 | — |
| | 15 | 36 | 22 | 20000 | 29 | 5.8E+05 | 5.76 | 1.35 | — |
| | 30 | 25 | 39 | 20000 | 32 | 6.4E+05 | 5.81 | 1.31 | — |
| PBS | 0 | 31 | 45 | 200000 | 38 | 7.6E+06 | 6.88 | 0.04 | 0.23 |
| + | 2.5 | 27 | 49 | 2000 | 38 | 7.6E+04 | 4.88 | 2.04 | 1.11 |
| 50 mM | 5 | 28 | 29 | 2000 | 28.5 | 5.7E+04 | 4.76 | 2.16 | 1.03 |
| NaCHO₃ | 10 | 6 | 13 | 20000 | 9.5 | 1.9E+05 | 5.28 | 1.64 | 0.58 |
| 0.25X | 15 | 7 | 11 | 20000 | 9 | 1.8E+05 | 5.26 | 1.66 | 0.51 |
| MIC | 30 | 9 | 6 | 20000 | 7.5 | 1.5E+05 | 5.18 | 1.74 | 0.63 |
| H₂O | 0 | 6 | 4 | 2000000 | 5 | 1.0E+07 | 7.00 | −0.03 | — |
| 0.25X | 2.5 | 65 | 74 | 20000 | 69.5 | 1.4E+06 | 6.14 | 0.83 | — |
| MIC | 5 | 32 | 32 | 20000 | 32 | 6.4E+05 | 5.81 | 1.16 | — |
| | 10 | 18 | 11 | 20000 | 14.5 | 2.9E+05 | 5.46 | 1.51 | — |
| | 15 | 50 | 56 | 2000 | 53 | 1.1E+05 | 5.03 | 1.94 | — |
| | 30 | 50 | 54 | 2000 | 52 | 1.0E+05 | 5.02 | 1.95 | — |
| H₂O | 0 | 49 | 56 | 200000 | 52.5 | 1.1E+07 | 7.02 | −0.01 | −0.02 |
| + | 2.5 | 12 | 11 | 20000 | 11.5 | 2.3E+05 | 5.36 | 1.65 | 0.78 |
| 50 mM | 5 | 13 | 12 | 20000 | 12.5 | 2.5E+05 | 5.40 | 1.61 | 0.41 |
| NaCHO₃ | 10 | 43 | 36 | 2000 | 39.5 | 7.9E+04 | 4.90 | 2.11 | 0.56 |
| 0.25X | 15 | 17 | 24 | 2000 | 20.5 | 4.1E+04 | 4.61 | 2.40 | 0.41 |
| MIC | 30 | 4 | 10 | 20000 | 7 | 1.4E+05 | 5.15 | 1.86 | −0.13 |
| CAMHB | 0 | 50 | 45 | 200000 | 47.5 | 9.5E+06 | 6.98 | 0.17 | — |
| 0.25X | 2.5 | 9 | 6 | 2000000 | 7.5 | 1.5E+07 | 7.18 | −0.03 | — |
| MIC | 5 | 9 | 8 | 2000000 | 8.5 | 1.7E+07 | 7.23 | −0.08 | — |
| | 10 | 8 | 13 | 2000000 | 10.5 | 2.1E+07 | 7.32 | −0.18 | — |
| | 15 | 37 | 30 | 200000 | 33.5 | 6.7E+06 | 6.83 | 0.32 | — |
| | 30 | 38 | 55 | 200000 | 46.5 | 9.3E+06 | 6.97 | 0.18 | — |
| CAMHB | 0 | 62 | 48 | 200000 | 55 | 1.1E+07 | 7.04 | 0.10 | −0.06 |
| + | 2.5 | 53 | 46 | 200000 | 49.5 | 9.9E+06 | 7.00 | 0.15 | 0.18 |
| 50 mM | 5 | 12 | 11 | 2000000 | 11.5 | 2.3E+07 | 7.36 | −0.22 | −0.13 |
| NaCHO₃ | 10 | 52 | 41 | 200000 | 46.5 | 9.3E+06 | 6.97 | 0.18 | 0.35 |
| 0.25X | 15 | 8 | 5 | 2000000 | 6.5 | 1.3E+07 | 7.11 | 0.03 | −0.29 |
| MIC | 30 | 28 | 29 | 200000 | 28.5 | 5.7E+06 | 6.76 | 0.39 | 0.21 |
| PBS | 0 | 17 | 19 | 200000 | 18 | 3.6E+06 | 6.56 | 0.56 | — |
| 0.5X MIC | 2.5 | 26 | 25 | 2000 | 25.5 | 5.1E+04 | 4.71 | 2.41 | — |
| | 5 | 9 | 7 | 2000 | 8 | 1.6E+04 | 4.20 | 2.91 | — |
| | 10 | 42 | 27 | 200 | 34.5 | 6.9E+03 | 3.84 | 3.28 | — |
| | 15 | 36 | 39 | 200 | 37.5 | 7.5E+03 | 3.88 | 3.24 | — |
| | 30 | 49 | 51 | 200 | 50 | 1.0E+04 | 4.00 | 3.11 | — |
| PBS | 0 | 30 | 32 | 200000 | 31 | 6.2E+06 | 6.79 | 0.13 | −0.24 |
| + | 2.5 | 8 | 15 | 20000 | 11.5 | 2.3E+05 | 5.36 | 1.56 | −0.65 |
| 50 mM | 5 | 6 | 8 | 2000 | 7 | 1.4E+04 | 4.15 | 2.77 | 0.06 |
| NaCHO₃ | 10 | 26 | 27 | 200 | 26.5 | 5.3E+03 | 3.72 | 3.19 | 0.11 |
| 0.5X MIC | 15 | 40 | 42 | 200 | 41 | 8.2E+03 | 3.91 | 3.01 | −0.04 |
| | 30 | 33 | 24 | 200 | 28.5 | 5.7E+03 | 3.76 | 3.16 | 0.24 |
| H₂O | 0 | 20 | 19 | 200000 | 19.5 | 3.9E+06 | 6.59 | 0.38 | — |
| 0.5X MIC | 2.5 | 15 | 30 | 2000 | 22.5 | 4.5E+04 | 4.65 | 2.32 | — |
| | 5 | 23 | 23 | 2000 | 23 | 4.6E+04 | 4.66 | 2.31 | — |
| | 10 | 9 | 6 | 2000 | 7.5 | 1.5E+04 | 4.18 | 2.79 | — |
| | 15 | 40 | 39 | 200 | 39.5 | 7.9E+03 | 3.90 | 3.07 | — |
| | 30 | 26 | 33 | 200 | 29.5 | 5.9E+03 | 3.77 | 3.20 | — |
| H₂O | 0 | 22 | 27 | 200000 | 24.5 | 4.9E+06 | 6.69 | 0.32 | −0.10 |
| + | 2.5 | 31 | 24 | 200 | 27.5 | 5.5E+03 | 3.74 | 3.27 | 0.91 |
| 50 mM | 5 | 25 | 28 | 200 | 26.5 | 5.3E+03 | 3.72 | 3.28 | 0.94 |
| NaCHO₃ | 10 | 26 | 25 | 200 | 25.5 | 5.1E+03 | 3.71 | 3.30 | 0.47 |
| 0.5X MIC | 15 | 10 | 21 | 200 | 15.5 | 3.1E+03 | 3.49 | 3.52 | 0.41 |
| | 30 | 18 | 16 | 200 | 17 | 3.4E+03 | 3.53 | 3.48 | 0.24 |
| CAMHB | 0 | 37 | 41 | 200000 | 39 | 7.8E+06 | 6.89 | 0.25 | — |
| 0.5X MIC | 2.5 | 36 | 46 | 200000 | 41 | 8.2E+06 | 6.91 | 0.23 | — |
| | 5 | 36 | 31 | 200000 | 33.5 | 6.7E+06 | 6.83 | 0.32 | — |
| | 10 | 8 | 6 | 2000000 | 7 | 1.4E+07 | 7.15 | 0.00 | — |
| | 15 | 6 | 7 | 2000000 | 6.5 | 1.3E+07 | 7.11 | 0.03 | — |
| | 30 | 47 | 47 | 200000 | 47 | 9.4E+06 | 6.97 | 0.17 | — |
| CAMHB | 0 | 48 | 39 | 200000 | 43.5 | 8.7E+06 | 6.94 | 0.21 | −0.05 |
| + | 2.5 | 50 | 56 | 200000 | 53 | 1.1E+07 | 7.03 | 0.12 | −0.11 |
| 50 mM | 5 | 32 | 28 | 200000 | 30 | 6.0E+06 | 6.78 | 0.37 | 0.05 |
| NaCHO₃ | 10 | 42 | 52 | 200000 | 47 | 9.4E+06 | 6.97 | 0.17 | 0.17 |
| 0.5X MIC | 15 | 35 | 28 | 200000 | 31.5 | 6.3E+06 | 6.80 | 0.35 | 0.31 |
| | 30 | 9 | 8 | 200000 | 8.5 | 1.7E+06 | 6.23 | 0.92 | 0.74 |
| PBS | 0 | 27 | 32 | 20000 | 29.5 | 5.9E+05 | 5.77 | 1.34 | — |
| 1X MIC | 2.5 | 15 | 17 | 200 | 16 | 3.2E+03 | 3.51 | 3.61 | — |
| | 5 | 23 | 10 | 200 | 16.5 | 3.3E+03 | 3.52 | 3.60 | — |
| | 10 | 9 | 7 | 200 | 8 | 1.6E+03 | 3.20 | 3.91 | — |
| | 15 | 8 | 3 | 200 | 5.5 | 1.1E+03 | 3.04 | 4.07 | — |
| | 30 | 4 | 1 | 200 | 2.5 | 5.0E+02 | 2.70 | 4.41 | — |
| PBS | 0 | 32 | 36 | 20000 | 34 | 6.8E+05 | 5.83 | 1.09 | −0.06 |
| + | 2.5 | 18 | 37 | 200 | 27.5 | 5.5E+03 | 3.74 | 3.18 | −0.24 |
| 50 mM | 5 | 8 | 10 | 200 | 9 | 1.8E+03 | 3.26 | 3.66 | 0.26 |
| NaCHO₃ | 10 | 24 | 31 | 200 | 27.5 | 5.5E+03 | 3.74 | 3.18 | −0.54 |
| 1X MIC | 15 | 3 | 16 | 200 | 9.5 | 1.9E+03 | 3.28 | 3.64 | −0.24 |
| | 30 | 14 | 4 | 200 | 9 | 1.8E+03 | 3.26 | 3.66 | −0.56 |
| H₂O | 0 | 15 | 15 | 20000 | 15 | 3.0E+05 | 5.48 | 1.49 | — |
| 1X MIC | 2.5 | 12 | 24 | 200 | 18 | 3.6E+03 | 3.56 | 3.41 | — |
| | 5 | 10 | 18 | 200 | 14 | 2.8E+03 | 3.45 | 3.52 | — |
| | 10 | 7 | 17 | 200 | 12 | 2.4E+03 | 3.38 | 3.59 | — |
| | 15 | 18 | 7 | 200 | 12.5 | 2.5E+03 | 3.40 | 3.57 | — |
| | 30 | 3 | 2 | 200 | 2.5 | 5.0E+02 | 2.70 | 4.27 | — |
| H₂O | 0 | 30 | 37 | 20000 | 33.5 | 6.7E+05 | 5.83 | 1.18 | −0.35 |
| + | 2.5 | 17 | 15 | 2000 | 16 | 3.2E+04 | 4.51 | 2.50 | −0.95 |
| 50 mM | 5 | 14 | 19 | 200 | 16.5 | 3.3E+03 | 3.52 | 3.49 | −0.07 |
| NaCHO₃ | 10 | 6 | 7 | 200 | 6.5 | 1.3E+03 | 3.11 | 3.89 | 0.27 |
| 1X MIC | 15 | 16 | 3 | 200 | 9.5 | 1.9E+03 | 3.28 | 3.73 | 0.12 |
| | 30 | 9 | 4 | 200 | 6.5 | 1.3E+03 | 3.11 | 3.89 | −0.41 |
| CAMHB | 0 | 56 | 53 | 200000 | 54.5 | 1.1E+07 | 7.04 | 0.11 | — |
| 1X MIC | 2.5 | 39 | 42 | 200000 | 40.5 | 8.1E+06 | 6.91 | 0.24 | — |
| | 5 | 6 | 11 | 2000000 | 8.5 | 1.7E+07 | 7.23 | −0.08 | — |
| | 10 | 63 | 50 | 200000 | 56.5 | 1.1E+07 | 7.05 | 0.09 | — |
| | 15 | 49 | 57 | 200000 | 53 | 1.1E+07 | 7.03 | 0.12 | — |
| | 30 | 35 | 24 | 200000 | 29.5 | 5.9E+06 | 6.77 | 0.38 | — |
| CAMHB | 0 | 68 | 64 | 200000 | 66 | 1.3E+07 | 7.12 | 0.03 | −0.08 |
| + | 2.5 | 34 | 29 | 200000 | 31.5 | 6.3E+06 | 6.80 | 0.35 | 0.11 |
| 50 mM | 5 | 32 | 35 | 200000 | 33.5 | 6.7E+06 | 6.83 | 0.32 | 0.40 |
| NaCHO₃ | 10 | 26 | 27 | 200000 | 26.5 | 5.3E+06 | 6.72 | 0.42 | 0.33 |
| 1X MIC | 15 | 53 | 51 | 20000 | 52 | 1.0E+06 | 6.02 | 1.13 | 1.01 |
| | 30 | 36 | 55 | 2000 | 45.5 | 9.1E+04 | 4.96 | 2.19 | 1.81 |

TABLE 14D
Data for time-kill study with SEQ ID NO: 1.
Isolate: Escherichia coli CDC 0451 (KPC)

| | | | | | | | | Log-kill | Log-CFU |
|---|---|---|---|---|---|---|---|---|---|
| | | | | | | | | (log₁₀ | (log₁₀ |
| | | | | | | | | CFU/ml) | CFU/ml) |
| | | | | | | | | relative | relative |
| | | | | | | | | to base | to media |
| | | | | | | | | media | without |
| | | Count | Count | | | | log₁₀ | with no | NaCHO₃ |
| | Time | 1 | 2 | | Average | Average | CFU/ | drug | at each |
| Condition | (min) | CFU | CFU | DF | CFU | CFU/ml | ml | at 0 min | time point |
| PBS | 0 | 8 | 8 | 2000000 | 8 | 1.6E+07 | 7.20 | — | — |
| | 2.5 | 7 | 11 | 2000000 | 9 | 1.8E+07 | 7.26 | −0.05 | — |
| | 5 | 5 | 8 | 2000000 | 6.5 | 1.3E+07 | 7.11 | 0.09 | — |
| | 10 | 10 | 6 | 2000000 | 8 | 1.6E+07 | 7.20 | 0.00 | — |
| | 15 | 8 | 6 | 2000000 | 7 | 1.4E+07 | 7.15 | 0.06 | — |
| | 30 | 12 | 9 | 2000000 | 10.5 | 2.1E+07 | 7.32 | −0.12 | — |
| PBS | 0 | 7 | 7 | 2000000 | 7 | 1.4E+07 | 7.15 | — | 0.06 |
| + | 2.5 | 7 | 12 | 2000000 | 9.5 | 1.9E+07 | 7.28 | −0.13 | −0.02 |
| 50 mM | 5 | 5 | 5 | 2000000 | 5 | 1.0E+07 | 7.00 | 0.15 | 0.11 |
| NaCHO₃ | 10 | 9 | 8 | 2000000 | 8.5 | 1.7E+07 | 7.23 | −0.08 | −0.03 |
| | 15 | 8 | 9 | 2000000 | 8.5 | 1.7E+07 | 7.23 | −0.08 | −0.08 |
| | 30 | 8 | 7 | 2000000 | 7.5 | 1.5E+07 | 7.18 | −0.03 | 0.15 |
| H₂O | 0 | 55 | 66 | 200000 | 60.5 | 1.2E+07 | 7.08 | — | — |
| | 2.5 | 9 | 8 | 2000000 | 8.5 | 1.7E+07 | 7.23 | −0.15 | — |
| | 5 | 7 | 5 | 2000000 | 6 | 1.2E+07 | 7.08 | 0.00 | — |
| | 10 | 8 | 4 | 2000000 | 6 | 1.2E+07 | 7.08 | 0.00 | — |
| | 15 | 49 | 17 | 200000 | 33 | 6.6E+06 | 6.82 | 0.26 | — |
| | 30 | 7 | 4 | 2000000 | 5.5 | 1.1E+07 | 7.04 | 0.04 | — |
| H₂O | 0 | 8 | 12 | 2000000 | 10 | 2.0E+07 | 7.30 | — | −0.22 |
| + | 2.5 | 7 | 10 | 2000000 | 8.5 | 1.7E+07 | 7.23 | 0.07 | 0.00 |
| 50 mM | 5 | 6 | 5 | 2000000 | 5.5 | 1.1E+07 | 7.04 | 0.26 | 0.04 |
| NaCHO₃ | 10 | 11 | 7 | 2000000 | 9 | 1.8E+07 | 7.26 | 0.05 | −0.18 |
| | 15 | 7 | 5 | 2000000 | 6 | 1.2E+07 | 7.08 | 0.22 | −0.26 |
| | 30 | 11 | 7 | 2000000 | 9 | 1.8E+07 | 7.26 | 0.05 | −0.21 |
| CAMHB | 0 | 7 | 12 | 2000000 | 9.5 | 1.9E+07 | 7.28 | — | — |
| | 2.5 | 11 | 6 | 2000000 | 8.5 | 1.7E+07 | 7.23 | 0.05 | — |
| | 5 | 53 | 53 | 200000 | 53 | 1.1E+07 | 7.03 | 0.25 | — |
| | 10 | 50 | 47 | 200000 | 48.5 | 9.7E+06 | 6.99 | 0.29 | — |
| | 15 | 6 | 9 | 2000000 | 7.5 | 1.5E+07 | 7.18 | 0.10 | — |
| | 30 | 46 | 48 | 200000 | 47 | 9.4E+06 | 6.97 | 0.31 | — |
| CAMHB | 0 | 13 | 5 | 2000000 | 9 | 1.8E+07 | 7.26 | — | 0.02 |
| + | 2.5 | 6 | 6 | 2000000 | 6 | 1.2E+07 | 7.08 | 0.18 | 0.15 |
| 50 mM | 5 | 10 | 7 | 2000000 | 8.5 | 1.7E+07 | 7.23 | 0.02 | −0.21 |
| NaCHO₃ | 10 | 7 | 11 | 2000000 | 9 | 1.8E+07 | 7.26 | 0.00 | −0.27 |
| | 15 | 3 | 11 | 2000000 | 7 | 1.4E+07 | 7.15 | 0.11 | 0.03 |
| | 30 | 41 | 46 | 200000 | 43.5 | 8.7E+06 | 6.94 | 0.32 | 0.03 |
| PBS | 0 | 11 | 11 | 2000000 | 11 | 2.2E+07 | 7.34 | −0.14 | — |
| 0.25X | 2.5 | 10 | 11 | 2000000 | 10.5 | 2.1E+07 | 7.32 | −0.12 | — |
| MIC | 5 | 49 | 49 | 200000 | 49 | 9.8E+06 | 6.99 | 0.21 | — |
| | 10 | 6 | 10 | 2000000 | 8 | 1.6E+07 | 7.20 | 0.00 | — |
| | 15 | 55 | 56 | 200000 | 55.5 | 1.1E+07 | 7.05 | 0.16 | — |
| | 30 | 5 | 3 | 2000000 | 4 | 8.0E+06 | 6.90 | 0.30 | — |
| PBS | 0 | 11 | 8 | 2000000 | 9.5 | 1.9E+07 | 7.28 | −0.13 | 0.06 |
| + | 2.5 | 8 | 16 | 2000000 | 12 | 2.4E+07 | 7.38 | −0.23 | −0.06 |
| 50 mM | 5 | 8 | 5 | 2000000 | 6.5 | 1.3E+07 | 7.11 | 0.03 | −0.12 |
| NaCHO₃ | 10 | 57 | 61 | 200000 | 59 | 1.2E+07 | 7.07 | 0.07 | 0.13 |
| 0.25X | 15 | 49 | 27 | 200000 | 38 | 7.6E+06 | 6.88 | 0.27 | 0.16 |
| MIC | 30 | 8 | 11 | 2000000 | 9.5 | 1.9E+07 | 7.28 | −0.13 | −0.38 |
| H₂O | 0 | 12 | 6 | 2000000 | 9 | 1.8E+07 | 7.26 | −0.17 | — |
| 0.25X | 2.5 | 37 | 38 | 200000 | 37.5 | 7.5E+06 | 6.88 | 0.21 | — |
| MIC | 5 | 29 | 28 | 200000 | 28.5 | 5.7E+06 | 6.76 | 0.33 | — |
| | 10 | 13 | 11 | 200000 | 12 | 2.4E+06 | 6.38 | 0.70 | — |
| | 15 | 4 | 5 | 200000 | 4.5 | 9.0E+05 | 5.95 | 1.13 | — |
| | 30 | 9 | 7 | 200000 | 8 | 1.6E+06 | 6.20 | 0.88 | — |
| H₂O | 0 | 6 | 5 | 2000000 | 5.5 | 1.1E+07 | 7.04 | 0.26 | 0.21 |
| + | 2.5 | 25 | 32 | 200000 | 28.5 | 5.7E+06 | 6.76 | 0.55 | 0.12 |
| 50 mM | 5 | 10 | 16 | 200000 | 13 | 2.6E+06 | 6.41 | 0.89 | 0.34 |
| NaCHO₃ | 10 | 5 | 6 | 200000 | 5.5 | 1.1E+06 | 6.04 | 1.26 | 0.34 |
| 0.25X | 15 | 9 | 5 | 200000 | 7 | 1.4E+06 | 6.15 | 1.15 | −0.19 |
| MIC | 30 | 7 | 6 | 200000 | 6.5 | 1.3E+06 | 6.11 | 1.19 | 0.09 |
| CAMHB | 0 | 6 | 9 | 2000000 | 7.5 | 1.5E+07 | 7.18 | 0.10 | — |
| 0.25X | 2.5 | 8 | 7 | 2000000 | 7.5 | 1.5E+07 | 7.18 | 0.10 | — |
| MIC | 5 | 7 | 11 | 2000000 | 9 | 1.8E+07 | 7.26 | 0.02 | — |
| | 10 | 5 | 5 | 2000000 | 5 | 1.0E+07 | 7.00 | 0.28 | — |
| | 15 | 38 | 45 | 200000 | 41.5 | 8.3E+06 | 6.92 | 0.36 | — |
| | 30 | 1 | 9 | 2000000 | 5 | 1.0E+07 | 7.00 | 0.28 | — |
| CAMHB | 0 | 56 | 55 | 200000 | 55.5 | 1.1E+07 | 7.05 | 0.21 | 0.13 |
| + | 2.5 | 6 | 10 | 2000000 | 8 | 1.6E+07 | 7.20 | 0.05 | −0.03 |
| 50 mM | 5 | 38 | 44 | 200000 | 41 | 8.2E+06 | 6.91 | 0.34 | 0.34 |
| NaCHO₃ | 10 | 11 | 11 | 2000000 | 11 | 2.2E+07 | 7.34 | −0.09 | −0.34 |
| 0.25X | 15 | 7 | 5 | 2000000 | 6 | 1.2E+07 | 7.08 | 0.18 | −0.16 |
| MIC | 30 | 12 | 6 | 2000000 | 9 | 1.8E+07 | 7.26 | 0.00 | −0.26 |
| PBS | 0 | 49 | 56 | 200000 | 52.5 | 1.1E+07 | 7.02 | 0.18 | — |
| 0.5X MIC | 2.5 | 7 | 10 | 2000 | 8.5 | 1.7E+04 | 4.23 | 2.97 | — |
| | 5 | 10 | 13 | 2000 | 11.5 | 2.3E+04 | 4.36 | 2.84 | — |
| | 10 | 8 | 10 | 2000 | 9 | 1.8E+04 | 4.26 | 2.95 | — |
| | 15 | 15 | 14 | 2000 | 14.5 | 2.9E+04 | 4.46 | 2.74 | — |
| | 30 | 17 | 28 | 2000 | 22.5 | 4.5E+04 | 4.65 | 2.55 | — |
| | 0 | 6 | 12 | 2000000 | 9 | 1.8E+07 | 7.26 | −0.11 | −0.23 |
| PBS | 2.5 | 34 | 39 | 2000 | 36.5 | 7.3E+04 | 4.86 | 2.28 | −0.63 |
| + | 5 | 5 | 6 | 2000 | 5.5 | 1.1E+04 | 4.04 | 3.10 | 0.32 |
| 50 mM | 10 | 7 | 6 | 2000 | 6.5 | 1.3E+04 | 4.11 | 3.03 | 0.14 |
| NaCHO₃ | 15 | 11 | 13 | 2000 | 12 | 2.4E+04 | 4.38 | 2.77 | 0.08 |
| 0.5X MIC | 30 | 19 | 16 | 2000 | 17.5 | 3.5E+04 | 4.54 | 2.60 | 0.11 |
| H₂O | 0 | 62 | 41 | 200000 | 51.5 | 1.0E+07 | 7.01 | 0.07 | — |
| 0.5X MIC | 2.5 | 32 | 31 | 200 | 31.5 | 6.3E+03 | 3.80 | 3.28 | — |
| | 5 | 39 | 26 | 200 | 32.5 | 6.5E+03 | 3.81 | 3.27 | — |
| | 10 | 35 | 23 | 200 | 29 | 5.8E+03 | 3.76 | 3.32 | — |
| | 15 | 37 | 47 | 200 | 42 | 8.4E+03 | 3.92 | 3.16 | — |
| | 30 | 46 | 49 | 200 | 47.5 | 9.5E+03 | 3.98 | 3.11 | — |
| H₂O | 0 | 11 | 11 | 2000000 | 11 | 2.2E+07 | 7.34 | −0.04 | −0.33 |
| + | 2.5 | 44 | 46 | 2000 | 45 | 9.0E+04 | 4.95 | 2.35 | −1.15 |
| 50 mM | 5 | 68 | 56 | 200 | 62 | 1.2E+04 | 4.09 | 3.21 | −0.28 |
| NaCHO₃ | 10 | 24 | 13 | 200 | 18.5 | 3.7E+03 | 3.57 | 3.73 | 0.20 |
| 0.5X MIC | 15 | 36 | 45 | 200 | 40.5 | 8.1E+03 | 3.91 | 3.39 | 0.02 |
| | 30 | 6 | 11 | 2000 | 8.5 | 1.7E+04 | 4.23 | 3.07 | −0.25 |
| CAMHB | 0 | 58 | 61 | 200000 | 59.5 | 1.2E+07 | 7.08 | 0.20 | — |
| 0.5X MIC | 2.5 | 50 | 63 | 200000 | 56.5 | 1.1E+07 | 7.05 | 0.23 | — |
| | 5 | 8 | 4 | 2000000 | 6 | 1.2E+07 | 7.08 | 0.20 | — |
| | 10 | 51 | 49 | 200000 | 50 | 1.0E+07 | 7.00 | 0.28 | — |
| | 15 | 10 | 5 | 2000000 | 7.5 | 1.5E+07 | 7.18 | 0.10 | — |
| | 30 | 39 | 41 | 200000 | 40 | 8.0E+06 | 6.90 | 0.38 | — |
| CAMHB | 0 | 5 | 5 | 2000000 | 5 | 1.0E+07 | 7.00 | 0.26 | 0.08 |
| + | 2.5 | 7 | 14 | 2000000 | 10.5 | 2.1E+07 | 7.32 | −0.07 | −0.27 |
| 50 mM | 5 | 7 | 14 | 2000000 | 10.5 | 2.1E+07 | 7.32 | −0.07 | −0.24 |
| NaCHO₃ | 10 | 8 | 5 | 2000000 | 6.5 | 1.3E+07 | 7.11 | 0.14 | −0.11 |
| 0.5X MIC | 15 | 8 | 8 | 2000000 | 8 | 1.6E+07 | 7.20 | 0.05 | −0.03 |
| | 30 | 51 | 39 | 200000 | 45 | 9.0E+06 | 6.95 | 0.30 | −0.05 |
| PBS | 0 | 27 | 26 | 200000 | 26.5 | 5.3E+06 | 6.72 | 0.48 | — |
| 1X MIC | 2.5 | 44 | 32 | 200 | 38 | 7.6E+03 | 3.88 | 3.32 | — |
| | 5 | 36 | 30 | 200 | 33 | 6.6E+03 | 3.82 | 3.38 | — |
| | 10 | 2 | 2 | 200 | 2 | 4.0E+02 | 2.60 | 4.60 | — |
| | 15 | 0 | 2 | 200 | 1 | 2.0E+02 | 2.30 | 4.90 | — |
| | 30 | 1 | 0 | 200 | 0.5 | 1.0E+02 | 2.00 | >5.20 | — |
| PBS | 0 | 7 | 10 | 2000000 | 8.5 | 1.7E+07 | 7.23 | −0.08 | −0.51 |
| + | 2.5 | 11 | 12 | 2000 | 11.5 | 2.3E+04 | 4.36 | 2.78 | −0.48 |
| 50 mM | 5 | 23 | 17 | 200 | 20 | 4.0E+03 | 3.60 | 3.54 | 0.22 |
| NaCHO₃ | 10 | 1 | 2 | 200 | 1.5 | 3.0E+02 | 2.48 | 4.67 | 0.12 |
| 1X MIC | 15 | 4 | 0 | 200 | 2 | 4.0E+02 | 2.60 | 4.54 | −0.30 |
| | 30 | 2 | 0 | 200 | 1 | 2.0E+02 | 2.30 | 4.85 | −0.30 |
| H₂O | 0 | 20 | 18 | 200000 | 19 | 3.8E+06 | 6.58 | 0.50 | — |
| 1X MIC | 2.5 | 0 | 1 | 200 | 0.5 | 1.0E+02 | 2.00 | >5.08 | — |
| | 5 | 0 | 0 | NA | 0 | <1.0+02 | <2.0 | >5.08 | — |
| | 10 | 0 | 0 | NA | 0) | <1.0+02 | <2.0 | >5.08 | — |
| | 15 | 0 | 1 | 200 | 0.5 | 1.0E+02 | 2.00 | >5.08 | — |
| | 30 | 1 | 0 | 200 | 0.5 | 1.0E+02 | 2.00 | >5.08 | — |
| H₂O | 0 | 51 | 40 | 200000 | 45.5 | 9.1E+06 | 6.96 | 0.34 | −0.38 |
| + | 2.5 | 22 | 32 | 2000 | 27 | 5.4E+04 | 4.73 | 2.57 | −2.73 |
| 50 mM | 5 | 14 | 15 | 200 | 14.5 | 2.9E+03 | 3.46 | 3.84 | −1.46 |
| NaCHO₃ | 10 | 3 | 8 | 200 | 5.5 | 1.1E+03 | 3.04 | 4.26 | −1.04 |
| 1X MIC | 15 | 2 | 0 | 200 | 1 | 2.0E+02 | 2.30 | 5.00 | −0.30 |
| | 30 | 0 | 0 | NA | 0 | <1.0 + 02 | <2.0 | >5.30 | 0.00 |
| CAMHB | 0 | 10 | 5 | 2000000 | 7.5 | 1.5E+07 | 7.18 | 0.10 | — |
| 1X MIC | 2.5 | 7 | 8 | 2000000 | 7.5 | 1.5E+07 | 7.18 | 0.10 | — |
| | 5 | 47 | 55 | 200000 | 51 | 1.0E+07 | 7.01 | 0.27 | — |
| | 10 | 32 | 44 | 200000 | 38 | 7.6E+06 | 6.88 | 0.40 | — |
| | 15 | 6 | 5 | 2000000 | 5.5 | 1.1E+07 | 7.04 | 0.24 | — |
| | 30 | 50 | 52 | 200000 | 51 | 1.0E+07 | 7.01 | 0.27 | — |
| CAMHB | 0 | 51 | 51 | 200000 | 51 | 1.0E+07 | 7.01 | 0.25 | 0.17 |
| + | 2.5 | 6 | 4 | 2000000 | 5 | 1.0E+07 | 7.00 | 0.26 | 0.18 |
| 50 mM | 5 | 5 | 9 | 2000000 | 7 | 1.4E+07 | 7.15 | 0.11 | −0.14 |
| NaCHO₃ | 10 | 50 | 61 | 200000 | 55.5 | 1.1E+07 | 7.05 | 0.21 | −0.16 |
| 1X MIC | 15 | 3 | 11 | 2000000 | 7 | 1.4E+07 | 7.15 | 0.11 | −0.10 |
| | 30 | 5 | 8 | 2000000 | 6.5 | 1.3E+07 | 7.11 | 0.14 | −0.11 |

TABLE 14E
Data for time-kill study with SEQ ID NO: 1. Isolate:
Pseudomonas aeruginosa CDC 0509

| | | | | | | | | Log-kill | Log-CFU |
|---|---|---|---|---|---|---|---|---|---|
| | | | | | | | | (log₁₀ | (log₁₀ |
| | | | | | | | | CFU/ml) | CFU/ml) |
| | | | | | | | | relative | relative |
| | | | | | | | | to base | to media |
| | | | | | | | | media | without |
| | | Count | Count | | | | log₁₀ | with no | NaCHO₃ |
| | Time | 1 | 2 | | Average | Average | CFU/ | drug at | at each |
| Condition | (min) | CFU | CFU | DF | CFU | CFU/ml | ml | 0 min | time point |
| PBS | 0 | 10 | 16 | 2000000 | 13 | 2.6E+07 | 7.41 | — | — |
| | 2.5 | 8 | 11 | 2000000 | 9.5 | 1.9E+07 | 7.28 | 0.14 | — |
| | 5 | 46 | 40 | 200000 | 43 | 8.6E+06 | 6.93 | 0.48 | — |
| | 10 | 35 | 26 | 200000 | 30.5 | 6.1E+06 | 6.79 | 0.63 | — |
| | 15 | 31 | 34 | 200000 | 32.5 | 6.5E+06 | 6.81 | 0.60 | — |
| | 30 | 40 | 37 | 200000 | 38.5 | 7.7E+06 | 6.89 | 0.53 | — |
| | 0 | 12 | 11 | 2000000 | 11.5 | 2.3E+07 | 7.36 | — | 0.05 |
| PBS | 2.5 | 46 | 62 | 200000 | 54 | 1.1E+07 | 7.03 | 0.33 | 0.25 |
| + | 5 | 47 | 45 | 200000 | 46 | 9.2E+06 | 6.96 | 0.40 | −0.03 |
| 50 mM | 10 | 44 | 37 | 200000 | 40.5 | 8.1E+06 | 6.91 | 0.45 | −0.12 |
| NaCHO₃ | 15 | 36 | 39 | 200000 | 37.5 | 7.5E+06 | 6.88 | 0.49 | −0.06 |
| | 30 | 34 | 26 | 200000 | 30 | 6.0E+06 | 6.78 | 0.58 | 0.11 |
| H₂O | 0 | 6 | 10 | 2000000 | 8 | 1.6E+07 | 7.20 | — | — |
| | 2.5 | 6 | 8 | 2000000 | 7 | 1.4E+07 | 7.15 | 0.06 | — |
| | 5 | 31 | 30 | 200000 | 30.5 | 6.1E+06 | 6.79 | 0.42 | — |
| | 10 | 28 | 25 | 200000 | 26.5 | 5.3E+06 | 6.72 | 0.48 | — |
| | 15 | 24 | 15 | 200000 | 19.5 | 3.9E+06 | 6.59 | 0.61 | — |
| | 30 | 13 | 15 | 200000 | 14 | 2.8E+06 | 6.45 | 0.76 | — |
| H₂O | 0 | 2 | 12 | 2000000 | 7 | 1.4E+07 | 7.15 | — | 0.06 |
| + | 2.5 | 51 | 49 | 200000 | 50 | 1.0E+07 | 7.00 | 0.15 | 0.15 |
| 50 mM | 5 | 4 | 5 | 2000000 | 4.5 | 9.0E+06 | 6.95 | 0.19 | −0.17 |
| NaCHO₃ | 10 | 30 | 30 | 200000 | 30 | 6.0E+06 | 6.78 | 0.37 | −0.05 |
| | 15 | 32 | 16 | 200000 | 24 | 4.8E+06 | 6.68 | 0.46 | −0.09 |
| | 30 | 18 | 20 | 200000 | 19 | 3.8E+06 | 6.58 | 0.57 | −0.13 |
| CAMHB | 0 | 61 | 56 | 200000 | 58.5 | 1.2E+07 | 7.07 | — | — |
| | 2.5 | 28 | 22 | 200000 | 25 | 5.0E+06 | 6.70 | 0.37 | — |
| | 5 | 36 | 26 | 200000 | 31 | 6.2E+06 | 6.79 | 0.28 | — |
| | 10 | 28 | 28 | 200000 | 28 | 5.6E+06 | 6.75 | 0.32 | — |
| | 15 | 23 | 26 | 200000 | 24.5 | 4.9E+06 | 6.69 | 0.38 | — |
| | 30 | 18 | 22 | 200000 | 20 | 4.0E+06 | 6.60 | 0.47 | — |
| CAMHB | 0 | 3 | 6 | 2000000 | 4.5 | 9.0E+06 | 6.95 | — | 0.11 |
| + | 2.5 | 29 | 46 | 200000 | 37.5 | 7.5E+06 | 6.88 | 0.08 | −0.18 |
| 50 mM | 5 | 8 | 6 | 2000000 | 7 | 1.4E+07 | 7.15 | −0.19 | −0.35 |
| NaCHO₃ | 10 | 32 | 35 | 200000 | 33.5 | 6.7E+06 | 6.83 | 0.13 | −0.08 |
| | 15 | 29 | 31 | 200000 | 30 | 6.0E+06 | 6.78 | 0.18 | −0.09 |
| | 30 | 25 | 15 | 200000 | 20 | 4.0E+06 | 6.60 | 0.35 | 0.00 |
| PBS | 0 | 23 | 23 | 200000 | 23 | 4.6E+06 | 6.66 | 0.75 | — |
| 0.25X | 2.5 | 0 | 4 | 200 | 2 | 4.0E+02 | 2.60 | 4.81 | — |
| MIC | 5 | 0 | 0 | NA | 0 | <1.0 + 02 | <2.0 | >5.41 | — |
| | 10 | 0 | 0 | NA | 0 | <1.0 + 02 | <2.0 | >5.41 | — |
| | 15 | 0 | 0 | NA | | <1.0 + 02 | <2.0 | >5.41 | — |
| | 30 | 1 | 1 | 200 | 1 | 2.0E+02 | 2.30 | 5.11 | — |
| PBS | 0 | 38 | 47 | 200000 | 42.5 | 8.5E+06 | 6.93 | 0.43 | −0.27 |
| + | 2.5 | 30 | 14 | 2000 | 22 | 4.4E+04 | 4.64 | 2.72 | −2.04 |
| 50 mM | 5 | 36 | 37 | 200 | 36.5 | 7.3E+03 | 3.86 | 3.50 | −1.86 |
| NaCHO₃ | 10 | 1 | 1 | 200 | 1 | 2.0E+02 | 2.30 | 5.06 | −0.30 |
| 0.25X | 15 | 0 | 0 | NA | 0 | <1.0 + 02 | <2.0 | >5.36 | 0.00 |
| MIC | 30 | 0 | 0 | NA | 0 | <1.0 + 02 | <2.0 | >5.36 | 0.30 |
| H₂O | 0 | 8 | 7 | 200000 | 7.5 | 1.5E+06 | 6.18 | 1.03 | — |
| 0.25X | 2.5 | 0 | 0 | NA | 0 | <1.0 + 02 | <2.0 | >5.20 | — |
| MIC | 5 | 1 | 0 | 200 | 0.5 | 1.0E+02 | 2.00 | >5.20 | — |
| | 10 | 0 | 0 | NA | 0 | <1.0 + 02 | <2.0 | >5.20 | — |
| | 15 | 0 | 0 | NA | 0 | <1.0 + 02 | <2.0 | >5.20 | — |
| | 30 | 0 | 0 | NA | 0 | <1.0 + 02 | <2.0 | >5.20 | — |
| H₂O | 0 | 24 | 20 | 200000 | 22 | 4.4E+06 | 6.64 | 0.50 | −0.47 |
| + | 2.5 | 0 | 2 | 200 | 1 | 2.0E+02 | 2.30 | 4.85 | −0.30 |
| 50 mM | 5 | 0 | 0 | NA | 0 | <1.0 + 02 | <2.0 | >5.15 | 0.00 |
| NaCHO₃ | 10 | 0 | 1 | 200 | 0.5 | 1.0E+02 | 2.00 | >5.15 | 0.00 |
| 0.25X | 15 | 1 | 0 | 200 | 0.5 | 1.0E+02 | 2.00 | >5.15 | 0.00 |
| MIC | 30 | 0 | 0 | NA | 0 | <1.0 + 02 | <2.0 | >5.15 | 0.00 |
| CAMHB | 0 | 10 | 7 | 2000000 | 8.5 | 1.7E+07 | 7.23 | −0.16 | — |
| 0.25X | 2.5 | 21 | 30 | 200000 | 25.5 | 5.1E+06 | 6.71 | 0.36 | — |
| MIC | 5 | 41 | 48 | 200000 | 44.5 | 8.9E+06 | 6.95 | 0.12 | — |
| | 10 | 31 | 29 | 200000 | 30 | 6.0E+06 | 6.78 | 0.29 | — |
| | 15 | 29 | 20 | 200000 | 24.5 | 4.9E+06 | 6.69 | 0.38 | — |
| | 30 | 19 | ND | 200000 | 19 | 3.8E+06 | 6.58 | 0.49 | — |
| CAMHB | 0 | 11 | 9 | 2000000 | 10 | 2.0E+07 | 7.30 | −0.35 | −0.07 |
| + | 2.5 | 15 | 9 | 2000000 | 12 | 2.4E+07 | 7.38 | −0.43 | −0.67 |
| 50 mM | 5 | 46 | 40 | 200000 | 43 | 8.6E+06 | 6.93 | 0.02 | 0.01 |
| NaCHO₃ | 10 | 41 | 27 | 200000 | 34 | 6.8E+06 | 6.83 | 0.12 | −0.05 |
| 0.25X | 15 | 33 | 41 | 200000 | 37 | 7.4E+06 | 6.87 | 0.09 | −0.18 |
| MIC | 30 | 16 | 22 | 200000 | 19 | 3.8E+06 | 6.58 | 0.37 | 0.00 |
| PBS | 0 | 35 | 28 | 20000 | 31.5 | 6.3E+05 | 5.80 | 1.62 | — |
| 0.5X MIC | 2.5 | 0 | 0 | NA | 0 | <1.0 + 02 | <2.0 | >5.41 | — |
| | 5 | 0 | 0 | NA | 0 | <1.0 + 02 | <2.0 | >5.41 | — |
| | 10 | 0 | 0 | NA | 0 | <1.0 + 02 | <2.0 | >5.41 | — |
| | 15 | 0 | 0 | NA | 0 | <1.0 + 02 | <2.0 | >5.41 | — |
| | 30 | 0 | 0 | NA | 0 | <1.0 + 02 | <2.0 | >5.41 | — |
| PBS | 0 | 34 | 23 | 200000 | 28.5 | 5.7E+06 | 6.76 | 0.61 | −0.96 |
| + | 2.5 | 7 | 3 | 2000 | 5 | 1.0E+04 | 4.00 | 3.36 | −2.00 |
| 50 mM | 5 | 1 | 3 | 200 | 2 | 4.0E+02 | 2.60 | 4.76 | −0.60 |
| NaCHO₃ | 10 | 1 | 0 | 200 | 0.5 | 1.0E+02 | 2.00 | >5.36 | 0.00 |
| 0.5X MIC | 15 | 0 | 0 | NA | 0 | <1.0 + 02 | <2.0 | >5.36 | 0.00 |
| | 30 | 1 | 0 | 200 | 0.5 | 1.0E+02 | 2.00 | >5.36 | 0.00 |
| H₂O | 0 | 35 | 34 | 2000 | 34.5 | 6.9E+04 | 4.84 | 2.37 | — |
| 0.5X MIC | 2.5 | 0 | 0 | NA | 0 | <1.0 + 02 | <2.0 | >5.20 | — |
| | 5 | 0 | 0 | NA | 0 | <1.0 + 02 | <2.0 | >5.20 | — |
| | 10 | 0 | 0 | NA | 0 | <1.0 + 02 | <2.0 | >5.20 | — |
| | 15 | 0 | 0 | NA | 0 | <1.0 + 02 | <2.0 | >5.20 | — |
| | 30 | 0 | 0 | NA | 0 | <1.0 + 02 | <2.0 | >5.20 | — |
| H₂O | 0 | 55 | 50 | 20000 | 52.5 | 1.1E+06 | 6.02 | 1.12 | −1.18 |
| + | 2.5 | 0 | 0 | NA | 0 | <1.0 + 02 | <2.0 | >5.15 | 0.00 |
| 50 mM | 5 | 0 | 0 | NA | 0 | <1.0 + 02 | <2.0 | >5.15 | 0.00 |
| NaCHO₃ | 10 | 0 | 0 | NA | 0 | <1.0 + 02 | <2.0 | >5.15 | 0.00 |
| 0.5X MIC | 15 | 0 | 0 | NA | 0 | <1.0 + 02 | <2.0 | >5.15 | 0.00 |
| | 30 | 0 | 0 | NA | 0 | <1.0 + 02 | <2.0 | >5.15 | 0.00 |
| CAMHB | 0 | 10 | 8 | 2000000 | 9 | 1.8E+07 | 7.26 | −0.19 | — |
| 0.5X MIC | 2.5 | 3 | 8 | 2000000 | 5.5 | 1.1E+07 | 7.04 | 0.03 | — |
| | 5 | 19 | 18 | 200000 | 18.5 | 3.7E+06 | 6.57 | 0.50 | — |
| | 10 | 22 | 27 | 200000 | 24.5 | 4.9E+06 | 6.69 | 0.38 | — |
| | 15 | 27 | 24 | 200000 | 25.5 | 5.1E+06 | 6.71 | 0.36 | — |
| | 30 | 32 | 26 | 200000 | 29 | 5.8E+06 | 6.76 | 0.30 | — |
| CAMHB | 0 | 36 | 50 | 200000 | 43 | 8.6E+06 | 6.93 | 0.02 | 0.32 |
| + | 2.5 | 43 | 32 | 200000 | 37.5 | 7.5E+06 | 6.88 | 0.08 | 0.17 |
| 50 mM | 5 | 21 | 41 | 200000 | 31 | 6.2E+06 | 6.79 | 0.16 | −0.22 |
| NaCHO₃ | 10 | 21 | 28 | 200000 | 24.5 | 4.9E+06 | 6.69 | 0.26 | 0.00 |
| 0.5X MIC | 15 | 21 | 21 | 200000 | 21 | 4.2E+06 | 6.62 | 0.33 | 0.08 |
| | 30 | 23 | 17 | 200000 | 20 | 4.0E+06 | 6.60 | 0.35 | 0.16 |
| PBS | 0 | 12 | 12 | 20000 | 12 | 2.4E+05 | 5.38 | 2.03 | — |
| 1X MIC | 2.5 | 0 | 0 | NA | 0 | <1.0 + 02 | <2.0 | >5.41 | — |
| | 5 | 0 | 1 | 200 | 0.5 | 1.0E+02 | 2.00 | >5.41 | — |
| | 10 | 0 | 1 | 200 | 0.5 | 1.0E+02 | 2.00 | >5.41 | — |
| | 15 | 0 | 0 | NA | 0 | <1.0 + 02 | <2.0 | >5.41 | — |
| | 30 | 0 | 0 | NA | 0 | <1.0 + 02 | <2.0 | >5.41 | — |
| PBS | 0 | 17 | 16 | 200000 | 16.5 | 3.3E+06 | 6.52 | 0.84 | −1.14 |
| + | 2.5 | 4 | 2 | 200 | 3 | 6.0E+02 | 2.78 | 4.58 | −0.78 |
| 50 mM | 5 | 0 | 0 | NA | 0 | <1.0 + 02 | <2.0 | >5.36 | 0.00 |
| NaCHO₃ | 10 | 0 | 0 | NA | 0 | <1.0 + 02 | <2.0 | >5.36 | 0.00 |
| 1X MIC | 15 | 0 | 0 | NA | 0 | <1.0 + 02 | <2.0 | >5.36 | 0.00 |
| | 30 | 0 | 0 | NA | 0 | <1.0 + 02 | <2.0 | >5.36 | 0.00 |
| H₂O | 0 | 12 | 6 | 2000 | 9 | 1.8E+04 | 4.26 | 2.95 | — |
| 1X MIC | 2.5 | 0 | 0 | NA | 0 | <1.0 + 02 | <2.0 | >5.20 | — |
| | 5 | 0 | 0 | NA | 0 | <1.0 + 02 | <2.0 | >5.20 | — |
| | 10 | 0 | 0 | NA | 0 | <1.0 + 02 | <2.0 | >5.20 | — |
| | 15 | 0 | 0 | NA | 0 | <1.0 + 02 | <2.0 | >5.20 | — |
| | 30 | 0 | 0 | NA | 0 | <1.0 + 02 | <2.0 | >5.20 | — |
| H₂O | 0 | 23 | ND | 20000 | 23 | 4.6E+05 | 5.66 | 1.48 | −1.41 |
| + | 2.5 | 0 | 0 | NA | 0 | <1.0 + 02 | <2.0 | >5.15 | 0.00 |
| 50 mM | 5 | 0 | 0 | NA | 0 | <1.0 + 02 | <2.0 | >5.15 | 0.00 |
| NaCHO₃ | 10 | 0 | 0 | NA | 0 | <1.0 + 02 | <2.0 | >5.15 | 0.00 |
| 1X MIC | 15 | 0 | 0 | NA | 0 | <1.0 + 02 | <2.0 | >5.15 | 0.00 |
| | 30 | 0 | 0 | NA | 0 | <1.0 + 02 | <2.0 | >5.15 | 0.00 |
| CAMHB | 0 | 11 | 6 | 2000000 | 8.5 | 1.7E+07 | 7.23 | −0.16 | — |
| 1X MIC | 2.5 | 45 | 48 | 200000 | 46.5 | 9.3E+06 | 6.97 | 0.10 | — |
| | 5 | 30 | 38 | 200000 | 34 | 6.8E+06 | 6.83 | 0.24 | — |
| | 10 | 32 | 23 | 200000 | 27.5 | 5.5E+06 | 6.74 | 0.33 | — |
| | 15 | 32 | 28 | 200000 | 30 | 6.0E+06 | 6.78 | 0.29 | — |
| | 30 | 12 | 15 | 200000 | 13.5 | 2.7E+06 | 6.43 | 0.64 | — |
| CAMHB | 0 | 6 | 8 | 2000000 | 7 | 1.4E+07 | 7.15 | −0.19 | 0.08 |
| + | 2.5 | 41 | 31 | 200000 | 36 | 7.2E+06 | 6.86 | 0.10 | 0.11 |
| 50 mM | 5 | 30 | 18 | 200000 | 24 | 4.8E+06 | 6.68 | 0.27 | 0.15 |
| NaCHO₃ | 10 | 29 | 30 | 200000 | 29.5 | 5.9E+06 | 6.77 | 0.18 | −0.03 |
| 1X MIC | 15 | 22 | 28 | 200000 | 25 | 5.0E+06 | 6.70 | 0.26 | 0.08 |
| | 30 | 17 | 27 | 200000 | 22 | 4.4E+06 | 6.64 | 0.31 | −0.21 |

Study 3. In this study, the in vitro killing efficacy was evaluated using the biofilm wire TK assay for 1, 5, and 10 mg/mL SEQ ID NO: 1 formulations made in either water, 50 or 100 mM bicarbonate, or PBS.

SEQ ID NO: 1 was stored at −20° C. prior to testing. SEQ ID NO: 1 was dissolved directly into Dulbecco's Phosphate Buffered Saline [PBS, pH 7.4.] at a concentration of 10 mg/mL, and the pH was adjusted back to 7.4+/−0.1 with 1 M sodium hydroxide. Separately, 50 mM and 100 mM sodium bicarbonate (solutions were prepared in water with no pH adjustment. SEQ ID NO: 1 was then dissolved at a concentration of 10 mg/mL into either 50 mM or 100 mM sodium bicarbonate, and the pH of these SEQ ID NO: 1 stock solutions were measured and recorded, but not adjusted prior to testing, details in Table 15. SEQ ID NO: 1 at 10 mg/mL was diluted further in the same vehicle it was resuspended in to create solutions of 5 mg/mL and 1 mg/mL.

TABLE 15
pH of Sodium Bicarbonate Solutions Containing SEQ ID NO: 1

| | SEQ ID NO: 1 | |
|---|---|---|
| Vehicle | (mg/mL) | pH |
| 50 mM sodium bicarbonate | 1 | 8.2 |
| 50 mM sodium bicarbonate | 5 | 7.7 |
| 50 mM sodium bicarbonate | 10 | 6.9 |
| 100 mM sodium bicarbonate | 1 | 8.4 |
| 100 mM sodium bicarbonate | 5 | 8.0 |
| 100 mM sodium bicarbonate | 10 | 7.3 |

### Organisms.

Test organisms consisted of Staphylococcus aureus NRS 382 (USA100 MRSA) and Escherichia coli CDC 0451 (KPC-3). Upon receipt at Micromyx, the isolates were streaked under suitable conditions onto agar medium appropriate to each organism and were incubated for 18 to 24 hr at 35° C. Colonies harvested from these growth plates were resuspended in the appropriate medium containing a cryoprotectant. Aliquots of each suspension were then frozen at −80° C.

Prior to testing, the isolates were sub-cultured onto Trypticase Soy agar with 5% sheep blood and incubated under optimal conditions for growth.

SEQ ID NO: 1 had an MIC of 4 and 1 μg/mL against S. aureus NRS382 (USA 100) and E. coli CDC 0451, respectively, as determined in cation-adjusted Mueller Hinton broth with 0.002% P-80 in the study.

### Test Medium.

Testing was conducted in either PBS, 50 mM sodium bicarbonate, or 100 mM sodium bicarbonate with and without 10, 5, and 1 mg/mL SEQ ID NO: 1. These three vehicles without SEQ ID NO: 1 served as the untreated controls. Double strength Dey/Engley neutralizing broth was used as a neutralizer for dilution and plating during determination of colony-forming units (CFU)/mL at each time point.

### Biofilm Assay.

In order to establish the biofilm on the wires, the following procedure was followed: An overnight culture of each isolate was made in Tryptic soy broth. K-wires that were cut to 6 mm and sterilized by autoclaving were placed in 24 well plates and inoculated with 1 mL of a normalized bacterial suspension containing approximately 1×10⁶ CFU/mL in cation-adjusted Mueller-Hinton broth. The plates containing the wires were incubated for 24 hr at 35° C. followed by removing the planktonic cells by carefully pipetting the medium from the wells. Fresh CAMHB (1 mL) was added to the wells containing the wires and the plates were incubated for an additional 24 hr at 35° C.

Separate 24 well-plates were prepared with wells containing either 1 mL of vehicle (PBS pH 7.4, 50 mM sodium bicarbonate or 100 mM sodium bicarbonate), or 1 mL of SEQ ID NO: 1 at 1, 5, or 10 mg/mL dissolved in the corresponding vehicle as described above. After the biofilm was established, the wires were removed from the wells using sterile forceps, rinsed with 1 mL of PBS, and placed in the appropriate treatment well containing 0, 1, 5, or 10 mg/mL SEQ ID NO: 1 in the indicated vehicle for either 0, 2.5, 5, 7.5, or 15 min at room temperature. After the designated time, each wire was removed, rinsed with 1 mL of PBS, and then placed in an Eppendorf tube with 1 mL of double strength Dey-Engley medium. The wires were then sonicated at room temperature for 10 min to dislodge bacteria using a Lab Line Instruments sonicator at a setting of 100%.

A 0.2 mL aliquot of sonicate was removed and pipetted into column 1 of a 96-well plate that contained 180 μL of double strength Dey-Engley medium in columns 2 through 5. Serial ten-fold dilutions were conducted across the plate on the Biomek 3000 followed by track dilution plating in which a 10 μl aliquot of the dilutions (undiluted through 104) were spotted in duplicate across the top of a TSA plate. The plate was then tilted at a 45-900 angle to allow the diluted sample to track across the agar surface to the opposite side of the plate. The plates were laid flat, allowed to dry at room temperature, then inverted and incubated at 35° C. for approximately 24 hr. Colonies were manually counted to determine the viable count in CFU/mL.

### Result

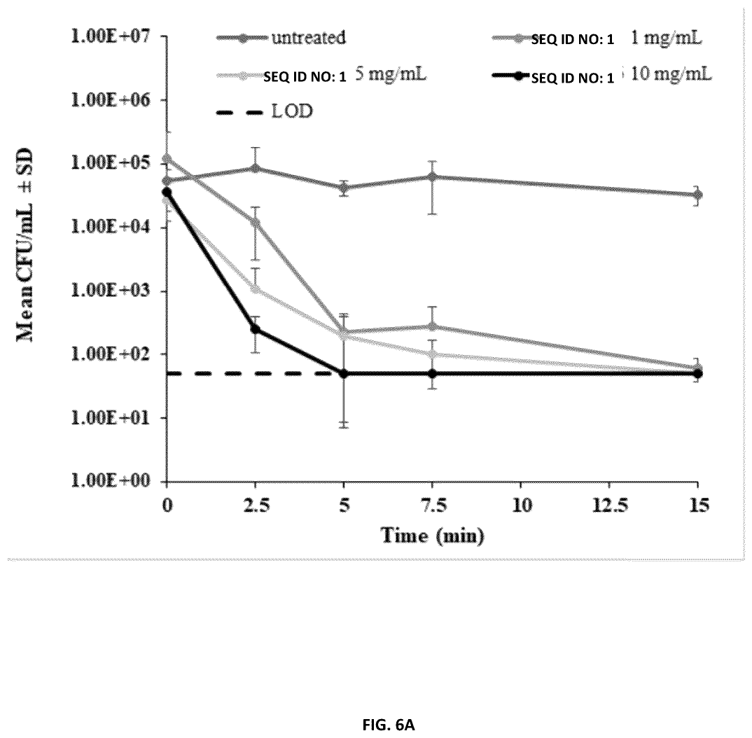

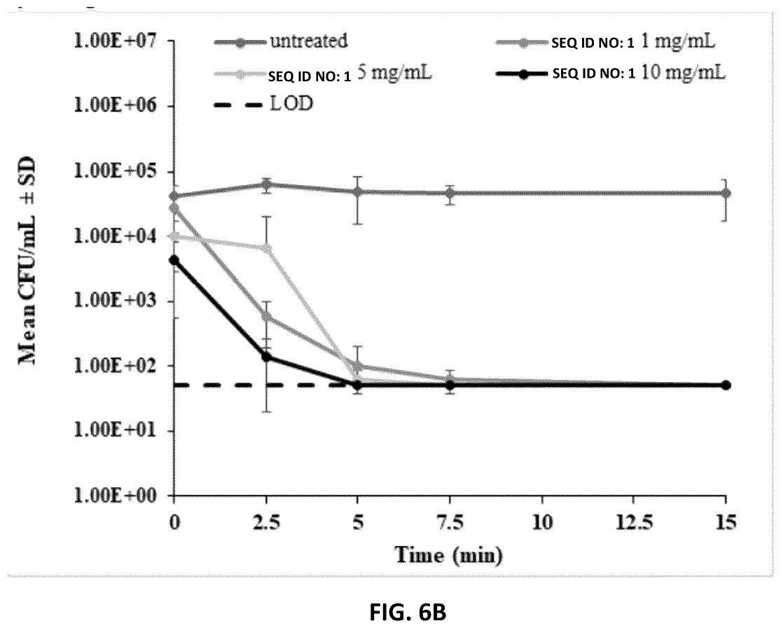

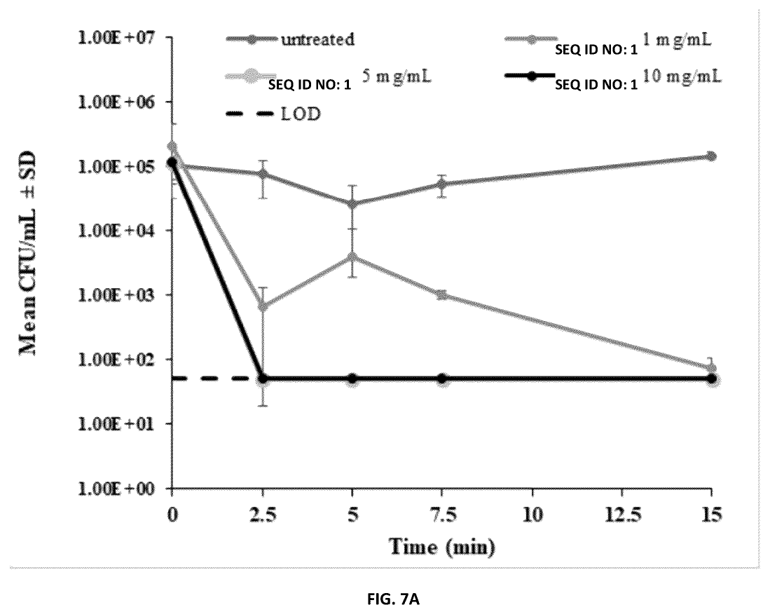

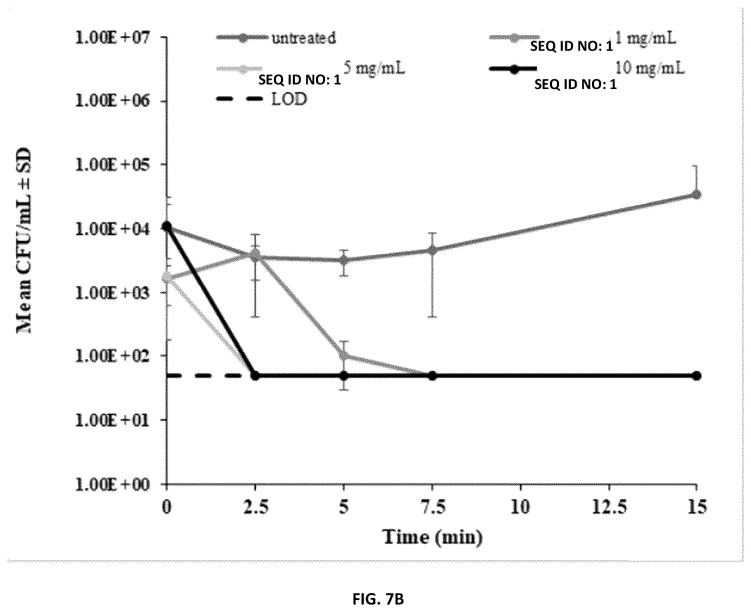

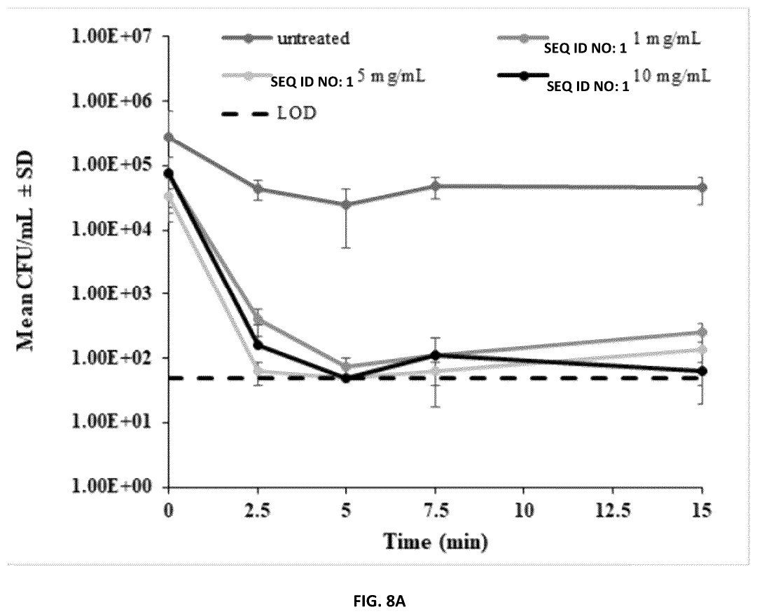

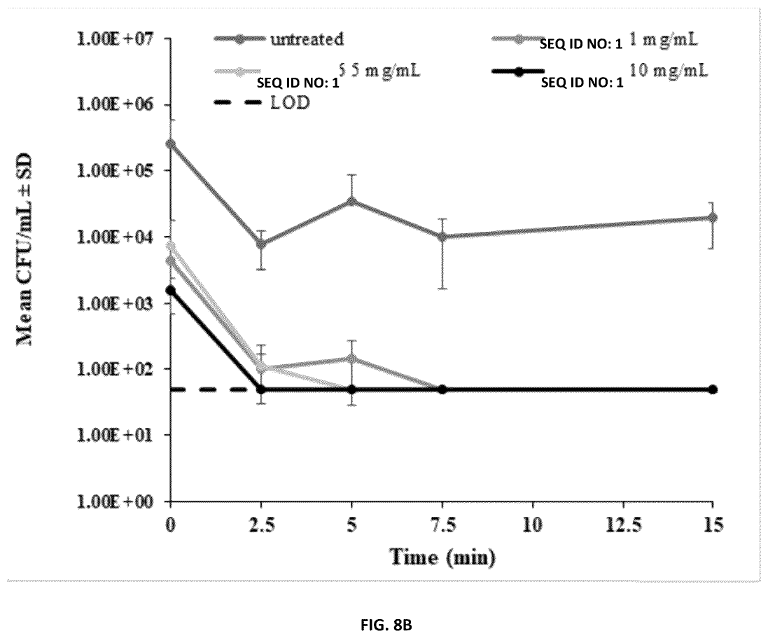

The activity of SEQ ID NO: 1 against the test isolates, S. aureus USA 100 (NRS 382) and E. coli CDC 0451 in the wire biofilm assay in PBS is presented in Table 16A and 16B respectively, along with corresponding FIGS. 6A and 6B. Activity in the assay when tested in 50 mM bicarbonate is shown in Table 17A and 17B, and FIGS. 7A and 7B for S. aureus USA 100 (NRS 382) and E. coli CDC 0451, respectively; and activity when tested in 100 mM bicarbonate is shown in Table 18A and 18B, with corresponding FIGS. 8A and 8B for S. aureus USA 100 (NRS 382) and E. coli CDC 0451, respectively

As indicated in the tables, organisms on wire biofilms were incubated with SEQ ID NO: 1 at 0, 1, 5 or 10 mg/mL in the indicated vehicle for 0 to 15 minutes; the accompanying graphs show the reduction in colony-forming units after each incubation.

When tested in PBS, Table 16A and FIG. 6A show that SEQ ID NO: 1 demonstrated bacterial killing at all concentrations and in a concentration-dependent fashion against biofilms of S. aureus USA 100 (NRS 382), with CFU reaching the limit of detection (50 CFU/mL) for the 10 mg/mL concentration within 5 min. Three-log killing, indicative of bactericidal activity, was demonstrated by SEQ ID NO: 1 at 5 mg/mL (215 min) and at 10 mg/mL (25 min). When tested against E. coli CDC 0451, killing at the limit of detection representing a 2.92 log-kill was observed for SEQ ID NO: 1 by 5 min at 10 mg/mL, 7.5 min at 5 mg/mL and 15 min at 1 mg/mL Table 16B and FIG. 6B.

The killing effect of SEQ ID NO: 1 appeared to be enhanced against both organisms in the presence of 50 mM bicarbonate, as shown in Table 17 and FIG. 7. The 5 and 10 mg/mL concentrations of SEQ ID NO: 1 reduced CFU levels of S. aureus USA 100 to the limit of detection reflecting a 3.31 log-kill by 2.5 min with no rebound observed, and the 1 mg/mL concentration reduced bacterial levels to near the limit of detection with a 3.13-log kill within 15 min as seen in Table 17A and FIG. 7A. Against E. coli CDC 0451, rapid killing was observed at the 5 and 10 mg/mL concentrations, with CFU at the limit of detection reflecting a 2.32 log-kill by 2.5 min; for the 1 mg/mL concentration, CFU reached the limit of detection at 7.5 min and no rebound was seen for any test concentrations Table 17B, and FIG. 7B.

In the presence of 100 mM bicarbonate, the killing activity of SEQ ID NO: 1 was further enhanced, as can be seen in Table 18 and FIG. 18. Against S. aureus USA 100, all three concentrations of SEQ ID NO: 1 had bactericidal activity with >3-log killing as seen in Table 18A and FIG. 8A. By 5 min the 1 mg/mL concentration had demonstrated a 3-log killing effect, and the 5 and 10 mg/mL concentrations had bactericidal effects by 2.5 min. Similarly, against E. coli CDC 0451, all three concentrations displayed bactericidal activity in this assay by 2.5 min as seen in Table 18B and FIG. 18B.

Only modest decreases in S. aureus or E. coli numbers were observed in the Untreated Controls over the course of the experiment (≤1.1 log₁₀ CFU decrease, Table 16-18 and FIG. 6-8).

In summary, SEQ ID NO: 1 was evaluated for activity in a study of a wire biofilm assay against S. aureus USA 100 and E. coli CDC 0451 in an assessment of the effect of bicarbonate concentrations on activity. There was a trend towards concentration dependent killing, with killing to the limit of detection achieved more rapidly with increasing concentration. Killing appeared to be slightly enhanced for SEQ ID NO: 1 in sodium bicarbonate relative to PBS, and a statistical analysis of the data may aid in this evaluation. Regardless of vehicle used or concentration tested, rapid killing to the limit of detection of the assay (50 CFU/mL) was observed for both S. aureus and E. coli.

TABLE 16A
Time-Kill activity of SEQ ID NO: 1 against Staphylococcus aureus
USA 100 in the wire biofilm study using PBS as the vehicle

| | | | | | | Log₁₀ |
|---|---|---|---|---|---|---|
| | | | | | | kill |
| | | | | | | relative |
| | | | | | Log₁₀ | to |
| | Treat- | Time | Mean | | CFU/ | untreated |
| Organism | ment | (min) | CFU/mL | SD | mL | at 0 min |
| S. | Untreated | 0 | 5.41E+04 | 2.83E+04 | 4.73 | — |
| aureus | | 2.5 | 8.44E+04 | 1.02E+05 | 4.93 | −0.20 |
| USA | | 5 | 4.31E+04 | 1.13E+04 | 4.63 | 0.10 |
| 100 | | 7.5 | 6.34E+04 | 4.68E+04 | 4.80 | −0.07 |
| NRS | | 15 | 3.36E+04 | 1.10E+04 | 4.53 | 0.20 |
| 382 | SEQ ID | 0 | 1.25E+05 | 1.97E+05 | 5.10 | −0.37 |
| | NO: 1 | 2.5 | 1.23E+04 | 9.17E+03 | 4.09 | 0.64 |
| | 1 mg/mL | 5 | 2.25E+02 | 2.18E+02 | 2.35 | 2.38 |
| | | 7.5 | 2.75E+02 | 2.87E+02 | 2.44 | 2.29 |
| | | 15 | 6.25E+01 | 2.50E+01 | 1.80 | 2.93 |
| | SEQ ID | 0 | 2.66E+04 | 8.63E+03 | 4.43 | 0.30 |
| | NO: 1 | 2.5 | 1.09E+03 | 1.21E+03 | 3.04 | 1.69 |
| | 5 mg/mL | 5 | 2.00E+02 | 1.91E+02 | 2.30 | 2.43 |
| | | 7.5 | 1.00E+02 | 7.07E+01 | 2.00 | 2.73 |
| | | 15 | 5.00E+01 | 0.00E+00 | 1.70 | 3.03 |
| | SEQ ID | 0 | 3.63E+04 | 2.35E+04 | 4.56 | 0.17 |
| | NO: 1 | 2.5 | 2.50E+02 | 1.41E+02 | 2.40 | 2.33 |
| | 10 mg/mL | 5 | 5.00E+01 | 0.00E+00 | 1.70 | 3.03 |
| | | 7.5 | 5.00E+01 | 0.00E+00 | 1.70 | 3.03 |
| | | 15 | 5.00E+01 | 0.00E+00 | 1.70 | 3.03 |

TABLE 16B
Time-kill activity of SEQ ID NO: 1 against Escherichia coli
CDC 0451 in the wire biofilm study using PBS as the vehicle

| | | | | | | Log₁₀ kill |
|---|---|---|---|---|---|---|
| | | | | | | relative |
| | | | | | | to |
| | | | | | Log₁₀ | untreated |
| | Treat- | Time | Mean | | CFU/ | at |
| Organism | ment | (min) | CFU/mL | SD | mL | 0 min |
| E. | Untreated | 0 | 4.21E+04 | 1.98E+04 | 4.62 | — |
| coli | | 2.5 | 6.44E+04 | 1.69E+04 | 4.81 | −0.19 |
| CDC | | 5 | 4.96E+04 | 3.44E+04 | 4.70 | −0.08 |
| 0451 | | 7.5 | 4.56E+04 | 1.51E+04 | 4.66 | −0.04 |
| | | 15 | 4.71E+04 | 2.97E+04 | 4.67 | −0.05 |
| | SEQ ID | 0 | 2.79E+04 | 1.65E+04 | 4.45 | 0.17 |
| | NO: 1 | 2.5 | 5.88E+02 | 3.97E+02 | 2.77 | 1.85 |
| | 1 mg/mL | 5 | 1.00E+02 | 1.00E+02 | 2.00 | 2.62 |
| | | 7.5 | 6.25E+01 | 2.50E+01 | 1.80 | 2.82 |
| | | 15 | 5.00E+01 | 0.00E+00 | 1.70 | 2.92 |
| | SEQ ID | 0 | 9.98E+03 | 7.16E+03 | 4.00 | 0.62 |
| | NO: 1 | 2.5 | 6.70E+03 | 1.32E+04 | 3.83 | 0.79 |
| | 5 mg/mL | 5 | 6.25E+01 | 2.50E+01 | 1.80 | 2.82 |
| | | 7.5 | 5.00E+01 | 0.00E+00 | 1.70 | 2.92 |
| | | 15 | 5.00E+01 | 0.00E+00 | 1.70 | 2.92 |
| | SEQ ID | 0 | 4.30E+03 | 3.76E+03 | 3.63 | 0.99 |
| | NO: 1 | 2.5 | 1.38E+02 | 1.18E+02 | 2.14 | 2.48 |
| | 10 mg/mL | 5 | 5.00E+01 | 0.00E+00 | 1.70 | 2.92 |
| | | 7.5 | 5.00E+01 | 0.00E+00 | 1.70 | 2.92 |
| | | 15 | 5.00E+01 | 0.00E+00 | 1.70 | 2.92 |

TABLE 17A
Time-kill activity of SEQ ID NO: 1 against
Staphylococcus aureus USA 100 in the
wire biofilm study using 50 mM bicarbonate as the vehicle

| | | | | | | Log₁₀ |
|---|---|---|---|---|---|---|
| | | | | | | kill |
| | | | | | | relative |
| | | | | | | to un- |
| | | | | | Log₁₀ | treated |
| | Treat- | Time | Mean | | CFU/ | at |
| Organism | ment | (min) | CFU/mL | SD | mL | 0 min |
| S. | Untreated | 0 | 1.03E+05 | 4.11E+04 | 5.01 | — |
| aureus | | 2.5 | 7.73E+04 | 4.58E+04 | 4.89 | 0.12 |
| USA | | 5 | 2.63E+04 | 2.44E+04 | 4.42 | 0.59 |
| 100 | | 7.5 | 5.29E+04 | 1.95E+04 | 4.72 | 0.29 |
| NRS | | 15 | 1.48E+05 | 1.85E+04 | 5.17 | −0.16 |
| 382 | SEQ ID | 0 | 2.05E+05 | 2.56E+05 | 5.31 | −0.30 |
| | NO: 1 | 2.5 | 6.50E+02 | 6.31E+02 | 2.81 | 2.20 |
| | 1 mg/mL | 5 | 3.93E+03 | 6.38E+03 | 3.59 | 1.42 |
| | | 7.5 | 1.01E+03 | 1.65E+02 | 3.01 | 2.00 |
| | | 15 | 7.50E+01 | 2.89E+01 | 1.88 | 3.13 |
| | SEQ ID | 0 | 1.13E+05 | 8.16E+04 | 5.05 | −0.04 |
| | NO: 1 | 2.5 | 5.00E+01 | 0.00E+00 | 1.70 | 3.31 |
| | 5 mg/mL | 5 | 5.00E+01 | 0.00E+00 | 1.70 | 3.31 |
| | | 7.5 | 5.00E+01 | 0.00E+00 | 1.70 | 3.31 |
| | | 15 | 5.00E+01 | 0.00E+00 | 1.70 | 3.31 |
| | SEQ ID | 0 | 1.19E+05 | 6.63E+04 | 5.07 | −0.06 |
| | NO: 1 | 2.5 | 5.00E+01 | 0.00E+00 | 1.70 | 3.31 |
| | 10 mg/mL | 5 | 5.00E+01 | 0.00E+00 | 1.70 | 3.31 |
| | | 7.5 | 5.00E+01 | 0.00E+00 | 1.70 | 3.31 |
| | | 15 | 5.00E+01 | 0.00E+00 | 1.70 | 3.31 |

TABLE 17B
Time-kill activity of SEQ ID NO: 1 against Escherichia coli
CDC 0451 in the wire biofilm study
using 50 mM bicarbonate as the vehicle

| | | | | | | Log₁₀ |
|---|---|---|---|---|---|---|
| | | | | | | kill |
| | | | | | | relative |
| | | | | | Log₁₀ | to un- |
| | Treat- | Time | Mean | | CFU/ | treated at |
| Organism | ment | (min) | CFU/mL | SD | mL | 0 min |
| E. | untreated | 0 | 1.05E+04 | 1.41E+04 | 4.02 | — |
| coli | | 2.5 | 3.50E+03 | 1.97E+03 | 3.54 | 0.48 |
| CDC | | 5 | 3.21E+03 | 1.38E+03 | 3.51 | 0.51 |
| 0451 | | 7.5 | 4.60E+03 | 4.20E+03 | 3.66 | 0.36 |
| | | 15 | 3.47E+04 | 6.36E+04 | 4.54 | −0.52 |
| | SEQ ID | 0 | 1.65E+03 | 1.02E+03 | 3.22 | 0.80 |
| | NO: 1 | 2.5 | 4.19E+03 | 3.78E+03 | 3.62 | 0.40 |
| | 1 mg/mL | 5 | 1.00E+02 | 7.07E+01 | 2.00 | 2.02 |
| | | 7.5 | 5.00E+01 | 0.00E+00 | 1.70 | 2.32 |
| | | 15 | 5.00E+01 | 0.00E+00 | 1.70 | 2.32 |
| | SEQ ID | 0 | 1.80E+03 | 1.62E+03 | 3.26 | 0.76 |
| | NO: 1 | 2.5 | 5.00E+01 | 0.00E+00 | 1.70 | 2.32 |
| | 5 mg/mL | 5 | 5.00E+01 | 0.00E+00 | 1.70 | 2.32 |
| | | 7.5 | 5.00E+01 | 0.00E+00 | 1.70 | 2.32 |
| | | 15 | 5.00E+01 | 0.00E+00 | 1.70 | 2.32 |
| | SEQ ID | 0 | 1.09E+04 | 2.01E+04 | 4.04 | −0.02 |
| | NO: 1 | 2.5 | 5.00E+01 | 0.00E+00 | 1.70 | 2.32 |
| | 10 mg/mL | 5 | 5.00E+01 | 0.00E+00 | 1.70 | 2.32 |
| | | 7.5 | 5.00E+01 | 0.00E+00 | 1.70 | 2.32 |
| | | 15 | 5.00E+01 | 0.00E+00 | 1.70 | 2.32 |

TABLE 18A
Time-kill activity of SEQ ID NO: 1 against
Staphylococcus aureus USA 100 in the wire
biofilm study using 100 mM bicarbonate as the vehicle

| | | | | | | Log10 |
|---|---|---|---|---|---|---|
| | | | | | | kill |
| | | | | | | relative |
| | | | | | | to un- |
| | | | | | Log10 | treated |
| | | Time | Mean | | CFU/ | at |
| Organism | Treatment | (min) | CFU/mL | SD | mL | 0 min |
| S. | Untreated | 0 | 2.83E+05 | 4.14E+05 | 5.45 | — |
| aureus | | 2.5 | 4.49E+04 | 1.53E+04 | 4.65 | 0.80 |
| USA | | 5 | 2.46E+04 | 1.94E+04 | 4.39 | 1.06 |
| 100 | | 7.5 | 4.83E+04 | 1.75E+04 | 4.68 | 0.77 |
| NRS | | 15 | 4.51E+04 | 1.98E+04 | 4.65 | 0.80 |
| 382 | SEQ ID | 0 | 7.34E+04 | 5.99E+04 | 4.87 | 0.58 |
| | NO: 1 | 2.5 | 4.00E+02 | 1.83E+02 | 2.60 | 2.85 |
| | 1 mg/mL | 5 | 7.50E+01 | 2.89E+01 | 1.88 | 3.57 |
| | | 7.5 | 1.13E+02 | 9.46E+01 | 2.05 | 3.40 |
| | | 15 | 2.63E+02 | 8.54E+01 | 2.42 | 3.03 |
| | SEQ ID | 0 | 3.31E+04 | 1.02E+04 | 4.52 | 0.93 |
| | NO: 1 | 2.5 | 6.25E+01 | 2.50E+01 | 1.80 | 3.65 |
| | 5 mg/mL | 5 | 5.00E+01 | 0.00E+00 | 1.70 | 3.75 |
| | | 7.5 | 6.25E+01 | 2.50E+01 | 1.80 | 3.65 |
| | | 15 | 1.38E+02 | 1.18E+02 | 2.14 | 3.31 |
| | SEQ ID | 0 | 7.78E+04 | 5.92E+04 | 4.89 | 0.56 |
| | NO: 1 | 2.5 | 1.63E+02 | 1.65E+02 | 2.21 | 3.24 |
| | 10 mg/mL | 5 | 5.00E+01 | 0.00E+00 | 1.70 | 3.75 |
| | | 7.5 | 1.13E+02 | 9.46E+01 | 2.05 | 3.40 |
| | | 15 | 6.25E+01 | 2.50E+01 | 1.80 | 3.65 |

Study 4. In the current study, 1, 3, and 10 mg/m SEQ ID NO: 1 formulations were evaluated in either 50, 100, 150 or 200 mM sodium bicarbonate for 0, 1, 2.5, and 5 m for in vitro killing efficacy using the biofilim wire TK assay.

SEQ ID NO: 1 was stored at −20° C. prior to testing. Sodium bicarbonate solutions of 50, 100, 150, or 200 mM were prepared in water with no pH adjustment. SEQ ID NO: 1 was then dissolved at a concentration of 10 mg/mL into either 50, 100, 150, or 200 nM sodium bicarbonate and was diluted further in the same vehicle to create solutions of 3 mg/mL and 1 mg/mL. The pH of these SEQ ID NO: 1 stock solutions were measured and recorded in Table 19, but not adjusted prior to testing.

TABLE 19
pH of Sodium Bicarbonate Solutions Containing SEQ ID NO: 1

| Sodium | SEQ ID NO: 1 | |
|---|---|---|
| Bicarbonate (mM) | (mg/mL) | pH |
| 50 | 0 | 8.5 |
| | 1 | 8.2 |
| | 3 | 7.9 |
| | 10 | 7.1 |
| 100 | 0 | 8.5 |
| | 1 | 8.3 |
| | 3 | 8.1 |
| | 10 | 7.6 |
| 150 | 0 | 8.5 |
| | 1 | 8.4 |
| | 3 | 8.3 |
| | 10 | 7.7 |
| 200 | 0 | 8.5 |
| | 1 | 8.4 |
| | 3 | 8.3 |
| | 10 | 7.8 |

### Organisms.

Test organisms consisted of Staphylococcus aureus NRS 382 (USA100 MRSA) from the Network on Antimicrobial Resistance in Staphylococcus, Escherichia coli CDC 0451 (KPC-3), and Pseudomonas aeruginosa CDC 0515 both from the Centers for Disease Control Antimicrobial Resistance Bank. Upon receipt at Micromyx, the isolates were streaked under suitable conditions onto agar medium appropriate to each organism and were incubated for 18 to 24 hr at 35° C. Colonies harvested from these growth plates were resuspended in the appropriate medium containing a cryoprotectant. Aliquots of each suspension were then frozen at −80° C.

Prior to testing, the isolates were sub-cultured onto Trypticase Soy agar with 5% sheep blood and incubated under optimal conditions for growth.

SEQ ID NO: 1 had an MIC of 4, 2, and 8 μg/mL against S. aureus NRS382 (USA 100), E. coli CDC 0451, and P. aeruginosa CDC 0515, respectively, as determined by broth microdilution in cation-adjusted Mueller Hinton broth with 0.002% P-80 in the studies above.

### Test Medium.

Testing was conducted in either 50, 100, 150, or 200 mM sodium bicarbonate with and without 1, 3, and 10 mg/mL SEQ ID NO: 1. These four vehicles without SEQ ID NO: 1 served as the untreated controls. Double strength Dey/Engley neutralizing broth was used as a neutralizer for dilution and plating during determination of colony-forming units (CFU)/mL at each time point.

### Biofilm Assay.

In order to establish the biofilm on the wires, the following procedure was followed: An overnight culture of each isolate was made in Tryptic soy broth. Stainless steel K-wires that were cut to 6 mm (0.9 mm diameter) and sterilized by autoclaving were placed in 24 well plates and inoculated with 1 mL of a normalized bacterial suspension containing approximately 1×10⁶ CFU/mL in cation-adjusted Mueller-Hinton broth (CAMHB II). The plates containing the wires were incubated for 24 hr at 35° C. followed by removing the planktonic cells by carefully pipetting the medium from the wells. Fresh CAMHB (1 mL) was added to the wells containing the wires and the plates were incubated for an additional 24 hr at 35° C.

Separate 24 well-plates were prepared with wells containing either 1 mL of vehicle (50, 100, 150, or 200 mM sodium bicarbonate), or 1 mL of SEQ ID NO: 1 at 1, 3, or 10 mg/mL dissolved in the corresponding vehicle as described above. After the biofilm was established, the wires were removed from the wells using sterile forceps, rinsed with 1 mL of Dulbecco's Phosphate Buffered Saline (DPBS, pH 7.4), and placed in the appropriate treatment well containing 0, 1, 3, or 10 mg/mL SEQ ID NO: 1 in the indicated vehicle for either 0, 1, 2.5, or 5 min at room temperature. After the designated time, each wire was removed, rinsed with 1 mL of PBS, and then placed in an Eppendorf tube with 1 mL of double strength Dey-Engley medium. The wires were then sonicated at room temperature for 10 min to dislodge bacteria using a Lab Line Instruments sonicator at a setting of 100%.

A 0.2 mL aliquot of sonicate was removed and pipetted into row A of a 96-well plate that contained 180 μL of double strength Dey-Engley medium in rows B through E. Serial tenfold dilutions were conducted manually down the plate followed by track dilution plating in which a 10 μl aliquot of the dilutions (undiluted through 10⁻⁴) were spotted in duplicate across the top of a TSA plate. The plate was then tilted at a 45-900 angle to allow the diluted sample to track across the agar surface to the opposite side of the plate. The plates were laid flat, allowed to dry at room temperature, then inverted and incubated at 35° C. for approximately 24 hr. Colonies were manually counted to determine the viable count in CFU/mL.

### Results

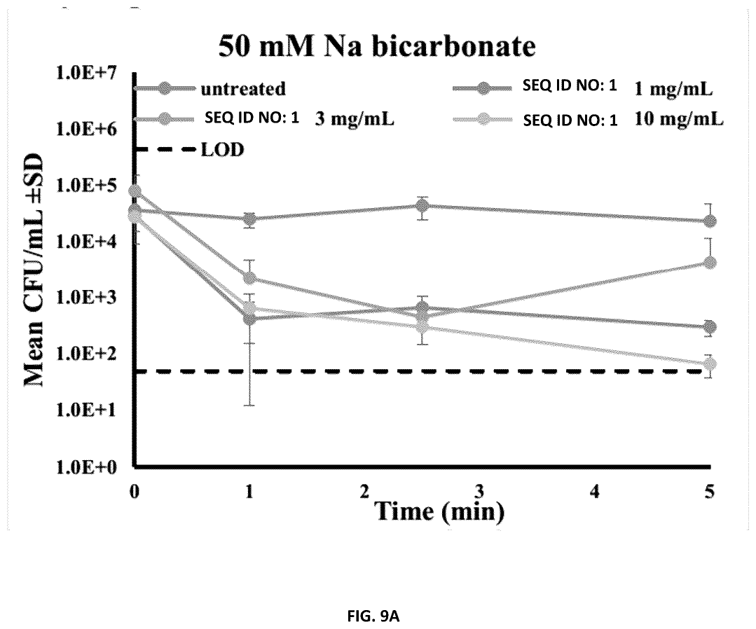

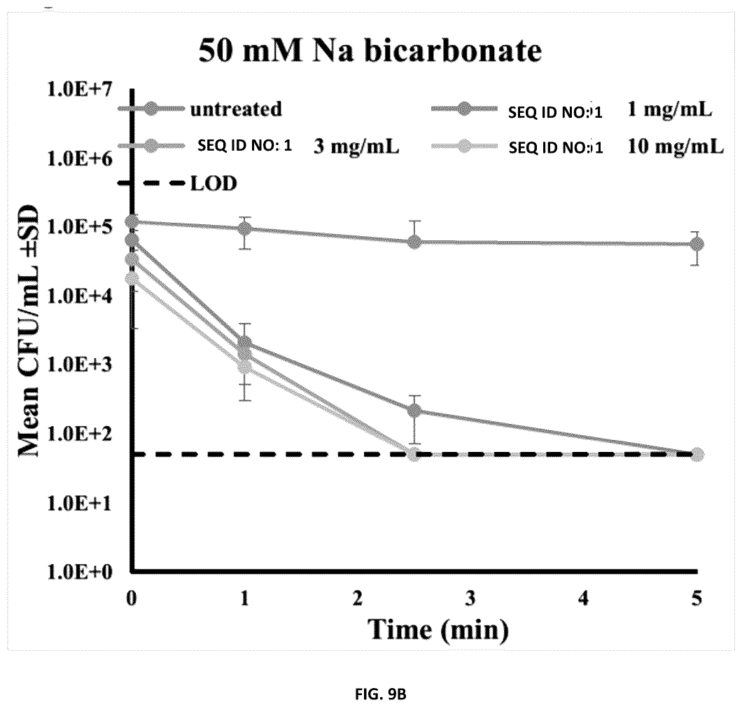

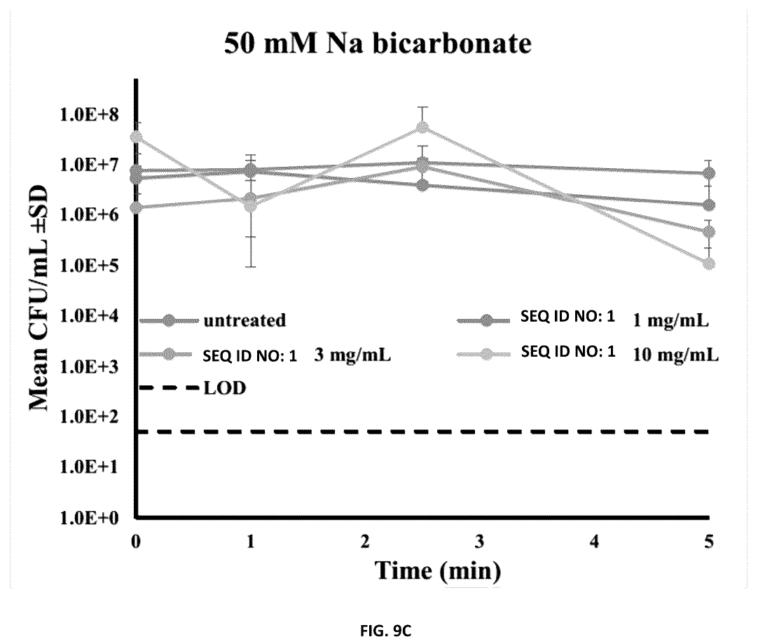

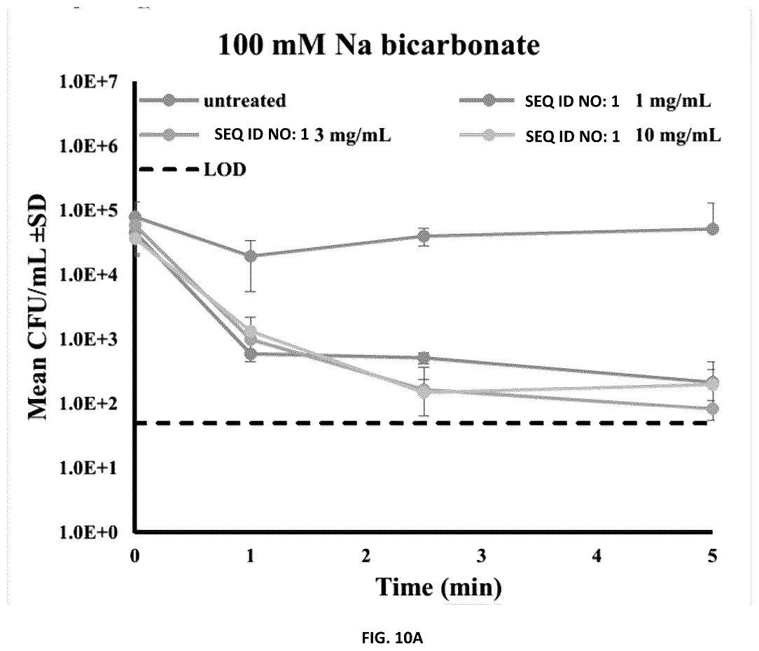

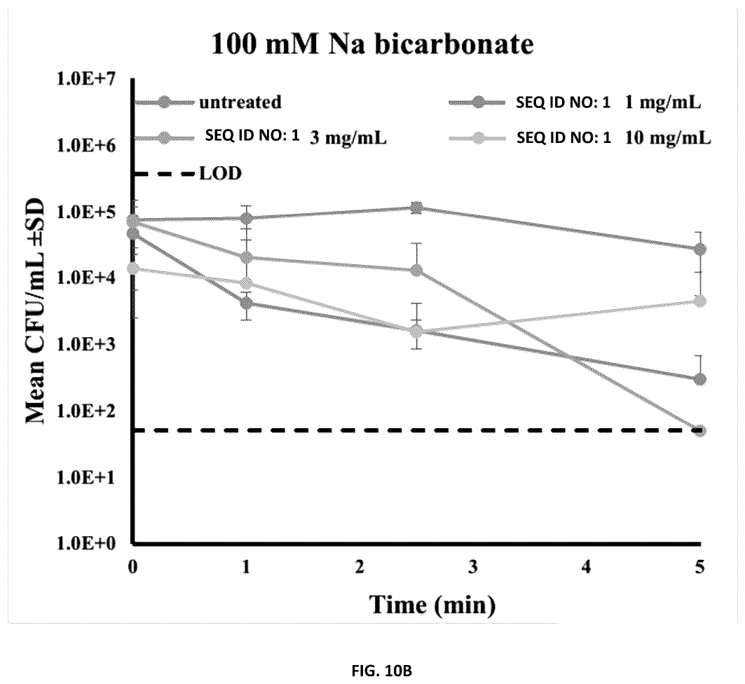

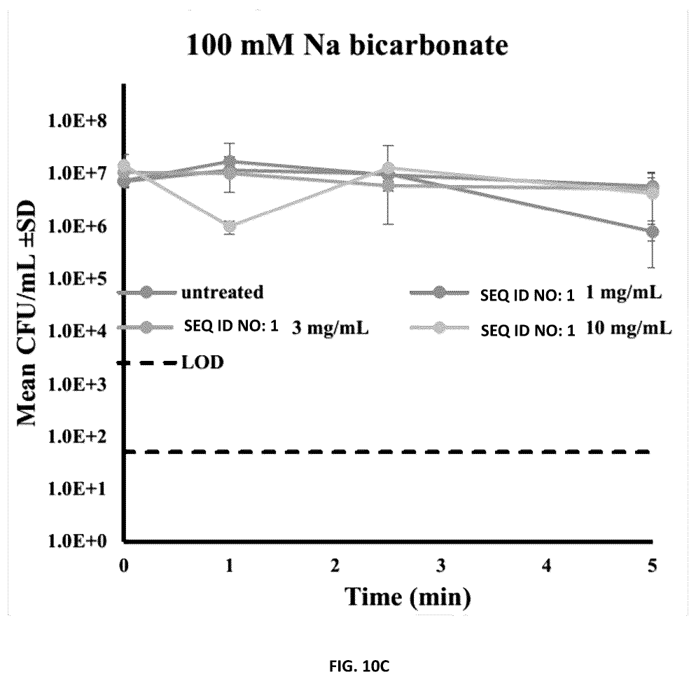

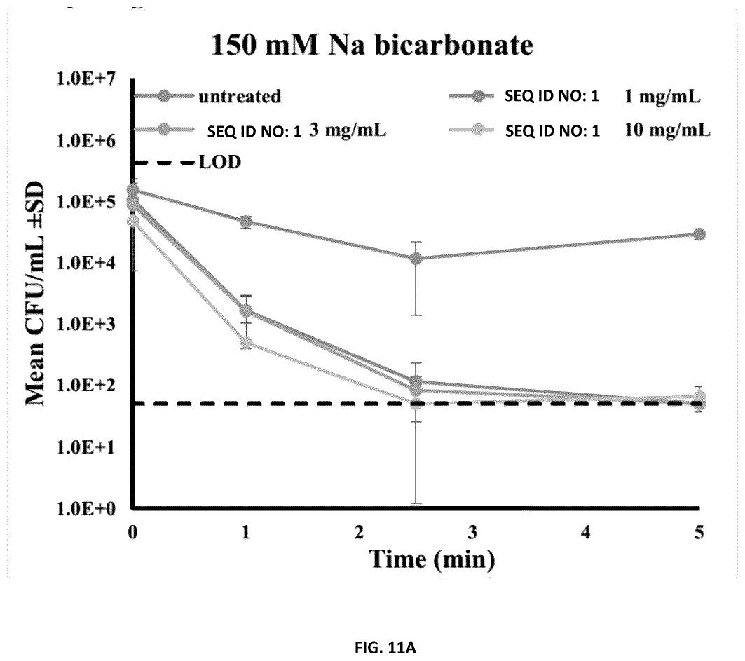

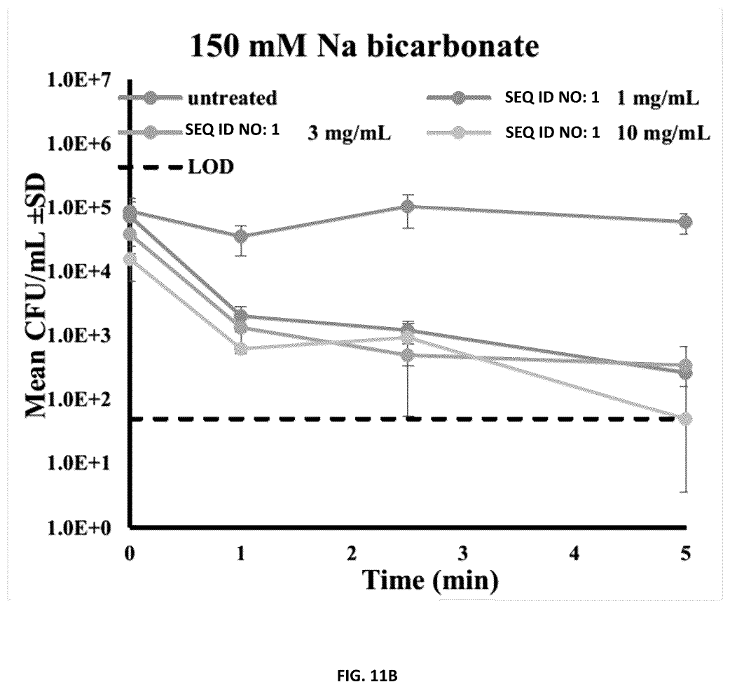

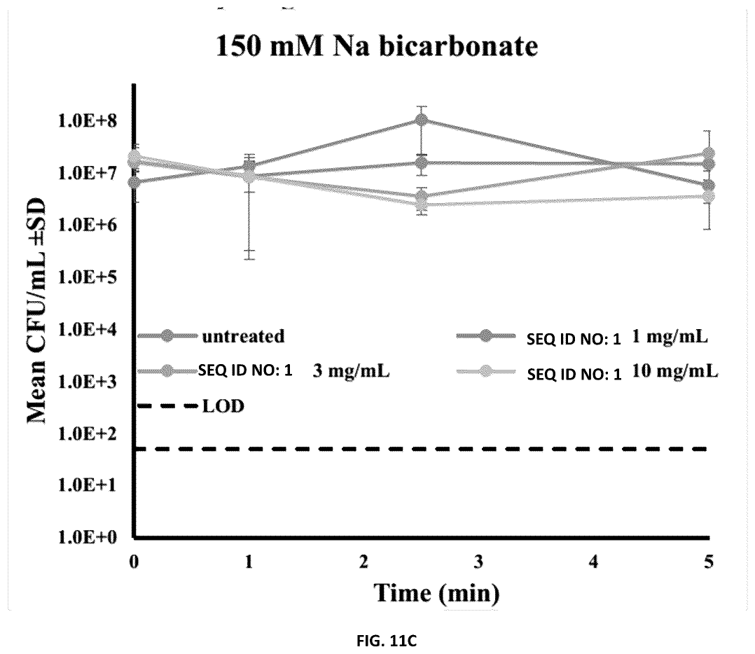

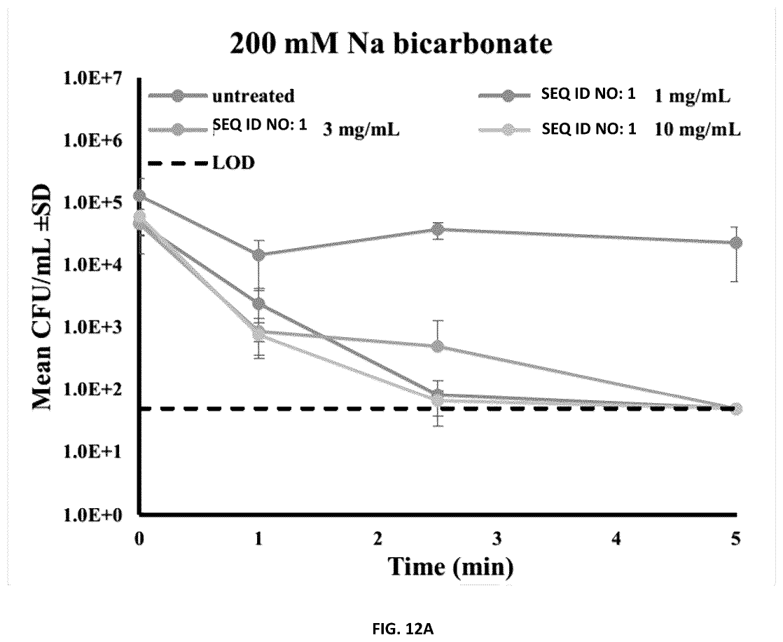

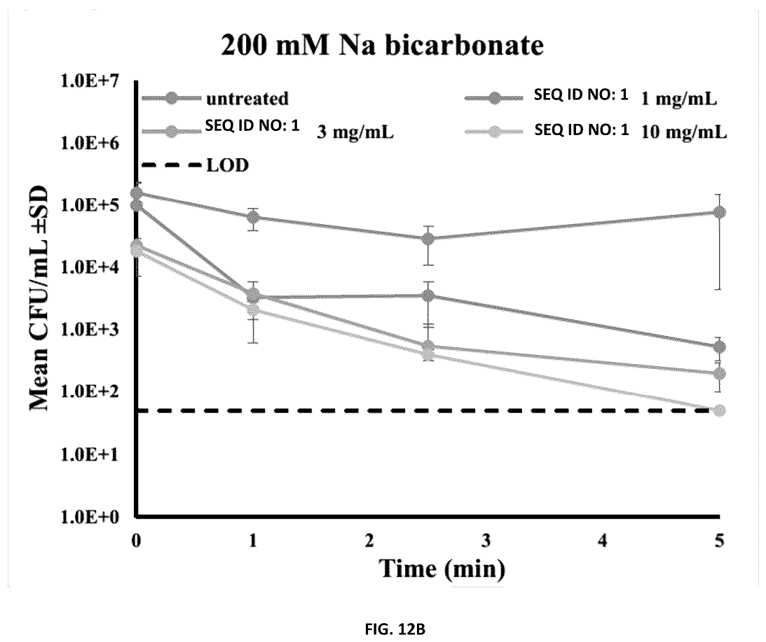

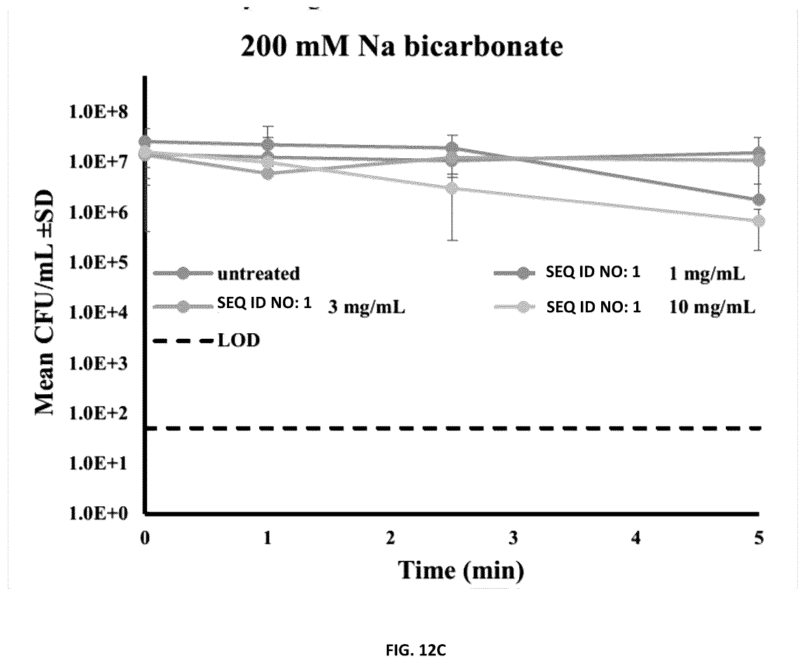

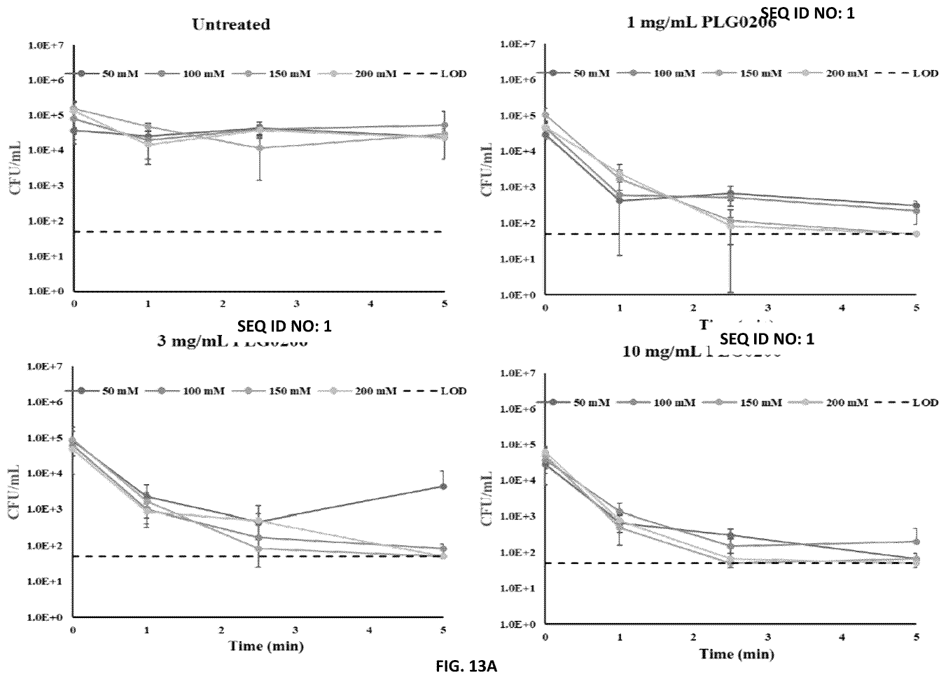

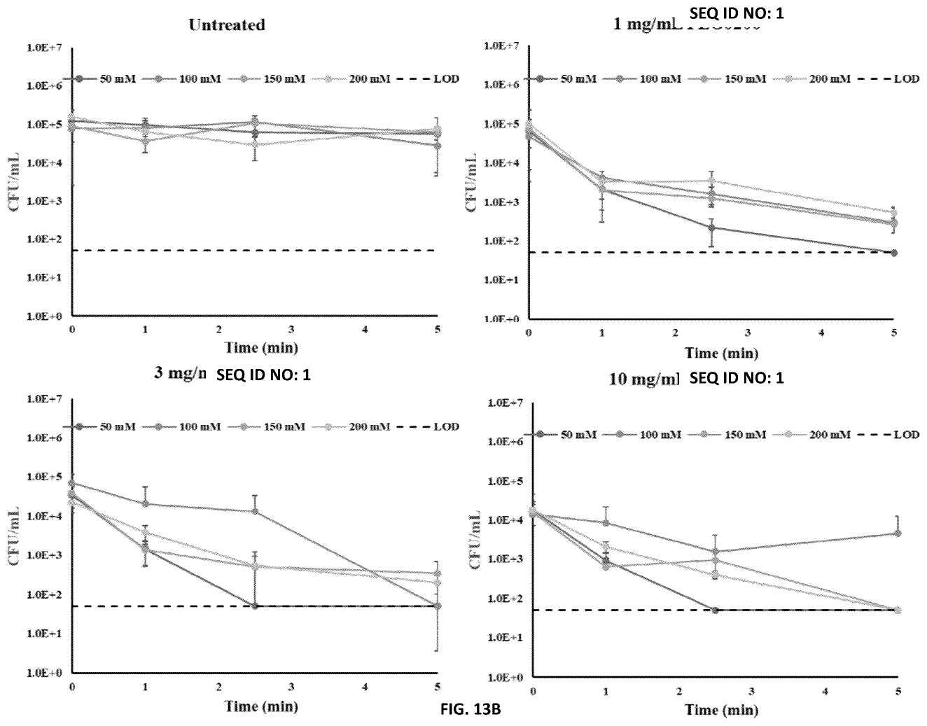

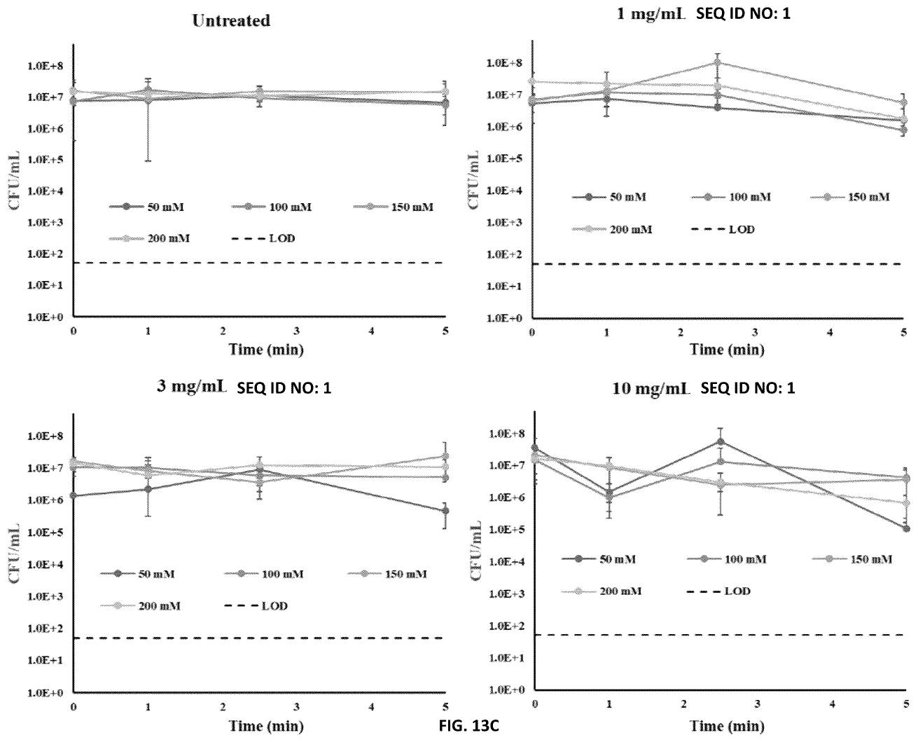

The activity of SEQ ID NO: 1 against the test isolates, S. aureus USA 100 (NRS 382), E. coli CDC 0451, and P. aeruginosa CDC 0515 in the wire biofilm assay in 50 mM sodium bicarbonate is presented in Table 20A, 20B, and 20C, respectively, along with corresponding FIGS. 9A, 9B, and 9C. The activity of SEQ ID NO: 1 in this assay against these organisms are presented in the same format in Table 21 and FIG. 10 (100 mM sodium bicarbonate), Table 22 and FIG. 11, (150 mM sodium bicarbonate), Table 23 and FIG. 12, (200 mM sodium bicarbonate). FIG. 13 contains the same data but is grouped by treatment instead of sodium bicarbonate concentration. As indicated in the tables, organisms on wire biofilms were incubated with SEQ ID NO: 1 at 0, 1, 3, or 10 mg/mL in the indicated vehicle for 0, 1, 2.5, or 5 min. The data is further outlined in Tables 24A-C.

S. aureus NRS382

Table 20A and FIG. 9A show that SEQ ID NO: 1 demonstrated bacterial killing at all concentrations against biofilms of S. aureus USA 100 (NRS 382) in 50 mM sodium bicarbonate. At 1 min there was between 1.22 and 1.94 logs of killing. At 2.5 min there was between 1.74 and 2.09 logs of killing when testing these concentrations of SEQ ID NO: 1 in 50 mM sodium bicarbonate. At 5 min, 10 mg/mL SEQ ID NO: 1 approached the LOD with 2.74 logs of killing with 2 out of the three tested wires at the LOD for the assay, while 1 mg/ML treatment led to a 2.09 log kill at this timepoint. The 3 mg/mL SEQ ID NO: 1 had an increase in CFU/mL at 5 min vs. 2.5 min, as 1 out of the 3 wires had a -2 log increase in CFU/mL while the other wires were approaching the LOD.

Table 21A and FIG. 10A show the SEQ ID NO: 1 killing in 100 mM sodium bicarbonate, which was similar at all concentrations of SEQ ID NO: 1 at the different timepoints. There were around 2 logs of killing at 1 min for all concentrations of SEQ ID NO: 1. At 2.5 and 5 min there were between 2.19 and 2.98 logs of killing for all concentrations of SEQ ID NO: 1 when tested in 100 mM sodium bicarbonate.

Table 22A and FIG. 11A show SEQ ID NO: 1 activity in 150 mM sodium bicarbonate. There were 1.97 (1 mg/mL SEQ ID NO: 1), 1.98 (3 mg/mL SEQ ID NO: 1) and 2.5 (10 mg/mL SEQ ID NO: 1) logs of kill for all concentrations of SEQ ID NO: 1 at the 1 min mark when testing in 150 mM sodium bicarbonate. At the 2.5 and 5 min mark, all concentrations were at or were approaching the LOD of 50 CFU/mL for the assay.

Table 23A and FIG. 12A show the killing in 200 mM sodium bicarbonate by SEQ ID NO: 1. There were between 1.62 (3 mg/mL SEQ ID NO: 1) and 1.88 logs (10 mg/mL SEQ ID NO: 1) of killing at the 1 min of treatment in all SEQ ID NO: 1 concentrations when testing in 200 mM sodium bicarbonate. At the 2.5 and 5 min mark, all SEQ ID NO: 1 concentrations were at or were approaching the LOD for the assay, with the exception of the 2.5 min SEQ ID NO: 1 3 mg/mL sample. This sample had 2 out of 3 wires at the LOD, but the last wire had 1.4×10³ CFU/mL recovered from the wire. FIG. 13A shows the same results for S. aureus NRS382 grouped by treatment condition instead of sodium bicarbonate concentration.

E. coli CDC 0451

Table 20B and FIG. 9B show SEQ ID NO: 1 killing of E. coli CDC 0451 in 50 mM sodium bicarbonate. At 1 min there were 1.76 (1 mg/mL), 1.92 (3 mg/mL), and 2.11 (10 mg/mL) logs of killing. At 2.5 min, 1 mg/mL SEQ ID NO: 1 caused 2.74 logs of killing, while the other two concentrations yielded recovery at the LOD. At 5 min, the LOD for recovery was reached by all three concentrations of SEQ ID NO: 1.

Table 21B and FIG. 10B show SEQ ID NO: 1 activity against E. coli CDC 0451 in 100 mM sodium bicarbonate. At 1 min there was between 0.57 and 1.25 logs of killing by SEQ ID NO: 1. At 2.5 min there was between 0.76 and 1.69 logs of killing. At 5 min, 1 mg/mL SEQ ID NO: 1 caused 2.4 logs of kill, while 3 mg/mL caused recovery to reach the LOD. The 10 mg/mL sample at 5 min had 1 wire with 1.1×10⁴ CFU/mL, while the other two wires were at or approaching the LOD for recovery.

Table 22B and FIG. 11B contain the data for E. coli CDC 0451 recovery from wires treated by SEQ ID NO: 1 in 150 mM sodium bicarbonate. At 1 min, there was between 1.64 and 2.15 logs of killing. At 2.5 min there was 1.86 (1 mg/mL), 2.25 (3 mg/mL), and 1.97 (10 mg/mL) logs of killing by SEQ ID NO: 1. At 5 min, there was 2.52 logs killing when treated with 1 mg/mL, 2.41 logs killing during 3 mg/mL treatment, while the 10 mg/mL treatment yielded recovery at the LOD.

Table 23B and FIG. 12B show SEQ ID NO: 1 activity in 200 mM sodium bicarbonate when testing against E. coli CDC 0451. At 1 min, there was between 1.62 and 1.88 logs of killing. At 2.5 min, there was 1.65 (1 mg/mL), 2.46 (3 mg/mL), and 2.60 (10 mg/mL) logs of killing by SEQ ID NO: 1. At 5 min, there was 2.47 logs of killing at 1 mg/mL and 2.90 logs of killing with 3 mg/mL SEQ ID NO: 1, while 10 mg/mL SEQ ID NO: 1 treatment yielded recovery at the LOD for the assay. FIG. 13B shows the same results for E. coli CDC 0451 grouped by treatment condition instead of sodium bicarbonate concentration.

Table 20C, 21C, 22C, and 23C, and FIG. 9C, FIG. 10C, FIG. 11C, and FIG. 12C show the recovery of P. aeruginosa CDC 0515 from wires treated in sodium bicarbonate with and without various concentrations of SEQ ID NO: 1. Biofilms formed by P. aeruginosa CDC 0515 on wires were very dense and had ˜ 2 logs increased CFU/mL recovery than the other two evaluated organisms. In addition, pellicle biofilms formed at the air liquid interface of the 24 well plates leading to increased difficulty of removing excess biofilm from the wires by washing in PBS prior to treatment. There was no observed consistent effect of SEQ ID NO: 1 on the viability of these P. aeruginosa wire biofilms in any of the evaluated conditions of this experiment, with killing rarely reaching 1 log. FIG. 13C shows the same results for P. aeruginosa CDC 0515 grouped by treatment condition instead of sodium bicarbonate concentration.

SEQ ID NO: 1 had an MIC of 8 μg/mL and 16 μg/mL in two previous microtiter plate biofilm studies. In those previous studies, mature biofilms were formed on polystyrene microtiter plates through growth in CAMHB, followed by exposure to SEQ ID NO: 1 and resazurin addition to measure cell viability. Exposure of these microtiter plate biofilms to 500 μg/mL of μg/mL for 15 minutes (10 minutes longer than the present study), resulted in a ca. 70% decrease in cell viability as measure by resazurin reduction. It is possible that longer exposure to μg/mL in the wire biofilm model would result in increased killing of this particularly mucoid isolate.

In summary, SEQ ID NO: 1 was evaluated for activity in a wire biofilm assay against S. aureus USA 100, E. coli CDC 0451, and P. aeruginosa CDC 0515 in the presence of 50-200 mM sodium bicarbonate. When testing SEQ ID NO: 1 against S. aureus USA100, killing was rapid, resulting in cell counts that were at or approaching the LOD by the 2.5 min and 5 min mark in 150 mM and 200 mM sodium bicarbonate. When testing against E. coli CDC 0451, killing was relatively similar in all sodium bicarbonate concentrations with rapid killing and cell counts at or near the LOD by the 5 min mark for 10 mg/mL SEQ ID NO: 1. No activity was observed when testing SEQ ID NO: 1 activity against the robust biofilms of P. aeruginosa CDC 0515.

TABLE 20A
Time-Kill activity of SEQ ID NO: 1 against Staphylococcus aureus
USA 100 in the wire biofilm study using 50 mM sodium
bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 50 mM | untreated | 0 | 3.67E+4 | 7.37E+3 | 4.56 | — |
| | | 1 | 2.53E+4 | 7.78E+3 | 4.40 | 0.16 |
| | | 2.5 | 4.38E+4 | 1.90E+4 | 4.64 | −0.08 |
| | | 5 | 2.34E+4 | 2.37E+4 | 4.37 | 0.20 |
| | SEQ ID | 0 | 2.93E+4 | 3.22E+4 | 4.47 | 0.10 |
| | NO: 1 | 1 | 4.17E+2 | 4.04E+2 | 2.62 | 1.94 |
| | 1 mg/mL | 2.5 | 6.67E+2 | 3.75E+2 | 2.82 | 1.74 |
| | | 5 | 3.00E+2 | 1.00E+2 | 2.48 | 2.09 |
| | SEQ ID | 0 | 8.07E+4 | 7.15E+4 | 4.91 | −0.34 |
| | NO: 1 | 1 | 2.22E+3 | 2.57E+3 | 3.35 | 1.22 |
| | 3 mg/mL | 2.5 | 4.50E+2 | 3.00E+2 | 2.65 | 1.91 |
| | | 5 | 4.28E+3 | 7.12E+3 | 3.63 | 0.93 |
| | SEQ ID | 0 | 2.75E+4 | 1.22E+4 | 4.44 | 0.12 |
| | NO: 1 | 1 | 6.50E+2 | 4.92E+2 | 2.81 | 1.75 |
| | 10 mg/mL | 2.5 | 3.00E+2 | 1.50E+2 | 2.48 | 2.09 |
| | | 5 | 6.67E+1 | 2.89E+1 | 1.82 | 2.74 |

TABLE 20B
Time-kill activity of SEQ ID NO: 1 against Escherichia coli
CDC 0451 in the wire biofilm study using 50 mM
sodium bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 50 mM | untreated | 0 | 1.20E+05 | 3.15E+04 | 5.08 | — |
| | | 1 | 9.50E+04 | 4.76E+04 | 4.98 | 0.10 |
| | | 2.5 | 6.08E+04 | 6.19E+04 | 4.78 | 0.29 |
| | | 5 | 5.65E+04 | 2.87E+04 | 4.75 | 0.33 |
| | SEQ ID | 0 | 6.52E+04 | 6.18E+04 | 4.81 | 0.26 |
| | NO: 1 | 1 | 2.10E+03 | 1.80E+03 | 3.32 | 1.76 |
| | 1 mg/mL | 2.5 | 2.17E+02 | 1.44E+02 | 2.34 | 2.74 |
| | | 5 | 5.00E+01 | 0.00E+00 | 1.70 | 3.38 |
| | SEQ ID | 0 | 3.42E+04 | 2.24E+04 | 4.53 | 0.54 |
| | NO: 1 | 1 | 1.43E+03 | 9.07E+02 | 3.16 | 1.92 |
| | 3 mg/mL | 2.5 | 5.00E+01 | 0.00E+00 | 1.70 | 3.38 |
| | | 5 | 5.00E+01 | 0.00E+00 | 1.70 | 3.38 |
| | SEQ ID | 0 | 1.79E+04 | 2.69E+04 | 4.25 | 0.82 |
| | NO: 1 | 1 | 9.33E+02 | 1.26E+02 | 2.97 | 2.11 |
| | 10 mg/mL | 2.5 | 5.00E+01 | 0.00E+00 | 1.70 | 3.38 |
| | | 5 | 5.00E+01 | 0.00E+00 | 1.70 | 3.38 |

TABLE 20C
Time-kill activity of SEQ ID NO: 1 against Pseudomonas aeruginosa
CDC 0515 in the wire biofilm study using 50 mM sodium
bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 50 mM | untreated | 0 | 7.67E+06 | 8.95E+06 | 6.88 | — |
| | | 1 | 7.97E+06 | 7.87E+06 | 6.90 | −0.02 |
| | | 2.5 | 1.09E+07 | 1.19E+07 | 7.04 | −0.15 |
| | | 5 | 6.72E+06 | 5.44E+06 | 6.83 | 0.06 |
| | SEQ ID | 0 | 5.38E+06 | 4.14E+06 | 6.73 | 0.15 |
| | NO: 1 | 1 | 7.25E+06 | 5.12E+06 | 6.86 | 0.02 |
| | 1 mg/mL | 2.5 | 3.90E+06 | 4.00E+06 | 6.59 | 0.29 |
| | | 5 | 1.57E+06 | 2.10E+06 | 6.20 | 0.69 |
| | SEQ ID | 0 | 1.38E+06 | 1.44E+05 | 6.14 | 0.74 |
| | NO: 1 | 1 | 2.15E+06 | 2.64E+06 | 6.33 | 0.55 |
| | 3 mg/mL | 2.5 | 9.03E+06 | 4.33E+06 | 6.96 | −0.07 |
| | | 5 | 4.58E+05 | 3.31E+05 | 5.66 | 1.22 |
| | SEQ ID | 0 | 3.52E+07 | 3.26E+07 | 7.55 | −0.66 |
| | NO: 1 | 1 | 1.47E+06 | 1.10E+06 | 6.17 | 0.72 |
| | 10 mg/mL | 2.5 | 5.46E+07 | 8.53E+07 | 7.74 | −0.85 |
| | | 5 | 1.08E+05 | 1.10E+05 | 5.03 | 1.85 |

TABLE 21A
Time-Kill activity of SEQ ID NO: 1 against Staphylococcus aureus
USA 100 in the wire biofilm study using 100 mM sodium
bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 100 | untreated | 0 | 7.98E+4 | 6.00E+4 | 4.90 | — |
| mM | | 1 | 1.97E+4 | 1.41E+4 | 4.29 | 0.61 |
| | | 2.5 | 4.02E+4 | 1.25E+4 | 4.60 | 0.30 |
| | | 5 | 5.21E+4 | 8.06E+4 | 4.72 | 0.19 |
| | SEQ ID | 0 | 4.58E+4 | 2.45E+4 | 4.66 | 0.24 |
| | NO: 1 | 1 | 6.00E+2 | 7.37E+2 | 2.78 | 2.12 |
| | 1 mg/mL | 2.5 | 5.17E+2 | 1.04E+2 | 2.71 | 2.19 |
| | | 5 | 2.17E+2 | 1.26E+2 | 2.34 | 2.57 |
| | SEQ ID | 0 | 5.92E+4 | 7.82E+3 | 4.77 | 0.13 |
| | NO: 1 | 1 | 1.00E+3 | 4.09E+2 | 3.00 | 1.90 |
| | 3 mg/mL | 2.5 | 1.67E+2 | 2.02E+2 | 2.22 | 2.68 |
| | | 5 | 8.33E+1 | 2.89E+1 | 1.92 | 2.98 |
| | SEQ ID | 0 | 3.68E+4 | 1.59E+4 | 4.57 | 0.34 |
| | NO: 1 | 1 | 1.35E+3 | 9.04E+2 | 3.13 | 1.77 |
| | 10 mg/mL | 2.5 | 1.50E+2 | 8.66E+1 | 2.18 | 2.73 |
| | | 5 | 2.00E+2 | 2.60E+2 | 2.30 | 2.60 |

TABLE 21B
Time-Kill activity of SEQ ID NO: 1against Escherichia coli
CDC 0451 in the wire biofilm study using 100 mM
sodium bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 100 | untreated | 0 | 7.55E+04 | 7.30E+04 | 4.88 | — |
| mM | | 1 | 8.02E+04 | 4.25E+04 | 4.90 | −0.03 |
| | | 2.5 | 1.17E+05 | 2.31E+04 | 5.07 | −0.19 |
| | | 5 | 2.75E+04 | 2.20E+04 | 4.44 | 0.44 |
| | SEQ ID | 0 | 4.70E+04 | 4.03E+04 | 4.67 | 0.21 |
| | NO: 1 | 1 | 4.22E+03 | 1.84E+03 | 3.62 | 1.25 |
| | 1 mg/mL | 2.5 | 1.62E+03 | 7.59E+02 | 3.21 | 1.67 |
| | | 5 | 3.00E+02 | 3.91E+02 | 2.48 | 2.40 |
| | SEQ ID | 0 | 7.02E+04 | 4.74E+04 | 4.85 | 0.03 |
| | NO: 1 | 1 | 2.05E+04 | 3.46E+04 | 4.31 | 0.57 |
| | 3 mg/mL | 2.5 | 1.31E+04 | 2.03E+04 | 4.12 | 0.76 |
| | | 5 | 5.00E+01 | 0.00E+00 | 1.70 | 3.18 |
| | SEQ ID | 0 | 1.40E+04 | 1.49E+04 | 4.15 | 0.73 |
| | NO: 1 | 1 | 8.45E+03 | 1.30E+04 | 3.93 | 0.95 |
| | 10 mg/mL | 2.5 | 1.55E+03 | 2.60E+03 | 3.19 | 1.69 |
| | | 5 | 4.55E+03 | 7.75E+03 | 3.66 | 1.22 |

TABLE 21C
Time-kill activity of SEQ ID NO: 1 against Pseudomonas aeruginosa
CDC 0515 in the wire biofilm study using 100 mM sodium
bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 100 | untreated | 0 | 7.27E+06 | 1.78E+06 | 6.86 | — |
| mM | | 1 | 1.69E+07 | 2.11E+07 | 7.23 | −0.37 |
| | | 2.5 | 9.37E+06 | 2.29E+06 | 6.97 | −0.11 |
| | | 5 | 5.73E+06 | 4.50E+06 | 6.76 | 0.10 |
| | SEQ ID | 0 | 7.08E+06 | 9.45E+06 | 6.85 | 0.01 |
| | NO: 1 | 1 | 1.15E+07 | 7.17E+06 | 7.06 | −0.20 |
| | 1 mg/mL | 2.5 | 9.77E+06 | 5.06E+06 | 6.99 | −0.13 |
| | | 5 | 7.82E+05 | 2.75E+05 | 5.89 | 0.97 |
| | SEQ ID | 0 | 1.05E+07 | 5.05E+06 | 7.02 | −0.16 |
| | NO: 1 | 1 | 1.01E+07 | 1.08E+07 | 7.00 | −0.14 |
| | 3 mg/mL | 2.5 | 5.89E+06 | 4.84E+06 | 6.77 | 0.09 |
| | | 5 | 5.07E+06 | 5.53E+06 | 6.70 | 0.16 |
| | SEQ ID | 0 | 1.43E+07 | 8.95E+06 | 7.16 | −0.30 |
| | NO: 1 | 1 | 9.80E+05 | 2.79E+05 | 5.99 | 0.87 |
| | 10 mg/mL | 2.5 | 1.29E+07 | 2.09E+07 | 7.11 | −0.25 |
| | | 5 | 4.17E+06 | 4.01E+06 | 6.62 | 0.24 |

TABLE 22A
Time-Kill activity of SEQ ID NO: 1 against Staphylococcus aureus
USA 100 in the wire biofilm study using 150 mM sodium
bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 150 | untreated | 0 | 1.57E+5 | 7.69E+4 | 5.19 | — |
| mM | | 1 | 4.73E+4 | 1.10E+4 | 4.68 | 0.52 |
| | | 2.5 | 1.17E+4 | 1.03E+4 | 4.07 | 1.13 |
| | | 5 | 2.95E+4 | 6.06E+3 | 4.47 | 0.73 |
| | SEQ ID | 0 | 1.08E+5 | 5.32E+4 | 5.03 | 0.16 |
| | NO: 1 | 1 | 1.68E+3 | 1.28E+3 | 3.23 | 1.97 |
| | 1 mg/mL | 2.5 | 1.17E+2 | 1.15E+2 | 2.07 | 3.13 |
| | | 5 | 5.00E+1 | 0.00E+0 | 1.70 | 3.50 |
| | SEQ ID | 0 | 8.80E+4 | 1.10E+5 | 4.94 | 0.25 |
| | NO: 1 | 1 | 1.63E+3 | 1.23E+3 | 3.21 | 1.98 |
| | 3 mg/mL | 2.5 | 8.33E+1 | 5.77E+1 | 1.92 | 3.27 |
| | | 5 | 5.00E+1 | 0.00E+0 | 1.70 | 3.50 |
| | SEQ ID | 0 | 4.82E+4 | 4.07E+4 | 4.68 | 0.51 |
| | NO: 1 | 1 | 5.00E+2 | 5.63E+2 | 2.70 | 2.50 |
| | 10 mg/mL | 2.5 | 5.00E+1 | 0.00E+0 | 1.70 | 3.50 |
| | | 5 | 6.67E+1 | 2.89E+1 | 1.82 | 3.37 |

TABLE 22B
Time-kill activity of SEQ ID NO: 1 against Escherichia coli
CDC 0451 in the wire biofilm study using 150 mM
sodium bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 150 | untreated | 0 | 8.92E+04 | 5.50E+04 | 4.95 | — |
| mM | | 1 | 3.58E+04 | 1.79E+04 | 4.55 | 0.40 |
| | | 2.5 | 1.06E+05 | 5.73E+04 | 5.03 | −0.08 |
| | | 5 | 6.07E+04 | 2.19E+04 | 4.78 | 0.17 |
| | SEQ ID | 0 | 7.42E+04 | 4.95E+04 | 4.87 | 0.08 |
| | NO: 1 | 1 | 2.05E+03 | 8.79E+02 | 3.31 | 1.64 |
| | 1 mg/mL | 2.5 | 1.23E+03 | 4.91E+02 | 3.09 | 1.86 |
| | | 5 | 2.67E+02 | 1.04E+02 | 2.43 | 2.52 |
| | SEQ ID | 0 | 3.90E+04 | 1.99E+04 | 4.59 | 0.36 |
| | NO: 1 | 1 | 1.35E+03 | 8.26E+02 | 3.13 | 1.82 |
| | 3 mg/mL | 2.5 | 5.00E+02 | 4.44E+02 | 2.70 | 2.25 |
| | | 5 | 3.50E+02 | 3.46E+02 | 2.54 | 2.41 |
| | SEQ ID | 0 | 1.60E+04 | 8.89E+03 | 4.20 | 0.75 |
| | NO: 1 | 1 | 6.33E+02 | 8.04E+02 | 2.80 | 2.15 |
| | 10 mg/mL | 2.5 | 9.50E+02 | 6.08E+02 | 2.98 | 1.97 |
| | | 5 | 5.00E+01 | 0.00E+00 | 1.70 | 3.25 |

TABLE 22C
Time-kill activity of SEQ ID NO: 1 against Pseudomonas aeruginosa
CDC 0515 in the wire biofilm study using 150 mM
sodium bicarbonate as the vehicle.

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 150 | untreated | 0 | 1.63E+07 | 1.81E+07 | 7.21 | — |
| mM | | 1 | 8.58E+06 | 1.12E+07 | 6.93 | 0.28 |
| | | 2.5 | 1.55E+07 | 6.73E+06 | 7.19 | 0.02 |
| | | 5 | 1.48E+07 | 1.21E+07 | 7.17 | 0.04 |
| | SEQ ID | 0 | 6.58E+06 | 3.88E+06 | 6.82 | 0.39 |
| | NO: 1 | 1 | 1.33E+07 | 9.13E+06 | 7.12 | 0.09 |
| | 1 mg/mL | 2.5 | 1.03E+08 | 8.17E+07 | 8.01 | −0.80 |
| | | 5 | 5.73E+06 | 4.92E+06 | 6.76 | 0.45 |
| | SEQ ID | 0 | 1.60E+07 | 5.16E+06 | 7.20 | 0.01 |
| | NO: 1 | 1 | 8.32E+06 | 8.01E+06 | 6.92 | 0.29 |
| | 3 mg/mL | 2.5 | 3.53E+06 | 1.72E+06 | 6.55 | 0.66 |
| | | 5 | 2.37E+07 | 3.97E+07 | 7.37 | −0.16 |
| | SEQ ID | 0 | 2.10E+07 | 8.85E+06 | 7.32 | −0.11 |
| | NO: 1 | 1 | 8.55E+06 | 8.33E+06 | 6.93 | 0.28 |
| | 10 mg/mL | 2.5 | 2.43E+06 | 9.25E+05 | 6.39 | 0.83 |
| | | 5 | 3.57E+06 | 3.71E+06 | 6.55 | 0.66 |

TABLE 23A
Time-Kill activity of SEQ ID NO: 1 against Staphylococcus aureus
USA 100 in the wire biofilm study using 200 mM sodium
bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 200 | untreated | 0 | 1.58E+05 | 7.51E+04 | 5.20 | — |
| mM | | 1 | 6.38E+04 | 2.49E+04 | 4.81 | 0.39 |
| | | 2.5 | 2.88E+04 | 1.78E+04 | 4.46 | 0.74 |
| | | 5 | 7.67E+04 | 7.22E+04 | 4.88 | 0.31 |
| | SEQ ID | 0 | 9.97E+04 | 1.26E+05 | 5.00 | 0.20 |
| | NO: 1 | 1 | 3.28E+03 | 2.68E+03 | 3.52 | 1.68 |
| | 1 mg/mL | 2.5 | 3.52E+03 | 2.43E+03 | 3.55 | 1.65 |
| | | 5 | 5.33E+02 | 2.08E+02 | 2.73 | 2.47 |
| | SEQ ID | 0 | 2.25E+04 | 6.50E+03 | 4.35 | 0.85 |
| | NO: 1 | 1 | 3.83E+03 | 1.94E+03 | 3.58 | 1.62 |
| | 3 mg/mL | 2.5 | 5.50E+02 | 6.61E+02 | 2.74 | 2.46 |
| | | 5 | 2.00E+02 | 1.00E+02 | 2.30 | 2.90 |
| | SEQ ID | 0 | 1.82E+04 | 1.10E+04 | 4.26 | 0.94 |
| | NO: 1 | 1 | 2.10E+03 | 6.54E+02 | 3.32 | 1.88 |
| | 10 mg/mL | 2.5 | 4.00E+02 | 8.66E+01 | 2.60 | 2.60 |
| | | 5 | 5.00E+01 | 0.00E+00 | 1.70 | 3.50 |

TABLE 23B
Time-kill activity of SEQ ID NO: 1 against Escherichia coli
CDC 0451 in the wire biofilm study using 200 mM
sodium bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 200 | untreated | 0 | 1.58E+05 | 7.51E+04 | 5.20 | — |
| mM | | 1 | 6.38E+04 | 2.49E+04 | 4.81 | 0.39 |
| | | 2.5 | 2.88E+04 | 1.78E+04 | 4.46 | 0.74 |
| | | 5 | 7.67E+04 | 7.22E+04 | 4.88 | 0.31 |
| | SEQ ID | 0 | 9.97E+04 | 1.26E+05 | 5.00 | 0.20 |
| | NO: 1 | 1 | 3.28E+03 | 2.68E+03 | 3.52 | 1.68 |
| | 1 mg/mL | 2.5 | 3.52E+03 | 2.43E+03 | 3.55 | 1.65 |
| | | 5 | 5.33E+02 | 2.08E+02 | 2.73 | 2.47 |
| | SEQ ID | 0 | 2.25E+04 | 6.50E+03 | 4.35 | 0.85 |
| | NO: 1 | 1 | 3.83E+03 | 1.94E+03 | 3.58 | 1.62 |
| | 3 mg/mL | 2.5 | 5.50E+02 | 6.61E+02 | 2.74 | 2.46 |
| | | 5 | 2.00E+02 | 1.00E+02 | 2.30 | 2.90 |
| | SEQ ID | 0 | 1.82E+04 | 1.10E+04 | 4.26 | 0.94 |
| | NO: 1 | 1 | 2.10E+03 | 6.54E+02 | 3.32 | 1.88 |
| | 10 mg/mL | 2.5 | 4.00E+02 | 8.66E+01 | 2.60 | 2.60 |
| | | 5 | 5.00E+01 | 0.00E+00 | 1.70 | 3.50 |

TABLE 23C
Time-kill activity of SEQ ID NO: 1 against Pseudomonas aeruginosa
CDC 0515 in the wire biofilm study using 200 mM sodium
bicarbonate as the vehicle

| Sodium | | | | | | |
|---|---|---|---|---|---|---|
| Bicar- | Treat- | | Mean | | log10 | Log10 |
| bonate | ment | Time | CFU/mL | SD | CFU/mL | kill |
| 200 | untreated | 0 | 1.44E+07 | 1.39E+07 | 7.16 | — |
| mM | | 1 | 1.25E+07 | 1.78E+07 | 7.10 | 0.06 |
| | | 2.5 | 1.07E+07 | 5.80E+06 | 7.03 | 0.13 |
| | | 5 | 1.53E+07 | 1.63E+07 | 7.18 | −0.03 |
| | SEQ ID | 0 | 2.55E+07 | 2.08E+07 | 7.41 | −0.25 |
| | NO: 1 | 1 | 2.22E+07 | 2.85E+07 | 7.35 | −0.19 |
| | 1 mg/mL | 2.5 | 1.92E+07 | 1.42E+07 | 7.28 | −0.13 |
| | | 5 | 1.76E+06 | 1.89E+06 | 6.25 | 0.91 |
| | SEQ ID | 0 | 1.39E+07 | 6.22E+06 | 7.14 | 0.01 |
| | NO: 1 | 1 | 5.93E+06 | 6.66E+06 | 6.77 | 0.38 |
| | 3 mg/mL | 2.5 | 1.24E+07 | 9.55E+06 | 7.09 | 0.07 |
| | | 5 | 1.07E+07 | 7.17E+06 | 7.03 | 0.13 |
| | SEQ ID | 0 | 1.61E+07 | 1.26E+07 | 7.21 | −0.05 |
| | NO: 1 | 1 | 9.82E+06 | 1.38E+06 | 6.99 | 0.16 |
| | 10 mg/mL | 2.5 | 3.01E+06 | 2.74E+06 | 6.48 | 0.68 |
| | | 5 | 6.65E+05 | 4.95E+05 | 5.82 | 1.33 |

TABLE 24A
CFU count and dilutions of dilutions of recovered S. aureus NRS382 from the wire biofilm assay

| | | Time | | CFU | CFU | | CFU/mL | CFU/mL | | Mean | |
|---|---|---|---|---|---|---|---|---|---|---|---|
| | Treatment | (min) | Rep | 1 | 2 | DF | 1 | 2 | CFU/mL | CFU/mL | SD |
| Sodium | None | 0 | A | 36 | 32 | 10 | 3.60E+04 | 3.20E+04 | 3.40E+04 | 3.67E+4 | 7.37E+3 |
| Bicarbonate | | | B | 29 | 33 | 10 | 2.90E+04 | 3.30E+04 | 3.10E+04 | | |
| (mM) | | | C | 37 | 53 | 10 | 3.70E+04 | 5.30E+04 | 4.50E+04 | | |
| 50 | | 1 | A | 34 | 33 | 10 | 3.40E+04 | 3.30E+04 | 3.35E+04 | 2.53E+4 | 7.78E+3 |
| | | | B | 32 | 17 | 10 | 3.20E+04 | 1.70E+04 | 2.45E+04 | | |
| | | | C | 15 | 21 | 10 | 1.50E+04 | 2.10E+04 | 1.80E+04 | | |
| | | 2.5 | A | 33 | 56 | 10 | 3.30E+04 | 5.60E+04 | 4.45E+04 | 4.38E+4 | 1.90E+4 |
| | | | B | 23 | 26 | 10 | 2.30E+04 | 2.60E+04 | 2.45E+04 | | |
| | | | C | 64 | 61 | 10 | 6.40E+04 | 6.10E+04 | 6.25E+04 | | |
| | | 5 | A | 47 | 48 | 10 | 4.70E+04 | 4.80E+04 | 4.75E+04 | 2.34E+4 | 2.37E+4 |
| | | | B | 0 | 0 | 1 | 0 | 0 | 5.00E+01 | | |
| | | | C | 24 | 21 | 10 | 2.40E+04 | 2.10E+04 | 2.25E+04 | | |
| | SEQ ID | 0 | A | 61 | 58 | 1 | 6.10E+03 | 5.80E+03 | 5.95E+03 | 2.93E+4 | 3.22E+4 |
| | NO: 1 | | B | 16 | 16 | 10 | 1.60E+04 | 1.60E+04 | 1.60E+04 | | |
| | 1 mg/mL | | C | 59 | 73 | 10 | 5.90E+04 | 7.30E+04 | 6.60E+04 | | |
| | | 1 | A | 0 | 1 | 1 | 0 | 1.00E+02 | 5.00E+01 | 4.17E+2 | 4.04E+2 |
| | | | B | 3 | 4 | 1 | 3.00E+02 | 4.00E+02 | 3.50E+02 | | |
| | | | C | 12 | 5 | 1 | 1.20E+03 | 5.00E+02 | 8.50E+02 | | |
| | | 2.5 | A | 5 | 4 | 1 | 5.00E+02 | 4.00E+02 | 4.50E+02 | 6.67E+2 | 3.75E+2 |
| | | | B | 8 | 14 | 1 | 8.00E+02 | 1.40E+03 | 1.10E+03 | | |
| | | | C | 5 | 4 | 1 | 5.00E+02 | 4.00E+02 | 4.50E+02 | | |
| | | 5 | A | 3 | 1 | 1 | 3.00E+02 | 1.00E+02 | 2.00E+02 | 3.00E+2 | 1.00E+2 |
| | | | B | 2 | 4 | 1 | 2.00E+02 | 4.00E+02 | 3.00E+02 | | |
| | | | C | 4 | 4 | 1 | 4.00E+02 | 4.00E+02 | 4.00E+02 | | |
| | SEQ ID | 0 | A | 68 | 74 | 1 | 6.80E+03 | 7.40E+03 | 7.10E+03 | 8.07E+4 | 7.15E+4 |
| | NO: 1 | | B | 18 | 12 | 100 | 1.80E+05 | 1.20E+05 | 1.50E+05 | | |
| | 3 mg/mL | | C | 9 | 8 | 100 | 9.00E+04 | 8.00E+04 | 8.50E+04 | | |
| | | 1 | A | 2 | 1 | 1 | 2.00E+02 | 1.00E+02 | 1.50E+02 | 2.22E+3 | 2.57E+3 |
| | | | B | 11 | 17 | 1 | 1.10E+03 | 1.70E+03 | 1.40E+03 | | |
| | | | C | 53 | 49 | 1 | 5.30E+03 | 4.90E+03 | 5.10E+03 | | |
| | | 2.5 | A | 10 | 5 | 1 | 1.00E+03 | 5.00E+02 | 7.50E+02 | 4.50E+2 | 3.00E+2 |
| | | | B | 3 | 6 | 1 | 3.00E+02 | 6.00E+02 | 4.50E+02 | | |
| | | | C | 2 | 1 | 1 | 2.00E+02 | 1.00E+02 | 1.50E+02 | | |
| | | 5 | A | 2 | 2 | 1 | 2.00E+02 | 2.00E+02 | 2.00E+02 | 4.28E+3 | 7.12E+3 |
| | | | B | 1 | 2 | 1 | 1.00E+02 | 2.00E+02 | 1.50E+02 | | |
| | | | C | 13 | 12 | 10 | 1.30E+04 | 1.20E+04 | 1.25E+04 | | |
| | SEQ ID | 0 | A | 42 | 29 | 10 | 4.20E+04 | 2.90E+04 | 3.55E+04 | 2.75E+4 | 1.22E+4 |
| | NO: 1 | | B | 18 | 9 | 10 | 1.80E+04 | 9.00E+03 | 1.35E+04 | | |
| | 10 mg/mL | | C | 40 | 27 | 10 | 4.00E+04 | 2.70E+04 | 3.35E+04 | | |
| | | 1 | A | 12 | 12 | 1 | 1.20E+03 | 1.20E+03 | 1.20E+03 | 6.50E+2 | 4.92E+2 |
| | | | B | 3 | 2 | 1 | 3.00E+02 | 2.00E+02 | 2.50E+02 | | |
| | | | C | 7 | 3 | 1 | 7.00E+02 | 3.00E+02 | 5.00E+02 | | |
| | | 2.5 | A | 1 | 2 | 1 | 1.00E+02 | 2.00E+02 | 1.50E+02 | 3.00E+2 | 1.50E+2 |
| | | | B | 6 | 3 | 1 | 6.00E+02 | 3.00E+02 | 4.50E+02 | | |
| | | | C | 2 | 4 | 1 | 2.00E+02 | 4.00E+02 | 3.00E+02 | | |
| | | 5 | A | 1 | 0 | 1 | 1.00E+02 | 0 | 5.00E+01 | 6.67E+1 | 2.89E+1 |
| | | | B | 0 | 0 | 1 | 0 | 0 | 5.00E+01 | | |
| | | | C | 2 | 0 | 1 | 2.00E+02 | 0 | 1.00E+02 | | |
| 100 | None | 0 | A | 17 | 23 | 10 | 1.70E+04 | 2.30E+04 | 2.00E+04 | 7.98E+4 | 6.00E+4 |
| | | | B | 16 | 12 | 100 | 1.60E+05 | 1.20E+05 | 1.40E+05 | | |
| | | | C | 77 | 82 | 10 | 7.70E+04 | 8.20E+04 | 7.95E+04 | | |
| | | 1 | A | 8 | 5 | 10 | 8.00E+03 | 5.00E+03 | 6.50E+03 | 1.97E+4 | 1.41E+4 |
| | | | B | 38 | 31 | 10 | 3.80E+04 | 3.10E+04 | 3.45E+04 | | |
| | | | C | 18 | 18 | 10 | 1.80E+04 | 1.80E+04 | 1.80E+04 | | |
| | | 2.5 | A | 41 | 38 | 10 | 4.10E+04 | 3.80E+04 | 3.95E+04 | 4.02E+4 | 1.25E+4 |
| | | | B | 30 | 26 | 10 | 3.00E+04 | 2.60E+04 | 2.80E+04 | | |
| | | | C | 62 | 44 | 10 | 6.20E+04 | 4.40E+04 | 5.30E+04 | | |
| | | 5 | 4 | 11 | 11 | 10 | 1.10E+04 | 1.10E+04 | 1.10E+04 | 5.21E+4 | 8.06E+4 |
| | | | B | 16 | 13 | 100 | 1.60E+05 | 1.30E+05 | 1.45E+05 | | |
| | | | C | 5 | 3 | 1 | 5.00E+02 | 3.00E+02 | 4.00E+02 | | |
| | SEQ ID | 0 | 4 | 25 | 29 | 10 | 2.50E+04 | 2.90E+04 | 2.70E+04 | 4.58E+4 | 2.45E+4 |
| | NO: 1 | | B | 64 | 83 | 10 | 6.40E+04 | 8.30E+04 | 7.35E+04 | | |
| | 1 mg/mL | | C | 39 | 35 | 10 | 3.90E+04 | 3.50E+04 | 3.70E+04 | | |
| | | 1 | A | 1 | 3 | 1 | 1.00E+02 | 3.00E+02 | 2.00E+02 | 6.00E+2 | 7.37E+2 |
| | | | B | 15 | 14 | 1 | 1.50E+03 | 1.40E+03 | 1.45E+03 | | |
| | | | C | 3 | 0 | 1 | 3.00E+02 | 0 | 1.50E+02 | | |
| | | 2.5 | A | 7 | 5 | 1 | 7.00E+02 | 5.00E+02 | 6.00E+02 | 5.17E+2 | 1.04E+2 |
| | | | B | 4 | 7 | 1 | 4.00E+02 | 7.00E+02 | 5.50E+02 | | |
| | | | C | 4 | 4 | 1 | 4.00E+02 | 4.00E+02 | 4.00E+02 | | |
| | | 5 | A | 1 | 1 | 1 | 1.00E+02 | 1.00E+02 | 1.00E+02 | 2.17E+2 | 1.26E+2 |
| | | | B | 2 | 5 | 1 | 2.00E+02 | 5.00E+02 | 3.50E+02 | | |
| | | | C | 2 | 2 | 1 | 2.00E+02 | 2.00E+02 | 2.00E+02 | | |
| | SEQ ID | 0 | A | 59 | 57 | 10 | 5.90E+04 | 5.70E+04 | 5.80E+04 | 5.92E+4 | 7.82E+3 |
| | NO: 1 | | B | 72 | 63 | 10 | 7.20E+04 | 6.30E+04 | 6.75E+04 | | |
| | 3 mg/mL | | C | 56 | 48 | 10 | 5.60E+04 | 4.80E+04 | 5.22E+04 | | |
| | | 1 | A | 9 | 9 | 1 | 9.00E+02 | 9.00E+02 | 9.00E+02 | 1.00E+3 | 4.09E+2 |
| | | | B | 21 | 8 | 1 | 2.10E+03 | 8.00E+02 | 1.45E+03 | | |
| | | | C | 10 | 3 | 1 | 1.00E+03 | 3.00E+02 | 6.50E+02 | | |
| | | 2.5 | A | 0 | 0 | 1 | 0 | 0 | 5.00E+01 | 1.67E+2 | 2.02E+2 |
| | | | B | 0 | 0 | 1 | 0 | 0 | 5.00E+01 | | |
| | | | C | 5 | 3 | 1 | 5.00E+02 | 3.00E+02 | 4.00E+02 | | |
| | | 5 | A | 0 | 0 | 1 | 0 | 0 | 5.00E+01 | 8.33E+1 | 2.89E+1 |
| | | | B | 1 | 1 | 1 | 1.00E+02 | 1.00E+02 | 1.00E+02 | | |
| | | | C | 1 | 1 | 1 | 1.00E+02 | 1.00E+02 | 1.00E+02 | | |
| | SEQ ID | 0 | A | 61 | 49 | 10 | 6.10E+04 | 4.90E+04 | 5.50E+04 | 3.68E+4 | 1.59E+4 |
| | NO: 1 | | B | 27 | 24 | 10 | 2.70E+04 | 2.40E+04 | 2.55E+04 | | |
| | 10 mg/mL | | C | 32 | 28 | 10 | 3.20E+04 | 2.80E+04 | 3.00E+04 | | |
| | | 1 | A | 14 | 15 | 1 | 1.40E+03 | 1.50E+03 | 1.45E+03 | 1.35E+3 | 9.04E+2 |
| | | | B | 4 | 4 | 1 | 4.00E+02 | 4.00E+02 | 4.00E+02 | | |
| | | | C | 23 | 21 | 1 | 2.30E+03 | 2.10E+03 | 2.20E+03 | | |
| | | 2.5 | A | 2 | 0 | 1 | 2.00E+02 | 0 | 1.00E+02 | 1.50E+2 | 8.66E+1 |
| | | | B | 4 | 1 | 1 | 4.00E+02 | 1.00E+02 | 2.50E+02 | | |
| | | | C | 1 | 1 | 1 | 1.00E+02 | 1.00E+02 | 1.00E+02 | | |
| | | 5 | A | 4 | 6 | 1 | 4.00E+02 | 6.00E+02 | 5.00E+02 | 2.00E+2 | 2.60E+2 |
| | | | B | 1 | 0 | 1 | 1.00E+02 | 0 | 5.00E+01 | | |
| | | | C | 0 | 0 | 1 | 0 | 0 | 5.00E+01 | | |
| 150 | None | 0 | A | 24 | 25 | 100 | 2.40E+05 | 2.50E+05 | 2.45E+05 | 1.57E+5 | 7.69E+4 |
| | | | B | 11 | 13 | 100 | 1.10E+05 | 1.30E+05 | 1.20E+05 | | |
| | | | C | 13 | 8 | 100 | 1.30E+05 | 8.00E+04 | 1.05E+05 | | |
| | | 1 | A | 58 | 62 | 10 | 5.80E+04 | 6.20E+04 | 6.00E+04 | 4.73E+4 | 1.10E+4 |
| | | | B | 42 | 40 | 10 | 4.20E+04 | 4.00E+04 | 4.10E+04 | | |
| | | | C | 44 | 38 | 10 | 4.40E+04 | 3.80E+04 | 4.10E+04 | | |
| | | 2.5 | A | 23 | 23 | 10 | 2.30E+04 | 2.30E+04 | 2.30E+04 | 1.17E+4 | 1.03E+4 |
| | | | B | 8 | 11 | 10 | 8.00E+03 | 1.10E+04 | 9.50E+03 | | |
| | | | C | 27 | 27 | 1 | 2.70E+03 | 2.70E+03 | 2.70E+03 | | |
| | | 5 | A | 23 | 25 | 10 | 2.30E+04 | 2.50E+04 | 2.40E+04 | 2.95E+4 | 6.06E+3 |
| | | | B | 45 | 27 | 10 | 4.50E+04 | 2.70E+04 | 3.60E+04 | | |
| | | | C | 30 | 27 | 10 | 3.00E+04 | 2.70E+04 | 2.85E+04 | | |
| | SEQ ID: 1 | 0 | A | 51 | 45 | 10 | 5.10E+04 | 4.50E+04 | 4.80E+04 | 1.08E+5 | 5.32E+4 |
| | 1 mg/mL | | B | 20 | 10 | 100 | 2.00E+05 | 1.00E+05 | 1.50E+05 | | |
| | | | C | 7 | 18 | 100 | 7.00E+04 | 1.80E+05 | 1.25E+05 | | |
| | | 1 | A | 8 | 8 | 1 | 8.00E+02 | 8.00E+02 | 8.00E+02 | 1.68E+3 | 1.28E+3 |
| | | | B | 31 | 32 | 1 | 3.10E+03 | 3.20E+03 | 3.15E+03 | | |
| | | | C | 9 | 13 | 1 | 9.00E+02 | 1.30E+03 | 1.10E+03 | | |
| | | 2.5 | A | 0 | 1 | 1 | 0 | 1.00E+02 | 5.00E+01 | 1.17E+2 | 1.15E+2 |
| | | | B | 0 | 1 | 1 | 0 | 1.00E+02 | 5.00E+01 | | |
| | | | C | 4 | 1 | 1 | 4.00E+02 | 1.00E+02 | 2.50E+02 | | |
| | | 5 | A | 0 | 0 | 1 | 0 | 0 | 5.00E+01 | 5.00E+1 | 0.00E+0 |
| | | | B | 1 | 0 | 1 | 1.00E+02 | 0 | 5.00E+01 | | |
| | | | C | 0 | 0 | 1 | 0 | 0 | 5.00E+01 | | |
| | SEQ ID | 0 | A | 19 | 24 | 100 | 1.90E+05 | 2.40E+05 | 2.15E+05 | 8.80E+4 | 1.10E+5 |
| | NO: 1 | | B | 32 | 25 | 10 | 3.20E+04 | 2.50E+04 | 2.85E+04 | | |
| | 3 mg/mL | | C | 19 | 22 | 10 | 1.90E+04 | 2.20E+04 | 2.05E+04 | | |
| | | 1 | A | 5 | 2 | 1 | 5.00E+02 | 2.00E+02 | 3.50E+02 | 1.63E+3 | 1.23E+3 |
| | | | B | 19 | 16 | 1 | 1.90E+03 | 1.60E+03 | 1.75E+03 | | |
| | | | C | 31 | 25 | 1 | 3.10E+03 | 2.50E+03 | 2.80E+03 | | |
| | | 2.5 | A | 0 | 0 | 1 | 0 | 0 | 5.00E+01 | 8.33E+1 | 5.77E+1 |
| | | | B | 0 | 3 | 1 | 0 | 3.00E+02 | 1.50E+02 | | |
| | | | C | 0 | 0 | 1 | 0 | 0 | 5.00E+01 | | |
| | | 5 | A | 0 | 0 | 1 | 0 | 0 | 5.00E+01 | 5.00E+1 | 0.00E+0 |
| | | | B | 0 | 0 | 1 | 0 | 0 | 5.00E+01 | | |
| | | | C | 0 | 0 | 1 | 0 | 0 | 5.00E+01 | | |
| | SEQ ID | 0 | A | 26 | 29 | 10 | 2.60E+04 | 2.90E+04 | 2.75E+04 | 4.82E+4 | 4.07E+4 |
| | NO: 1 | | B | 22 | 22 | 10 | 2.20E+04 | 2.20E+04 | 2.20E+04 | | |
| | 10 mg/mL | | C | 10 | 9 | 100 | 1.00E+05 | 9.00E+04 | 9.50E+04 | | |
| | | 1 | A | 13 | 10 | 1 | 1.30E+03 | 1.00E+03 | 1.15E+03 | 5.00E+2 | 5.63E+2 |
| | | | B | 2 | 1 | 1 | 2.00E+02 | 1.00E+02 | 1.50E+02 | | |
| | | | C | 2 | 2 | 1 | 2.00E+02 | 2.00E+02 | 2.00E+02 | | |
| | | 2.5 | A | 0 | 0 | 1 | 0 | 0 | 5.00E+01 | 5.00E+1 | 0.00E+0 |
| | | | B | 0 | 0 | 1 | 0 | 0 | 5.00E+01 | | |
| | | | C | 1 | 0 | 1 | 1.00E+02 | 0 | 5.00E+01 | | |
| | | 5 | A | 0 | 0 | 1 | 0 | 0 | 5.00E+01 | 6.67E+1 | 2.89E+1 |
| | | | B | 1 | 1 | 1 | 1.00E+02 | 1.00E+02 | 1.00E+02 | | |
| | | | C | 0 | 0 | 1 | 0 | 0 | 5.00E+01 | | |
| 200 | None | 0 | A | 49 | 59 | 10 | 4.90E+04 | 5.90E+04 | 5.40E+04 | 1.31E+5 | 1.16E+5 |
| | | | B | 22 | 31 | 100 | 2.20E+05 | 3.10E+05 | 2.65E+05 | | |
| | | | C | 72 | 78 | 10 | 7.20E+04 | 7.80E+04 | 7.50E+04 | | |
| | | 1 | A | 8 | 7 | 10 | 8.00E+03 | 7.00E+03 | 7.50E+03 | 1.47E+4 | 1.07E+4 |
| | | | B | 42 | 12 | 10 | 4.20E+04 | 1.20E+04 | 2.70E+04 | | |
| | | | C | 7 | 12 | 10 | 7.00E+03 | 1.20E+04 | 9.50E+03 | | |
| | | 2.5 | A | 47 | 53 | 10 | 4.70E+04 | 5.30E+04 | 5.00E+04 | 3.77E+4 | 1.14E+4 |
| | | | B | 27 | 28 | 10 | 2.70E+04 | 2.80E+04 | 2.75E+04 | | |
| | | | C | 36 | 35 | 10 | 3.60E+04 | 3.50E+04 | 3.55E+04 | | |
| | | 5 | A | 22 | 29 | 10 | 2.20E+04 | 2.90E+04 | 2.55E+04 | 2.30E+4 | 1.74E+4 |
| | | | B | 43 | 35 | 10 | 4.30E+04 | 3.50E+04 | 3.90E+04 | | |
| | | | C | 44 | 45 | 1 | 4.40E+03 | 4.50E+03 | 4.45E+03 | | |
| | SEQ ID | 0 | A | 34 | 26 | 10 | 3.40E+04 | 2.60E+04 | 3.00E+04 | 4.73E+4 | 1.83E+4 |
| | NO: 1 | | B | 64 | 69 | 10 | 6.40E+04 | 6.90E+04 | 6.65E+04 | | |
| | 1 mg/mL | | C | 39 | 52 | 10 | 3.90E+04 | 5.20E+04 | 4.55E+04 | | |
| | | 1 | A | 36 | 49 | 1 | 3.60E+03 | 4.90E+03 | 4.25E+03 | 2.42E+3 | 1.83E+3 |
| | | | B | 22 | 26 | 1 | 2.20E+03 | 2.60E+03 | 2.40E+03 | | |
| | | | C | 4 | 8 | 1 | 4.00E+02 | 8.00E+02 | 6.00E+02 | | |
| | | 2.5 | A | 2 | 1 | 1 | 2.00E+02 | 1.00E+02 | 1.50E+02 | 8.33E+1 | 5.77E+1 |
| | | | B | 0 | 0 | 1 | 0 | 0 | 5.00E+01 | | |
| | | | C | 1 | 0 | 1 | 1.00E+02 | 0 | 5.00E+01 | | |
| | | 5 | A | 0 | 0 | 1 | 0 | 0 | 5.00E+01 | 5.00E+1 | 0.00E+0 |
| | | | B | 0 | 0 | 1 | 0 | 0 | 5.00E+01 | | |
| | | | C | 0 | 0 | 1 | 0 | 0 | 5.00E+01 | | |
| | SEQ ID | 0 | A | 23 | 47 | 10 | 2.30E+04 | 4.70E+04 | 3.50E+04 | 4.62E+4 | 1.56E+4 |
| | NO: 1 | | B | 44 | 35 | 10 | 4.40E+04 | 3.50E+04 | 3.95E+04 | | |
| | 3 mg/mL | | C | 74 | 54 | 10 | 7.40E+04 | 5.40E+04 | 6.40E+04 | | |
| | | 1 | A | 15 | 15 | 1 | 1.50E+03 | 1.50E+03 | 1.50E+03 | 8.67E+2 | 5.51E+2 |
| | | | B | 4 | 6 | 1 | 4.00E+02 | 6.00E+02 | 5.00E+02 | | |
| | | | C | 6 | 6 | 1 | 6.00E+02 | 6.00E+02 | 6.00E+02 | | |
| | | 2.5 | A | 0 | 0 | 1 | 0 | 0 | 5.00E+01 | 5.00E+2 | 7.79E+2 |
| | | | B | 0 | 0 | 1 | 0 | 0 | 5.00E+01 | | |
| | | | C | 14 | 14 | 1 | 1.40E+03 | 1.40E+03 | 1.40E+03 | | |
| | | 5 | A | 0 | 0 | 1 | 0 | 0 | 5.00E+01 | 5.00E+1 | 0.00E+0 |
| | | | B | 0 | 0 | 1 | 0 | 0 | 5.00E+01 | | |
| | | | C | 0 | 0 | 1 | 0 | 0 | 5.00E+01 | | |
| | SEQ ID | 0 | A | 40 | 41 | 10 | 4.00E+04 | 4.10E+04 | 4.05E+04 | 6.10E+4 | 1.83E+4 |
| | NO: 1 | | B | 54 | 80 | 10 | 5.40E+04 | 8.00E+04 | 6.70E+04 | | |
| | 10 mg/mL | | C | 72 | 79 | 10 | 7.20E+04 | 7.90E+04 | 7.55E+04 | | |
| | | 1 | A | 5 | 14 | 1 | 5.00E+02 | 1.40E+03 | 9.50E+02 | 7.67E+2 | 4.07E+2 |
| | | | B | 13 | 8 | 1 | 1.30E+03 | 8.00E+02 | 1.05E+03 | | |
| | | | C | 4 | 2 | 1 | 4.00E+02 | 2.00E+02 | 3.00E+02 | | |
| | | 2.5 | A | 0 | 0 | 1 | 0 | 0 | 5.00E+01 | 6.67E+1 | 2.89E+1 |
| | | | B | 2 | 0 | 1 | 2.00E+02 | 0 | 1.00E+02 | | |
| Na | | 2.5 | A | 47 | 53 | 10 | 4.70E+04 | 5.30E+04 | 5.00E+04 | 3.77E+4 | 1.14E+4 |
| Bicarbonate | | | B | 27 | 28 | 10 | 2.70E+04 | 2.80E+04 | 2.75E+04 | | |
| (mM) | | | C | 36 | 35 | 10 | 3.60E+04 | 3.50E+04 | 3.55E+04 | | |
| | | 5 | A | 22 | 29 | 10 | 2.20E+04 | 2.90E+04 | 2.55E+04 | 2.30E+4 | 1.74E+4 |
| | | | B | 43 | 35 | 10 | 4.30E+04 | 3.50E+04 | 3.90E+04 | | |
| | | | C | 44 | 45 | 1 | 4.40E+03 | 4.50E+03 | 4.45E+03 | | |
| | SEQ ID | 0 | A | 34 | 26 | 10 | 3.40E+04 | 2.60E+04 | 3.00E+04 | 4.73E+4 | 1.83E+4 |
| | NO: 1 | | B | 64 | 69 | 10 | 6.40E+04 | 6.90E+04 | 6.65E+04 | | |
| | 1 mg/mL | | C | 39 | 52 | 10 | 3.90E+04 | 5.20E+04 | 4.55E+04 | | |
| | | 1 | A | 36 | 49 | 1 | 3.60E+03 | 4.90E+03 | 4.25E+03 | 2.42E+3 | 1.83E+3 |
| | | | B | 22 | 26 | 1 | 2.20E+03 | 2.60E+03 | 2.40E+03 | | |
| | | | C | 4 | 8 | 1 | 4.00E+02 | 8.00E+02 | 6.00E+02 | | |
| | | 2.5 | A | 2 | 1 | 1 | 2.00E+02 | 1.00E+02 | 1.50E+02 | 8.33E+1 | 5.77E+1 |
| | | | B | 0 | 0 | 1 | 0 | 0 | 5.00E+01 | | |
| | | | C | 1 | 0 | 1 | 1.00E+02 | 0 | 5.00E+01 | | |
| | | 5 | A | 0 | 0 | 1 | 0 | 0 | 5.00E+01 | 5.00E+1 | 0.00E+0 |
| | | | B | 0 | 0 | 1 | 0 | 0 | 5.00E+01 | | |
| | | | C | 0 | 0 | 1 | 0 | 0 | 5.00E+01 | | |
| | SEQ ID | 0 | A | 23 | 47 | 10 | 2.30E+04 | 4.70E+04 | 3.50E+04 | 4.62E+4 | 1.56E+4 |
| | NO: 1 | | B | 44 | 35 | 10 | 4.40E+04 | 3.50E+04 | 3.95E+04 | | |
| | 3 mg/mL | | C | 74 | 54 | 10 | 7.40E+04 | 5.40E+04 | 6.40E+04 | | |
| | | 1 | A | 15 | 15 | 1 | 1.50E+03 | 1.50E+03 | 1.50E+03 | 8.67E+2 | 5.51E+2 |
| | | | B | 4 | 6 | 1 | 4.00E+02 | 6.00E+02 | 5.00E+02 | | |
| | | | C | 6 | 6 | 1 | 6.00E+02 | 6.00E+02 | 6.00E+02 | | |
| | | 2.5 | A | 0 | 0 | 1 | 0 | 0 | 5.00E+01 | 5.00E+2 | 7.79E+2 |
| | | | B | 0 | 0 | 1 | 0 | 0 | 5.00E+01 | | |
| | | | C | 14 | 14 | 1 | 1.40E+03 | 1.40E+03 | 1.40E+03 | | |
| | | 5 | A | 0 | 0 | 1 | 0 | 0 | 5.00E+01 | 5.00E+1 | 0.00E+0 |
| | | | B | 0 | 0 | 1 | 0 | 0 | 5.00E+01 | | |
| | | | C | 0 | 0 | 1 | 0 | 0 | 5.00E+01 | | |
| | SEQ ID | 0 | A | 40 | 41 | 10 | 4.00E+04 | 4.10E+04 | 4.05E+04 | 6.10E+4 | 1.83E+4 |
| | NO: 1 | | B | 54 | 80 | 10 | 5.40E+04 | 8.00E+04 | 6.70E+04 | | |
| | 10 mg/mL | | C | 72 | 79 | 10 | 7.20E+04 | 7.90E+04 | 7.55E+04 | | |
| | | 1 | A | 5 | 14 | 1 | 5.00E+02 | 1.40E+03 | 9.50E+02 | 7.67E+2 | 4.07E+2 |
| | | | B | 13 | 8 | 1 | 1.30E+03 | 8.00E+02 | 1.05E+03 | | |
| | | | C | 4 | 2 | 1 | 4.00E+02 | 2.00E+02 | 3.00E+02 | | |
| | | 2.5 | A | 0 | 0 | 1 | 0 | 0 | 5.00E+01 | 6.67E+1 | 2.89E+1 |
| | | | B | 2 | 0 | 1 | 2.00E+02 | 0 | 1.00E+02 | | |
| | | | C | 0 | 0 | 1 | 0 | 0 | 5.00E+01 | | |
| | | 5 | A | 0 | 0 | 1 | 0 | 0 | 5.00E+01 | 5.00E+1 | 0.00E+0 |
| | | | B | 0 | 0 | 1 | 0 | 0 | 5.00E+01 | | |
| | | | C | 0 | 0 | 1 | 0 | 0 | 5.00E+01 | | |

TABLE 24B
CFU count and dilutions of dilutions of recovered Escherichia coli CDC 0451 from the wire biofilm assay

| Sodium | | | | | | | | | | | |
|---|---|---|---|---|---|---|---|---|---|---|---|
| Bicarbonate | | Time | | CFU | CFU | | CFU/mL | CFU/mL | | Mean | |
| (mM) | Treatment | (min) | Rep | 1 | 2 | DF | 1 | 2 | CFU/mL | CFU/mL | SD |
| 50 | None | 0 | A | 96 | 73 | 10 | 9.60E+04 | 7.30E+04 | 8.45E+04 | 1.20E+5 | 3.15E+4 |
| | | | B | 17 | 12 | 100 | 1.70E+05 | 1.20E+05 | 1.45E+05 | | |
| | | | C | 14 | 12 | 100 | 1.40E+05 | 1.20E+05 | 1.30E+05 | | |
| | | 1 | A | 17 | 13 | 100 | 1.70E+05 | 1.30E+05 | 1.50E+05 | 9.50E+4 | 4.76E+4 |
| | | | B | 62 | 72 | 10 | 6.20E+04 | 7.20E+04 | 6.70E+04 | | |
| | | | C | 68 | X | 10 | 6.80E+04 | | 6.80E+04 | | |
| | | 2.5 | A | 11 | 10 | 10 | 1.10E+04 | 1.00E+04 | 1.05E+04 | 6.08E+4 | 6.19E+4 |
| | | | B | 43 | 41 | 10 | 4.30E+04 | 4.10E+04 | 4.20E+04 | | |
| | | | C | 16 | 10 | 100 | 1.60E+05 | 1.00E+05 | 1.30E+05 | | |
| | | 5 | A | 10 | 6 | 100 | 1.00E+05 | 6.00E+04 | 8.00E+04 | 5.65E+4 | 2.87E+4 |
| | | | B | 27 | 22 | 10 | 2.70E+04 | 2.20E+04 | 2.45E+04 | | |
| | | | C | 11 | 2 | 100 | 1.10E+05 | 2.00E+04 | 6.50E+04 | | |
| | SEQ ID | 0 | A | 17 | 10 | 100 | 1.70E+05 | 1.00E+05 | 1.35E+05 | 6.52E+4 | 6.18E+4 |
| | NO: 1 | | B | 39 | 47 | 10 | 3.90E+04 | 4.70E+04 | 4.30E+04 | | |
| | 1 mg/mL | | C | 24 | 11 | 10 | 2.40E+04 | 1.10E+04 | 1.75E+04 | | |
| | | 1 | A | 28 | 29 | 1 | 2.80E+03 | 2.90E+03 | 2.85E+03 | 2.10E+3 | 1.80E+3 |
| | | | B | 32 | 36 | 1 | 3.20E+03 | 3.60E+03 | 3.40E+03 | | |
| | | | C | 1 | 0 | 1 | 1.00E+02 | 0.00E+00 | 5.00E+01 | | |
| | | 2.5 | A | 3 | 3 | 1 | 3.00E+02 | 3.00E+02 | 3.00E+02 | 2.17E+2 | 1.44E+2 |
| | | | B | 3 | 3 | 1 | 3.00E+02 | 3.00E+02 | 3.00E+02 | | |
| | | | C | 1 | 0 | 1 | 1.00E+02 | 0.00E+00 | 5.00E+01 | | |
| | | 5 | A | 0 | 0 | 1 | 5.00E+01 | 5.00E+01 | 5.00E+01 | 5.00E+1 | 0.00E+0 |
| | | | B | 0 | 0 | 1 | 5.00E+01 | 5.00E+01 | 5.00E+01 | | |
| | | | C | 0 | 0 | 1 | 5.00E+01 | 5.00E+01 | 5.00E+01 | | |
| | SEQ ID | 0 | A | 13 | 18 | 10 | 1.30E+04 | 1.80E+04 | 1.55E+04 | 3.42E+4 | 2.24E+4 |
| | NO: 1 | | B | 27 | 29 | 10 | 2.70E+04 | 2.90E+04 | 2.80E+04 | | |
| | 3 mg/mL | | C | 62 | 56 | 10 | 6.20E+04 | 5.60E+04 | 5.90E+04 | | |
| | | 1 | A | 18 | 30 | 1 | 1.80E+03 | 3.00E+03 | 2.40E+03 | 1.43E+3 | 9.07E+2 |
| | | | B | 11 | 15 | 1 | 1.10E+03 | 1.50E+03 | 1.30E+03 | | |
| | | | C | 5 | 7 | 1 | 5.00E+02 | 7.00E+02 | 6.00E+02 | | |
| | | 2.5 | A | 0 | 0 | 1 | 5.00E+01 | 5.00E+01 | 5.00E+01 | 5.00E+1 | 0.00E+0 |
| | | | B | 0 | 0 | 1 | 5.00E+01 | 5.00E+01 | 5.00E+01 | | |
| | | | C | 0 | 0 | 1 | 5.00E+01 | 5.00E+01 | 5.00E+01 | | |
| | | 5 | A | 0 | 0 | 1 | 5.00E+01 | 5.00E+01 | 5.00E+01 | 5.00E+1 | 0.00E+0 |
| | | | B | 0 | 0 | 1 | 5.00E+01 | 5.00E+01 | 5.00E+01 | | |
| | | | C | 0 | 0 | 1 | 5.00E+01 | 5.00E+01 | 5.00E+01 | | |
| | SEQ ID | 0 | A | 11 | 6 | 1 | 1.10E+03 | 6.00E+02 | 8.50E+02 | 1.79E+4 | 2.69E+4 |
| | NO: 1 | | B | 32 | 47 | 1 | 3.20E+03 | 4.70E+03 | 3.95E+03 | | |
| | 10 mg/mL | | C | 49 | X | 10 | 4.90E+04 | | 4.90E+04 | | |
| | | 1 | A | 11 | 8 | 1 | 1.10E+03 | 8.00E+02 | 9.50E+02 | 9.33E+2 | 1.26E+2 |
| | | | B | 9 | 7 | 1 | 9.00E+02 | 7.00E+02 | 8.00E+02 | | |
| | | | C | 10 | 11 | 1 | 1.00E+03 | 1.10E+03 | 1.05E+03 | | |
| | | 2.5 | A | 0 | 0 | 1 | 5.00E+01 | 5.00E+01 | 5.00E+01 | 5.00E+1 | 0.00E+0 |
| | | | B | 0 | 0 | 1 | 5.00E+01 | 5.00E+01 | 5.00E+01 | | |
| | | | C | 1 | 0 | 1 | 1.00E+02 | 0.00E+00 | 5.00E+01 | | |
| | | 5 | A | 0 | 0 | 1 | 5.00E+01 | 5.00E+01 | 5.00E+01 | 5.00E+1 | 0.00E+0 |
| | | | B | 1 | 0 | 1 | 1.00E+02 | 0.00E+00 | 5.00E+01 | | |
| | | | C | 0 | 0 | 1 | 5.00E+01 | 5.00E+01 | 5.00E+01 | | |
| 100 | None | 0 | A | 10 | 13 | 10 | 1.00E+04 | 1.30E+04 | 1.15E+04 | 7.55E+4 | 7.30E+4 |
| | | | B | 58 | 62 | 10 | 5.80E+04 | 6.20E+04 | 6.00E+04 | | |
| | | | C | 18 | 13 | 100 | 1.80E+05 | 1.30E+05 | 1.55E+05 | | |
| | | 1 | A | 13 | 11 | 100 | 1.30E+05 | 1.10E+05 | 1.20E+05 | 8.02E+4 | 4.25E+4 |
| | | | B | 29 | 42 | 10 | 2.90E+04 | 4.20E+04 | 3.55E+04 | | |
| | | | C | 12 | 5 | 100 | 1.20E+05 | 5.00E+04 | 8.50E+04 | | |
| | | 2.5 | A | 12 | 14 | 100 | 1.20E+05 | 1.40E+05 | 1.30E+05 | 1.17E+5 | 2.31E+4 |
| | | | B | 12 | 6 | 100 | 1.20E+05 | 6.00E+04 | 9.00E+04 | | |
| | | | C | 12 | 14 | 100 | 1.20E+05 | 1.40E+05 | 1.30E+05 | | |
| | | 5 | A | 54 | 48 | 10 | 5.40E+04 | 4.80E+04 | 5.10E+04 | 2.75E+4 | 2.20E+4 |
| | | | B | 21 | 27 | 10 | 2.10E+04 | 2.70E+04 | 2.40E+04 | | |
| | | | C | 78 | 69 | 1 | 7.80E+03 | 6.90E+03 | 7.35E+03 | | |
| | SEQ ID | 0 | A | 93 | 94 | 10 | 9.30E+04 | 9.40E+04 | 9.35E+04 | 4.70E+4 | 4.03E+4 |
| | NO: 1 | | B | 23 | 28 | 10 | 2.30E+04 | 2.80E+04 | 2.55E+04 | | |
| | 1 mg/mL | | C | 19 | 25 | 10 | 1.90E+04 | 2.50E+04 | 2.20E+04 | | |
| | | 1 | A | 63 | 62 | 1 | 6.30E+03 | 6.20E+03 | 6.25E+03 | 4.22E+3 | 1.84E+3 |
| | | | B | 37 | 38 | 1 | 3.70E+03 | 3.80E+03 | 3.75E+03 | | |
| | | | C | 25 | 28 | 1 | 2.50E+03 | 2.80E+03 | 2.65E+03 | | |
| | | 2.5 | A | 16 | 19 | 1 | 1.60E+03 | 1.90E+03 | 1.75E+03 | 1.62E+3 | 7.59E+2 |
| | | | B | 18 | 28 | 1 | 1.80E+03 | 2.80E+03 | 2.30E+03 | | |
| | | | C | 8 | 8 | 1 | 8.00E+02 | 8.00E+02 | 8.00E+02 | | |
| | | 5 | A | 0 | 0 | 1 | 5.00E+01 | 5.00E+01 | 5.00E+01 | 3.00E+2 | 3.91E+2 |
| | | | B | 1 | 1 | 1 | 1.00E+02 | 1.00E+02 | 1.00E+02 | | |
| | | | C | 7 | 8 | 1 | 7.00E+02 | 8.00E+02 | 7.50E+02 | | |
| | SEQ ID | 0 | A | 10 | 9 | 100 | 1.00E+05 | 9.00E+04 | 9.50E+04 | 7.02E+4 | 4.74E+4 |
| | NO: 1 | | B | 11 | 9 | 100 | 1.10E+05 | 9.00E+04 | 1.00E+05 | | |
| | 3 mg/mL | | C | 17 | 14 | 10 | 1.70E+04 | 1.40E+04 | 1.55E+04 | | |
| | | 1 | A | 6 | 6 | 1 | 6.00E+02 | 6.00E+02 | 6.00E+02 | 2.05E+4 | 3.46E+4 |
| | | | B | 6 | 3 | 1 | 6.00E+02 | 3.00E+02 | 4.50E+02 | | |
| | | | C | 57 | 64 | 10 | 5.70E+04 | 6.40E+04 | 6.05E+04 | | |
| | | 2.5 | A | 5 | 4 | 1 | 5.00E+02 | 4.00E+02 | 4.50E+02 | 1.31E+4 | 2.03E+4 |
| | | | B | 36 | 37 | 10 | 3.60E+04 | 3.70E+04 | 3.65E+04 | | |
| | | | C | 32 | 16 | 1 | 3.20E+03 | 1.60E+03 | 2.40E+03 | | |
| | | 5 | A | 0 | 0 | 1 | 5.00E+01 | 5.00E+01 | 5.00E+01 | 5.00E+1 | 0.00E+0 |
| | | | B | 0 | 0 | 1 | 5.00E+01 | 5.00E+01 | 5.00E+01 | | |
| | | | C | 0 | 0 | 1 | 5.00E+01 | 5.00E+01 | 5.00E+01 | | |
| | SEQ ID | 0 | A | 6 | 4 | 1 | 6.00E+02 | 4.00E+02 | 5.00E+02 | 1.40E+4 | 1.49E+4 |
| | NO: 1 | | B | 12 | 11 | 10 | 1.20E+04 | 1.10E+04 | 1.15E+04 | | |
| | 10 mg/mL | | C | 26 | 34 | 10 | 2.60E+04 | 3.40E+04 | 3.00E+04 | | |
| | | 1 | A | 19 | 28 | 10 | 1.90E+04 | 2.80E+04 | 2.35E+04 | 8.45E+3 | 1.30E+4 |
| | | | B | 8 | 11 | 1 | 8.00E+02 | 1.10E+03 | 9.50E+02 | | |
| | | | C | 6 | 12 | 1 | 6.00E+02 | 1.20E+03 | 9.00E+02 | | |
| | | 2.5 | A | 0 | 0 | 1 | 5.00E+01 | 5.00E+01 | 5.00E+01 | 1.55E+3 | 2.60E+3 |
| | | | B | 44 | 47 | 1 | 4.40E+03 | 4.70E+03 | 4.55E+03 | | |
| | | | C | 1 | 0 | 1 | 1.00E+02 | 0.00E+00 | 5.00E+01 | | |
| | | 5 | A | 11 | 16 | 10 | 1.10E+04 | 1.60E+04 | 1.35E+04 | 4.55E+3 | 7.75E+3 |
| | | | B | 0 | 0 | 1 | 5.00E+01 | 5.00E+01 | 5.00E+01 | | |
| | | | C | 2 | 0 | 1 | 2.00E+02 | 0.00E+00 | 1.00E+02 | | |
| 150 | None | 0 | A | 14 | 16 | 100 | 1.40E+05 | 1.60E+05 | 1.50E+05 | 8.92E+4 | 5.50E+4 |
| | | | B | 66 | 83 | 10 | 6.60E+04 | 8.30E+04 | 7.45E+04 | | |
| | | | C | 39 | 47 | 10 | 3.90E+04 | 4.70E+04 | 4.30E+04 | | |
| | | 1 | A | 61 | 52 | 10 | 6.10E+04 | 5.20E+04 | 5.65E+04 | 3.58E+4 | 1.79E+4 |
| | | | B | 21 | 30 | 10 | 2.10E+04 | 3.00E+04 | 2.55E+04 | | |
| | | | C | 25 | 26 | 10 | 2.50E+04 | 2.60E+04 | 2.55E+04 | | |
| | | 2.5 | A | 14 | 10 | 100 | 1.40E+05 | 1.00E+05 | 1.20E+05 | 1.06E+5 | 5.73E+4 |
| | | | B | 39 | 47 | 10 | 3.90E+04 | 4.70E+04 | 4.30E+04 | | |
| | | | C | 12 | 19 | 100 | 1.20E+05 | 1.90E+05 | 1.55E+05 | | |
| | | 5 | A | 10 | 7 | 100 | 1.00E+05 | 7.00E+04 | 8.50E+04 | 6.07E+4 | 2.19E+4 |
| | | | B | 55 | 54 | 10 | 5.50E+04 | 5.40E+04 | 5.45E+04 | | |
| | | | C | 42 | 43 | 10 | 4.20E+04 | 4.30E+04 | 4.25E+04 | | |
| | SEQ ID | 0 | A | 12 | 14 | 100 | 1.20E+05 | 1.40E+05 | 1.30E+05 | 7.42E+4 | 4.95E+4 |
| | NO: 1 | | B | 62 | 52 | 10 | 6.20E+04 | 5.20E+04 | 5.70E+04 | | |
| | 1 mg/mL | | C | 34 | 37 | 10 | 3.40E+04 | 3.70E+04 | 3.55E+04 | | |
| | | 1 | A | 7 | 14 | 1 | 7.00E+02 | 1.40E+03 | 1.05E+03 | 2.05E+3 | 8.79E+2 |
| | | | B | 20 | 34 | 1 | 2.00E+03 | 3.40E+03 | 2.70E+03 | | |
| | | | C | 22 | 26 | 1 | 2.20E+03 | 2.60E+03 | 2.40E+03 | | |
| | | 2.5 | A | 8 | 11 | 1 | 8.00E+02 | 1.10E+03 | 9.50E+02 | 1.23E+3 | 4.91E+2 |
| | | | B | 13 | 23 | 1 | 1.30E+03 | 2.30E+03 | 1.80E+03 | | |
| | | | C | 8 | 11 | 1 | 8.00E+02 | 1.10E+03 | 9.50E+02 | | |
| | | 5 | A | 5 | 2 | 1 | 5.00E+02 | 2.00E+02 | 3.50E+02 | 2.67E+2 | 1.04E+2 |
| | | | B | 4 | 2 | 1 | 4.00E+02 | 2.00E+02 | 3.00E+02 | | |
| | | | C | 0 | 3 | 1 | 0.00E+00 | 3.00E+02 | 1.50E+02 | | |
| | SEQ ID | 0 | A | 43 | 57 | 10 | 4.30E+04 | 5.70E+04 | 5.00E+04 | 3.90E+4 | 1.99E+4 |
| | NO: 1 | | B | 14 | 18 | 10 | 1.40E+04 | 1.80E+04 | 1.60E+04 | | |
| | 3 mg/mL | | C | 53 | 49 | 10 | 5.30E+04 | 4.90E+04 | 5.10E+04 | | |
| | | 1 | A | 4 | 12 | 1 | 4.00E+02 | 1.20E+03 | 8.00E+02 | 1.35E+3 | 8.26E+2 |
| | | | B | 22 | 24 | 1 | 2.20E+03 | 2.40E+03 | 2.30E+03 | | |
| | | | C | 9 | 10 | 1 | 9.00E+02 | 1.00E+03 | 9.50E+02 | | |
| | | 2.5 | A | 9 | 11 | 1 | 9.00E+02 | 1.10E+03 | 1.00E+03 | 5.00E+2 | 4.44E+2 |
| | | | B | 2 | 1 | 1 | 2.00E+02 | 1.00E+02 | 1.50E+02 | | |
| | | | C | 3 | 4 | 1 | 3.00E+02 | 4.00E+02 | 3.50E+02 | | |
| | | 5 | A | 2 | 1 | 1 | 2.00E+02 | 1.00E+02 | 1.50E+02 | 3.50E+2 | 3.46E+2 |
| | | | B | 3 | 0 | 1 | 3.00E+02 | 0.00E+00 | 1.50E+02 | | |
| | | | C | 11 | 4 | 1 | 1.10E+03 | 4.00E+02 | 7.50E+02 | | |
| | SEQ ID | 0 | A | 19 | 19 | 10 | 1.90E+04 | 1.90E+04 | 1.90E+04 | 1.60E+4 | 8.89E+3 |
| | NO: 1 | | B | 62 | 58 | 1 | 6.20E+03 | 5.80E+03 | 6.00E+03 | | |
| | 10 mg/mL | | C | 21 | 25 | 10 | 2.10E+04 | 2.50E+04 | 2.30E+04 | | |
| | | 1 | A | 13 | 18 | 1 | 1.30E+03 | 1.80E+03 | 1.55E+03 | 6.33E+2 | 8.04E+2 |
| | | | B | 2 | 4 | 1 | 2.00E+02 | 4.00E+02 | 3.00E+02 | | |
| | | | C | 0 | 1 | 1 | 0.00E+00 | 1.00E+02 | 5.00E+01 | | |
| | | 2.5 | A | 2 | 3 | 1 | 2.00E+02 | 3.00E+02 | 2.50E+02 | 9.50E+2 | 6.08E+2 |
| | | | B | 14 | 13 | 1 | 1.40E+03 | 1.30E+03 | 1.35E+03 | | |
| | | | C | 15 | 10 | 1 | 1.50E+03 | 1.00E+03 | 1.25E+03 | | |
| | | 5 | A | 0 | 0 | 1 | 5.00E+01 | 5.00E+01 | 5.00E+01 | 5.00E+1 | 0.00E+0 |
| | | | B | 0 | 0 | 1 | 5.00E+01 | 5.00E+01 | 5.00E+01 | | |
| | | | C | 0 | 0 | 1 | 5.00E+01 | 5.00E+01 | 5.00E+01 | | |
| 200 | None | 0 | A | 21 | 26 | 100 | 2.10E+05 | 2.60E+05 | 2.35E+05 | 1.58E+5 | 7.51E+4 |
| | | | B | 12 | 19 | 100 | 1.20E+05 | 1.90E+05 | 1.55E+05 | | |
| | | | C | 10 | 7 | 100 | 1.00E+05 | 7.00E+04 | 8.50E+04 | | |
| | | 1 | A | 10 | 8 | 100 | 1.00E+05 | 8.00E+04 | 9.00E+04 | 6.38E+4 | 2.49E+4 |
| | | | B | 63 | 59 | 10 | 6.30E+04 | 5.90E+04 | 6.10E+04 | | |
| | | | C | 44 | 37 | 10 | 4.40E+04 | 3.70E+04 | 4.05E+04 | | |
| | | 2.5 | A | 52 | 45 | 10 | 5.20E+04 | 4.50E+04 | 4.85E+04 | 2.88E+4 | 1.78E+4 |
| | | | B | 27 | 21 | 10 | 2.70E+04 | 2.10E+04 | 2.40E+04 | | |
| | | | C | 12 | 16 | 10 | 1.20E+04 | 1.60E+04 | 1.40E+04 | | |
| | | 5 | A | 31 | 36 | 10 | 3.10E+04 | 3.60E+04 | 3.35E+04 | 7.67E+4 | 7.22E+4 |
| | | | B | 39 | 34 | 10 | 3.90E+04 | 3.40E+04 | 3.65E+04 | | |
| | | | C | 17 | 15 | 100 | 1.70E+05 | 1.50E+05 | 1.60E+05 | | |
| | SEQ ID | 0 | A | 20 | 29 | 100 | 2.00E+05 | 2.90E+05 | 2.45E+05 | 9.97E+4 | 1.26E+5 |
| | NO: 1 | | B | 23 | 34 | 10 | 2.30E+04 | 3.40E+04 | 2.85E+04 | | |
| | 1 mg/mL | | C | 26 | 25 | 10 | 2.60E+04 | 2.50E+04 | 2.55E+04 | | |
| | | 1 | A | 54 | 69 | 1 | 5.40E+03 | 6.90E+03 | 6.15E+03 | 3.28E+3 | 2.68E+3 |
| | | | B | 8 | 9 | 1 | 8.00E+02 | 9.00E+02 | 8.50E+02 | | |
| | | | C | 33 | 24 | 1 | 3.30E+03 | 2.40E+03 | 2.85E+03 | | |
| | | 2.5 | A | 36 | 34 | 1 | 3.60E+03 | 3.40E+03 | 3.50E+03 | 3.52E+3 | 2.43E+3 |
| | | | B | 62 | 57 | 1 | 6.20E+03 | 5.70E+03 | 5.95E+03 | | |
| | | | C | 16 | 6 | 1 | 1.60E+03 | 6.00E+02 | 1.10E+03 | | |
| | | 5 | A | 4 | 2 | 1 | 4.00E+02 | 2.00E+02 | 3.00E+02 | 5.33E+2 | 2.08E+2 |
| | | | B | 9 | 3 | 1 | 9.00E+02 | 3.00E+02 | 6.00E+02 | | |
| | | | C | 8 | 6 | 1 | 8.00E+02 | 6.00E+02 | 7.00E+02 | | |
| | SEQ ID | 0 | A | 17 | 21 | 10 | 1.70E+04 | 2.10E+04 | 1.90E+04 | 2.25E+4 | 6.50E+3 |
| | NO: 1 | | B | 16 | 21 | 10 | 1.60E+04 | 2.10E+04 | 1.85E+04 | | |
| | 3 mg/mL | | C | 25 | 35 | 10 | 2.50E+04 | 3.50E+04 | 3.00E+04 | | |
| | | 1 | A | 55 | 52 | 1 | 5.50E+03 | 5.20E+03 | 5.35E+03 | 3.83E+3 | 1.94E+3 |
| | | | B | 19 | 14 | 1 | 1.90E+03 | 1.40E+03 | 1.65E+03 | | |
| | | | C | 44 | 46 | 1 | 4.40E+03 | 4.60E+03 | 4.50E+03 | | |
| | | 2.5 | A | 0 | 0 | 1 | 5.00E+01 | 5.00E+01 | 5.00E+01 | 5.50E+2 | 6.61E+2 |
| | | | B | 4 | 2 | 1 | 4.00E+02 | 2.00E+02 | 3.00E+02 | | |
| | | | C | 14 | 12 | 1 | 1.40E+03 | 1.20E+03 | 1.30E+03 | | |
| | | 5 | A | 4 | 2 | 1 | 4.00E+02 | 2.00E+02 | 3.00E+02 | 2.00E+2 | 1.00E+2 |
| | | | B | 2 | 0 | 1 | 2.00E+02 | 0.00E+00 | 1.00E+02 | | |
| | | | C | 2 | 2 | 1 | 2.00E+02 | 2.00E+02 | 2.00E+02 | | |
| | SEQ ID | 0 | A | 29 | 30 | 10 | 2.90E+04 | 3.00E+04 | 2.95E+04 | 1.82E+4 | 1.10E+4 |
| | NO: 1 | | B | 72 | 80 | 1 | 7.20E+03 | 8.00E+03 | 7.60E+03 | | |
| | 10 mg/mL | | C | 20 | 15 | 10 | 2.00E+04 | 1.50E+04 | 1.75E+04 | | |
| | | 1 | A | 27 | 21 | 1 | 2.70E+03 | 2.10E+03 | 2.40E+03 | 2.10E+3 | 6.54E+2 |
| | | | B | 17 | 34 | 1 | 1.70E+03 | 3.40E+03 | 2.55E+03 | | |
| | | | C | 11 | 16 | 1 | 1.10E+03 | 1.60E+03 | 1.35E+03 | | |
| | | 2.5 | A | 3 | 6 | 1 | 3.00E+02 | 6.00E+02 | 4.50E+02 | 4.00E+2 | 8.66E+1 |
| | | | B | 3 | 3 | 1 | 3.00E+02 | 3.00E+02 | 3.00E+02 | | |
| | | | C | 4 | 5 | 1 | 4.00E+02 | 5.00E+02 | 4.50E+02 | | |
| | | 5 | A | 0 | 0 | 1 | 5.00E+01 | 5.00E+01 | 5.00E+01 | 5.00E+1 | 0.00E+0 |
| | | | B | 0 | 0 | 1 | 5.00E+01 | 5.00E+01 | 5.00E+01 | | |
| | | | C | 0 | 0 | 1 | 5.00E+01 | 5.00E+01 | 5.00E+01 | | |

TABLE 24C
CFU count and dilutions of dilutions of recovered Pseudomonas aeruginosa CDC 0515 from the wire biofilm assay

| Na | | | | | | | | | | | |
|---|---|---|---|---|---|---|---|---|---|---|---|
| Bicarbonate | | Time | | CFU | CFU | | CFU/mL | CFU/mL | | Mean | |
| (mM) | Treatment | (min) | Rep | 1 | 2 | DF | 1 | 2 | CFU/mL | CFU/mL | SD |
| 50 | None | 0 | A | 24 | 25 | 1000 | 2.40E+06 | 2.50E+06 | 2.45E+06 | 7.67E+6 | 8.95E+6 |
| | | | B | 28 | 23 | 1000 | 2.80E+06 | 2.30E+06 | 2.55E+06 | | |
| | | | C | 18 | 18 | 10000 | 1.80E+07 | 1.80E+07 | 1.80E+07 | | |
| | | 1 | A | 42 | 45 | 1000 | 4.20E+06 | 4.50E+06 | 4.35E+06 | 7.97E+6 | 7.87E+6 |
| | | | B | 18 | 16 | 10000 | 1.80E+07 | 1.60E+07 | 1.70E+07 | | |
| | | | C | 25 | 26 | 1000 | 2.50E+06 | 2.60E+06 | 2.55E+06 | | |
| | | 2.5 | A | 60 | 59 | 1000 | 6.00E+06 | 5.90E+06 | 5.95E+06 | 1.09E+7 | 1.19E+7 |
| | | | B | 25 | 21 | 1000 | 2.50E+06 | 2.10E+06 | 2.30E+06 | | |
| | | | C | 23 | 26 | 10000 | 2.30E+07 | 2.60E+07 | 2.45E+07 | | |
| | | 5 | A | 12 | 14 | 10000 | 1.20E+07 | 1.40E+07 | 1.30E+07 | 6.72E+6 | 5.44E+6 |
| | | | B | 35 | 36 | 1000 | 3.50E+06 | 3.60E+06 | 3.55E+06 | | |
| | | | C | 35 | 37 | 1000 | 3.50E+06 | 3.70E+06 | 3.60E+06 | | |
| | SEQ ID | 0 | A | 13 | 27 | 1000 | 1.30E+06 | 2.70E+06 | 2.00E+06 | 5.38E+6 | 4.14E+6 |
| | NO: 1 | | B | 10 | 10 | 10000 | 1.00E+07 | 1.00E+07 | 1.00E+07 | | |
| | 1 mg/mL | | C | 34 | 49 | 1000 | 3.40E+06 | 4.90E+06 | 4.15E+06 | | |
| | | 1 | A | 23 | 41 | 1000 | 2.30E+06 | 4.10E+06 | 3.20E+06 | 7.25E+6 | 5.12E+6 |
| | | | B | 13 | 13 | 10000 | 1.30E+07 | 1.30E+07 | 1.30E+07 | | |
| | | | C | 45 | 66 | 1000 | 4.50E+06 | 6.60E+06 | 5.55E+06 | | |
| | | 2.5 | A | 12 | 5 | 10000 | 1.20E+07 | 5.00E+06 | 8.50E+06 | 3.90E+6 | 4.00E+6 |
| | | | B | 11 | 14 | 1000 | 1.10E+06 | 1.40E+06 | 1.25E+06 | | |
| | | | C | 21 | 18 | 1000 | 2.10E+06 | 1.80E+06 | 1.95E+06 | | |
| | | 5 | A | 28 | 43 | 100 | 2.80E+05 | 4.30E+05 | 3.55E+05 | 1.57E+6 | 2.10E+6 |
| | | | B | 36 | 35 | 100 | 3.60E+05 | 3.50E+05 | 3.55E+05 | | |
| | | | C | 37 | 43 | 1000 | 3.70E+06 | 4.30E+06 | 4.00E+06 | | |
| | SEQ ID | 0 | A | 12 | 14 | 1000 | 1.20E+06 | 1.40E+06 | 1.30E+06 | 1.38E+6 | 1.44E+5 |
| | NO: 1 | | B | 15 | 16 | 1000 | 1.50E+06 | 1.60E+06 | 1.55E+06 | | |
| | 3 mg/mL | | C | 10 | 16 | 1000 | 1.00E+06 | 1.60E+06 | 1.30E+06 | | |
| | | 1 | A | 56 | 55 | 100 | 5.60E+05 | 5.50E+05 | 5.55E+05 | 2.15E+6 | 2.64E+6 |
| | | | B | 52 | 52 | 1000 | 5.20E+06 | 5.20E+06 | 5.20E+06 | | |
| | | | C | 63 | 78 | 100 | 6.30E+05 | 7.80E+05 | 7.05E+05 | | |
| | | 2.5 | A | 51 | 70 | 1000 | 5.10E+06 | 7.00E+06 | 6.05E+06 | 9.03E+6 | 4.33E+6 |
| | | | B | 11 | 17 | 10000 | 1.10E+07 | 1.70E+07 | 1.40E+07 | | |
| | | | C | 78 | 63 | 1000 | 7.80E+06 | 6.30E+06 | 7.05E+06 | | |
| | | 5 | A | 10 | 6 | 100 | 1.00E+05 | 6.00E+04 | 8.00E+04 | 4.58E+5 | 3.31E+5 |
| | | | B | 78 | 61 | 100 | 7.80E+05 | 6.10E+05 | 6.95E+05 | | |
| | | | C | 57 | 63 | 100 | 5.70E+05 | 6.30E+05 | 6.00E+05 | | |
| | SEQ ID | 0 | A | 62 | 81 | 10000 | 6.20E+07 | 8.10E+07 | 7.15E+07 | 3.52E+7 | 3.26E+7 |
| | NO: 1 | | B | 81 | 83 | 1000 | 8.10E+06 | 8.30E+06 | 8.20E+06 | | |
| | 10 mg/mL | | C | 27 | 25 | 10000 | 2.70E+07 | 2.50E+07 | 2.60E+07 | | |
| | | 1 | A | 15 | 15 | 1000 | 1.50E+06 | 1.50E+06 | 1.50E+06 | 1.47E+6 | 1.10E+6 |
| | | | B | 45 | 26 | 100 | 4.50E+05 | 2.60E+05 | 3.55E+05 | | |
| | | | C | 30 | 21 | 1000 | 3.00E+06 | 2.10E+06 | 2.55E+06 | | |
| | | 2.5 | A | 4 | 12 | 10000 | 4.00E+06 | 1.20E+07 | 8.00E+06 | 5.46E+7 | 8.53E+7 |
| | | | B | 158 | 148 | 10000 | 1.58E+08 | 1.48E+08 | 1.53E+08 | | |
| | | | C | 27 | 28 | 1000 | 2.70E+06 | 2.80E+06 | 2.75E+06 | | |
| | | 5 | A | 26 | 21 | 100 | 2.60E+05 | 2.10E+05 | 2.35E+05 | 1.08E+5 | 1.10E+5 |
| | | | B | 51 | 50 | 10 | 5.10E+04 | 5.00E+04 | 5.05E+04 | | |
| | | | C | 35 | 40 | 10 | 3.50E+04 | 4.00E+04 | 3.75E+04 | | |
| 100 | None | 0 | A | 78 | 97 | 1000 | 7.80E+06 | 9.70E+06 | 8.75E+06 | 7.27E+6 | 1.78E+6 |
| | | | B | 46 | 60 | 1000 | 4.60E+06 | 6.00E+06 | 5.30E+06 | | |
| | | | C | 76 | 79 | 1000 | 7.60E+06 | 7.90E+06 | 7.75E+06 | | |
| | | 1 | A | 68 | 84 | 1000 | 6.80E+06 | 8.40E+06 | 7.60E+06 | 1.69E+7 | 2.11E+7 |
| | | | B | 19 | 23 | 1000 | 1.90E+06 | 2.30E+06 | 2.10E+06 | | |
| | | | C | 42 | 40 | 10000 | 4.20E+07 | 4.00E+07 | 4.10E+07 | | |
| | | 2.5 | A | 54 | 81 | 1000 | 5.40E+06 | 8.10E+06 | 6.75E+06 | 9.37E+6 | 2.29E+6 |
| | | | B | 91 | 129 | 1000 | 9.10E+06 | 1.29E+07 | 1.10E+07 | | |
| | | | C | 101 | 106 | 1000 | 1.01E+07 | 1.06E+07 | 1.04E+07 | | |
| | | 5 | A | 98 | 86 | 1000 | 9.80E+06 | 8.60E+06 | 9.20E+06 | 5.73E+6 | 4.50E+6 |
| | | | B | 68 | 62 | 100 | 6.80E+05 | 6.20E+05 | 6.50E+05 | | |
| | | | C | 74 | 73 | 1000 | 7.40E+06 | 7.30E+06 | 7.35E+06 | | |
| | SEQ ID | 0 | A | 19 | 12 | 1000 | 1.90E+06 | 1.20E+06 | 1.55E+06 | 7.08E+6 | 9.45E+6 |
| | NO: 1 | | B | 21 | 15 | 10000 | 2.10E+07 | 1.50E+07 | 1.80E+07 | | |
| | 1 mg/mL | | C | 21 | 13 | 1000 | 2.10E+06 | 1.30E+06 | 1.70E+06 | | |
| | | 1 | A | 67 | 45 | 1000 | 6.70E+06 | 4.50E+06 | 5.60E+06 | 1.15E+7 | 7.17E+6 |
| | | | B | 17 | 22 | 10000 | 1.70E+07 | 2.20E+07 | 1.95E+07 | | |
| | | | C | 14 | 5 | 10000 | 1.40E+07 | 5.00E+06 | 9.50E+06 | | |
| | | 2.5 | A | 17 | 14 | 10000 | 1.70E+07 | 1.40E+07 | 1.55E+07 | 9.77E+6 | 5.06E+6 |
| | | | B | 55 | 64 | 1000 | 5.50E+06 | 6.40E+06 | 5.95E+06 | | |
| | | | C | 83 | 74 | 1000 | 8.30E+06 | 7.40E+06 | 7.85E+06 | | |
| | | 5 | A | 10 | 11 | 1000 | 1.00E+06 | 1.10E+06 | 1.05E+06 | 7.82E+5 | 2.75E+5 |
| | | | B | 48 | 52 | 100 | 4.80E+05 | 5.20E+05 | 5.00E+05 | | |
| | | | C | 73 | 86 | 100 | 7.30E+05 | 8.60E+05 | 7.95E+05 | | |
| | SEQ ID | 0 | A | 12 | 11 | 10000 | 1.20E+07 | 1.10E+07 | 1.15E+07 | 1.05E+7 | 5.05E+6 |
| | NO: 1 | | B | 53 | 48 | 1000 | 5.30E+06 | 4.80E+06 | 5.05E+06 | | |
| | 3 mg/mL | | C | 12 | 18 | 10000 | 1.20E+07 | 1.80E+07 | 1.50E+07 | | |
| | | 1 | A | 37 | 23 | 1000 | 3.70E+06 | 2.30E+06 | 3.00E+06 | 1.01E+7 | 1.08E+7 |
| | | | B | 47 | 46 | 1000 | 4.70E+06 | 4.60E+06 | 4.65E+06 | | |
| | | | C | 23 | 22 | 10000 | 2.30E+07 | 2.20E+07 | 2.25E+07 | | |
| | | 2.5 | A | 11 | 7 | 10000 | 1.10E+07 | 7.00E+06 | 9.00E+06 | 5.89E+6 | 4.84E+6 |
| | | | B | 84 | 83 | 1000 | 8.40E+06 | 8.30E+06 | 8.35E+06 | | |
| | | | C | 26 | 37 | 100 | 2.60E+05 | 3.70E+05 | 3.15E+05 | | |
| | | 5 | A | 14 | 8 | 10000 | 1.40E+07 | 8.00E+06 | 1.10E+07 | 5.07E+6 | 5.53E+6 |
| | | | B | 43 | 40 | 1000 | 4.30E+06 | 4.00E+06 | 4.15E+06 | | |
| | | | C | 53 | 57 | 10 | 5.30E+04 | 5.70E+04 | 5.50E+04 | | |
| | SEQ ID | 0 | A | 23 | 16 | 10000 | 2.30E+07 | 1.60E+07 | 1.95E+07 | 1.43E+7 | 8.95E+6 |
| | NO: 1 | | B | 16 | 23 | 10000 | 1.60E+07 | 2.30E+07 | 1.95E+07 | | |
| | 10 mg/mL | | C | 41 | 39 | 1000 | 4.10E+06 | 3.90E+06 | 4.00E+06 | | |
| | | 1 | A | 70 | 88 | 100 | 7.00E+05 | 8.80E+05 | 7.90E+05 | 9.80E+5 | 2.79E+5 |
| | | | B | 14 | 12 | 1000 | 1.40E+06 | 1.20E+06 | 1.30E+06 | | |
| | | | C | 10 | 7 | 1000 | 1.00E+06 | 7.00E+05 | 8.50E+05 | | |
| | | 2.5 | A | 38 | 36 | 10000 | 3.80E+07 | 3.60E+07 | 3.70E+07 | 1.29E+7 | 2.09E+7 |
| | | | B | 14 | 13 | 1000 | 1.40E+06 | 1.30E+06 | 1.35E+06 | | |
| | | | C | 17 | 24 | 100 | 1.70E+05 | 2.40E+05 | 2.05E+05 | | |
| | | 5 | A | 32 | 46 | 1000 | 3.20E+06 | 4.60E+06 | 3.90E+06 | 4.17E+6 | 4.01E+6 |
| | | | B | 84 | 82 | 1000 | 8.40E+06 | 8.20E+06 | 8.30E+06 | | |
| | | | C | 29 | 31 | 100 | 2.90E+05 | 3.10E+05 | 3.00E+05 | | |
| 150 | None | 0 | A | 13 | 17 | 1000 | 1.30E+06 | 1.70E+06 | 1.50E+06 | 1.63E+7 | 1.81E+7 |
| | | | B | 39 | 34 | 10000 | 3.90E+07 | 3.40E+07 | 3.65E+07 | | |
| | | | C | 11 | 11 | 10000 | 1.10E+07 | 1.10E+07 | 1.10E+07 | | |
| | | 1 | A | 17 | 26 | 10000 | 1.70E+07 | 2.60E+07 | 2.15E+07 | 8.58E+6 | 1.12E+7 |
| | | | B | 24 | 34 | 1000 | 2.40E+06 | 3.40E+06 | 2.90E+06 | | |
| | | | C | 16 | 11 | 1000 | 1.60E+06 | 1.10E+06 | 1.35E+06 | | |
| | | 2.5 | A | 17 | 25 | 10000 | 1.70E+07 | 2.50E+07 | 2.10E+07 | 1.55E+7 | 6.73E+6 |
| | | | B | 22 | 13 | 10000 | 2.20E+07 | 1.30E+07 | 1.75E+07 | | |
| | | | C | 6 | 10 | 10000 | 6.00E+06 | 1.00E+07 | 8.00E+06 | | |
| | | 5 | A | 23 | 23 | 10000 | 2.30E+07 | 2.30E+07 | 2.30E+07 | 1.48E+7 | 1.21E+7 |
| | | | B | 85 | 88 | 100 | 8.50E+05 | 8.80E+05 | 8.65E+05 | | |
| | | | C | 25 | 16 | 10000 | 2.50E+07 | 1.60E+07 | 2.05E+07 | | |
| | SEQ ID | 0 | A | 13 | 9 | 10000 | 1.30E+07 | 9.00E+06 | 1.10E+07 | 6.58E+6 | 3.88E+6 |
| | NO: 1 | | B | 36 | 39 | 1000 | 3.60E+06 | 3.90E+06 | 3.75E+06 | | |
| | 1 mg/mL | | C | 45 | 55 | 1000 | 4.50E+06 | 5.50E+06 | 5.00E+06 | | |
| | | 1 | A | 19 | 21 | 10000 | 1.90E+07 | 2.10E+07 | 2.00E+07 | 1.33E+7 | 9.13E+6 |
| | | | B | 30 | 28 | 1000 | 3.00E+06 | 2.80E+06 | 2.90E+06 | | |
| | | | C | 18 | 16 | 10000 | 1.80E+07 | 1.60E+07 | 1.70E+07 | | |
| | | 2.5 | A | 11 | 6 | 10000 | 1.10E+07 | 6.00E+06 | 8.50E+06 | 1.03E+8 | 8.17E+7 |
| | | | B | T | T | 10000 | 1.50E+08 | 1.50E+08 | 1.50E+08 | | |
| | | | C | T | T | 10000 | 1.50E+08 | 1.50E+08 | 1.50E+08 | | |
| | | 5 | A | 9 | 13 | 10000 | 9.00E+06 | 1.30E+07 | 1.10E+07 | 5.73E+6 | 4.92E+6 |
| | | | B | 16 | 9 | 1000 | 1.60E+06 | 9.00E+05 | 1.25E+06 | | |
| | | | C | 46 | 53 | 1000 | 4.60E+06 | 5.30E+06 | 4.95E+06 | | |
| | SEQ ID | 0 | A | 97 | 110 | 1000 | 9.70E+06 | 1.10E+07 | 1.04E+07 | 1.60E+7 | 5.16E+6 |
| | NO: 1 | | B | 15 | 19 | 10000 | 1.50E+07 | 1.90E+07 | 1.70E+07 | | |
| | 3 mg/mL | | C | 20 | 21 | 10000 | 2.00E+07 | 2.10E+07 | 2.05E+07 | | |
| | | 1 | A | 42 | 51 | 1000 | 4.20E+06 | 5.10E+06 | 4.65E+06 | 8.32E+6 | 8.01E+6 |
| | | | B | 28 | 28 | 1000 | 2.80E+06 | 2.80E+06 | 2.80E+06 | | |
| | | | C | 18 | 17 | 10000 | 1.80E+07 | 1.70E+07 | 1.75E+07 | | |
| | | 2.5 | A | 47 | 41 | 1000 | 4.70E+06 | 4.10E+06 | 4.40E+06 | 3.53E+6 | 1.72E+6 |
| | | | B | 47 | 46 | 1000 | 4.70E+06 | 4.60E+06 | 4.65E+06 | | |
| | | | C | 15 | 16 | 1000 | 1.50E+06 | 1.60E+06 | 1.55E+06 | | |
| | | 5 | A | 72 | 67 | 10000 | 7.20E+07 | 6.70E+07 | 6.95E+07 | 2.37E+7 | 3.97E+7 |
| | | | B | 52 | 65 | 100 | 5.20E+05 | 6.50E+05 | 5.85E+05 | | |
| | | | C | 88 | 104 | 100 | 8.80E+05 | 1.04E+06 | 9.60E+05 | | |
| | SEQ ID | 0 | A | 12 | 14 | 10000 | 1.20E+07 | 1.40E+07 | 1.30E+07 | 2.10E+7 | 8.85E+6 |
| | NO: 1 | | B | 34 | 27 | 10000 | 3.40E+07 | 2.70E+07 | 3.05E+07 | | |
| | 10 mg/mL | | C | 16 | 23 | 10000 | 1.60E+07 | 2.30E+07 | 1.95E+07 | | |
| | | 1 | A | 17 | 28 | 1000 | 1.70E+06 | 2.80E+06 | 2.25E+06 | 8.55E+6 | 8.33E+6 |
| | | | B | 51 | 57 | 1000 | 5.10E+06 | 5.70E+06 | 5.40E+06 | | |
| | | | C | 17 | 19 | 10000 | 1.70E+07 | 1.90E+07 | 1.80E+07 | | |
| | | 2.5 | A | 31 | 39 | 1000 | 3.10E+06 | 3.90E+06 | 3.50E+06 | 2.43E+6 | 9.25E+5 |
| | | | B | 17 | 20 | 1000 | 1.70E+06 | 2.00E+06 | 1.85E+06 | | |
| | | | C | 17 | 22 | 1000 | 1.70E+06 | 2.20E+06 | 1.95E+06 | | |
| | | 5 | A | 5 | 10 | 10000 | 5.00E+06 | 1.00E+07 | 7.50E+06 | 3.57E+6 | 3.71E+6 |
| | | | B | 12 | 12 | 100 | 1.20E+05 | 1.20E+05 | 1.20E+05 | | |
| | | | C | 41 | 21 | 1000 | 4.10E+06 | 2.10E+06 | 3.10E+06 | | |
| 200 | None | 0 | A | 18 | 23 | 1000 | 1.80E+06 | 2.30E+06 | 2.05E+06 | 1.44E+7 | 1.39E+7 |
| | | | B | 13 | 10 | 10000 | 1.30E+07 | 1.00E+07 | 1.15E+07 | | |
| | | | C | 25 | 34 | 10000 | 2.50E+07 | 3.40E+07 | 2.95E+07 | | |
| | | 1 | A | 12 | 17 | 1000 | 1.20E+06 | 1.70E+06 | 1.45E+06 | 1.25E+7 | 1.78E+7 |
| | | | B | 29 | 31 | 1000 | 2.90E+06 | 3.10E+06 | 3.00E+06 | | |
| | | | C | 32 | 34 | 10000 | 3.20E+07 | 3.40E+07 | 3.30E+07 | | |
| | | 2.5 | A | 17 | 15 | 10000 | 1.70E+07 | 1.50E+07 | 1.60E+07 | 1.07E+7 | 5.80E+6 |
| | | | B | 46 | 44 | 1000 | 4.60E+06 | 4.40E+06 | 4.50E+06 | | |
| | | | C | 11 | 12 | 10000 | 1.10E+07 | 1.20E+07 | 1.15E+07 | | |
| | | 5 | A | 35 | 33 | 10000 | 3.50E+07 | 3.30E+07 | 3.40E+07 | 1.53E+7 | 1.63E+7 |
| | | | B | 41 | 35 | 1000 | 4.10E+06 | 3.50E+06 | 3.80E+06 | | |
| | | | C | 79 | 82 | 1000 | 7.90E+06 | 8.20E+06 | 8.05E+06 | | |
| | SEQ ID | 0 | A | 17 | 14 | 1000 | 1.70E+06 | 1.40E+06 | 1.55E+06 | 2.55E+7 | 2.08E+7 |
| | NO: 1 | | B | 43 | 29 | 10000 | 4.30E+07 | 2.90E+07 | 3.60E+07 | | |
| | 1 mg/mL | | C | 42 | 36 | 10000 | 4.20E+07 | 3.60E+07 | 3.90E+07 | | |
| | | 1 | A | 62 | 69 | 1000 | 6.20E+06 | 6.90E+06 | 6.55E+06 | 2.22E+7 | 2.85E+7 |
| | | | B | 54 | 56 | 10000 | 5.40E+07 | 5.60E+07 | 5.50E+07 | | |
| | | | C | 46 | 52 | 1000 | 4.60E+06 | 5.20E+06 | 4.90E+06 | | |
| | | 2.5 | A | 18 | 12 | 10000 | 1.80E+07 | 1.20E+07 | 1.50E+07 | 1.92E+7 | 1.42E+7 |
| | | | B | 76 | 73 | 1000 | 7.60E+06 | 7.30E+06 | 7.45E+06 | | |
| | | | C | 31 | 39 | 10000 | 3.10E+07 | 3.90E+07 | 3.50E+07 | | |
| | | 5 | A | 79 | 71 | 10 | 7.90E+04 | 7.10E+04 | 7.50E+04 | 1.76E+6 | 1.89E+6 |
| | | | B | 41 | 35 | 1000 | 4.10E+06 | 3.50E+06 | 3.80E+06 | | |
| | | | C | 12 | 16 | 1000 | 1.20E+06 | 1.60E+06 | 1.40E+06 | | |
| | SEQ ID | 0 | A | 16 | 25 | 10000 | 1.60E+07 | 2.50E+07 | 2.05E+07 | 1.39E+7 | 6.22E+6 |
| | NO: 1 | | B | 85 | 78 | 1000 | 8.50E+06 | 7.80E+06 | 8.15E+06 | | |
| | 3 mg/mL | | C | 11 | 15 | 10000 | 1.10E+07 | 1.50E+07 | 1.30E+07 | | |
| | | 1 | A | 32 | 35 | 1000 | 3.20E+06 | 3.50E+06 | 3.35E+06 | 5.93E+6 | 6.66E+6 |
| | | | B | 10 | 9 | 1000 | 1.00E+06 | 9.00E+05 | 9.50E+05 | | |
| | | | C | 10 | 17 | 10000 | 1.00E+07 | 1.70E+07 | 1.35E+07 | | |
| | | 2.5 | A | 10 | 12 | 10000 | 1.00E+07 | 1.20E+07 | 1.10E+07 | 1.24E+7 | 9.55E+6 |
| | | | B | 43 | 28 | 1000 | 4.30E+06 | 2.80E+06 | 3.55E+06 | | |
| | | | C | 27 | 18 | 10000 | 2.70E+07 | 1.80E+07 | 2.25E+07 | | |
| | | 5 | A | 22 | 16 | 10000 | 2.20E+07 | 1.60E+07 | 1.90E+07 | 1.07E+7 | 7.17E+6 |
| | | | B | 59 | 66 | 1000 | 5.90E+06 | 6.60E+06 | 6.25E+06 | | |
| | | | C | 69 | 70 | 1000 | 6.90E+06 | 7.00E+06 | 6.95E+06 | | |
| | SEQ ID | 0 | A | 106 | 96 | 1000 | 1.06E+07 | 9.60E+06 | 1.01E+07 | 1.61E+7 | 1.26E+7 |
| | NO: 1 | | B | 76 | 76 | 1000 | 7.60E+06 | 7.60E+06 | 7.60E+06 | | |
| | 10 mg/mL | | C | 34 | 27 | 10000 | 3.40E+07 | 2.70E+07 | 3.05E+07 | | |
| | | 1 | A | 107 | 121 | 1000 | 1.07E+07 | 1.21E+07 | 1.14E+07 | 9.82E+6 | 1.38E+6 |
| | | | B | 95 | 83 | 1000 | 9.50E+06 | 8.30E+06 | 8.90E+06 | | |
| | | | C | 92 | 91 | 1000 | 9.20E+06 | 9.10E+06 | 9.15E+06 | | |
| | | 2.5 | A | 25 | 33 | 1000 | 2.50E+06 | 3.30E+06 | 2.90E+06 | 3.01E+6 | 2.74E+6 |
| | | | B | 34 | 32 | 100 | 3.40E+05 | 3.20E+05 | 3.30E+05 | | |
| | | | C | 55 | 61 | 1000 | 5.50E+06 | 6.10E+06 | 5.80E+06 | | |
| | | 5 | A | 13 | 10 | 1000 | 1.30E+06 | 1.00E+06 | 1.15E+06 | 6.65E+5 | 4.95E+5 |
| | | | B | 18 | 14 | 100 | 1.80E+05 | 1.40E+05 | 1.60E+05 | | |
| | | | C | 68 | 69 | 100 | 6.80E+05 | 6.90E+05 | 6.85E+05 | | |

### Example 4: Peptide Compositions for Treating Periprosthetic Joint Infection (PA)

This example provides exemplary pharmaceutical formulations of the peptide corresponding to SEQ ID NO: 1, aqueous sodium bicarbonate, and water for treating periprosthetic joint infection (PJI) for patients after Total Knee Arthroplasty (TKA) due to susceptible organisms in patients undergoing a Debridement, Antibiotics, and Implant Retention (DAIR) procedure.

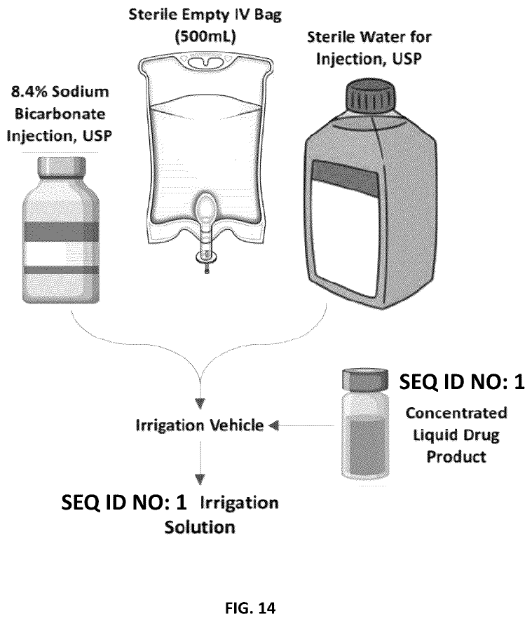

An exemplary kit is depicted in FIG. 14, where the pharmaceutical formulation for irrigation comprises sodium bicarbonate and water for injection for mixing as the irrigation vehicle for final combination with SEQ ID NO: 1. The vial of the concentrated SEQ ID NO: 1 (40.7 mg/mL, pH 5) was mixed with 75 mL aqueous 8.4% sodium bicarbonate and 425 mL of water in a 500 mL sterile IV bag to result in the pharmaceutical formulation. The peptide was diluted in irrigation solution to a final concentration of 3 mg/mL, 5 mg/mL, and 10 mg/mL.

The stability of irrigation solutions of SEQ ID NO: 1 at 1 mg/mE, 3 mg/mL and 10 mg/mL with sodium bicarbonate in IV bags were assessed at monitored room temperature (MRT) under ambient conditions and refrigerated conditions (2-8° C.). The monitored room temperature MRT pharmaceutical formulations were sampled periodically up to 8 hours at testing points 0, 4, and 8 hours. The refrigerated pharmaceutical formulations were sampled periodically up to 48 hours at testing points 0, 12, 24, and 48 hours. Control bags of just the 8.4% sodium bicarbonate and water for injection (no peptide present) was tested. The study provides support for diluted irrigation solution before administration into a patient at a clinical site. The collected samples analyzed appearance, pH, HPLC-UV assay/related substances and osmolality.

The irrigation solutions were prepared by mixing SEQ ID NO 1 with WFI in an IV bag. Subsequently, the 8.4% sodium bicarbonate was added into the IV bag and mixed. The final amount irrigation solution was 150 mL, 22.5 mL of sodium bicarbonate was added, and the amount SEQ ID NO: 1 varied for the final concentration.

TABLE 25A
Stability Results for 1 mg/mL Irrigation Solution at 2-8° C.

| | | Time (hours) | | | |
|---|---|---|---|---|---|
| Test | Acceptance Criteria | 0 | 12 | 24 | 48 |
| Appearance | Clear, colorless solution. Free of | Conforms | Conforms | Conforms | Conforms |
| | particulates | | | | |
| pH | | 8.1 | 8.1 | 8.0 | 8.3 |
| HPLC Assay | 90.0-110.0% | 101.5 | 103.1 | 102.5 | 102.4 |
| HPLC | % | 0.9 | 1.0 | 1.0 | 1.1 |
| Impurities | | | | | |
| Osmolality | mOsmol/kg | 365 | 343 | 335 | 337 |

TABLE 25B
Stability Results for 3 mg/mL Irrigation Solution at 2-8° C.

| | | Time (hours) | | | |
|---|---|---|---|---|---|
| Test | Acceptance Criteria | 0 | 12 | 24 | 48 |
| Appearance | Clear, colorless solution. Free of | Conforms | Conforms | Conforms | Conforms |
| | particulates | | | | |
| pH | | 8.2 | 7.9 | 8.1 | 8.1 |
| HPLC Assay | 90.0-110.0% | 103.9 | 105.9 | 103.2 | 105.0 |
| HPLC | % | 0.9 | 1.1 | 1.0 | 1.1 |
| Impurities | | | | | |
| Osmolality | mOsmol/kg | 288 | 342 | 335 | 281 |

TABLE 25C
Stability Results for 10 mg/mL Irrigation Solution at 2-8° C.

| | | Time (hours) | | | |
|---|---|---|---|---|---|
| Test | Acceptance Criteria | 0 | 12 | 24 | 48 |
| Appearance | Clear, colorless solution. Free of | Conforms | Conforms | Conforms | Conforms |
| | particulates | | | | |
| pH | | 7.7 | 7.5 | 7.7 | 8.0 |
| HPLC Assay | 90.0-110.0% | 104.1 | 101.3 | 101.2 | 102.7 |
| HPLC | % | 1.0 | 1.3 | 1.3 | 1.1 |
| Impurities | | | | | |
| Osmolality | mOsmol/kg | 317 | 340 | 334 | 278 |

TABLE 26A
Stability Results for 3 mg/mL Irrigation Solution at MRT

| | | Time (hours) | | |
|---|---|---|---|---|
| Test | Acceptance Criteria | 0 | 4 | 8 |
| Appearance | Clear, colorless solution. | Conforms | Conforms | Conforms |
| | Free of particulates | | | |
| pH | | 8.1 | 8.3 | 8.2 |
| HPLC Assay | 90.0-110.0% | 110.1 | 110.5^{A} | 109.9^{A} |
| HPLC Impurities | % | 1.0 | 1.1 | 1.1 |
| Osmolality | mOsmol/kg | 331 | 297 | 414 |

^{A}The values exceed the 110% LC limit likely due to higher concentration of SEQ ID NO: 1 or lower volume of sodium bicarbonate when preparing sample bag.

TABLE 26B
Stability Results for 3 mg/mL Irrigation Solution at MRT

| | | Time (hours) | | |
|---|---|---|---|---|
| Test | Acceptance Criteria | 0 | 4 | 8 |
| Appearance | Clear, colorless solution. | Conforms | Conforms | Conforms |
| | Free of particulates | | | |
| pH | | 8.2 | 7.3 | 8.1 |
| HPLC Assay | 90.0-110.0% | 106.3 | 106.4 | 100.7 |
| HPLC Impurities | % | 0.9 | 1.0 | 0.9 |
| Osmolality | mOsmol/kg | 285 | 296 | 415 |

TABLE 26C
Stability Results for 10 mg/mL Irrigation Solution at MRT

| | | Time (hours) | | |
|---|---|---|---|---|
| Test | Acceptance Criteria | 0 | 4 | 8 |
| Appearance | Clear, colorless solution. | Conforms | Conforms | Conforms |
| | Free of particulates | | | |
| pH | | 7.7 | 7.9 | 7.8 |
| HPLC Assay | 90.0-110.0% | 99.0 | 93.6 | 96.2 |
| HPLC Impurities | % | 1.0 | 3.7 | 2.3 |
| Osmolality | mOsmol/kg | 317 | 319 | 412 |

The control bags for both the refrigerated and MRT conditions showed 000 impurities at 48 hours and 8 hour time points. The stability of all irrigation solutions indicate that the irrigation solutions are stable at MRT for 8 hours and 48 hours when stored at (2-8° C.). Overall, the irrigation solutions with SEQ ID NO: 1 showed no signs of chemical degradation.

### Example 5: Peptide Compositions for Treating Periprosthetic Joint Infection (PJI) in Rabbit Models

This example evaluates pharmaceutical formulations of the peptide corresponding to SEQ ID NO: 1, aqueous sodium bicarbonate, and water for treating periprosthetic knee joint infection in rabbit models.

### SUMMARY

The objective of the study was to evaluate the efficacy of SEQ ID NO: 1 in a periprosthetic joint infection model.

A bone tunnel in the tibial canal was created using a drill with a 1.2 mm or 1.6 mm tungsten carbide drill bit. The bone tunnel was then dried and treated to stimulate acute human PJI following primary arthroplasty. A Kirschner wire implant of 2 cm long with a 0.3 cm long top hook was placed in the bone tunnel and the wound was closed. Prior to closure of the superficial skin layer, 0.1 mL of 2×10⁶ planktonic Staphylococcus aureus SH1000 (CFU/rabbit) in saline was injected into the joint space. A closure was performed, and a biofilm was allowed to become established over a period of 2 days.

At 2 days post infection, irrigation and debridement (I&D) was performed on the infected joint. Treatment with a formulation comprising SEQ ID NO: 1 in NaHCO₃ in water (aqueous sodium bicarbonate) was administered at 1 mg/mL and 5 mg/mL concentration of SEQ ID NO: 1 for 15 minute exposure time. Additionally, an intravenous administration of an antibiotic was administered. Bacterial burden was determined by colony forming unit (CFU) analysis. The experimental groups are shown in Table 27.

TABLE 27
Group and Formulations Tested

| Group | | SEQ ID NO: 1 | Exposure | | |
|---|---|---|---|---|---|
| No. | Vehicle | Conc. (mg/mL) | Time (min) | IV Antibiotic | n |
| 1 | NaHCO₃ in | 0 | 15 | Cefazolin | 8 |
| | water | | | | |
| 2 | NaHCO₃ in | 1.0 | 15 | Cefazolin | 8 |
| | water | | | | |
| 3 | NaHCO₃ in | 5.0 | 15 | Cefazolin | 8 |
| | water | | | | |

Animals were observed up to 28 days. The primary endpoint of these experimental trials was survival/mortality. When an animal was sick or needed to be euthanized, the implant and a part of the tibia was collected post mortem and bacterial burden was determined by CFU analysis.

The following parameters and endpoints were evaluated in this study: X-ray for proper implant placement, bacterial burden by colony forming unit analysis (CFU), general clinical signs, detailed clinical signs, body weights, body weight changes, temperature, mortality, blood culture, erythrocyte sedimentation rate (ESR), and C-reactive protein levels (CRP). Rabbit observed weight decreases, temperatures, and pain scores were typical for this animal model.

All enrolled animals survived to study endpoints or reached humane endpoint status per testing facility IACUC regulations. In-life examinations were as expected with this model and were similar for all experimental groups. Clinical observations were as expected with this model and were similar for all experimental groups. Attending veterinarians were notified when animals exceeded a pain score of 1. After observing the animals, veterinarians advised treatment and observations continue per Testing Facility Protocol. Body weights were considered typical for this infection model and for species, strain, sex, and age of animals used in the study.

Due to the nature of this infection model and from anesthesia, there was a notable weight decrease from days 1 to 10. This was due to inappetence, which is common for this model and from recovery from anesthesia. The weight loss was not considered to be test related. The mean body weight was similar between all groups and there were no statistically significant bodyweight differences in comparison to the vehicle control group for both SEQ ID NO: 1 concentrations. This was also the case for the mean body weight change.

Temperature was considered to be typical for this animal infection model and for the species, strain, sex, and age of animals used in the study. After infection on Day 0, there was an increase in temperature that was typically observed in this model. There were no statistically significant differences in comparison to the control group.

For implant bacterial burden by colony forming unit (CFUs) analysis in NaHCO₃ vehicle (water) used in combination with cefazolin (Group 1) resulted in a 3.1 log reduction in bacterial burden compared to untreated I&D alone. NaHCO₃ combination treatment with SEQ ID NO: 1+cefazolin (Group 2) resulted in a 3.1 log reduction in bacterial burden compared to I&D alone animals. There were no significant differences between the vehicle control group in NaHCO₃+cefazolin (Group 1) and SEQ ID NO: 1+cefazolin treatment groups (Groups 2 and 3).

For the bone bacterial burden, there were no differences between the control group (Group 1) and SEQ ID NO: 1+cefazolin treatment groups (Group 2 and 3). However, there were significant differences between untreated (I&D alone) animals from a previous study and all NaHCO₃ treatment groups. NaHCO₃+cefazolin (group 1) resulted in 3.2 reduction in bacterial burden. This was further improved with addition of 1 mg/mL SEQ ID NO: 1 (NaHCO₃)+cefazolin treatment with 3.6 log reduction in bacterial burden. 5 mg/mL SEQ ID NO: 1 in NaHCO₃+cefazolin had similar results with 3.4 log reduction in bacterial burden.

For the total bacterial burden which includes the implant and bone CFUs, there were no differences between control group (Group 1) and SEQ NO ID: 1+cefazolin treatment groups (Group 2 and 3). When comparing treatment groups to I&D alone untreated animal groups, significant reductions were observed between I&D alone and all NaHCO₃ treatment groups with over a 2 log reduction in bacterial burden. NaHCO₃+cefazolin (Group 1) resulted in a 3.1 log reduction in bacterial burden. 1 mg/mL SEQ ID NO: 1 (NaHCO₃)+cefazolin resulted in a 3.2 log reduction in bacterial burden. Similar results were observed with 5 mg/mL SEQ ID NO: 1 (NaHCO₃)+cefazolin with a 3.1 log reduction in bacterial burden.

For animal study survival, all NaHCO₃ treatment groups resulted in similar survival to SEQ ID NO: 1+cefazolin (PBS) treatment group from a previous study with reduced survival in the control NaHCO₃+cefazolin treatment group (Group 1). All NaHCO₃ treatment groups (Group 1, 2, and 3) achieved disease free survival for 28 days.

Blood culture from survival studies were all negative. Erythrocyte sedimentation rate was similar amongst treatment groups with no difference between the control group (Group 1) and SEQ ID NO: 1 treatment groups (Group 2 and 3). ESR levels for the longest surviving treatment groups NaHCO₃+cefazolin (Group 1) and SEQ ID NO: 1+cefazolin (NaHCO₃) (Groups 2 and 3) leveled off around day 7 post infection and decreased to normal levels (0-22 mm/h) by day 14 post infection.

C-reactive protein levels were similar amongst treatment groups peaking at 3 days post infection and returning to normal by day 14 post infection. There were no differences between the control group (Group 1) and SEQ ID NO: 1 treatment groups (Group 2 and 3).

In summary, for this survival study, more than a 2 log reduction in implant bacterial burden was achieved for NaHCO₃+cefazolin (Group 1), 1.0 mg/mL and 5 mg/mL SEQ ID NO: 1 treatment in NaHCO₃(15 minutes) (Groups 2 and 3). The control group NaHCO₃+cefazolin (Group 1) performed equally well to SEQ ID NO: 1 treatment groups (Group 2 and 3) in reducing bacterial burden. However, 7 out of 8 animals (88%) in this treatment group had formation of large abscess at the site of infection. 1 mg/mL SEQ ID NO: 1+cefazolin treatment (Group 2) resulted in 4 out of 8 animals (50%) with a large abscess at the site of infection. 5 mg/mL SEQ ID NO: 1 (Group 3) resulted in only 2 out of 7 animals (24%) with formation of a medium to large abscess. For this survival study, disease free survival was achieved with 1 mg/mL SEQ ID NO: 1+cefazolin (NaHCO₃) and 5 mg/mL SEQ ID NO: 1+cefazolin (NaHCO₃) with 5 out 8 (62.5%) and 4 out 7 (57%) animals respectfully surviving out to day 28.

### Materials and Methods

One 50 mL vial of 8.4% sodium bicarbonate (NaHCO₃) was dissolved into 1 L sterile water for irrigation resulting in 50 mEq/L NaHCO₃ vehicle. In a small beaker, SEQ ID NO: 1 was added to 50 mEq/L NaHCO₃ and mixed for 5-10 minutes to dissolve using a sterile magnetic stir bar or paddle. The solution was visually inspected for complete dissolution. The pH of the solution was tested. 1% sodium hydroxide or 1% acetic acid was used to adjust the pH to 7.9. The final volume was adjusted quantity sufficient (QS) with 50 mEq/L NaHCO₃ vehicle and the final pH was measured and recorded. The SEQ ID NO: 1 solution was transferred to a sterile 15 mL tube and left at room temperature for treatment. Reserved samples of SEQ ID NO: 1 was stored at −20° C.

The test formulations were prepared at concentrations of 1.0 mg/mL and 5 mg/mL in vehicle (50 mEq/L NaHCO₃), by formulating under aseptic conditions. SEQ ID NO: 1 was added while mixing to approximately 90% of volume with vehicle and mixed until the solution was clear.

### Surgical Procedure.

The left hind limb was shaved and prepared according to Testing Facility SOP using hanging left prep with chlorohexidine scrub and betadine solution. The animal was placed in dorsal recumbency and draped for sterile surgery. The left knee was opened via incision. Vessels in the joint capsule was cauterized to prevent excess blood loss. The joint capsule was open via incision below the patellar tendon. The patellar tendon was dislocated to expose the joint space. The fat pad was removed from the knee area. The tibial canal was located using a 22 gauge needle. The space was widened using a drill with 1.2 mm or 1.6 mm tungsten carbide drill bit. The bone tunnel was dried and treated to simulate acute human PJI following primary arthroplasty. A Kirschner wire implant 2 cm long with a 0.3 cm top hook was placed in the bone tunnel. Proper placement of the Kirschner wire implant was verified by extending the leg and verifying it did not come into comprising contact with any bone, ligaments, and tendons. The surgical field was then irrigated with saline and the wound closed with a continuous suture with 4-0 Vicryl. Prior to closure of the superficial skin layer, 0.1 mL of 2×10⁶ planktonic SH1000 Staphylococcus aureus (CFU per rabbit) in saline was injected into the joint space. A closure was performed with a continuous subcutaneous suture as well as another continuous suture over the outer layer with 4-0 Vicryl. A biofilm was allowed to establish over a period of 2 days. At 2 days post infection, the joint space was irrigated and debrided. The test article was administered by intraarticular injection into the joint space. Two mL of test article at 1 mg/mL and 5 mg/mL concentrations were administered by intraarticular injection into the joint space for 15 minute with no PBS rinse. The test article was kept at room temperature prior to dosing. Surgical sutures were removed two weeks after second surgery date.

One animal was removed from the 5 mg/mL SEQ ID NO: 1 and NaHCO₃ due to surgical complications. There were no unscheduled deaths during the course of this study. The Kirschner wire implant and part of the proximal tibia were collected from the animals post-mortem. Samples were prepared for CFU analysis according to Test Facility SOP.

### Body Weight and Body Weight Change

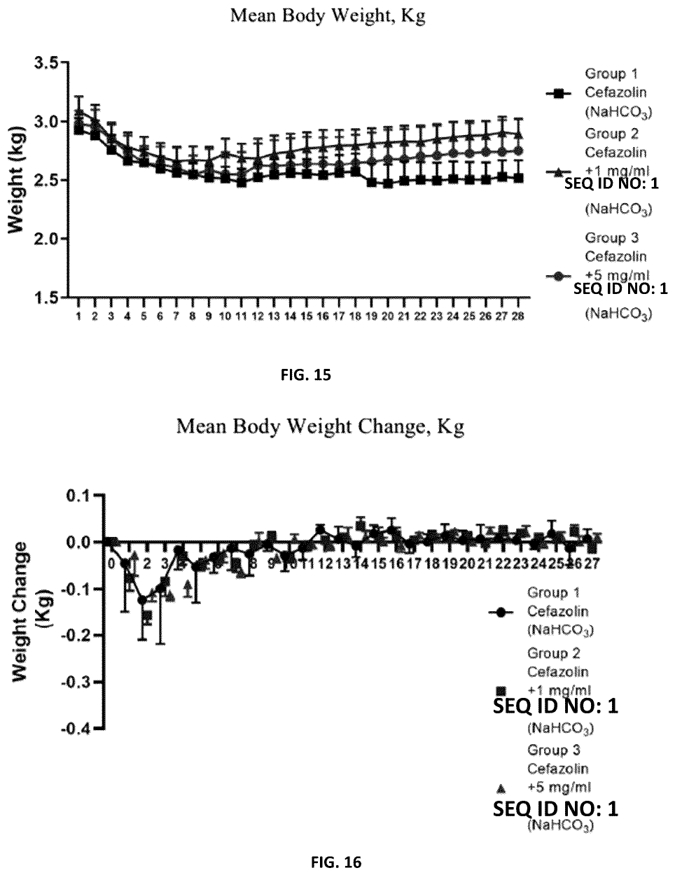

Body weights in FIG. 15 were considered typical for this infection model and for the species, strain, sex and age of animals used in the study. Due to the nature of this infection and from anesthesia, there was a notable weight decrease from days 1 to 10. This was due to inappetence, which was common for this model and from recovery from anesthesia. The weight loss was not considered to be test article (pharmaceutical formulation) related. The mean body weigh was similar between all groups. There were no statistically significant bodyweight differences in comparison to the control group (Group 1) for both SEQ ID NO: 1 concentrations. This was also the case for the mean body weight change. For the body weight change in FIG. 16, results were typical for the species, strain, sex, and age of the animals used in the study. There were no significant differences between treatment groups.

### Temperature and Temperature Change

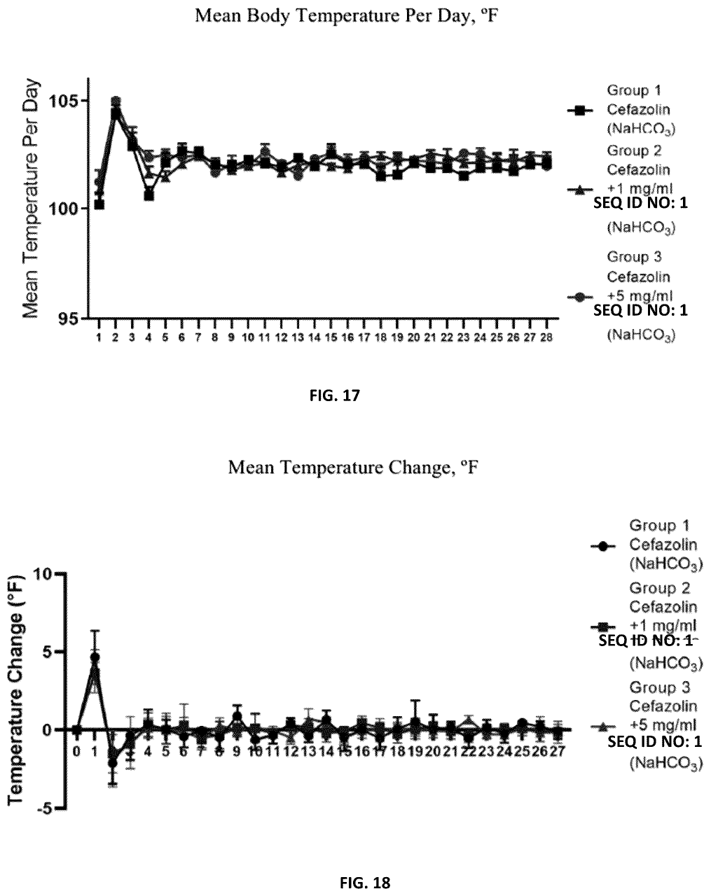

Temperatures were considered to be typical for this animal infection model and for the species, strain, sex and age of the animals used in the study. After infection on Day 0, there was an increase in temperature that was typical for this model. There were no statistically significant differences in comparison to the control group (Group 1) as seen in FIG. 17. The mean temperature change per day was considered typical for this animal infection model and for the species, strain, sex, and age of the animals used in the study. After infection on Day 0, there was a notable temperature change that was typical for this model. There were no differences in comparison to the control group (Group 1) as seen in FIG. 18.

### Implant Colony Forming Unit Analysis

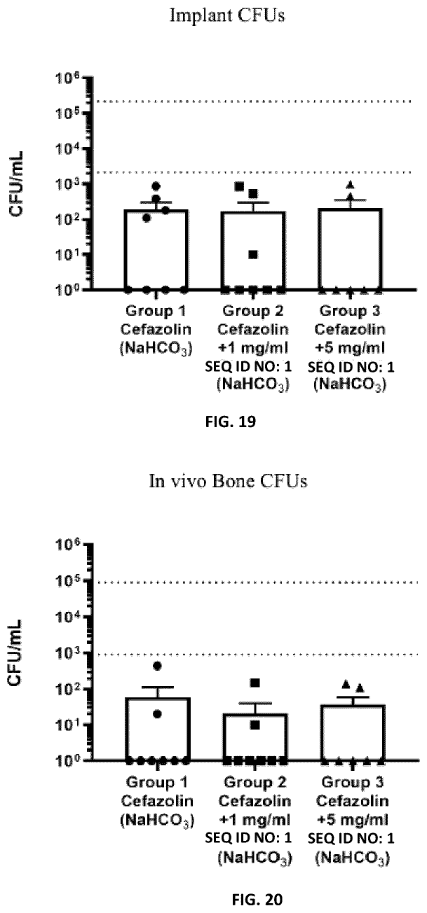

For colony forming unit analysis of Kirschner wire implants, several animals resulted in culture negative implants (*0 CFU/mL). 1 mg/mL and 5 mg/mL SEQ ID NO: 1+cefazolin treatment alone was not in comparison to NaHCO₃+cefazolin vehicle control as seen in FIG. 19. However, NaHCO₃ in combination with cefazolin (Group 1) resulted in a 3.1 log reduction in bacterial burden inn comparison to untreated animals from a previous study. Similarly, 1 mg/mL SEQ ID NO: 1 treatment (NaHCO₃) in combination with cefazolin (Group 2) resulted in a 3.1 log reduction in comparison to untreated animals. Treatment with higher concentrations of SEQ ID NO: 1 did not have a greater benefit. 5 mg/mL SEQ ID NO: 1+cefazolin treatment (Group 3) resulted in a 3.0 log reduction in bacterial burden compared to untreated animals.

### Bone Colony Forming Unit Analysis.

For the bone colony forming unit analysis in FIG. 20, there were no significant differences between treatment groups and the control group. However, when comparing untreated animal groups (I&D alone) from a previous study, the use of NaHCO₃+cefazolin (Group 1) had a greater impact on bone bacterial burden as well as several culture negative animals. NaHCO₃+cefazolin treatment (Group 1) resulted in a 3.2 log reduction in bacterial burden compared to I&D alone animals from a previous study. Treatment with 1 mg/mL SEQ ID NO: 1 in NaHCO₃ in combination with cefazolin (Group 2) resulted in an even greater reduction in bacterial burden (3.6 log reduction). Similar results were observed with 5 mg/mL SEQ ID NO: 1+cefazolin (Group 3) with a 3.4 log reduction in bacterial burden compared to I&D alone animals.

### Total Implant and Bone Bacterial Burden.

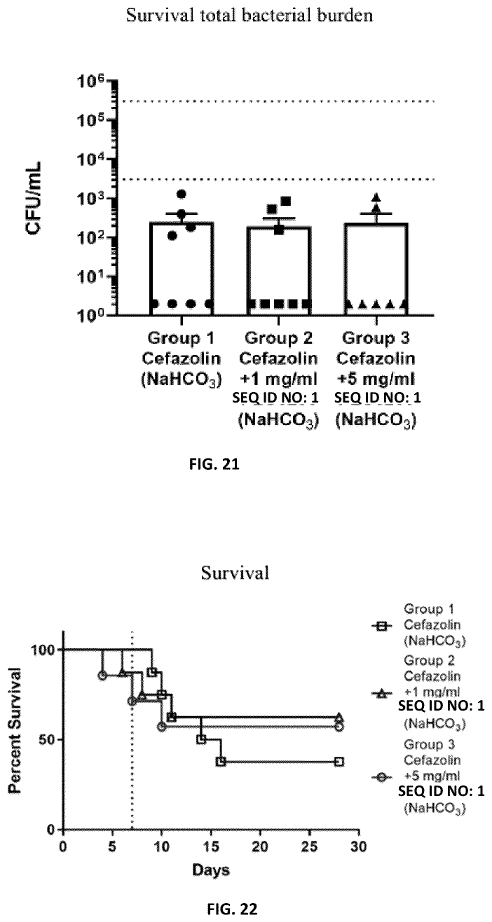

For the total bacterial burden which includes implant and bone CFUs, there were no significant reductions between treatment groups as seen in FIG. 21. However, significant reductions were observed between I&D alone animals from a previous study and all NaHCO₃ treatment groups with over a 2 log reduction in bacterial burden. NaHCO₃+cefazolin (Group 1) resulted in a 3.1 log reduction in bacterial burden compared to I&D alone. 1 mg/mL SEQ ID NO: 1+cefazolin (NaHCO₃) treatment group (Group 2) resulted in a 3.2 log reduction in bacterial burden compared to I&D alone. Similar results were observed with 5 mg/mL SEQ ID NO: 1+cefazolin (NaHCO₃) treatment group (Group 3) with a 3.1 log reduction in bacterial burden. All NaHCO₃ treatment groups achieved at least a 2 log reduction in total bacterial burden.

Upon completion on life studies, 7 out of 8 (88%) NaHCO₃+cefazolin treatment (Group 1) had formation of a large abscess at the site of infection. 1 mg/mL SEQ ID NO: 1 (NaHCO₃)+cefazolin treatment animals had 4 out of 8 (50%) with a large abscess at the site of infection. Higher dose (5 mg/mL) SEQ ID NO: 1 had a markable improvement with only 2 out of 7 (24%) animals with medium to large abscess formation at the site of infection.

### Survival

All NaHCO₃ treatment groups resulted in similar survival wit decreased survival in the NaHCO₃+cefazolin treatment group (Group 1) as seen in FIG. 22. In comparison to I&D alone animals from a previous study, 1 and 5 mg/mL SEQ ID NO: 1+cefazolin (NaHCO₃) treatment had a greater overall survival in comparison to I&D. The blood culture from the survival studies were all negative.

### Erythrocyte Sedimentation Rate

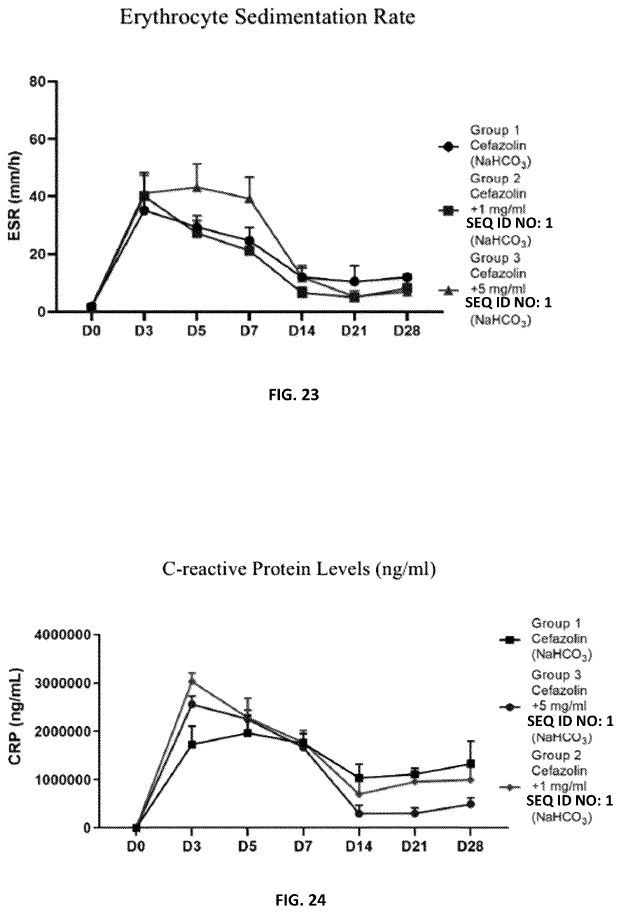

Erythrocyte sedimentation rate for the survival study was similar amongst most treatment groups with no differences between experimental groups as seen in FIG. 23. ESR returned to normal (0-20 mm/h) starting on day 14 post infection.

### C-Reactive Protein

For C-reactive protein, there were no differences between treatment groups as seen in FIG. 24. CRP levels increased for the first 7 days of infection and returned back to normal by 14 days post infection.

In summary, for this survival study, at least a 2 log reduction in implant bacterial burden was achieved for NaHCO₃+cefazolin (Group 1), 1 mg/mL and 5 mg/mL SEQ ID NO: 1 treatment in NaHCO₃(15 minutes) (Groups 2 and 3). The control group NaHCO₃+cefazolin (Group 1) performed equally well to SEQ ID NO: 1 treatment groups (Groups 2 and 3) in reducing bacterial burden. However, 7 our 8 animals (88%) in this treatment group had formation of large abscess at the site of infection. 1 mg/mL SEQ ID NO: 1+cefazolin treatment (Group 2) resulted in 4 out of 8 animals (50%) with large abscess at the site of infection. 5 mg/mL SEQ ID NO: 1+cefazolin (Group 3) resulted in 2 out of 7 animals (28%) with formation of medium to large abscess. For this survival study, disease free survival was achieved with 3 out of 8 (38%) animals surviving out to day 28, 1 mg/mL SEQ ID NO: 1+cefazolin (NaHCO₃) and 5 mg/mL SEQ ID NO: 1+cefazolin (NaHCO₃) with 5 out 8 (62.5%) and 4 out of 7 (57%) animals respectfully surviving out to day 28.

### Example 6: Peptide Composition for Treating PJI in Human Model

This evaluates exemplary pharmaceutical formulations of the peptide corresponding to SEQ ID NO: 1, aqueous sodium bicarbonate, and water for treating periprosthetic joint infection (PJI) in human patients.

For assessing the irrigation formulation of SEQ ID NO: 1 and aqueous sodium bicarbonate in human patients undergoing a DAIR procedure for PJI after TKA, the irrigation solution will be administered to the implant of the patient after debridement. There will be two doses, the first one will be after debridement prior to closure on Day 1 of the DAIR procedure. Each patient will receive a 500 mL of the irrigation solution at dose concentration of 3 mg/mL (Cohort 1) or 10 mg/mL (Cohort 2) as a single intra-articular irrigation to the wound cavity for 15 to 18 minutes. The irrigation administration will follow a 4-step protocol: 1. Pulse lavage with 3 L of normal saline; 2. Lavage with 1 L dilute, povidone iodine (22.5 mL povidone iodine/L normal saline) left in the wound for 3 minutes; 3. Pulse lavage with 3L of normal saline; and 4. Lavage and scrub the retained-in-place prosthetic and surrounding wound in effort to remove the biofilm with 500 mL of the irrigation formulation comprising SEQ ID NO: 1 and sodium bicarbonate at the wound cavity for 15-18 minutes. The wound is suctioned and closed after the irrigation protocol.

Pharmacokinetic (PK), safety, and efficacy endpoints will be measured as part of the assessment. For systemic PK values (e.g., maximum concentration (Cmax), Time to Cmax, elimination half-life, clearance, volume of distribution, area under the concentration curve (AUC), AUC/minimum inhibitory concentration (MIC), Cmax/MIC, and time above MIC), intra-articular irrigation using the irrigation formulation after DAIR on patients is measured on Day 1. Blood biomarkers (e.g., C-reactive protein, interleukin-6, D-dimers, white blood cell count, neutrophil percentage), synovial fluid biomarkers of PJI e.g., erythrocyte, sedimentation rate, C-reactive protein, interleukin-6, D-dimers, white blood cell count, neutrophil percentage, soluble intracellular adhesion molecule-1 (ICAM-1), alpha defensin, and leukocyte esterase), daily step counts, clinical assessment (chemistry, hematology, coagulation, and urinalysis), vital signs, physical examination, incidence of adverse effects (serious AEs and treatment-emergent AEs) are measured on Day 1 and subsequent milestones. The efficacy of the irrigation formulation of SEQ ID NO: 1 and aqueous sodium bicarbonate will be assessed on Days 21, 42, 90, 180, 270, and 365 post-DAIR procedure. The efficacy of the irrigation formulation on recurrence (relapse/reinfection) rates in patients will be assessed on Days 42, 90, 180, 270, and 365 post-DAIR procedure.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A pharmaceutical formulation comprising:
(a) a peptide or pharmaceutically acceptable salt thereof comprising at least about 70% sequence identity to a polypeptide sequence of:
Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1);
Arg-Val-Val-Arg-Val-Val-Arg-Arg-Val-Val-Arg-Arg (SEQ ID NO: 15);
Arg-Val-Val-Arg-Val-Val-Arg-Arg-Trp-Val-Arg-Arg (SEQ ID NO: 16);
Arg-Trp-Trp-Arg-Trp-Trp-Arg-Arg-Trp-Trp-Arg-Arg (SEQ ID NO: 17);
Trp-Arg-Arg-Trp-Trp-Arg-Arg-Trp-Trp-Arg-Trp-Trp-Arg-Arg-Trp-Trp-Arg-Arg (SEQ ID NO: 18);
Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19);
Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20);
Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21);
Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22);
Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23);
Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24);
Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and
(b) aqueous sodium bicarbonate.

2. A pharmaceutical formulation consisting of:
(a) a peptide or pharmaceutically acceptable salt thereof comprising at least about 70% sequence identity to a polypeptide sequence of:
Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1);
Arg-Val-Val-Arg-Val-Val-Arg-Arg-Val-Val-Arg-Arg (SEQ ID NO: 15);
Arg-Val-Val-Arg-Val-Val-Arg-Arg-Trp-Val-Arg-Arg (SEQ ID NO: 16);
Arg-Trp-Trp-Arg-Trp-Trp-Arg-Arg-Trp-Trp-Arg-Arg (SEQ ID NO: 17);
Trp-Arg-Arg-Trp-Trp-Arg-Arg-Trp-Trp-Arg-Trp-Trp-Arg-Arg-Trp-Trp-Arg-Arg (SEQ ID NO: 18);
Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19);
Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20);
Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21);
Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22);
Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23);
Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24);
Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and
(b) aqueous sodium bicarbonate.

3. The pharmaceutical formulation of claim 1 or 2, wherein the pharmaceutical formulation comprises a pH from about 7 to about 11.

4. The pharmaceutical formulation of claim 3, wherein the pharmaceutical formulation comprises the pH from about 8 to about 11.

5. The pharmaceutical formulation of claim 3, wherein the pharmaceutical formulation comprises the pH from about 7 to about 10.

6. The pharmaceutical formulation of claim 3, wherein the pharmaceutical formulation comprises the pH at least about 7.5 or at least about 7.9.

7. The pharmaceutical formulation of any one of claims 1-6, wherein the peptide or pharmaceutically acceptable salt thereof is present in the pharmaceutical formulation at a concentration from about 0.1 mg/mL to about 100 mg/mL.

8. The pharmaceutical formulation of claim 7, wherein the peptide or pharmaceutically acceptable salt thereof is present in the pharmaceutical formulation at a concentration from about 1 mg/mL to about 10 mg/mL.

9. The pharmaceutical formulation of claim 7, wherein the peptide or pharmaceutically acceptable salt thereof is present in the pharmaceutical formulation at a concentration of about 1 mg/mL, about 3 mg/mL, about 5 m/mL, or about 10 mg/mL.

10. The pharmaceutical formulation of any one of claims 1-9, wherein the aqueous sodium bicarbonate is present in the pharmaceutical formulation at a concentration from about 25 mM to about 300 mM.

11. The pharmaceutical formulation of claim 10, wherein the aqueous sodium bicarbonate is present in the pharmaceutical formulation at a concentration of about 150 mM.

12. The pharmaceutical formulation of any one of claims 1-9, wherein the aqueous sodium bicarbonate is present in the pharmaceutical formulation at a concentration from about 5 mg/mL to about 50 mg/mL.

13. The pharmaceutical formulation of claim 12, wherein the aqueous sodium bicarbonate is present in the pharmaceutical formulation at a concentration at about 12.6 mg/mL.

14. The pharmaceutical formulation of claim 1-13, wherein the aqueous sodium bicarbonate has a concentration of about 1 mEq/L to about 200 mEq/mL.

15. The pharmaceutical formulation of claim 14, wherein the aqueous sodium bicarbonate has a concentration of about 50 mEq/L.

16. The pharmaceutical formulation of any one of claims 1-15, wherein the pharmaceutical formulation comprises an osmolality of at least about 30 milliosmoles per kilogram (mOsm/kg) to at least about 800 mOsm/kg.

17. The pharmaceutical formulation of claim 16, wherein the pharmaceutical formulation comprises an osmolality of about 50 mOsm/kg to about 500 mOsm/kg.

18. The pharmaceutical formulation of claim 16, wherein the pharmaceutical formulation comprises an osmolality of about 200 mOsm/kg to about 500 mOsm/kg.

19. The pharmaceutical formulation of any one of claims 1-18, wherein the peptide or salt thereof comprises the polypeptide of sequence Arg Arg Trp Val Arg Arg Val Arg Arg Val Trp Arg Arg Val Val Arg Val Val Arg Arg Trp Val Arg Arg (SEQ ID NO: 1).

20. The pharmaceutical formulation of any one of claims 1-18, wherein the peptide or salt thereof comprises the polypeptide of sequence Arg-Trp-Trp-Arg-Trp-Trp-Arg-Arg-Trp-Trp-Arg-Arg (SEQ ID NO: 17).

21. The pharmaceutical formulation of any one of claims 1-20, wherein the aqueous sodium bicarbonate provides a synergistic effect with the peptide or pharmaceutically acceptable salt thereof.

22. The pharmaceutical formulation of claim 21, wherein the synergistic effect comprises reduces a bacterial burden a greater extent as compared to administering (a) or (b) alone to a subject, reduces the incident of abscesses in a subject, or increases the survivability of a subject.

23. A kit comprising:
(i) a first container comprising a peptide or pharmaceutically acceptable salt thereof comprising at least about 70% sequence identity to a polypeptide
(SEQ ID NO: 1)
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(SEQ ID NO: 15)
Arg-Val-Val-Arg-Val-Val-Arg-Arg-Val-Val-Arg-Arg;
(SEQ ID NO: 16)
Arg-Val-Val-Arg-Val-Val-Arg-Arg-Trp-Val-Arg-Arg;
(SEQ ID NO: 17)
Arg-Trp-Trp-Arg-Trp-Trp-Arg-Arg-Trp-Trp-Arg-Arg;
(SEQ ID NO: 18)
Trp-Arg-Arg-Trp-Trp-Arg-Arg-Trp-Trp-Arg-Trp-Trp-
Arg-Arg-Trp-Trp-Arg-Arg;
(SEQ ID NO: 19)
Arg-Arg-Val-Val-Arg-Arg-Val-Arg-Arg-Val-Val-Arg-
Arg-Val-Val-Arg-Val-Val-Arg-Arg-Val-Val-Arg-Arg;
(SEQ ID NO: 20)
Arg-Arg-Trp-Trp-Arg-Arg-Trp-Arg-Arg-Trp-Trp-Arg-
Arg-Trp-Trp-Arg-Trp-Trp-Arg-Arg-Trp-Trp-Arg-Arg;
(SEQ ID NO: 21)
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
(SEQ ID NO: 22)
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(SEQ ID NO: 23)
Arg-Arg-Val-Val-Arg-Arg-Val-Arg-Arg-Val-Val-Arg-
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg;
(SEQ ID NO: 24)
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
(SEQ ID NO: 25)
Arg-Val-Val-Arg-Val-Val-Arg-Arg-Trp-Val-Arg-Arg-
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Arg-Val-Val,
or any combination thereof; and
(ii) a second container comprising aqueous sodium bicarbonate.

24. The kit of claim 23, wherein kit further comprising (iii) a third container comprising water.

25. The kit of claim 23 or 24, wherein the kit further comprising (iv) a mixing container.

26. The kit of any one of claims 23-25, wherein the mixing container is an intravenous bag, a lavage bottle, or a joint irrigation system.

27. The kit of any one of claims 23-26, wherein the peptide or pharmaceutically acceptable salt thereof is present at a concentration from about 0.1 mg/mL to about 100 mg/mL.

28. The kit of claim 27, wherein the peptide or pharmaceutically acceptable salt thereof is present at a concentration at about 15 mg/mL, about 30 mg/mL, about 40 mg/mL, about 70 mg/mL, or about 80 mg/mL.

29. The kit of any one of claims 23-22, wherein the peptide or pharmaceutically acceptable salt thereof is present at a pH of about 3 to about 7.

30. The kit of claim 29, wherein the peptide or pharmaceutically acceptable salt thereof is present at a pH of about 5.

31. The kit of any one of claims 23-30, wherein the aqueous sodium bicarbonate is present a concentration from about 25 mM to about 300 mM.

32. The kit of any one of claims 23-30, wherein the aqueous sodium bicarbonate is present in at a concentration from about 50 mM to about 200 mM.

33. The kit of any one of claims 23-30, wherein the aqueous sodium bicarbonate is 8.4% sodium bicarbonate.

34. The kit of any one of claims 23-33, wherein the aqueous sodium bicarbonate is at a concentration of about 1 mEq/L to about 200 mEq/L.

35. The kit of claim 34, wherein the aqueous sodium bicarbonate is at a concentration of about 50 mEq/L.

36. The kit of any one of claims 23-35, wherein the peptide or pharmaceutically acceptable salt thereof comprises the polypeptide of sequence Arg Arg Trp Val Arg Arg Val Arg Arg Val Trp Arg Arg Val Val Arg Val Val Arg Arg Trp Val Arg Arg (SEQ ID NO: 1).

37. The kit of any one of claims 23-35, wherein the peptide or pharmaceutically acceptable salt thereof comprises the polypeptide of sequence Arg-Trp-Trp-Arg-Trp-Trp-Arg-Arg-Trp-Trp-Arg-Arg (SEQ ID NO: 17).

38. A method of making a pharmaceutical formulation from a kit, wherein the kit comprises:
(i) a first container comprising a peptide or pharmaceutically acceptable salt thereof comprising at least about 70% sequence identity to a polypeptide sequence of:
(SEQ ID NO: 1)
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(SEQ ID NO: 15)
Arg-Val-Val-Arg-Val-Val-Arg-Arg-Val-Val-Arg-Arg;
(SEQ ID NO: 16)
Arg-Val-Val-Arg-Val-Val-Arg-Arg-Trp-Val-Arg-Arg;
(SEQ ID NO: 17)
Arg-Trp-Trp-Arg-Trp-Trp-Arg-Arg-Trp-Trp-Arg-Arg;
(SEQ ID NO: 18)
Trp-Arg-Arg-Trp-Trp-Arg-Arg-Trp-Trp-Arg-Trp-Trp-
Arg-Arg-Trp-Trp-Arg-Arg;
(SEQ ID NO: 19)
Arg-Arg-Val-Val-Arg-Arg-Val-Arg-Arg-Val-Val-Arg-
Arg-Val-Val-Arg-Val-Val-Arg-Arg-Val-Val-Arg-Arg;
(SEQ ID NO: 20)
Arg-Arg-Trp-Trp-Arg-Arg-Trp-Arg-Arg-Trp-Trp-Arg-
Arg-Trp-Trp-Arg-Trp-Trp-Arg-Arg-Trp-Trp-Arg-Arg;
(SEQ ID NO: 21)
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
(SEQ ID NO: 22)
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Val-Arg-Arg;
(SEQ ID NO: 23)
Arg-Arg-Val-Val-Arg-Arg-Val-Arg-Arg-Val-Val-Arg-
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg;
(SEQ ID NO: 24)
Arg-Val-Val-Arg-Val-Val-Arg-Arg-Val-Val-Arg-Arg-
Val-Arg-Arg-Val-Val-Arg-Arg-Val-Val-Arg-Val-Val-
Arg-Arg-Val-Val-Arg-Arg-Val-Arg-Arg-Val-Val-Arg-
Arg-Val-Val-Arg-Val-Val-Arg-Arg-Val-Val-Arg-Arg;
or
(SEQ ID NO: 25)
Arg-Val-Val-Arg-Val-Val-Arg-Arg-Trp-Val-Arg-Arg-
Val-Arg-Arg-Val-Trp-Arg-Arg-Val-Val-Arg-Val-Val-
Arg-Arg-Trp-Val-Arg-Arg-Val-Arg-Arg-Val-Trp-Arg-
Arg-Val-Val-Arg-Val-Val-Arg-Arg-Trp-Arg-Val-Val;
(ii) a second container comprising aqueous sodium bicarbonate;
(iii) a third container comprising water; and
(iv) a mixing container;
wherein method comprises adding the first container, the second container, and the third container into the mixing container, thereby resulting in the pharmaceutical formulation.

39. The method of claim 38, wherein the pharmaceutical formulation comprises a pH from about 5 to about 11.

40. The method of claim 39, wherein the pharmaceutical formulation comprises the pH from about 7 to about 11.

41. The method of claim 39, wherein the pharmaceutical formulation comprises the pH from about 7 to about 10.

42. The method of claim 38, wherein the pharmaceutical formulation comprises the pH at least about 8.

43. The method of any one of claims 38-42, wherein the peptide or pharmaceutically acceptable salt thereof is present in the pharmaceutical formulation at a concentration from about 1 mg/mL to about 10 mg/mL.

44. The method of any one of claims 38-42, wherein the peptide or pharmaceutically acceptable salt thereof is present in the pharmaceutical formulation at a concentration of about 1 mg/mL, about 3 mg/mL, about 5 m/mL, or about 10 mg/mL.

45. The method of any one of claims 38-44, wherein the aqueous sodium bicarbonate is present in the pharmaceutical formulation at a concentration from about 25 mM to about 300 mM.

46. The method of claim 45, wherein the aqueous sodium bicarbonate is present in the pharmaceutical formulation at a concentration from about 50 mM to about 200 mM.

47. The method of any one of claims 38-46, wherein the aqueous sodium bicarbonate is present in the pharmaceutical formulation at a concentration from about 5 mg/mL to about 50 mg/mL.

48. The method of claim 47, wherein the aqueous sodium bicarbonate is present in the pharmaceutical formulation at a concentration at about 12.6 mg/mL.

49. The method of any one of claims 38-48, wherein the aqueous sodium bicarbonate is present at a concentration of about 1 mEq/L to about 200 mEq/L.

50. The method of claim 49, wherein the aqueous sodium bicarbonate is present at a concentration of about 50 mEq/L.

51. The method of any one of claims 36-50, wherein the pharmaceutical formulation comprises an osmolality of at least about 30 milliosmoles per kilogram (mOsm/kg) to at least about 800 mOsm/kg.

52. The method of claim 51, wherein the pharmaceutical formulation comprises an osmolality of about 50 mOsm/kg to about 500 mOsm/kg.

53. The method of claim 51, wherein the pharmaceutical formulation comprises an osmolality of about 200 mOsm/kg to about 500 mOsm/kg.

54. The method of any one of claims 36-53, wherein the peptide or salt thereof comprises the polypeptide of sequence Arg Arg Trp Val Arg Arg Val Arg Arg Val Trp Arg Arg Val Val Arg Val Val Arg Arg Trp Val Arg Arg (SEQ ID NO: 1).

55. The method of any one of claims 36-53, wherein the peptide or pharmaceutically acceptable salt thereof comprises the polypeptide of sequence Arg-Trp-Trp-Arg-Trp-Trp-Arg-Arg-Trp-Trp-Arg-Arg (SEQ ID NO: 17).

56. A method of preventing or treating an infection in a subject in need thereof, wherein the method comprising locally administering of a pharmaceutical formulation to a site of infection, wherein administration comprises washing, irrigating, debriding, aspirating, or a combination thereof the site of infection, thereby preventing or treating the infection, wherein the pharmaceutical formulation comprises:
(a) a peptide or pharmaceutically acceptable salt thereof comprising at least about 70% sequence identity to a polypeptide sequence of:
Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1) Arg-Val-Val-Arg-Val-Val-Arg-Arg-Val-Val-Arg-Arg (SEQ ID NO: 15);
Arg-Val-Val-Arg-Val-Val-Arg-Arg-Trp-Val-Arg-Arg (SEQ ID NO: 16);
Arg-Trp-Trp-Arg-Trp-Trp-Arg-Arg-Trp-Trp-Arg-Arg (SEQ ID NO: 17);
Trp-Arg-Arg-Trp-Trp-Arg-Arg-Trp-Trp-Arg-Trp-Trp-Arg-Arg-Trp-Trp-Arg-Arg (SEQ ID NO: 18);
Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19);
Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20);
Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21);
Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22);
Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23);
Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24);
Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and
(b) an aqueous carrier, wherein the aqueous carrier provides a synergistic effect with the peptide or pharmaceutically acceptable salt thereof, wherein the synergistic effect comprises reduces a bacterial burden in the subject to a greater extent as compared to administering (a) or (b) alone.

57. A method of preventing or treating an infection in a subject in need thereof, wherein the method comprising locally administering of a pharmaceutical formulation to a site of infection, wherein administration comprises washing, irrigating, debriding, aspirating, or a combination thereof the site of infection, thereby preventing or treating the infection, wherein the pharmaceutical formulation consists of:
(a) a peptide or pharmaceutically acceptable salt thereof comprising at least about 70% sequence identity to a polypeptide sequence of:
Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1);
Arg-Val-Val-Arg-Val-Val-Arg-Arg-Val-Val-Arg-Arg (SEQ ID NO: 15);
Arg-Val-Val-Arg-Val-Val-Arg-Arg-Trp-Val-Arg-Arg (SEQ ID NO: 16);
Arg-Trp-Trp-Arg-Trp-Trp-Arg-Arg-Trp-Trp-Arg-Arg (SEQ ID NO: 17);
Trp-Arg-Arg-Trp-Trp-Arg-Arg-Trp-Trp-Arg-Trp-Trp-Arg-Arg-Trp-Trp-Arg-Arg (SEQ ID NO: 18);
Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19);
Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20);
Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21);
Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22);
Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23);
Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24);
Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or combination thereof; and
(b) an aqueous carrier, wherein the aqueous carrier provides a synergistic effect with the peptide or pharmaceutically acceptable salt thereof, wherein the synergistic effect comprises reduces a bacterial burden in the subject to a greater extent as compared to administering (a) or (b) alone.

58. The method of claim 56 or 57, wherein the aqueous carrier comprises sodium bicarbonate, chlorocyclohexidine, chlorhexidine gluconate, normal saline, phosphate buffered saline, povidone-iodine (PVP-I), ethanol, acetic acid, sodium acetate, benzalkonium chloride, sodium lauryl sulfate, citric acid, sodium citrate; or any combination thereof.

59. A method of preventing or treating an infection in a subject in need thereof, wherein the method comprising locally administering of a pharmaceutical formulation to a site of infection, wherein locally administration comprises washing, irrigating, debriding, aspirating, or a combination thereof of the site of infection, thereby preventing or treating the infection, wherein the pharmaceutical formulation comprises:
(a) a peptide or pharmaceutically acceptable salt thereof comprising at least about 70% sequence identity to a polypeptide sequence of:
Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1);
Arg-Val-Val-Arg-Val-Val-Arg-Arg-Val-Val-Arg-Arg (SEQ ID NO: 15);
Arg-Val-Val-Arg-Val-Val-Arg-Arg-Trp-Val-Arg-Arg (SEQ ID NO: 16);
Arg-Trp-Trp-Arg-Trp-Trp-Arg-Arg-Trp-Trp-Arg-Arg (SEQ ID NO: 17);
Trp-Arg-Arg-Trp-Trp-Arg-Arg-Trp-Trp-Arg-Trp-Trp-Arg-Arg-Trp-Trp-Arg-Arg (SEQ ID NO: 18);
Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19);
Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20);
Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21);
Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22);
Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23);
Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24);
Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and
(b) aqueous sodium bicarbonate.

60. A method of preventing or treating an infection in a subject in need thereof, wherein the method comprising locally administering of a pharmaceutical formulation to a site of infection, wherein locally administration comprises washing, irrigating, debriding, aspirating, or a combination thereof of the site of infection, thereby preventing or treating the infection, wherein the pharmaceutical formulation consists of:
(a) a peptide or pharmaceutically acceptable salt thereof comprising at least about 70% sequence identity to a polypeptide sequence of:
Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 1);
Arg-Val-Val-Arg-Val-Val-Arg-Arg-Val-Val-Arg-Arg (SEQ ID NO: 15);
Arg-Val-Val-Arg-Val-Val-Arg-Arg-Trp-Val-Arg-Arg (SEQ ID NO: 16);
Arg-Trp-Trp-Arg-Trp-Trp-Arg-Arg-Trp-Trp-Arg-Arg (SEQ ID NO: 17);
Trp-Arg-Arg-Trp-Trp-Arg-Arg-Trp-Trp-Arg-Trp-Trp-Arg-Arg-Trp-Trp-Arg-Arg (SEQ ID NO: 18);
Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 19);
Arg-Arg-Trp-Trp-Arg-Arg-Trp-Arg-Arg-Trp-Trp-Arg-Arg-Trp-Trp-Arg-Trp-Trp-Arg-Arg-Trp-Trp-Arg-Arg (SEQ ID NO: 20);
Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 21);
Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg (SEQ ID NO: 22);
Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 23);
Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg-Val-Arg-Arg-Val-Val-Arg-Arg-Val-Val-Arg-Val-Val-Arg-Arg-Val-Val-Arg-Arg (SEQ ID NO: 24);
Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Val-Arg-Arg-Val-Arg-Arg-Val-Trp-Arg-Arg-Val-Val-Arg-Val-Val-Arg-Arg-Trp-Arg-Val-Val (SEQ ID NO: 25), or any combination thereof; and
(b) aqueous sodium bicarbonate.

61. The method of any one of claims 56-60, wherein the infection further comprises a biofilm.

62. The method of any one of claims 56-59, wherein the method prevents formation of a biofilm.

63. The method of any one of claims 56-59, wherein administration of the pharmaceutical formulation results in at least partially penetrating, inhibiting formation of, or destroys the biofilm.

64. The method of any one of claims 56-62, wherein the site of infection does not comprise a prosthesis.

65. The method of any one of claims 56-62, wherein the site of infection comprises a prosthesis implanted in the subject.

66. The method of claim 65, wherein the washing, irrigating, debridement, aspirating, or a combination thereof of the site of infection occurs on the implanted prosthesis at the site of infection.

67. The method of claim 65 or 66, wherein the implanted prosthesis is a knee prosthesis, shoulder prosthesis, hip prosthesis, elbow prosthesis, ankle prosthesis, wrist prosthesis, or spine prosthesis.

68. The method of any one of claims 56-67, wherein the infection is periprosthetic joint infection (PJI).

69. The method of claim 68, wherein the periprosthetic joint infection is first stage periprosthetic joint infection or second stage periprosthetic joint infection.

70. The method of any one of claims 56-69, wherein the infection is a shoulder infection, knee infection, acute infection, chronic infection, or any combination thereof.

71. The method of any one of claims 56-68, wherein the method occurs prior, during, or subsequent to a total knee arthroplasty.

72. The method of any one of claims 65-71, wherein the method occurs prior, during, or subsequent to a debridement, antibiotics, and implant retention (DAIR) procedure.

73. The method of any one of claims 56-72, wherein the infection is a bacterial infection, wherein the bacterial species is selected from the group consisting of: Staphylococcus aureus, Staphylococcus epidermidis, Staphylococcus lugdenensis, Staphylococcus haemolyticus, Staphylococcus hominis, Staphylococcus saprophyticus, Staphylococcus simulans, Staphylococcus warnerii, Staphylococcus capitis, Staphylococcus caprae, Staphylococcus pettenkoferi, Streptococcus pyogenes, Streptococcus agalactiae, Streptococcus pneumoniae, Group C streptococci, Streptococcus constellatus, Enterococcus faecalis, Enterococcus faecium, Corynebacterium jeikeium, Lactobacillus acidophilus, Listeria monocytogenes, Escherichia coli, Klebsiella pneumoniae, Klebsiella oxytoca, Acinetobacter baumannii, Acinetobacter nosocomialis, Acinetobacter pittii, Acinetobacter haemolyticus, Acinetobacter radioresistens, Acinetobacter ursingii, Pseudomonas aeruginosa, Enterobacter cloacae, Enterobacter aerogenes, Stenotrophomonas maltophilia, Citrobacter freundii, Citrobacter koseri, Citrobacter sedlakii, Citrobacter braakii, Morganella morganii, Providencia rettgeri, Providencia stuartii, Salmonella typhimurium, Shigella dysenteriae, Moraxella catarrhalis, Neisseria gonorrhoeae, Propionibacterium acnes, Clostridioides difficile, Clostridioides perfringens, Bacteroides fragilis, Prevotella bivia, Eggerthella lenta, Peptostreptococcus anaerobius, and any combination thereof.

74. The method of any one of claims 56-73, wherein the pharmaceutical formulation comprises a pH from about 7 to about 11.

75. The method of claim 74, wherein the pharmaceutical formulation comprises the pH from about 8 to about 11.

76. The method of claim 74, wherein the pharmaceutical formulation comprises the pH from about 7 to about 10.

77. The method of any one of claims 56-73, wherein the pharmaceutical formulation comprises the pH at least about 7.5 or at least about 7.9.

78. The method of any one of claims 56-77, wherein the peptide or pharmaceutically acceptable salt thereof is present in the pharmaceutical formulation at a concentration from about 0.1 mg/mL to about 100 mg/mL.

79. The method of claim 78, wherein the peptide or pharmaceutically acceptable salt thereof is present in the pharmaceutical formulation at a concentration from about 1 mg/mL to about 10 mg/mL.

80. The method of claim 78, wherein the peptide or pharmaceutically acceptable salt thereof is present in the pharmaceutical formulation at a concentration of about 1 mg/mL, about 3 mg/mL, about 5 m/mL, or about 10 mg/mL.

81. The method of any one of claims 56-80, wherein the aqueous sodium bicarbonate is present in the pharmaceutical formulation at a concentration from about 25 mM to about 300 mM.

82. The method of claim 81, wherein the aqueous sodium bicarbonate is present in the pharmaceutical formulation at a concentration from about 50 mM to about 200 mM.

83. The method of any one of claims 56-80, wherein the aqueous sodium bicarbonate is present in the pharmaceutical formulation at a concentration from about 5 mg/mL to about 50 mg/mL.

84. The method of claim 83, wherein the aqueous sodium bicarbonate is present in the pharmaceutical formulation at a concentration at about 12.6 mg/mL.

85. The method of any one of claims 56-84, wherein the aqueous sodium bicarbonate is at a concentration of about 1 mEq/L to about 200 mEq/L.

86. The method of claim 85, wherein the aqueous sodium bicarbonate is at a concentration of about 50 mEq/L.

87. The method of any one of claims 56-86, wherein the pharmaceutical formulation comprises an osmolality of at least about 30 milliosmoles per kilogram (mOsm/kg) to at least about 800 mOsm/kg.

88. The method of claim 87, wherein the pharmaceutical formulation comprises an osmolality of about 50 mOsm/kg to about 500 mOsm/kg.

89. The method of claim 87 wherein the pharmaceutical formulation comprises an osmolality of about 200 mOsm/kg to about 500 mOsm/kg.

90. The method of any one of claims 56-89, wherein the peptide or salt thereof comprises the polypeptide of sequence Arg Arg Trp Val Arg Arg Val Arg Arg Val Trp Arg Arg Val Val Arg Val Val Arg Arg Trp Val Arg Arg (SEQ ID NO: 1).

91. The method of any one of claims 56-89, wherein the peptide or salt thereof comprises the polypeptide of sequence Arg-Trp-Trp-Arg-Trp-Trp-Arg-Arg-Trp-Trp-Arg-Arg (SEQ ID NO: 17).

92. The method of any one of claims 56-91, wherein pharmaceutical formulation is administered once.

93. The method of any one of claims 56-91, wherein pharmaceutical formulation is administered more than one time.

94. The method of any one of claims 56-91, wherein pharmaceutical formulation is administered two or more times.


SEQUENCE LISTING (25 sequences)

SEQ ID NO: 1 - protein, 24 aa, synthetic construct
RRWVRRVRRVWRRVVRVVRRWVRR

SEQ ID NO: 2 - protein, 14 aa, synthetic construct
IRRRRRRIRRRRRR

SEQ ID NO: 3 - protein, 21 aa, synthetic construct
IRRRIRRIRRRIRRIRRRIRR

SEQ ID NO: 4 - protein, 21 aa, synthetic construct
IRRIIRRIRRIIRRIRRIIRR

SEQ ID NO: 5 - protein, 21 aa, synthetic construct
VWRWVRRVWRWVRRVWRWVRR

SEQ ID NO: 6 - protein, 14 aa, synthetic construct
VWRWVRRVWRWVRR

SEQ ID NO: 7 - protein, 14 aa, synthetic construct
VVRVVRRVVRVVRR

SEQ ID NO: 8 - protein, 21 aa, synthetic construct
VVRVVRVVVRVVRVVVRVVRV

SEQ ID NO: 9 - protein, 11 aa, synthetic construct
RSRVVRSWSRV

SEQ ID NO: 10 - protein, 84 aa, synthetic construct
RFVRRVRRFVRRVRRFVRRVRRFVRRVRRFVRRVRRFVRRVRRFVRRVRRFVRRVRRFVRRVRRFVRRVRRFVRRVRRFVRRVR

SEQ ID NO: 11 - protein, 20 aa, synthetic construct
RRTYSRSRRTYSRSRRTYSR

SEQ ID NO: 12 - protein, 28 aa, synthetic construct
KVVSSIIEIISSVVKVVSSIIEIISSVV

SEQ ID NO: 13 - protein, 20 aa, synthetic construct
KKTHTKTKKTHTKTKKTHTK

SEQ ID NO: 14 - protein, 21 aa, synthetic construct
VVRVVRRVVRVVRRVVRVVRR

SEQ ID NO: 15 - protein, 12 aa, synthetic construct
RVVRVVRRVVRR

SEQ ID NO: 16 - protein, 12 aa, synthetic construct
RVVRVVRRWVRR

SEQ ID NO: 17 - protein, 12 aa, synthetic construct
RWWRWWRRWWRR

SEQ ID NO: 18 - protein, 18 aa, synthetic construct
WRRWWRRWWRWWRRWWRR

SEQ ID NO: 19 - protein, 24 aa, synthetic construct
RRVVRRVRRVVRRVVRVVRRVVRR

SEQ ID NO: 20 - protein, 24 aa, synthetic construct
RRWWRRWRRWWRRWWRWWRRWWRR

SEQ ID NO: 21 - protein, 36 aa, synthetic construct
VRRVVRRVVRVVRRVVRRVRRVVRRVVRVVRRVVRR

SEQ ID NO: 22 - protein, 36 aa, synthetic construct
VRRVWRRVVRVVRRWVRRVRRVWRRVVRVVRRWVRR

SEQ ID NO: 23 - protein, 42 aa, synthetic construct
RRVVRRVRRVVRRVVRVVRRVVRRVRRVVRRVVRVVRRVVRR

SEQ ID NO: 24 - protein, 48 aa, synthetic construct
RVVRVVRRVVRRVRRVVRRVVRVVRRVVRRVRRVVRRVVRVVRRVVRR

SEQ ID NO: 25 - protein, 48 aa, synthetic construct
RVVRVVRRWVRRVRRVWRRVVRVVRRWVRRVRRVWRRVVRVVRRWRVV